SEQUENCE LISTING (11 sequences)

SEQ ID NO: 1 - DNA, 12 bp, Homo Sapian
ggcggcggcggc

SEQ ID NO: 2 - DNA, 12 bp, Homo sapians
cggcggcggcgg

SEQ ID NO: 3 - DNA, 12 bp, Homo sapians
cggaggcggcgg

SEQ ID NO: 4 - DNA, 12 bp, Homo sapians
aggcggcggcgg

SEQ ID NO: 5 - DNA, 12 bp, Homo sapians
aggaggcggcgg

SEQ ID NO: 6 - DNA, 12 bp, Homo sapians
gcggcggcggcg

SEQ ID NO: 7 - DNA, 12 bp, Homo sapians
gcggcagcggcg

SEQ ID NO: 8 - DNA, 109 bp, Homo sapians
ctaggttgaaagtactttgacggcggcggcggtcaatcttacggcggcggcggacatagatacggcggcggcggtagaaactacggcggcggcggattagaatagtaaa

SEQ ID NO: 9 - DNA, 109 bp, Homo sapians
ctagggcgcacgtacttcgacaacgtcagcgttcagcgttccaacgtcagcgtacagcgatccaacgtcagcgttctgcgctacaacgtcagcgtatccgcgtagcaca

SEQ ID NO: 10 - RNA, 30 nt, Homo sapians
uagaaacuacggcggcggcggaaucguaga

SEQ ID NO: 11 - RNA, 30 nt, Homo sapians
uagacccugcaacgucagcguagucguagc

(19) United States
(12) Patent Application Publication
(10) Pub. No.: US 2015/0219624 A1
(43) Pub. Date: Aug. 6, 2015

(54) METHODS FOR IDENTIFYING ANTI-CANCER COMPOUNDS

(71) Applicant: MEMORIAL SLOAN KETTERING CANCER CENTER, New York, NY (US)

(72) Inventors: Hans-Guido WENDEL, New York, NY (US); Andrew Wolfe, New York, NY (US); Kamini Sing, New York, NY (US); Yi Zhong, New York, NY (US); Phillip Drewe, New York, NY (US)

(73) Assignee: MEMORIAL SLOAN KETTERING CANCER CENTER, New York, NY (US)

(21) Appl. No.: 14/693,832

(22) Filed: Apr. 22, 2015

Related U.S. Application Data
(63) Continuation-in-part of application No. PCT/US14/68875, filed on Dec. 5, 2014.
(60) Provisional application No. 61/912,420, filed on Dec. 5, 2013.

(51) Int. Cl. G01N 33/50; G01N 33/574; A61K 31/429; A61K 31/357; A61K 31/58

(52) U.S. Cl. CPC G01N 33/5011; A61K 31/357; A61K 31/58; A61K 31/429; G01N 33/57496; G01N 2333/914; G01N 2500/10; G01N 2500/02; G01N 33/582

(57) ABSTRACT

Methods are provided for identifying agents capable of modulating cap-dependent RNA translation by comparing translation efficiency in the presence and absence of the agent in an in-vitro or in-vivo translation system that comprises eIF4A and an mRNA having one or more eIF4A-dependent translation-controlling motifs. The modulation of translation in the presence of the agent indicates the agent as capable of modulating cap-dependent mRNA translation. The method can be used to identify anti-cancer agents and oncogenes that may be responsible for tumorigenesis.

### CROSS-REFERENCE TO RELATED APPLICATIONS

This application claims priority to International application PCT/US2014/68875, filed Dec. 5, 2014, which in turn claims priority to U.S. Patent Application Ser. No. 61/912,420, filed Dec. 5, 2013, both of which are incorporated herein by reference in their entireties.

### GOVERNMENT SUPPORT

This research was supported by funding from the National Cancer Institute Grants R01-CA142798-01 and U01CA105492-08, and National Institutes of Health Grant GM-073855. The U.S. Government has certain rights in the invention.

### BACKGROUND OF THE INVENTION

The initiation of cap-dependent translation involves ˜13 tightly controlled protein factors (reviewed in (Jackson et al., 2010)). Among these, eIF4E binds the mRNA cap structure and interacts with a scaffold (eIF4G) and the eIF4A RNA helicase (a DEAD box protein also known as DDX2). During initiation these and other factors form the eIF4F complex and together with the 40S ribosomal unit proceed to a transcript's 5′UTR for a translation start site. The eIF4A RNA helicase is directly involved in scanning and recent studies have defined co-factors and the molecular mechanics of its helicase activity (Marintchev, 2009, 2013; Parsyan et al., 2011; Svitkin, 2001). However, the precise mRNA features that necessitate the eIF4A helicase action are not known.

The activation of protein translation contributes to malignant transformation. For example, activation of the RAS, ERK, and AKT signaling pathways stimulates cap-dependent translation (reviewed in (Blagden and Willis, 2011; D'Ambrogio et al., 2013; Guertin and Sabatini, 2007). Moreover, the rate limiting eIF4E translation factor is expressed at high levels in many cancers and can transform rodent fibroblasts and promote tumor development in vivo (Lazaris-Karatzas et al., 1990; Ruggero et al., 2004; Wendel et al., 2004). Accordingly, cap-dependent translation is an emerging target for cancer therapies (see recent review by (Blagden and Willis, 2011). Notably, three distinct natural compounds target the eIF4A helicase and these are silvestrol isolated from plants in the Malaysian rainforest (Cencic, 2009), pateamine A found in marine sponges off the coast of New Zealand (Northcote et al., 1991), and hippuristanol which is produced by pacific corals (Li et al., 2009b). These compounds show promising preclinical activity against different cancers (Bordeleau et al., 2005; Bordeleau et al., 2006; Cencic et al., 2007; Schatz et al., 2011; Tsumuraya et al., 2011a). Other strategies to inhibit translation include rapamycin and mTORC1 kinase inhibitors (Hsieh et al., 2012; Thoreen et al., 2009), inhibitors of the eIF4E kinase MNK1/2 (Furic et al., 2010; Ueda et al., 2004; Wendel et al., 2007), a peptide (4EGI-1) that interferes with the eIF4E-eIF4G interaction (Moerke et al., 2007), and the anti-viral ribavirin that may bind eIF4E directly (Kentsis et al., 2004; Yan et al., 2005).

The recently developed transcriptome-scale ribosome footprinting technology greatly facilitates the study of protein translation. Briefly, the technology is based on the identification of ribosome-protected RNA fragments in relation to total transcript levels using deep sequencing (Ingolia et al., 2009). The technology has been applied to explore translational effects in various biological contexts, and perhaps the most relevant to this study are reports of the translational effects of mTORC1 inhibition on mRNAs harboring TOP- and TOP-like sequences (Hsieh et al., 2012; Thoreen et al., 2012).

### BRIEF DESCRIPTION OF THE INVENTION

In one embodiment, a method is provided for identifying an agent capable of modulating cap-dependent mRNA translation. The method comprises comparing translation efficiency in the presence and absence of the agent in an in-vitro or in-vivo translation system comprising eIF4A and an mRNA having one or more eIF4A-dependent translation-controlling motifs. eIF4A refers to eIF4A1 or eIF4A2, and RNA helicases include, but are not limited to, eIF4A1, eIF4A2, DHX9 or DHX36. The modulation of translation in the presence of the agent indicates the agent as capable of modulating cap-dependent mRNA translation. In one embodiment, modulating is decreasing, suppressing or inhibiting cap-dependent mRNA translation. In one embodiment, the agent stabilizes the binding of eIF4A to the eIF4A-dependent translation-controlling motif of the mRNA. In one embodiment, the eIF4A-mRNA complex stabilizing motif of the mRNA is located in the 5′ UTR.

In one embodiment, the eIF4A-dependent translation-controlling motif comprises a G-quadruplex structure. In one embodiment, the G-quadruplex structure comprises a (GGC/A)₄ motif. In one embodiment, the (GGC/A)₄ motif comprises GGCGGCGGCGGC (SEQ ID NO:1). In one embodiment, the eIF4A-dependent translation-controlling motif comprises a sequence selected from SEQ ID NO:4, SEQ ID NO:5, SEQ ID NO:6, SEQ ID NO:7, SEQ ID NO:8, SEQ ID NO:9 or SEQ ID NO:10. In one embodiment, the eIF4A-dependent translation-controlling motif comprises a sequence selected from among SEQ ID NO:10 to SEQ ID NO:62. In one embodiment, the eIF4A-dependent translation-controlling motif is at least one sequence selected from SEQ ID NO:1 or from among SEQ ID NO:4 to SEQ ID NO:62.

In one embodiment of the methods described herein, the mRNA encodes a transcription factor. In one embodiment, the mRNA encodes an oncogene. In other embodiments, the mRNA encodes NOTCH1, BCL11B, MYC, CDK6, RUNX1, BCL2 or MDM2. In other embodiments, the mRNA is from a gene selected from Table 3A. In other embodiments, the mRNA is from a gene selected from Table 3B. In other embodiments, the mRNA is from a gene selected from Table 3C.

In one embodiment of the method, the agent suppresses the growth of cancer cells in vitro or in vivo. In one embodiment, the agent interferes with eIF4A activity. In one embodiment, the agent increases eIF4A activity. In one embodiment, the agent inhibits eIF4A helicase activity. In one embodiment, the agent increases eIF4A helicase activity. In one embodiment, the agent promotes the stabilizing the binding of eIF4A with an eIF4A-dependent translation-controlling motif. In one embodiment, the agent does not trigger feedback activation of Akt.

In one embodiment, the modulation of translation in the foregoing method is measured by a fluorescence reporter assay. In one embodiment, the assay comprises renilla luciferase expression.

In one embodiment, a method is provided for identifying an agent that modulates eIF4A activity, the method comprising comparing translation efficiency in the presence and absence of the agent in an in-vitro or in-vivo translation system comprising eIF4A and an mRNA having one or more eIF4A-dependent translation-controlling motifs, wherein the increase or decrease in translation efficiency in the presence of the agent indicates the agent as capable of increasing or decreasing eIF4A activity.

In one embodiment, a method is provided for identifying an agent that inhibits eIF4A activity, the method comprising comparing translation efficiency in the presence and absence of the agent in an in-vitro or in-vivo translation system comprising eIF4A and an mRNA having one or more eIF4A-dependent translation-controlling motifs, wherein a decrease in translation efficiency in the presence of the agent indicates the agent as capable of inhibiting eIF4A activity.

In one embodiment, a method is provided for determining whether an mRNA sequence comprises at least one eIF4A-dependent translation-controlling motif, the method comprising comparing translation efficiency in the presence and absence of an agent that inhibits eIF4A activity in an in-vivo translation system comprising eIF4A and an mRNA having one or more eIF4A-dependent translation-controlling motifs, wherein a decrease in translation efficiency in the presence of the agent indicates the mRNA sequence possesses at least one eIF4A-dependent translation-controlling motif.

In one embodiment, a method is provided for determining whether a cancer or tumor is susceptible to an agent that inhibits eIF4A activity, the method comprising identifying the presence of at least one eIF4A-dependent translation-controlling motif in mRNA from the cancer or tumor, wherein the presence of the at least one eIF4A-dependent translation-controlling motif indicates susceptibility of the cancer or tumor to the agent. In one embodiment, the level of expression of MYC is not predictive of the susceptibility of a cancer or tumor to an agent that inhibits eIF4A activity.

In one embodiment, methods are provided for 1) measuring the effect of known RNA helicases such as eIF4A, DHX9 or DHX36 on G-quadruplex unwinding; 2) investigating the effect of other cofactors/inhibitors required for eIF4A activity; 3) a screening method to identify other proteins that can unwind G-quadruplexes; or 4) identifying and establishing the effect of small molecules that stabilize the G-quadruplex structure, by utilizing a fluorescence resonance energy transfer (FRET)-based assay utilizing an oligonucleotide comprising a G-quadruplex labeled with a fluorophore at the 5′ or 3′ end of the oligonucleotide, and a fluorescence quencher at the other end. The aforementioned uses are merely non-limiting examples.

In one embodiment, a method for preventing, treating or intervening in the recurrence of a cancer in a subject is provided. The method comprises administering to the subject an agent that blocks eIF4a helicase activity, thereby preventing, treating or intervening in the recurrence of the cancer. In one embodiment, the agent that blocks eIF4A helicase inhibits the translation of an oncogenic mRNA. In one embodiment, the oncogenic mRNA comprises an eIF4A-dependent translation-controlling motif. In one embodiment, the eIF4A-dependent translation-controlling motif is a G-quadruplex motif. In one embodiment, the eIF4A-dependent translation-controlling motif is selected from among SEQ ID NOs:1-62. In one embodiment, the oncogenic mRNA comprises a G-quadruplex motif. In one embodiment, the oncogenic mRNA is from an oncogene, which by way of non-limiting example is selected from among Tables 3A, 3B and 3C. In one embodiment, the oncogene is NOTCH1, BCL11B, MYC, CDK6, RUNX1, BCL2 or MDM2.

In the foregoing embodiments, the cancer is, by way of non-limiting examples, T-cell acute lymphoblastic leukemia, small cell lung cancer, renal cell carcinoma, squamous cell carcinoma of the head and neck, neuroblastoma and pancreatic cancer. In one embodiment the subject has cancer. In one embodiment, the subject is at risk for developing cancer. In one embodiment, the subject is in remission from cancer. In other embodiments, the cancer is transformed follicular lymphoma, mantel cell lymphoma, breast cancer, ovarian cancer, hepatocellular carcinoma, and non-small cell lung cancer, as well as gastric cancer, Ewing sarcoma and lung adenocarcinoma.

In one embodiment, a method is provided for preventing, treating or intervening in the recurrence of a cancer in a subject having an eIF4A dependent cancer. The method comprises administering to the subject an agent that blocks eIF4a helicase activity, thereby preventing, treating or intervening in the recurrence of the cancer. In one embodiment, the agent that blocks eIF4A helicase inhibits the translation of an oncogenic mRNA. In one embodiment, the oncogenic mRNA comprises an eIF4A-dependent translation-controlling motif. In one embodiment, the eIF4A-dependent translation-controlling motif is a G-quadruplex motif. In one embodiment, the eIF4A-dependent translation-controlling motif is selected from among SEQ ID NOs:1-62. In one embodiment, the oncogenic mRNA comprises a G-quadruplex motif. In one embodiment, the oncogenic mRNA is from an oncogene. In one embodiment, the oncogene is selected from among Tables 3A, 3B and 3C. In one embodiment, the oncogene is NOTCH1, BCL11B, MYC, CDK6, RUNX1, BCL2 or MDM2.

In the foregoing embodiments, the cancer is, by way of non-limiting examples, T-cell acute lymphoblastic leukemia, small cell lung cancer, renal cell carcinoma, squamous cell carcinoma of the head and neck, neuroblastoma and pancreatic cancer. In one embodiment the subject has cancer. In one embodiment, the subject is at risk for developing cancer. In one embodiment, the subject is in remission from cancer. In other embodiments, the cancer is transformed follicular lymphoma, mantel cell lymphoma, breast cancer, ovarian cancer, hepatocellular carcinoma, and non-small cell lung cancer, as well as gastric cancer, Ewing sarcoma and lung adenocarcinoma.

In another embodiment, a method is provided for inhibiting in a subject the translation of an oncogene that comprises an eIF4A-dependent translation-controlling motif. The method comprises administering to the subject an agent that blocks eIF4a helicase, thereby inhibiting translation of the oncogene. In one embodiment, translation of the oncogene causes cancer in the subject. In another embodiment, the eIF4A-dependent translation-controlling motif is a G-quadruplex motif. In this embodiment, the eIF4A-dependent translation-controlling motif is selected from among SEQ ID NOs:1-62. In one embodiment, the mRNA of the oncogene comprises a G-quadruplex motif. In one embodiment, the oncogene is selected from among Tables 3A, 3B and 3C. In one embodiment, the oncogene is NOTCH1, BCL11B, MYC, CDK6, RUNX1, BCL2 or MDM2.

In the foregoing embodiments, the cancer is, by way of non-limiting examples, T-cell acute lymphoblastic leukemia, small cell lung cancer, renal cell carcinoma, squamous cell carcinoma of the head and neck, neuroblastoma and pancreatic cancer. In one embodiment the subject has cancer. In one embodiment, the subject is at risk for developing cancer. In one embodiment, the subject is in remission from cancer. In other embodiments, the cancer is transformed follicular lymphoma, mantel cell lymphoma, breast cancer, ovarian cancer, hepatocellular carcinoma, and non-small cell lung cancer, as well as gastric cancer, Ewing sarcoma and lung adenocarcinoma.

In one embodiment, a method for inhibiting in a subject eIF4A dependent mRNA translation is provided. The method comprises administering to the subject an agent that blocks eIF4a helicase, thereby inhibiting mRNA translation. In one embodiment, the mRNA translation causes cancer in the subject. In one embodiment, the mRNA comprises an eIF4A-dependent translation-controlling motif. In one embodiment, the eIF4A-dependent translation-controlling motif is selected from among SEQ ID NOs:1-62. In one embodiment, the eIF4A-dependent translation-controlling motif is a G-quadruplex motif. In one embodiment, the mRNA encodes an oncogenic protein. In one embodiment, the oncogenic protein is encoded by an oncogene selected from among Tables 3A, 3B and 3C. In one embodiment, the oncogene is NOTCH1, BCL11B, MYC, CDK6, RUNX1, BCL2 or MDM2.

In the foregoing embodiments, the cancer is, by way of non-limiting examples, T-cell acute lymphoblastic leukemia, small cell lung cancer, renal cell carcinoma, squamous cell carcinoma of the head and neck, neuroblastoma and pancreatic cancer. In one embodiment the subject has cancer. In one embodiment, the subject is at risk for developing cancer. In one embodiment, the subject is in remission from cancer. In other embodiments, the cancer is transformed follicular lymphoma, mantel cell lymphoma, breast cancer, ovarian cancer, hepatocellular carcinoma, and non-small cell lung cancer, as well as gastric cancer, Ewing sarcoma and lung adenocarcinoma.

In one embodiment, a method for preventing in a subject the translation of an mRNA comprising an eIF4A-dependent translation-controlling motif. The method comprises administering to the subject an agent that blocks eIF4a helicase activity, thereby inhibiting translation of the mRNA. In one embodiment, the eIF4A-dependent translation-controlling motif is a G-quadruplex motif. In one embodiment, the eIF4A-dependent translation-controlling motif is selected from among SEQ ID NOs:1-62. In one embodiment, the mRNA is from an oncogene selected from among Tables 3A, 3B and 3C. In one embodiment, the oncogene is NOTCH1, BCL11B, MYC, CDK6, RUNX1, BCL2 or MDM2. In one embodiment, the translation of the mRNA causes cancer.

In the foregoing embodiments, the cancer is, by way of non-limiting examples, T-cell acute lymphoblastic leukemia, small cell lung cancer, renal cell carcinoma, squamous cell carcinoma of the head and neck, neuroblastoma and pancreatic cancer. In one embodiment the subject has cancer. In one embodiment, the subject is at risk for developing cancer. In one embodiment, the subject is in remission from cancer. In other embodiments, the cancer is transformed follicular lymphoma, mantel cell lymphoma, breast cancer, ovarian cancer, hepatocellular carcinoma, and non-small cell lung cancer, as well as gastric cancer, Ewing sarcoma and lung adenocarcinoma.

In any of the foregoing embodiments, the agent blocks the activity of eIF4A helicase. In any of the foregoing embodiments, the agent blocks the translation of an mRNA comprising an eIF4A-dependent translation-controlling motif. In any of the foregoing embodiments, the eIF4A-dependent translation-controlling motif is a G-quadruplex motif. In any of the foregoing embodiments, the eIF4A-dependent translation-controlling motif is selected from among SEQ ID NOs:1-62.

Non-limiting examples of aforementioned agents include a rocaglamide, such as silvestrol, CR-31-B, or an analogue or derivative thereof. In other embodiments, the agent is hippuristanol, pateamine A, or an analogue or derivative thereof.

U.S. Patent Application Ser. No. 61/912,420, filed Dec. 5, 2013, is incorporated herein by reference in its entirety.

### BRIEF DESCRIPTION OF THE FIGURES

FIG. 1 shows that translational activation contributes to T-ALL pathogenesis and maintenance;

FIG. 2 shows that silvestrol blocks cap-dependent translation and has single-agent activity against T-ALL;

FIG. 3 shows that transcriptome-scale ribosome footprinting can be used to define silvestrol's effects on translation;

FIG. 4 shows that silvestrol alters the distribution of ribosomes across many mRNAs;

FIG. 5 shows that many cancer genes are differentially affected by silvestrol;

FIG. 6 shows the validation of selected silvestrol targets;

FIG. 7 is a diagram depicting an eIF4A dependent mechanism of translational control;

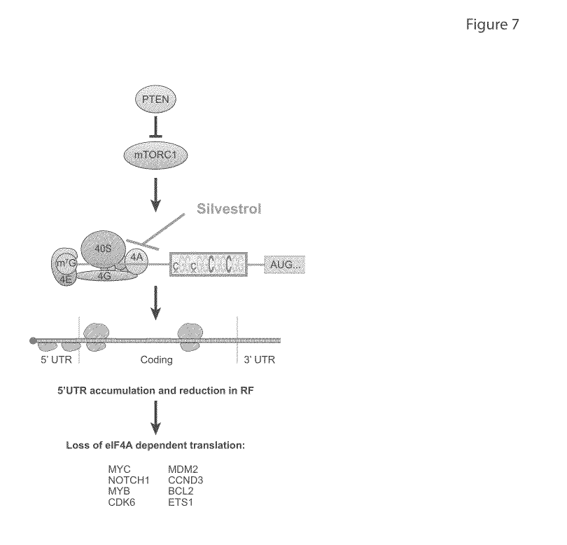

FIG. 8 shows the PI3K pathway and translational activation in T-ALL;

FIG. 9 shows testing silvestrol and the synthetic analogue CR-31-B in T-ALL;

FIG. 10 shows ribosome profiling quality control data and effects on translation;

FIG. 11 shows analysis of genes with differential ribosomal distribution;

FIG. 12 shows gene ontology analysis of silvestrol sensitive genes;

FIG. 13 illustrates exploring the relative contribution of MYC and other silvestrol targets in T-ALL;

FIG. 14 illustrates a FRET-based assay for measuring the effect of RNA helicases on G-G-quadruplex unwinding, screening proteins that can unwind G-quadruplexes and identify small molecules that stabilize the G-quadruplex structure;

FIG. 15 shows the sensitivity of several small cell lung cancer lines to silvestrol;

FIG. 16 shows the sensitivity of several renal cell carcinoma cell lines to silvestrol;

FIG. 17 shows the sensitivity to silvestrol of a number of cancer cell lines;

FIG. 18 shows that the sensitivity of cancer cell lines to silvestrol is not predicted by MYC expression;

FIG. 19 shows activity of hippuristanol and panteamine A in the reporter assay;

FIG. 20 shows in vitro data on silvestrol on a number of lung cancer cell lines and key target proteins;

FIG. 21 shows the effect of silvestrol on lung cancer cells in the presence and absence of serum, with and without MG-132, and the effect on key target proteins;

FIG. 22 shows transcripts of KRAS, and the presence of G-quadruplex structures;

FIG. 23 compares the G-quadruplex structures in NRAS and KRAS;

FIG. 24 shows the effect of silvestrol on KRAS protein levels in PANC1 cells and the effect of various compounds on PANC1 and MiaPaca2 cells;

FIG. 25 shows the in vivo activity of silvestrol on MiaPaca2 xenografts;

FIG. 26 shows the effect in individual animals xenografted with H82 small cell lung cancer cells and treated with silvestrol, etoposide or both; and

FIG. 27 shows a summary of in vivo data for two dose levels of silvestrol and the effects on key target proteins.

### DETAILED DESCRIPTION OF THE INVENTION

A mechanism of translational control has been identified that is characterized by a requirement for eIF4A/DDX2 RNA helicase activity and underlies the anticancer effects of silvestrol and related compounds. eIF4A refers to eIF4A1 or eIF4A2, and RNA helicases include, but are not limited to, eIF4A1, eIF4A2, DHX9 or DHX36. In one embodiment, activation of cap-dependent translation contributes to T-cell leukemia (T-ALL) development and maintenance. Accordingly, inhibition of the translation initiation factor eIF4A with silvestrol produces powerful therapeutic effects. By using transcriptome-scale ribosome footprinting on silvestrol-treated T-ALL cells to identify silvestrol-sensitive transcripts, the features of eIF4A-dependent translation embodied herein were identified. These features include, in one embodiment, a long 5′UTR and a 12-mer sequence motif that encodes a guanine quartet (GGC)₄. RNA folding algorithms pinpoint the (GGC)₄ motif as a common site of RNA G-quadruplex structures within the 5′UTR. In T-ALL these structures mark highly silvestrol-sensitive transcripts that include key oncogenes and transcription factors and contribute to the drug's anti-leukemic action. Hence, the eIF4A-dependent translation of G-quadruplex containing transcripts is shown as a gene-selective and therapeutically targetable mechanism of translational control.

Thus, in one embodiment, a method for identifying an agent capable of modulating cap-dependent mRNA translation is provided, the method comprising comparing translation efficiency in the presence and absence of the agent in an in-vitro or in-vivo translation system comprising eIF4A and an mRNA having one or more eIF4A-dependent translation-controlling motifs, wherein the modulation of translation in the presence of the agent indicates the agent as capable of modulating cap-dependent mRNA translation. In some embodiments, modulating is decreasing, suppressing or inhibiting cap-dependent mRNA translation.

eIF4A-dependent translation-controlling motifs are typically present in the 5′ UTR of the mRNA. In certain embodiments, the eIF4A-dependent translation-controlling motif comprises a G-quadruplex structure. In some embodiments, the G-quadruplex structure is a (GGC/A)₄ motif (i.e., four occurrences of (G, G, C or A), each occurrence independently selected from either GGC or GGA). In some embodiments, the (GGC/A)₄ motif is GGCGGCGGCGGC (SEQ ID NO:1). In some embodiments, the eIF4A-dependent translation-controlling motif comprises GGGAC (SEQ ID NO:2) motif or GGGCC (SEQ ID NO:3). In other embodiments the eIF4A-dependent translation-controlling motif comprises SEQ ID NO:4, SEQ ID NO:5, SEQ ID NO:6, SEQ ID NO:7, SEQ ID NO:8, SEQ ID NO:9 or SEQ ID NO:10. In other embodiments, the eIF4A-dependent translation-controlling motif comprises a sequence selected from among SEQ ID NO:10 to SEQ ID NO:62. In other embodiments, the eIF4A-dependent translation-controlling motif is at least one sequence selected from SEQ ID NO:1 or from SEQ ID NO:4 to SEQ ID NO:62.

The mRNA may have one or more eIF4A-dependent translation-controlling motifs. In one embodiment, the eIF4A-dependent translation-controlling motif is at least one (GGC/A)₄ motif. In another embodiment, the eIF4A-dependent translation-controlling motif is at least one GGGAC (SEQ ID NO:2) motif. In another embodiment, the eIF4A-dependent translation-controlling motif is at least one GGGCC (SEQ ID NO:3) motif. In another embodiment, the eIF4A-dependent translation-controlling motif is at least one 12-mer motif. In other embodiments, the mRNA may comprise 2, 3, 4, 5, 6, 7, 8, 9, 10, or more eIF4A-dependent translation-controlling motifs. In another embodiment, each eIF4A-dependent translation-controlling motif is independently selected from among SEQ ID NO:1 through and including SEQ ID NO:62.

In one embodiment, an agent identified by the methods of the invention may interfere with eIF4A activity. In one embodiment, the agent may increase eIF4A activity. In one embodiment, the agent may inhibit eIF4A helicase activity. In another embodiment, the agent may increase eIF4A helicase activity. In another embodiment, the agent can promote the stabilizing the binding of eIF4A with an eIF4A-dependent translation-controlling motif.

In another embodiment, the agent does not trigger feedback activation of Akt.

In another embodiment, the mRNA encodes a transcription factor. In another embodiment, the mRNA encodes an oncogene. In another embodiment, the mRNA encodes NOTCH1, BCL11B, MYC, CDK6, RUNX1, BCL2 or MDM2. In another embodiment the mRNA is from a gene selected from Table 3A. In another embodiment, the mRNA is from a gene selected from Table 3B. In another embodiment, the mRNA is from a gene selected from Table 3C.

The agent identified by the methods herein may be used to treat cancer. In one embodiment, the cancer is a result of the overexpression an oncogene or transcription factor. The oncogene or transcription factor may be selected from those described herein, such as but not limited to NOTCH1, BCL11B, MYC, CDK6, RUNX1, BCL2 or MDM2, or any described in Table 3A, 3B or 3C.

Cancer includes cancerous and precancerous conditions, including, for example, premalignant and malignant hyperproliferative diseases such as cancers of the breast, ovary, germ cell, skin, prostate, colon, bladder, cervix, uterus, stomach, lung, esophagus, blood and lymphatic system, larynx, oral cavity, as well as metaplasias, dysplasias, neoplasias, leukoplakias and papillomas of the mucous membranes, and in the treatment of Kaposi's sarcoma. These are also referred to herein as dysproliferative diseases or dysproliferation. Non-limiting examples of other cancers, tumors, malignancies, neoplasms, and other dysproliferative diseases that can be treated according to the invention include leukemias, such as myeloid and lymphocytic leukemias, lymphomas, myeloproliferative diseases, and solid tumors, such as but not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In one embodiment, the compounds and uses embodied herein are directed to small cell lung cancer. In one embodiment, the compounds and uses embodied herein are directed to renal cancers. In one embodiment, the compounds and uses embodied herein are directed to neuroblastoma. In one embodiment, the compounds and uses embodied herein are directed to pancreatic cancers.

In one embodiment the agent suppresses the growth of cancer cells in vitro or in vivo.

The method of carrying out the translation assay using an in-vitro or in-vivo assay described herein may be accomplished by any of a number of methods know in the art. In one embodiment, the modulation of translation is measured by a fluorescence reporter assay. In one embodiment, the fluorescence reporter assay comprises renilla luciferase expression.

As mentioned above, certain mRNAs have longer 5′ UTRs and the eIF4A-dependent translation-controlling motif is present in the 5′ UTR. In one embodiment, the eIF4A-dependent translation-controlling motif comprises a 12-mer and the mRNA is from a gene selected from Table 3A. In another embodiment, the eIF4A-dependent translation-controlling motif comprises a 9-mer and the mRNA is from a gene selected from Table 3B. In another embodiment, eIF4A-dependent translation-controlling motif comprises a (GGC)₄ motif and the mRNA is from a gene selected from Table 3C.

In another embodiment, a method for identifying an agent that modulates eIF4A activity is provided. The method comprises comparing translation efficiency in the presence and absence of the agent in an in-vitro or in-vivo translation system comprising eIF4A and an mRNA having one or more eIF4A-dependent translation-controlling motifs. An increase or decrease in translation efficiency in the presence of the agent indicates the agent as capable of increasing or decreasing eIF4A activity, respectively. The in-vitro or in-vivo translation system may be one from among those described here. The mRNA may be among those described herein. The eIF4A-dependent translation-controlling motifs may be among those described herein.

In another embodiment, a method is provided for identifying an agent that inhibits eIF4A activity, the method comprising comparing translation efficiency in the presence and absence of the agent in an in-vitro translation system comprising eIF4A and an mRNA having one or more eIF4A-dependent translation-controlling motifs, wherein a decrease in translation efficiency in the presence of the agent indicates the agent as capable of inhibiting eIF4A activity. The in-vitro or in-vivo translation system may be one from among those described here. The mRNA may be among those described herein. The eIF4A-dependent translation-controlling motifs may be among those described herein.

In another embodiment, a method is described for determining whether an mRNA sequence comprises at least one eIF4A-dependent translation-controlling motif. In this method, translation efficiency is compared in the presence and absence of an agent that inhibits eIF4A activity in an in-vitro translation system comprising eIF4A and an mRNA having one or more eIF4A-dependent translation-controlling motifs, wherein a decrease in translation efficiency in the presence of the agent indicates the mRNA sequence possesses at least one eIF4A-dependent translation-controlling motif. By way of non-limiting example, the agent is selected from among silvestrol(methyl(1R,2R,3S,3aR,8bS)-6-[[(2S,3R,6R)-6-R1R)-1,2-dihydroxyethyl]-3-methoxy-1,4-dioxan-2-yl]oxy]-1,8b-dihydroxy-8-methoxy-3a-(4-methoxyphenyl)-3-phenyl-2,3-dihydro-1H-cyclopenta[b][1]benzofuran-2-carboxylate), pateamine A ((3S,6Z,8E,11S,15R,17S)-15-amino-3-[(1E,3E,5E)-7-(dimethylamino)-2,5-dimethylhepta-1,3,5-trienyl]-9,11,17-trimethyl-4,12-dioxa-20-thia-21-azabicyclo[16.2.1]henicosa-1(21),6,8,18-tetraene-5,13-dione), hippuristanol, (±)-CR-31-B, among other rocaglamide((1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide) derivatives.

Methods are also provided for determining whether a cancer or tumor is susceptible to an agent that inhibits eIF4A activity. In one embodiment, the method comprising identifying the presence of at least one eIF4A-dependent translation-controlling motif in mRNA from the cancer or tumor, wherein the presence of the at least one eIF4A-dependent translation-controlling motif indicates susceptibility of the cancer or tumor to the agent. In other embodiments, the eIF4A-dependent translation-controlling motifs are among those described herein above. In one embodiment, the presence of MYC is not predictive of the susceptibility of a cancer or tumor to an agent that inhibits eIF4A activity.

In another embodiment, a method for determining whether a patient having cancer or a tumor will respond to treatment with an eIF4A inhibitor is provided comprising the steps of 1) obtaining a sample of the cancer or tumor from the patient; and 2) identifying the presence of at least one eIF4A-dependent translation-controlling motif in mRNA from the cancer or tumor, wherein the presence of the at least one eIF4A-dependent translation-controlling motif indicates that the patient will respond to the treatment. In the foregoing embodiments, identifying the presence of at least one eIF4A-dependent translation-controlling motif in mRNA from the cancer or tumor can be performed by comparing translation efficiency in the presence and absence of an eIF4A inhibitor agent in an in-vitro or in-vivo translation system comprising eIF4A and mRNA from the cancer or tumor, wherein a decrease in translation efficiency in the presence of the agent indicates the presence of an eIF4A-dependent translation-controlling motif in mRNA from the cancer or tumor. In another embodiment, identifying the presence of at least one eIF4A-dependent translation-controlling motif in mRNA from the cancer or tumor can be performed by identifying a G-quadruplex motif in at least one oncogene in the cancer or tumor. In certain embodiments, the motif is selected from among those described in SEQ ID NO:1 and in any one of SEQ ID NO:4-62. In certain embodiments, the expression of MYC is not correlated with responsiveness or sensitivity of a patient's cancer or tumor to an agent that inhibits eIF4A activity.

In another embodiment, a method is provided for determining whether a patient having cancer or a tumor will respond to treatment with an eIF4A inhibitor comprising the steps of 1) obtaining a sample of the cancer or tumor from the patient; and 2) identifying the presence of at least one oncogene in the cancer or tumor described in Table 3A, 3B or 3C herein, wherein the presence of said at least one oncogene indicates that the patient will respond to the treatment. In one embodiment, the presence or expression of MYC is not correlated with responsiveness or sensitivity to the treatment.

Furthermore, in other embodiments, methods to determine the level of expression of eIF4E, eIF4A, eIF4G, or eIF4B, and presence of the eIF4F complex indicate sensitivity to silvestrol and other eIF4A inhibitors, and such methods carried out in any format will be useful or determining if a tumor or patient's cancer will be sensitive to silvestrol. In another embodiment, measuring the expression of Mdr1/p-glycoprotein, a resistance marker for silvestrol, indicates the eIF4A inhibitors may be less effective and require a different dosing regimen, such as but not limited to dose level and dosing frequency. In another embodiment, expression of other helicases, e.g. DHX9 and DHX36, may causes resistance to silvestrol and thus useful in identifying cancers or tumors that may not be sensitive to silvestrol, to guide the chemotherapeutic regimen to the optimal benefit of the patient.

In one embodiment, methods are provided for 1) measuring the effect of known RNA helicases such as eIF4A, DHX9 or DHX36 on G-quadruplex unwinding; 2) investigating the effect of other cofactors/inhibitors required for eIF4A activity; 3) a screening method to identify other proteins that can unwind G-quadruplexes; and 4) identifying and establishing the effect of small molecules that stabilize the G-quadruplex structure. These methods among others may be achieved by use of a fluorescence resonance energy transfer (FRET)-based assay utilizing an oligonucleotide comprising a G-quadruplex labeled with a fluorophore at the 5′ or 3′ end of the oligonucleotide, and a fluorescence quencher at the other. In one non-limiting example, a FRET-labeled GC-quadruplex is 5′-UAGAA ACUAC GGCGG CGGCG GAAUC GUAGA (SEQ ID NO:65) and a mutant oligonucleotide without the G-quadruplex is UAGACCCUGCAACGUCAGCGUAGUCGUAGC (SEQ ID NO:66). The 5′-end is labeled with fluorophore FAM and quencher BHQ1 on the 3′end. When folded, the labeled G-quadruplex RNA oligonucleotide will exhibit minimum baseline fluorescence. Addition of specific RNA helicase such as EIF4A with ATP and/or small molecules results in unwinding and increase in fluorescence signal measured in real time. The aforementioned FRET-labeled G-quadruplex containing oligonucleotide is merely one example and those comprising other G-quadruplexes such as but not limited to SEQ ID NOS:1-64, and in particular SEQ ID NOS:1-62 may be employed for this purpose, with other fluorophores and quencher pairs well known in the art.

This assay can therefore be used for the aforementioned purpose as well as various other purposes such as but not limited to 1) measuring the effect of known RNA helicases such as eIF4A, DHX9 or DHX36 on G-quadruplex unwinding; 2) investigating the effect of other cofactors/inhibitors required for eIF4A activity; 3) a screening method to identify other proteins that can unwind G-quadruplexes; and 4) identifying and establishing the effect of small molecules that stabilize the G-quadruplex structure.

In addition to the various embodiments described above, methods are also provided for treating a subject having cancer, and for preventing cancer in a subject at risk or recurrence in a patient in remission. Based on the findings herein that translation of oncogenes comprising an eIF4A-dependent translation-controlling motifs is dependent on eIF4A helicase activity, blocking eIF4A helicase activity is a means to prevent oncogenic protein production and prevent oncogenesis. As described herein, numerous cancer-related genes including oncogenes and transcription factors are dependent on eIF4A for translation. Heretofore, the role of eIF4A was unclear but the present studies show, inter alia, that specific motifs on oncogenic mRNAs depend on eIF4A for translation, thus blocking eIF4A helicase is a heretofore unappreciated anti-cancer mechanism. Use of agents that target eIF4A dependent translation can thus stop translation of oncogenic mRNA sequences.

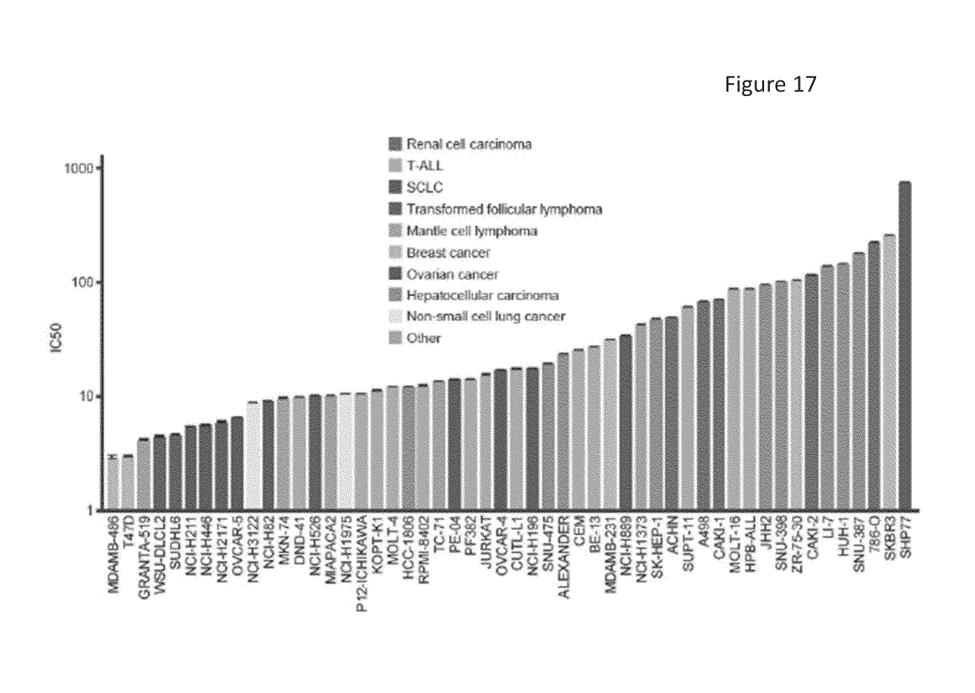

In further embodiments, methods are provided for reducing or preventing recurrence of cancer in a patient in remission or otherwise considered cured. In these embodiments, the cancer is any among those described herein among others, and by way of non-limiting examples, T-cell acute lymphoblastic leukemia, small cell lung cancer, renal cell carcinoma, squamous cell carcinoma of the head and neck, neuroblastoma and pancreatic cancer. In other embodiments, the cancer is transformed follicular lymphoma, mantel cell lymphoma, breast cancer, ovarian cancer, hepatocellular carcinoma, and non-small cell lung cancer, as well as gastric cancer, Ewing sarcoma and lung adenocarcinoma. In one embodiment the subject has cancer. Other cancers are described in FIG. 17 are included herein, as well as the cell lines representative of such cancers. In one embodiment, the subject is at risk for developing cancer. In one embodiment, the subject is in remission from cancer.

Among these methods, administering to the subject an agent that blocks eIF4a helicase activity prevents, treats or intervenes in the recurrence of the cancer. In one embodiment, a method for preventing, treating or intervening in the recurrence of a cancer in a subject is provided. The method comprises administering to the subject an agent that blocks eIF4a helicase activity, thereby preventing, treating or intervening in the recurrence of the cancer. In one embodiment, the agent that blocks eIF4A helicase inhibits the translation of an oncogenic mRNA. In one embodiment, the oncogenic mRNA comprises an eIF4A-dependent translation-controlling motif. In one embodiment, the eIF4A-dependent translation-controlling motif is a G-quadruplex motif. In one embodiment, the eIF4A-dependent translation-controlling motif is selected from among SEQ ID NOs:1-62. In one embodiment, the oncogenic mRNA comprises a G-quadruplex motif. In one embodiment, the oncogenic mRNA is from an oncogene, which by way of non-limiting example is selected from among Tables 3A, 3B and 3C. In one embodiment, the oncogene is NOTCH1, BCL11B, MYC, CDK6, RUNX1, BCL2 or MDM2.

In one embodiment, a method is provided for preventing, treating or intervening in the recurrence of a cancer in a subject having an eIF4A dependent cancer. The method comprises administering to the subject an agent that blocks eIF4a helicase activity, thereby preventing, treating or intervening in the recurrence of the cancer. In one embodiment, the agent that blocks eIF4A helicase inhibits the translation of an oncogenic mRNA. In one embodiment, the oncogenic mRNA comprises an eIF4A-dependent translation-controlling motif. In one embodiment, the eIF4A-dependent translation-controlling motif is a G-quadruplex motif. In one embodiment, the eIF4A-dependent translation-controlling motif is selected from among SEQ ID NOs:1-62. In one embodiment, the oncogenic mRNA comprises a G-quadruplex motif. In one embodiment, the oncogenic mRNA is from an oncogene. In one embodiment, the oncogene is selected from among Tables 3A, 3B and 3C. In one embodiment, the oncogene is NOTCH1, BCL11B, MYC, CDK6, RUNX1, BCL2 or MDM2.

In another embodiment, a method is provided for inhibiting in a subject the translation of an oncogene that comprises an eIF4A-dependent translation-controlling motif. The method comprises administering to the subject an agent that blocks eIF4a helicase, thereby inhibiting translation of the oncogene. In one embodiment, translation of the oncogene causes cancer in the subject. In another embodiment, the eIF4A-dependent translation-controlling motif is a G-quadruplex motif. In this embodiment, the eIF4A-dependent translation-controlling motif is selected from among SEQ ID NOs:1-62. In one embodiment, the mRNA of the oncogene comprises a G-quadruplex motif. In one embodiment, the oncogene is selected from among Tables 3A, 3B and 3C. In one embodiment, the oncogene is NOTCH1, BCL11B, MYC, CDK6, RUNX1, BCL2 or MDM2.

In one embodiment, a method for inhibiting in a subject eIF4A dependent mRNA translation is provided. The method comprises administering to the subject an agent that blocks eIF4a helicase, thereby inhibiting mRNA translation. In one embodiment, the mRNA translation causes cancer in the subject. In one embodiment, the mRNA comprises an eIF4A-dependent translation-controlling motif. In one embodiment, the eIF4A-dependent translation-controlling motif is selected from among SEQ ID NOs:1-62. In one embodiment, the eIF4A-dependent translation-controlling motif is a G-quadruplex motif. In one embodiment, the mRNA encodes an oncogenic protein. In one embodiment, the oncogenic protein is encoded by an oncogene selected from among Tables 3A, 3B and 3C. In one embodiment, the oncogene is NOTCH1, BCL11B, MYC, CDK6, RUNX1, BCL2 or MDM2.

In one embodiment, a method for preventing in a subject the translation of an mRNA comprising an eIF4A-dependent translation-controlling motif. The method comprises administering to the subject an agent that blocks eIF4a helicase activity, thereby inhibiting translation of the mRNA. In one embodiment, the eIF4A-dependent translation-controlling motif is a G-quadruplex motif. In one embodiment, the eIF4A-dependent translation-controlling motif is selected from among SEQ ID NOs:1-62. In one embodiment, the mRNA is from an oncogene selected from among Tables 3A, 3B and 3C. In one embodiment, the oncogene is NOTCH1, BCL11B, MYC, CDK6, RUNX1, BCL2 or MDM2. In one embodiment, the translation of the mRNA causes cancer.

In any of these embodiments, the agent blocks the activity of eIF4A helicase. In any of the foregoing embodiments, the agent blocks the translation of an mRNA comprising an eIF4A-dependent translation-controlling motif. In any of the foregoing embodiments, the eIF4A-dependent translation-controlling motif is a G-quadruplex motif. In any of the foregoing embodiments, the eIF4A-dependent translation-controlling motif is selected from among SEQ ID NOs:1-62.

Non-limiting examples of aforementioned agents include a rocaglamide, such as silvestrol, CR-31-B, or any active analogue or derivative thereof. In other embodiments, the agent is hippuristanol, pateamine A, or any active analogue or derivative thereof. Other examples of suitable agents include those described in WO2011/140334 (based on PCT/US2011/035351).

Hallmark features are described here of eIF4A-dependent translation and defines specific 5′UTR elements that confer a requirement for that RNA helicase. The key features are longer 5′UTRs, a 12-mer (GGC)₄ motif, and related 9-mer variant motifs. Importantly, the 12-mer and 9-mer motifs precisely localize to between 53% and 65% of all predicted RNA G-quadruplex structures (depending on the analysis tool). The 9-mer sequences require neighboring nucleotides to complete the structure as the minimal number is 12 nucleotides, and it was frequently observed that more than 12 nucleotides contribute to the G-quadruplex. Moreover, most of the remaining G-quadruplexes are based on highly similar sequence elements. On the other hand IRES mRNAs are somewhat protected, while TOP, TOP-like, or PRTE elements do not appear to influence the eIF4A requirement. This is distinct from mTORC1 inhibition, which affects a different set of transcripts marked by TOP and TOP-like elements (Thoreen et al., 2012). These findings identify sequence motifs that represent translational control elements encoded in the 5′UTR of several hundred transcripts and that confer a requirement for eIF4A RNA helicase action.

RNA G-quadruplex structures are typically made from at least two stacks of four guanosines exhibiting non-Watson-Crick interactions (e.g. hydrogen bonds) and connected by one or more linker nucleotides (reviewed in (Bugaut and Balasubramanian, 2012)). In the examples herein, the linker is most often a cytosine and less frequently an adenosine. There is variation in the exact structural composition and sequence requirement as our examples illustrate. The minimum requirement for the structure is a (GGC/A)₄ sequence and neighboring nucleotides can complete the structure.

The cap-binding protein eIF4E is limiting for cap-dependent translation and its signaling control by mTORC1 and 4E-BP has been studied in great detail (Jackson et al., 2010). The results described here indicate that for a set of mRNAs the eIF4A helicase activity is required and represents the point of attack for three natural compounds, silvestrol, hippuristanol, and pateamine (Cencic et al., 2007). Moving forward, an intriguing question concerns the physiological control of eIF4A activity (Parsyan et al., 2011). In this regard, recent studies have defined the mechanics of eIF4A action (Marintchev, 2013; Marintchev et al., 2009), identified mutually exclusive potentially regulatory interactions between eIF4A and the eIF4B, eIF4G, and eIF4H factors (Rozovsky et al., 2008), and further implicated S6 kinase in the phosphorylation and signaling control of eIF4B (Kroczynska, 2009; Shahbazian et al., 2010; Shahbazian et al., 2006). The data herein indicate that these interactions define a broadly relevant layer of translational control that is distinct from the control of eIF4E by 4E-BP and mTORC1, and that is specifically aimed at a subset of transcripts.

In one embodiment, the novel sequence motifs and/or G-quadruplex structures are present in a large number of transcription factors, several known oncogenes, but also some tumor suppressor genes. A number of examples are listed and suggest that an eIF4A dependent program of translational control may have broad ramification on a cell's biology. Several genetic lesions implicated in translational activation can promote T-ALL development (e.g. PTEN, IL7R) (Palomero et al., 2007; Zenatti et al., 2011; Zhang et al., 2012).

### Examples

### Materials and Methods

Ribosome Footprinting. KOPTK1 cells were treated with silvestrol or DMSO for 45 minutes, followed by cycloheximide treatment for 10 minutes and then harvested for total RNA and ribosome footprint fragment isolation. Total RNA was isolated using RNA isolation kit from Qiagen (74104) and subjected to RNA sequencing. Ribosome protected fragments were isolated following published protocol (Ingolia et al., 2009). Briefly cell lysates were subjected to ribosome footprinting by nuclease treatment. Footprint fragments were purified by one step sucrose cushion and gel extraction. Deep sequencing libraries were generated from these fragments. Both total RNA and footprint fragment libraries were analyzed by sequencing on the HiSeq 2000 platform.

Sequence Alignment. Sequences were aligned to the transcripts available from the human genome sequence hg19 from UCSC public database. Ribosome footprint (RF) reads were aligned to reference genome hg19 using PALMapper (Jean et al., 2010). Only the uniquely aligned reads were used for analysis. Read length of 25- to 35-bp was selected and used to analyze the translation effect of silvestrol. Total mRNA sequencing reads were aligned to the hg19 reference using STAR (Dobin et al., 2013). The splice alignment was used, and only used the uniquely aligned reads with maximum 3 mismatches.

Footprint Profile Analysis. The genome annotation was from GENCODE project (http://www.gencodegenes.org/releases/14.html). Ribosome footprint intensity (reads per million, RPM) and the expression value (reads per kilobase per million, RPKM) were measured from total mRNA-seq data and translation values were measured from ribosome footprint data. To evaluate the translation efficiency (TE) change between silvestrol- and vehicle-treated samples, TE was calculated as RPKMfootprint/RPKMmRNA (as Thoreen et al. did recently (Thoreen et al., 2012)). Changes in ribosome footprint profiles were determined by using DEXSeq algorithm (Anders et al., 2012). DEXSeq accounts for the discrete nature of the read counts and models biological variability to avoid false positives. Ratio of TEsilvestrol/TEcontrol of all the genes was plotted and color-highlighted according to the statistical significance of DEXSeq test.

Ribosome distribution analysis. The ribosomal distribution change was evaluated between silvestrol treated samples and controls. A BED file containing all non-overlapped exonic regions was generated based on genome annotation. Then the BED file and footprint BAM files were given as an input to SAMTOOLS (Li et al., 2009a) to generate new BAM files that only included exonic alignment. The exonic BAM files were input for two conditions to rDiff (Drewe et al., 2013) to identify genes that presented significant change in ribosomal distribution.

### Additional Experimental Procedures

(Non-radioactive) Metabolic labeling of nascent protein. KOPTK1 cells were labeled for nascent protein synthesis using Click-iTR AHA (L-azidohomoalanine) metabolic labeling reagent obtained from Invitrogen (cat no. C10102) as per manufacturer's instructions. Briefly, following silvestrol, Cycloheximide or DMSO treated cells were incubated in methionine free medium for 30 min prior to AHA labeling for 1 hr. Cells were fixed with 4% paraformaldehyde in PBS for 15 min, permeablized with 0.25% Triton X-100 in PBS for 15 min followed by one wash with 3% BSA. Cells were then stained using Alexa Fluor 488 Alkyne (Invitrogen cat no. A10267) with Click-iT Cell reaction Buffer Kit (Invitrogen cat no. C10269). Changes in mean fluorescence intensity as a measure of newly synthesized protein was detected by Flow cytometry analysis.

Polysome profiling. KOPTK1 cells were treated with silvestrol or DMSO for 45 minutes, followed by cycloheximide treatment for 10 minutes. Cell pellet was lysed in polysome lysis buffer (300 mM NaCl, 15 mM Tris-HCl (pH 7.5), 15 mM MgCl2, 1% TritonX-100, 0.1 mg/ml Cycloheximide, 1 mg/ml Heparin). Polysome fractions were isolated using 4 ml 10-50% sucrose density gradients (300 mM NaCl, 100 mM MgCl2, 15 mM Tris-HCl (pH 7.5), 1 mg/ml Cycloheximide, 10 mg/ml Heparin). Gradients were centrifuged in an SW40Ti rotor at 35,000 rpm for 2 hrs. Fractions of 100 ul were collected manually from the top, and optical density (OD) at 254 nM was measured.

Sequence Alignment. The human genome sequence hg19 was downloaded from UCSC public database: http://hgdownload.cse.ucsc.edu/goldenPath/hg19/chromosomes. Ribosome footprint (RF) reads were aligned to reference genome hg19 using PALMapper (Jean et al., 2010). PALMapper clips the linker sequence (5′-CTGTAGGCACCATCAAT-3′), which is technically introduced during RF library construction, and trims the remaining sequence from the 3′ end while aligning the reads to reference sequence. Briefly, the parameters for PALMapper were set as follows: maximum number of mismatches: 2; maximum number of gaps: 0; minimum aligning length: 15; maximum intron length (splice alignment): 10000; minimum length of a splicing read aligned to either side of the intron boundary: 10. Only the uniquely aligned reads were used for further analysis.

To remove ribosome RNA contamination, the footprint reads were also aligned to a ribosome sequence database using PALMapper with the same parameters except allowing splice alignment. The human ribosome sequences were retrieved from BioMart Ensembl (Flicek et al., 2013) and SILVA (Quast et al., 2013) databases and merged the results into a single FASTA file, which was used as reference sequence to align against. The rRNA-aligned reads were filtered out from hg19-aligned reads.

After removing the rRNA contamination, a portion of reads were observed that were dominated by linker sequence and Illumina P7 adapter. These reads can also be trimmed during mapping and cause false alignment. Therefore, a search was undertaken for a string of 1˜8 nt from linker sequence around the trimming site (±2 bp) allowing 1 nt mismatch. The read was removed if there was no such linker sequence. Finally, reads ≦24-bp and ≧36-bp were filtered out, and the remaining reads with aligned length from 25- to 35-bp were used to analyze the translational effects of silvestrol.

Total mRNA sequencing reads were aligned to the hg19 reference using STAR (Dobin et al., 2013). The splice alignment was performed and only use the uniquely aligned reads with maximum 3 mismatches. rRNA contaminating reads were also filtered out using the same strategy described before.

Footprint Profile Analysis. For each gene, only the number of aligned reads were counted that were mapped within exonic regions. The genome annotation was downloaded from GENCODE project (http://www.gencodegenes.org/releases/14.html). Ribosome footprint intensity (reads per million, RPM) was calculated as RPM=Ci/(N/106), where Ci is the read count for gene i, and N is the library size of silvestrol- or vehicle-treated samples. In order to eliminate the effluence of rRNA contamination, the library size was calculated after read filtering described previously. Similarly, the expression value measured from total mRNA-seq data and translation value measured from ribosome footprint data (both were referred as reads per kilobase per million, RPKM) were calculated as RPKM=Ci/(Ki·N/106), where Ki is the non-overlapped exonic region of each gene. To evaluate the translation efficiency (TE) change between silvestrol- and vehicle-treated samples, TE=RPKMfootprint/RPKMmRNA was calculated as Thoreen et al did recently (Thoreen et al., 2012).

To detect the genes that ribosome footprint profiles were significantly changed between silvestrol treated sample and control, DEXSeq (Anders et al., 2012) was used to perform the statistical test. DEXSeq accounts for the discrete nature of the read counts and it also models the biological variability which has been demonstrated in other applications to be crucial to avoid a great number of false positives. Here, DEXSeq was used in a specific way: the footprint and mRNA-seq read counts were fit into DEXseq framework, in which silvestrol treatment and control are two biological conditions, and then tested whether footprint (consisting 2 replicates for each condition) and mRNA-seq (The 3 replicates were split and recombined into two combinations such that each of them consists of two replicates) read counts were significantly different in the two conditions. The log-ratio of normalized read counts of silvestrol treated sample to control indicated whether ribosome footprint profile was increased or decreased. In the end, the ratio of TEsilvestrol/TEcontrol of all the genes was plotted, and color-highlighted them according to the statistical significance of the DEXSeq test.

In addition to studying the translation efficiency, the ribosomal distribution change was also evaluated between silvestrol treated sample and control. First, a BED file contained all non-overlapped exonic regions was generated based on genome annotation. Then the BED file and footprint BAM files were given as an input to SAMTOOLS (Li et al., 2009) to generate new BAM files only included exonic alignment. The exonic BAM files of two conditions to rDiff (Drewe et al., 2013) were input to identify genes that presented significant change in ribosomal distribution. In detail, a nonparametric test was performed implemented in rDiff to detect differential read densities. rDiff takes relevant read information, such as the mapping location and the read structure, to measure the significance of changes in the read density within a given gene between two conditions. The minimal read length was set to 25-bp, and number of permutation was set to 10000.

To plot the ribosomal distribution curves for multiple genes, read coverage of each transcript was normalized by the mean coverage value of that particular transcript. Then the UTR and coding exon length were normalized in proportion to the overall average length of corresponding regions of a group of genes. Finally all the normalized transcripts were averaged together in a vectorized way to plot the coverage distribution. The ribosomal distribution curves for a single gene were plotted in a similar way but without normalizing the read coverage, and the coverage was smoothed using ‘moving average’ smoothing algorithm.

Motif analysis. The transcripts of each gene were quantified based on the total mRNA-seq data using MISO (Katz et al., 2010). The 5′UTR of most abundant transcript was collected for predicting motifs. Both the significant genes with increased or decreased TE and altered ribosomal distribution and the corresponding background gene sets were predicted by DREME (Bailey, 2011). Over- and under-represented motifs were determined with three different settings: searching for motifs of length greater than or equal to six, nine and twelve base pairs. The predicted consensus sequences with P<1×10-4 were considered as significant motifs. The secondary structure of different gene sets was predicted using RNAfold (Hofacker, 2003) based on the same 5′UTR prepared before.

5′UTR sequences for respective group of targets were subjected to motif prediction using online available program RegRNA (A Regulatory RNA motifs and Elements Finder) (http://regrna.mbc.nctu.edu.tw/html/prediction.html) and looked specifically for motifs that occur in 5′UTR. Statistical significance for the results obtained was calculated using Fisher's exact test for count data.

T-ALL samples. Thirty-six bone marrow biopsies were collected from patients with T-ALL at multiple organizations (Universitair Ziekenhuis (UZ) Ghent, Ghent, Belgium; UZ Leuven, Leuven, Belgium; Hôpital Purpan, Toulouse, France; Centre Hospitalier Universitaire (CHU) de Nancy-Brabois, Vandoeuvre-Les-Nancy, France). The QIAamp DNA Mini kit was used to obtain genomic DNA (Qiagen 51304). The Medical Ethical Commission of Ghent University Hospital (Ghent, Belgium, B67020084745) approved this study.

Mutation analysis. NOTCH1 (exons 26, 27, 28 and 34), FBXW7 (exons 7, 8, 9, 10 and 11), PTEN (exons 1 till 9) and IL7R (exon 6) were amplified and sequenced using primers as reported in (Mavrakis et al., 2011; Shochat et al., 2011; Zuurbier et al., 2012). FBXW7, PTEN and IL7R amplification were performed using 20 ng of genomic DNA, 1×KapaTaq reaction buffer (KapaBiosystems), 1U KapaTaq DNA polymerase, 0.2 mM dNTP, 2.5 μM MgCl2, 0.2 mM forward and reverse primer in a 25 μl PCR reaction. For NOTCH1 amplification, the PCRx enhancer system (Invitrogen) was used for the PCR reaction. Reactions contained 20 ng of DNA, 2.5 U KapaTaq DNA Polymerase, 1×PCRx Amplification Buffer, 2×PCRx Enhancer Solution, 0.2 mM dNTP, 1.5 mM MgSO4 and 0.2 mM of each primer. The PCR steps were: 95° C. for 10 minutes, (96° C. for 15 sec, 57° C. for 1 minute, then 72° C. for 1 min) for 40 cycles, then 72° C. for 10 minutes. Purified PCR products were analyzed using the Applied Biosystems 3730XL DNA Analyze.

Array Complete Genomic Hybridization. PTEN deletions and MYC amplifications were detected by array CGH analysis using SurePrint G3 Human 4×180K CGH Microarrays (Agilent Technologies). First, random prime labeling of the T-ALL DNA sample and a control human reference DNA was performed with Cy3 and Cy5 dyes (Perkin Elmer), respectively. The subsequent hybridization protocol was performed according to the manufacturer's instructions (Agilent Technologies). The data was analyzed using arrayCGHbase (Menten et al., 2005).

Immunohistochemistry and Tissue Microarrays. T-cell acute lymphoblastic leukemia tissue microarrays were made as previously published (Schatz et al., 2011) using an automated tissue arrayer (Beecher Instruments, ATA-27). T-ALL samples were ascertained at Memorial Sloan-Kettering Cancer Center and were approved with an Institutional Review Board Waiver and approval of the Human Biospecimen Utilization Committee. All cancer biopsies were evaluated at MSKCC, and the histological diagnoses were based on haematoxylin and eosin (H&E) staining. TMAs were stained with the c-MYC polyclonal antibody (Epitomics 51242) using Discovery XT (Ventana) for 1 hour and a secondary anti-rabbit antibody (Vector Laboratories) for 1 hour. Histological images were captured using a Zeiss Axiocam MRc through a Zeiss Achropla lens on an Axioskop 40 microscope. Images were processed for brightness and contrast using Axiovision Rel. 4.6. Cores were scored as 0, 1, or 2 reflecting the fraction of positive cells.

Generation of mice. The ICN-driven mouse T-ALL model has been reported (Pear et al., 1996; Wendel et al., 2004). Data were analyzed in Kaplan-Meier format using the log-rank (Mantel-Cox) test for statistical significance. The surface marker analysis was as described (Wendel et al., 2004). ShRNAs against Pten and Fbxw7 have been reported in (Mavrakis et al., 2011).

Tumor transplantation. Leukemic bone marrow from mice expressing the ICN and IK6 was infected with OMOMYC and selected using puromycin. 2,000,000 cells were injected into syngeneic recipients via tail vein. Mice were monitored by blood analysis. Upon leukemia detection, tamoxifen (50 mg/kg) or vehicle treatment was performed on alternating days until mice were moribund. Severe leukemia reflects >100,000 blasts/μl and led to rapid demise of animals if untreated, whereas complete remission was defined as the absence of GFP positive leukemic blasts in the blood and bone marrow.

Real-Time Quantitative PCR. Total RNA was extracted using AllPrep DNA/RNA/Protein Mini Kit (Qiagen 80004). Normal CD3+ T-cell RNA mixed from healthy donors was purchased from Miltenyi Biotec (130-093-164). cDNA was made using SuperScript III First-Strand (Invitrogen 18080-400). Analysis was performed by ΔΔCt. Applied Biosystems Taqman GeneExpression Assays: human Myc Hs00153408_m1, hsa-miR-19b RT and TM 396, Rnu6b RT and TM 001093, and mouse Myc Mm00487804_m1.

T-ALL cell lines. T-ALL cell lines were cultured in RPMI-1640 (Invitrogen, CA), 20% fetal calf serum, 1% penicillin/streptomycin, and 1% L-glutamine. The MOHITO line was supplemented with 5 ng/mL IL2 (Fitzgerald 30R-A1022 and 10 ng/mL of IL7 (Fitzgerald 30R-AI084X).

Immunoblots. Lysates were made using Laemli lysis buffer. 30 ug of protein was loaded onto SDS-PAGE gels then transferred onto Immobilon-FL Transfer Membranes (Millipore IPFL00010). The antibodies used were α-Tubulin (Sigma T5168), β-actin (Sigma A5316), Myc (Santa Cruz Biotechnology sc-40), p-Akt 308 (Cell Signaling 9275), Akt (Cell Signaling 9272), S6 (Cell Signaling 2317), and p-S6 (Cell Signaling 2215), Notch1 (Cell signaling 3608), Myb (Santa Cruz Biotechnology, sc-517), CDK6 (Cell Signaling 3136), EZH2 (Cell Signaling 5246), Mdm2 (Santa Cruz Biotechnology, sc-965), Bcl2 (Santa Cruz Biotechnology, sc-509), Run×1 (Cell Signaling 4336), and GAPDH (Cell Signaling 5174).

Luciferase assays. Four tandem repeats of the (CGG)4 12-mer motif (GQs) or random sequence matched for length and GC content (random) were cloned into the 5′UTR of Renilla luciferase plasmid pGL4.73. Empty firefly luciferase plasmid pGL4.13 or HCV-IRES firefly were used as internal controls. Luciferase assays were performed using Dual-Luciferase Reporter Assay System (Promega E1960) following the manufacturer's instructions. GQs sequence:

(SEQ ID NO: 63)

CTAGGTTGAAAGTACTTTGACGGCGGCGGCGGTCAATCTTACGGCGGCGG
CGGACATAGATACGGCGGCGGCGGTAGAAACTACGGCGGCGGCGGATTA
GAATAGTAAA

Random Sequence:

(SEQ ID NO: 64)

CTAGGGCGCACGTACTTCGACAACGTCAGCGTTCAGCGTTCCAACGTCAG
CGTACAGCGATCCAACGTCAGCGTTCTGCGCTACAACGTCAGCGTATCCG
CGTAGCACA

Statistical analysis. All Kaplan-Meier curves were analyzed using the Mantel-Cox test. The significance of xenografted tumor size differences was determined using two-way repeated measures ANOVA tests. RT-PCRs were analyzed with two tailed t-tests.

Xenografts. 5,000,000 KOPT-K1 cells in 30% matrigel (BD 354234) were injected subcutaneously into C.B-17 scid mice. When tumors were readily visible, the mice were injected on 7 consecutive days with either 0.5 mg/kg silvestrol, 0.2 mg/kg (±)-CR-31-B, or every other day with 1 mg tamoxifen. Tumor size was measured daily by caliper. P-values were calculated using 2-way repeated measures ANOVA.

Silvestrol and (±)-CR-31-B. Each was suspended in DMSO for in vitro experiments and 5.2% Tween 80 5.2% PEG 400 for in vivo experiments. Cycloheximide (C7698) and Rapamycin (R8781) were purchased from Sigma.

Toxicity studies. Eight week-old C57Bl/6NTac female mice were randomly assigned to either control or treatment groups. Each treatment group received one daily dose of test article through i.p. injection over 5 consecutive days. Toxicity was monitored by weight loss and daily clinical observation for the 14 days following test article administration. 24 hours after the last test article administration 4 mice in each group were sacrificed and clinical chemistry, hematology and tissue specific histopathology were done at autopsy. The remaining mice (n=2 per group) were kept under observation for an additional 13 days; at that point all mice were sacrificed and clinical chemistry, hematology and tissue specific histopathology were done at time of autopsy.

References for Materials and Methods:
Anders, S., Reyes, A., and Huber, W. (2012). Detecting differential usage of exons from RNA-seq data. Genome research 22, 2008-2017.
Bailey, T. L. (2011). DREME: motif discovery in transcription factor ChIP-seq data. Bioinformatics 27, 1653-1659.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Drewe, P., Stegle, O., Hartmann, L., Kahles, A., Bohnert, R., Wachter, A., Borgwardt, K., and Ratsch, G. (2013). Accurate detection of differential RNA processing. Nucleic acids research 41, 5189-5198.
Flicek, P., Ahmed, I., Amode, M. R., Barrell, D., Beal, K., Brent, S., Carvalho-Silva, D., Clapham, P., Coates, G., Fairley, S., et al. (2013). Ensembl 2013. Nucleic acids research 41, D48-55.
Hofacker, I. L. (2003). Vienna RNA secondary structure server. Nucleic acids research 31, 3429-3431.
Jean, G., Kahles, A., Sreedharan, V. T., De Bona, F., and Ratsch, G. (2010). RNA-Seq read alignments with PALMapper. Current protocols in bioinformatics/editoral board, Andreas D Baxevanis [et al] Chapter 11, Unit 11 16.
Katz, Y., Wang, E. T., Airoldi, E. M., and Burge, C. B. (2010). Analysis and design of RNA sequencing experiments for identifying isoform regulation. Nature methods 7, 1009-1015.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Mavrakis, K. J., Van Der Meulen, J., Wolfe, A. L., Liu, X., Mets, E., Taghon, T., Khan, A. A., Setty, M., Rondou, P., Vandenberghe, P., et al. (2011). A cooperative microRNA-tumor suppressor gene network in acute T-cell lymphoblastic leukemia (T-ALL). Nat Genet 43, 673-678.
Menten, B., Pattyn, F., De Preter, K., Robbrecht, P., Michels, E., Buysse, K., Mortier, G., De Paepe, A., van Vooren, S., Vermeesch, J., et al. (2005). arrayCGHbase: an analysis platform for comparative genomic hybridization microarrays. BMC bioinformatics 6, 124.
Pear, W. S., Aster, J. C., Scott, M. L., Hasserjian, R. P., Soffer, B., Sklar, J., and Baltimore, D. (1996). Exclusive development of T cell neoplasms in mice transplanted with bone marrow expressing activated Notch alleles. The Journal of experimental medicine 183, 2283-2291.
Quast, C., Pruesse, E., Yilmaz, P., Gerken, J., Schweer, T., Yarza, P., Peplies, J., and Glockner, F. O. (2013). The SILVA ribosomal RNA gene database project: improved data processing and web-based tools. Nucleic acids research 41, D590-596.
Schatz, J. H., Oricchio, E., Wolfe, A. L., Jiang, M., Linkov, I., Maragulia, J., Shi, W., Zhang, Z., Rajasekhar, V. K., Pagano, N. C., et al. (2011). Targeting cap-dependent translation blocks converging survival signals by AKT and PIM kinases in lymphoma. The Journal of experimental medicine 208, 1799-1807.
Shochat, C., Tal, N., Bandapalli, O. R., Palmi, C., Ganmore, I., to Kronnie, G., Cario, G., Cazzaniga, G., Kulozik, A. E., Stanulla, M., et al. (2011). Gain-of-function mutations in interleukin-7 receptor-alpha (IL7R) in childhood acute lymphoblastic leukemias. The Journal of experimental medicine 208, 901-908.
Thoreen, C. C., Chantranupong, L., Keys, H. R., Wang, T., Gray, N. S., and Sabatini, D. M. (2012). A unifying model for mTORC1-mediated regulation of mRNA translation. Nature 485, 109-113.
Wendel, H. G., De Stanchina, E., Fridman, J. S., Malina, A., Ray, S., Kogan, S., Cordon-Cardo, C., Pelletier, J., and Lowe, S. W. (2004). Survival signalling by Akt and eIF4E in oncogenesis and cancer therapy. Nature 428, 332-337.
Zuurbier, L., Petricoin, E. F., Vuerhard, M. J., Calvert, V., Kooi, C., Buijs-Gladdines, J., Smits, W. K., Sonneveld, E., Veerman, A. J., Kamps, W. A., et al. (2012). The significance of PTEN and AKT aberrations in pediatric T-cell acute lymphoblastic leukemia. Haematologica. 2012 September; 97(9):1405-13.

### Example 1

### Cap-Dependent Translation in Oncogenesis

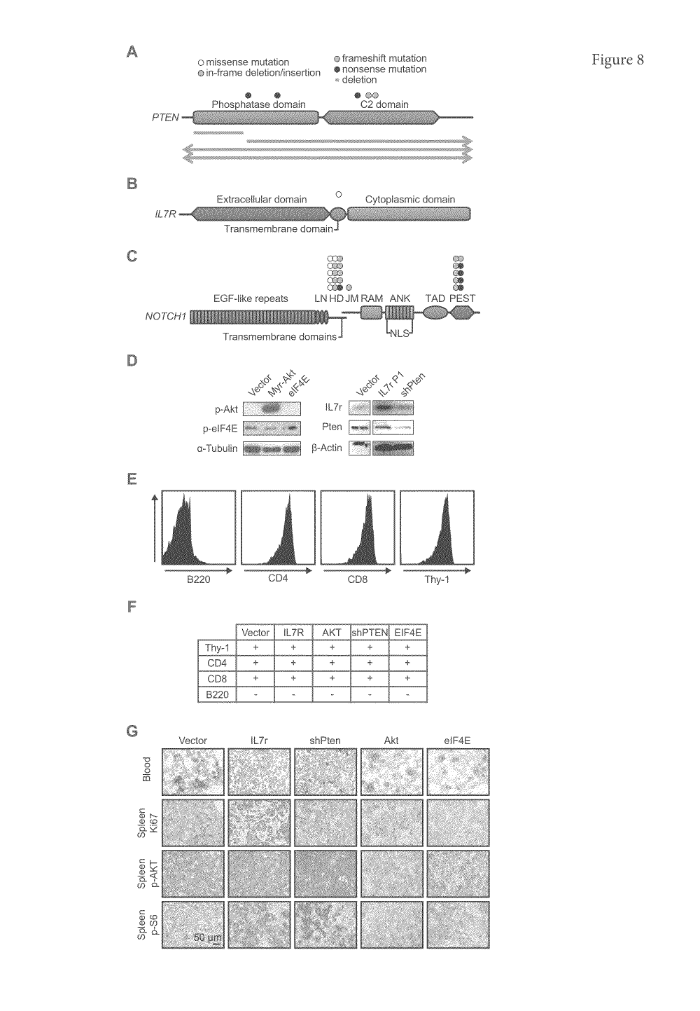

NOTCH-driven T-ALL exemplifies the frequent activation of AKT/mTORC1 and cap-dependent translation seen in cancer. For example, in a small series of pediatric T-ALLs the common NOTCH1 HD and PEST domain mutations were confirmed (56%; 20/36 samples) (O'Neil et al., 2007; Weng et al., 2006), PTEN mutations (14%; 5/36), and PTEN deletions (11%; 4/36), resulting in mono- (16%) or bi-allelic (6%) PTEN loss (Gutierrez et al., 2009; Palomero et al., 2007; Zhang et al., 2012), and occasional IL7R mutation (3%) (Zenatti et al., 2011) (FIG. 8 A-C, Table 1).

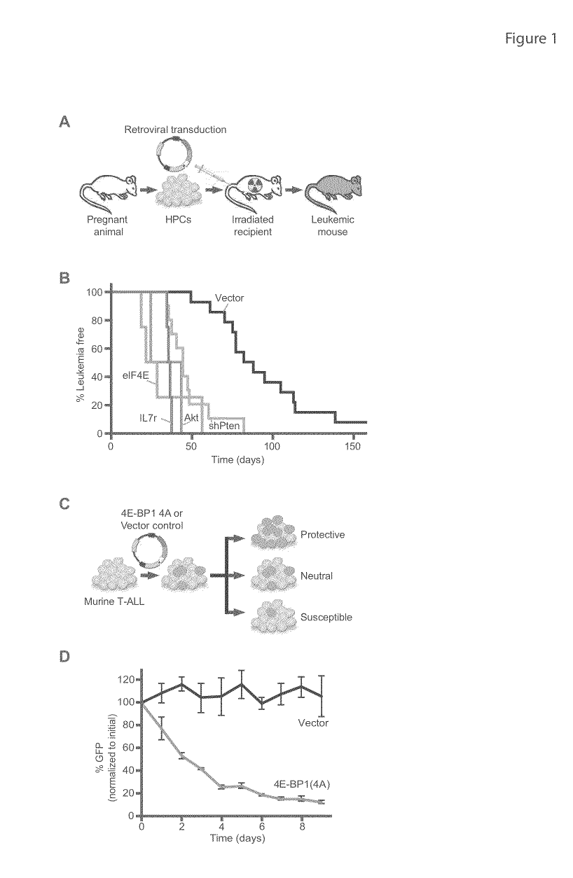

These mutations contribute to T-cell leukemogenesis. Briefly, murine hematopoietic precursor cells (HPCs) expressing Notchl intracellular fragment (ICN) alone or in combination with additional alleles were transplanted and disease latency measured in recipient animals (FIG. 1A) (Mavrakis et al., 2011; Pear et al., 1996). Notch-ICN caused T-ALL in three months (n=14, mean latency 91.5 days), while co-expression of a short-hairpin RNA (shRNA) against Pten cut average latency in half (n=10, p<0.0001; mean latency 47.1 days) (FIG. 1B, FIG. 8D). Similarly, expression of the mutant IL7r allele (IL7R/p.L242-L243insNPC (Zenatti et al., 2011), n=4, p<0.0001, mean latency 35.5 days), or the Akt oncogene (n=4, p<0.0001, mean latency 33.5 days) dramatically accelerated leukemia onset. Notably, expression of the cap-binding protein eIF4E was sufficient to reduce latency to one month (n=4, p<0.0001, mean latency 30.75 days). Pathologically all leukemias were composed of CD4/CD8 double positive cells, and immunohistochemistry showed abundant Ki67 expression and increased S6 phosphorylation in the PTEN deficient, IL7R and AKT expressing T-ALLs (FIG. 8 E/F). Hence, the cap-binding protein eIF4E is sufficient to promote NOTCH-induced T-ALL.

A genetic approach was then used to test the requirement for eIF4E in maintaining the leukemic cells. Briefly, the 4E-binding protein (4E-BP) sequesters eIF4E and blocks cap-dependent translation (Rousseau et al., 1996). 4E-BP is negatively regulated by sequential phosphorylation at several serine residues by mTORC1, and mutation of these sites results in a constitutively active 4E-BP1 (4E-BP1(4A)) allele (Rong et al., 2008). In mixed populations of murine T-ALL cells where a fraction of cells express either 4E-BP1(4A) and GFP or an empty vector and compete with un-transduced parental cells, rapid elimination was seen of 4E-BP1(4A)/GFP expressing cells from the culture (FIG. 1D/E). Hence, eIF4E activity is required to maintain T-ALL, which indicates that targeting translation might be a therapeutic strategy.

FIG. 1 depicts the translational activation in T-ALL pathogenesis and maintenance. A) Diagram of the NOTCH-ICN-driven murine T-ALL model. B) Kaplan-Meier analysis showing time to leukemia development after transplantation of HPC transduced with NOTCH1-ICN and empty vector (black, n=9), eIF4E (green, n=4), IL7r p.L242-L243insNPC (P1) (blue, n=4), shPten (orange, n=10), or Akt (red, n=4). C) Experimental design of competition experiments and potential outcomes. D) Results as percentage of each starting GFP positive population of murine T-ALL cells partially transduced with vector/GFP or the constitutive inhibitory 4E-binding protein (4E-BP1 (4A)).

FIG. 8 depicts the PI3K pathway and translational activation in T-ALL. A-C) Diagram of mutations in human T-ALL affecting PTEN (A), IL7R (B), and NOTCH1 (C). D) Immunoblots of lysates from ICN-driven murine leukemia with the additional indicated construct, probed as indicated. E) Representative FACS profiles measuring levels of the indicated markers in murine leukemia; F) Surface marker expression on murine leukemic cells of indicated genotype (+ and − indicate < or ≧50% positive cells). G) Representative histology detailing the pathological appearance of murine T-ALLs harboring the indicated genes and stained as indicated.

### Example 2

### Silvestrol Blocks Cap-Dependent Translation and is Active Against T-Cell Leukemia

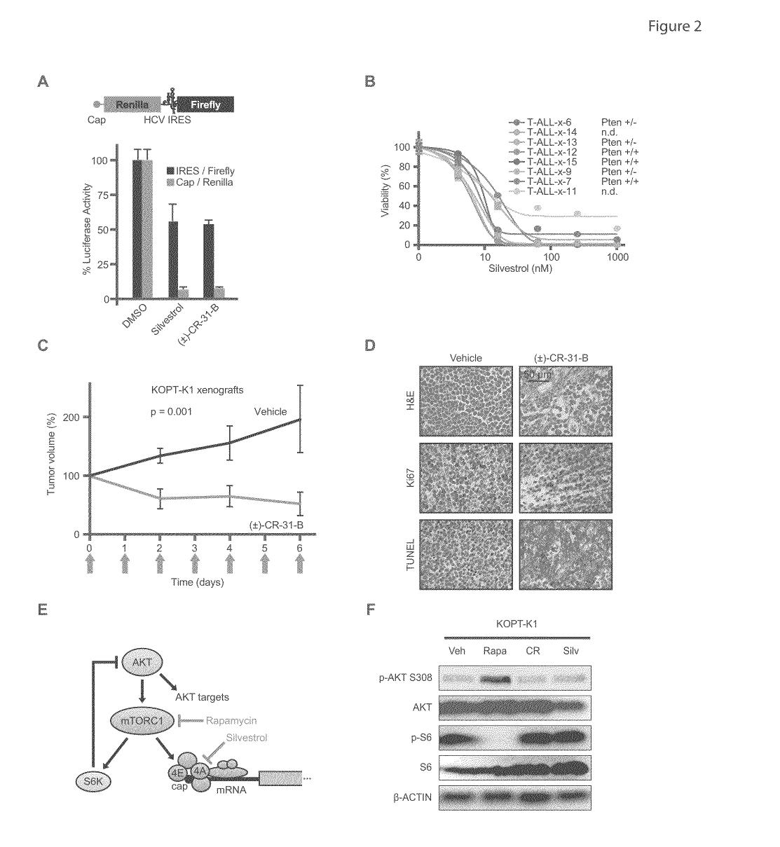

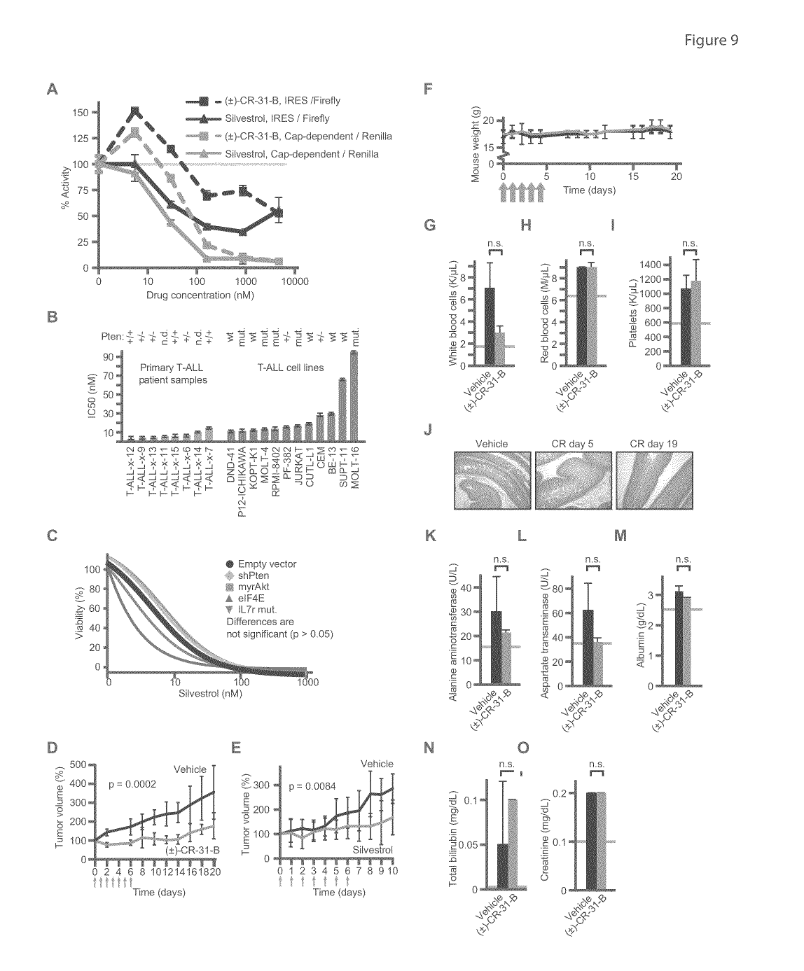

Based on this genetic evidence a pharmacological inhibitor was then tested. Silvestrol is perhaps the best-characterized inhibitor of the eIF4F complex, it does not target eIF4E and instead blocks the eIF4A RNA helicase by stabilizing its mRNA bound form (Bordeleau et al., 2008; Cencic, 2009). Silvestrol, and a synthetic rocaglamide analogue (±)-CR-31-B (CR) bind the same site on eIF4A (Rodrigo et al., 2012; Sadlish et al., 2013). In a dual-luciferase reporter assay, where renilla and firefly luciferase are either capped or under control of an internal ribosomal entry site (IRES) element, both drugs were confirmed to preferentially block cap-dependent over IRES-dependent translation (Bordeleau et al., 2006) (FIG. 2A, FIG. 9A).

Silvestrol has excellent single-agent activity against T-ALL in vitro and in vivo. Silvestrol was tested against primary human T-ALL samples in vitro and observed efficient apoptosis induction with IC50 values ranging from 3 to 13 nM; and confirmed activity in established cell lines (FIG. 2B, FIG. 9B). The results were similar for similar the analogue CR (not shown). Notably, silvestrol showed equal activity against PTEN wild type and PTEN mutant cell lines and primary T-ALL cells. The least sensitive line (MOLT-16) carries a c-MYC translocation (Shima-Rich et al., 1997). Similarly, murine T-ALL cells engineered to express Akt, mutant IL7R, eIF4E, or an shRNA against Pten showed no significant difference in sensitivity indicating that silvestrol can overcome their activity (Figure S2C). In vivo both silvestrol and CR were effective against xenografted T-ALL cells (FIG. 2C, Figure S2D/E). Treatment of KOPT-K1 tumor (˜1 cm3) bearing NOD/SCID mice with systemic administration of silvestrol (0.5 mg/kg, d 0-6) and CR (0.2 mg/kg, d 0-6) produced a significant delay in tumor growth (Silvestrol: n=7, p<0.001; CR: n=8, p<0.001) (FIG. 2C, FIG. 9D/E). Pathologic analysis of treated tumors showed diffuse apoptosis by TUNEL and loss of proliferation by Ki-67 (FIG. 2D). Notably, no severe toxicity, death, or weight loss was observed. CR treatment at therapeutic doses showed a reversible drop in white cell count with a nadir on day 19, and no other changes in blood counts or bone marrow cytology, or serum chemistry (FIG. 9F-O, Table 2). No changes were observed in intestinal histology, which is a major concern with gamma secretase-inhibitors (FIG. 9J) (Real et al., 2009). Hence, single agent silvestrol or CR treatment is effective against T-ALL and is safe in vivo.

Silvestrol acts in a manner that is distinct from mTORC1 inhibitors. For example, the predominant inhibition of S6 kinase instead of 4E-BP and feedback activation of AKT (S308 phosphorylation) are thought to hinder the therapeutic effect of rapamycin (FIG. 2E) (Choo et al., 2008; Kang et al., 2013; Thoreen et al., 2009); 2)(O'Reilly et al., 2006; Sun et al., 2005; Thoreen et al., 2009; Wan et al., 2007). This feedback mechanism is active in KOPT-K1 cells, where Rapamycin-induced loss of ribosomal S6 phosphorylation and feedback activation of AKT (T308) was observed (FIG. 2F). By contrast, selective inhibition of eIF4A with silvestrol or CR does not affect S6 kinase activity and did not lead to phosphorylation of AKT (T308). Hence, selective inhibition of eIF4A is sufficient for therapeutic activity and avoids feedback activation of upstream AKT signaling.

FIG. 2 shows silvestrol blocks cap-dependent translation and has single-agent activity against T-ALL. A) Diagram of the dual reporter system expressing a capped renilla luciferase transcript (red) and firefly luciferase under control of the hepatitis C IRES-element (black); (below) Relative levels of renilla luciferase (red, cap-dependent) and firefly luciferase (black, IRES dependent) upon treatment with vehicle (DMSO), silvestrol or the synthetic analogue (±)-CR-31-B. B) Viability of T-ALL primary patient samples treated with silvestrol for 48 hours; PTEN status is indicated. C) Tumor size of KOPT-K1 xenografts upon treatment with (±)-CR-31-B (0.2 mg/kg) or vehicle (see Figure S2 for longer follow up and silvestrol treatment data). D) Immunohistochemistry of (±)-CR-31-B treated KOPT-K1 tumors stained as indicated. E) Simplified diagram of rapamycin and silvestrol mechanism of action. F) Lysates of KOPT-K1 cells treated with vehicle (Veh), Rapamycin (Rapa: 25 nM), (±)-CR-31-B (CR: 25 nM), or silvestrol (Silv: 25 nM) for 48 hours and probed as indicated.

FIG. 9 shows testing silvestrol and the synthetic analogue (±)-CR-31-B in T-ALL. A) Dual luciferase reporter assay, shown are relative levels of each firefly (cap-dependent) and renilla (IRES-dependent) luciferase upon treatment with silvestrol or (±)-CR-31-B. B) IC50 values for silvestrol and CR in a panel of human T-ALL lines. C) Silvestrol effect on murine T-ALLs with the indicated genetic lesions; curves are mean of triplicates and differences between the genotypes did not reach significance. D) KOPT-K1 xenograft studies. Shown is the tumor volume during and after systemic treatment with CR or vehicle (intraperitoneal injection, 0.2 mg/kg on days indicated by red arrows). E) Tumor volume upon intraperitoneal treatment with vehicle or silvestrol (0.5 mg/kg on days indicated by red arrows). F-0) Toxicity studies with (±)-CR-31-B. F) Animal weights during and after CR treatment (intraperitoneal injection, 0.2 mg/kg on days indicated by red arrows), red=CR, black=vehicle. G-I) Counts of white blood cells (G), red cells (H), and platelets (I) 14 days after cessation of CR treatment, blue lines indicate the species and strain specific reference range, n.s. indicates not significant. J) Representative histology of gastrointestinal tract (small intestine) on the indicated days during and after (±)-CR-31-B treatment; K-O) Serum levels of alanine aminotransferase (ALT) (K), aspartate transaminase (AST) (L), albumin (M), total bilirubin (N), and creatinine (O) two weeks after cessation of treatment with 0.2 mg/kg CR or vehicle, blue lines indicate the species and strain specific reference range, n.s. indicates not significant.

### Example 3

### Transcriptome-Scale Ribosome Footprinting Defines Silvestrol-Sensitive Translation

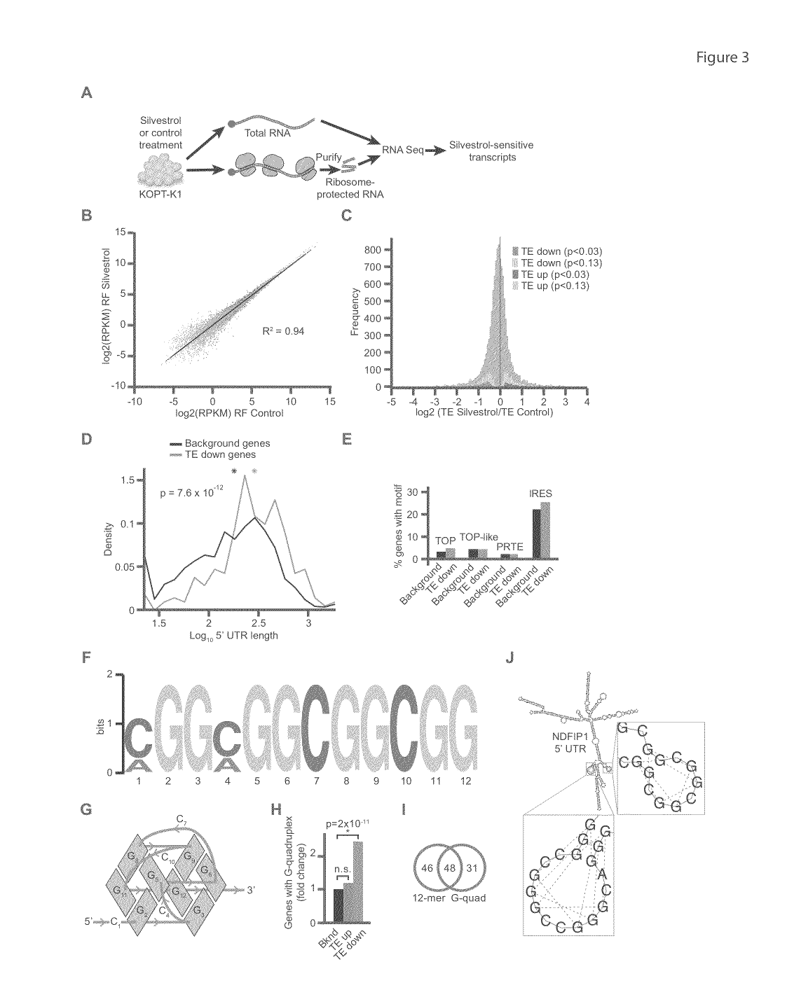

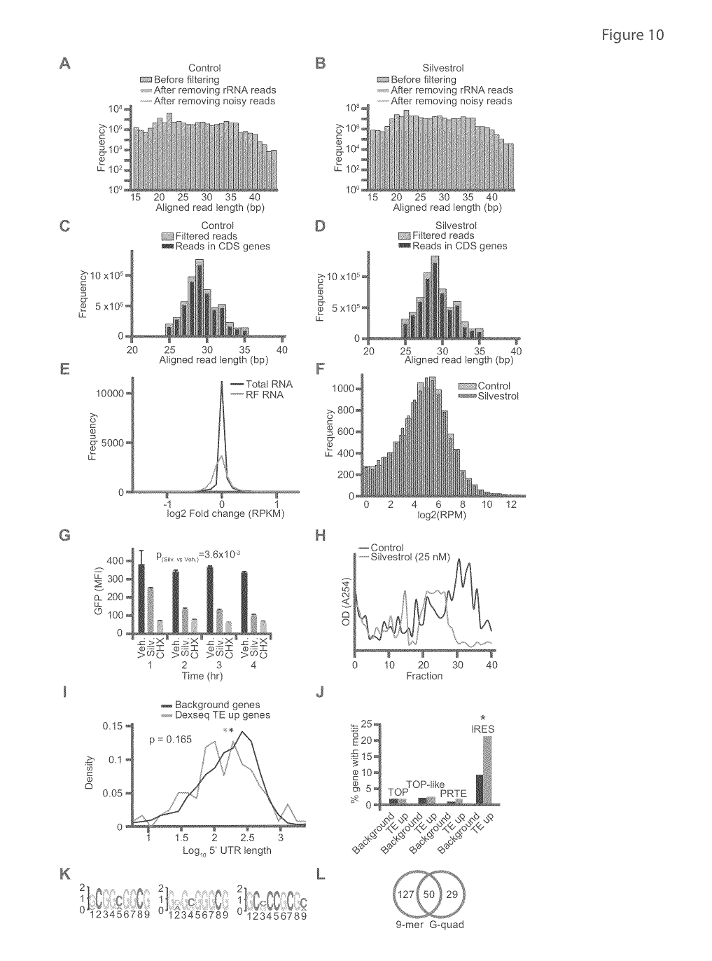

Next, use of the recently developed ribosome footprinting technology (Ingolia et al., 2009) was employed to measure precisely how silvestrol affected protein translation. Briefly, KOPT-K1 cells were treated with 25 nM of silvestrol or vehicle, cells collected after 45 minutes, then isolated and deep-sequenced total RNA and ribosome footprints (RFs) prepared (FIG. 3A). The early time point was chosen to capture effects on translation and minimize secondary transcriptional changes and cell death. First, RFs per mRNA were determined which, after correcting for transcript levels and length, indicated changes in translational efficiency (TE). Out of six measurements two outliers were removed (see methods); the remaining two biological replicates showed excellent consistency of read counts for each gene (Control: R2=0.90; Silvestrol: R2=0.88; data not shown). Raw reads were aligned to the human reference genome hg19 allowing for splicing alignment and using only uniquely aligned reads. Reads mapping to ribosomal RNAs, non-coding RNAs (Guttman et al., 2013) were then removed, and linkers used in library generation, incomplete and spurious alignments, and aligned lengths between 25 and 35 nucleotides (see methods) (FIG. 10A/B). The majority of the remaining reads now mapped to protein coding genes (FIG. 10C/D). The total number of RF reads that mapped to exons was 3.2 million in control and 3.4 million Silvestrol samples and this corresponded to ˜11,128 protein coding genes.

Silvestrol produced an immediate and broad inhibitory effect on cap-dependent translation. RF reads were fewer in number and showed a wider variation between control and silvestrol than total RNA sequences indicating minimal transcriptional variation (FIG. 10E). The number of ribosomes occupying a given transcript is given as gene specific RF reads per one million total reads (RPM). The RPM frequency distribution of control and silvestrol samples were overlapping, indicating that silvestrol equally affected mRNAs with high and low ribosome occupancy (FIG. 10F). Measurements of nascent protein synthesis with L-azidohomoalanine (AHA) labeling confirmed a broad inhibitory effect on translation (max. reduction with silvestrol ˜60%; p(Silv. vs. Veh.)=3.6×10−3, and 80% with cycloheximide, p(CHX vs. Veh.)=2×10−4) (FIG. 10G). Consistently, analyses of polyribosome bound RNA indicated loss of polyribosome bound RNA (fractions 30-40) upon silvestrol treatment (FIG. 10H).

Silvestrol affected the translational efficiency of specific sets of mRNAs. To calculate the translational efficiency (TE) for each mRNA the RF frequency was normalized to the length of the corresponding mRNA yielding an RF density (expressed as RPKM: reads per kilobase per million reads), and was corrected for total mRNA expression. Overall RPKM values for RF from vehicle and silvestrol treated samples were significantly correlated (R2=0.94) indicating a broad inhibitory effect on translation (FIG. 3B). The DERseq algorithm (Differential Expression-normalized Ribosome-occupancy) was used, based on the reported DEXseq algorithm (Anders et al., 2012), to identify mRNAs that were strongly affected by silvestrol (see method). A cut-off at p<0.03 (corresponding to a Z-score >2.5) was used to define groups of mRNAs whose translational efficiency (TE) was either most (TE down; red) or least (TE up; blue) affected by silvestrol compared to most other mRNAs (background; grey) (FIG. 3C, see also U.S. application Ser. No. 61/912,420, filed Dec. 5, 2013; and Wolfe A L, Singh K, Zhong Y, Drewe P, Rajasekhar V K, Sanghvi V R, Mavrakis K J, Jiang M, Roderick J E, Van der Meulen J, Schatz J H, Rodrigo C M, Zhao C, Rondou P, de Stanchina E, Teruya-Feldstein J, Kelliher M A, Speleman F, Porco J A Jr, Pelletier J, Ratsch G, Wendel H. RNA G-quadruplexes cause eIF4A-dependent oncogene translation in cancer. Nature. 2014 Sep. 4; 513(7516):65-70. doi: 10.1038/nature13485. Epub 2014 Jul. 27, both of which are incorporated herein by reference in their entireties) (Thoreen et al., 2012). The TE down group included 281 mRNAs (220 have annotated 5′UTRs), TE up included 190 mRNAs, and the background list included 2243 mRNAs. These groups were used to define the characteristics of differentially affected mRNAs.

FIG. 3 depicts transcriptome-scale ribosome footprinting defines silvestrol's effects on translation. A) Schematic of the ribosome footprinting experiments (see text for details). B) Ribosome density for transcripts across control and silvestrol samples (ribosomal footprint (RF) reads per kilobase per million reads (RPKM)). The correlation (R2=0.94) indicates a broad effect on translation and transcripts with significantly differential changes in ribosome density are indicated as red and blue dots. C) Frequency distribution of the ratio of translational efficiency (TE=foot print density corrected for total mRNA abundance) in control and silvestrol treated samples (TESilvestrol/TEcontrol). Red and blue areas indicate groups of more (TE down) or less (TE up) affected mRNAs with a cut-off at p<0.03; a second cut-off is indicated light blue/red for p<0.13). D) Comparison of 5′UTRs lengths for TE down versus background genes. Mathematical density is scaled such that all values on the x-axis sum to 1; red: TE down, black: background genes, *: mean value. E) Prevalence of the indicated 5′UTR motifs among the TE down and background genes. F) A consensus 12-mer motif enriched in the TE down genes. G) Illustration of base-pair interactions in a predicted G-quadruplex based on the sequence motif. H) Enrichment of predicted 5′UTR G-quadruplex structures in the TE down gene set (* indicates p<0.05). I) Venn diagram indicating the overlap of genes containing 12-mer motifs and G-quadruplexes in TE down genes. J) Schematic of the NDFIP1 5′UTR showing a G-quadruplex region matching the 12-mer (GGC)4 motif.

FIG. 10 depicts ribosome profiling quality control data and effects on translation. A and B) Read counts by length of mapped sequence before and after filtering rRNA, linker reads, non-coding RNAs, short mapped sequences (“noisy” reads; see text and method for details). C and D) Read length frequency histograms and mapping analysis of ribosome footprint data after quality control filtering for vehicle treated cells (C) or silvestrol treated cells (D). E) Silvestrol induced changes in total RNA (log 2 Fold change RPKM) and ribosome protected RNA (RF). F) Histogram of all genes' ribosome footprint intensity (measured as unique read number per million per gene, RPM) for silvestrol and vehicle treated cells indicating silvestrol affected mRNAs were broadly distributed (see text for details). G) Mean fluorescence intensity of incorporated L-azidohomoalanine (AHA) in newly synthesized proteins in KOPTK1 cells treated with vehicle (DMSO), silvestrol (Silv. 25 nM), or Cycloheximide (CHX 100 nM) for the indicated time period. H) Polyribosome profiles of silvestrol (25 nM) or vehicle (DMSO) treated KOPT-K1 cells showing OD254 absorption across the ribosome containing fractions. I) Length comparison of 5′UTRs of TE up genes and a background gene set; *: mean J) Percentage of TE up genes and background genes containing the indicated sequence motifs; *: p<0.001. K) Consensus logos showing the three most significant 9-mer motifs enriched in TE down genes. The TE up genes do not have a motif. L) Venn diagram indicating the overlap between genes containing 9-mers and G-quadruplexes in TE down genes.

### Example 4

### Hallmarks of eIF4A-Dependent and Silvestrol-Sensitive Transcripts

5′UTR length has been implicated in translational control (Hay and Sonenberg, 2004), although a recent study found no effects of UTR length on mTORC1-dependent translation (Thoreen et al., 2012). Comparing the 5′UTR length across TE up, TE down, and background groups (as described in U.S. application Ser. No. 61/912,420, filed Dec. 5, 2013; and Wolfe et al., Nature. 2014 Sep. 4; 513(7516):65-70), it was observed that mRNAs with longer 5′UTRs were significantly enriched among the most silvestrol-sensitive mRNAs (TE down: mean UTR length 368 nucleotides; background: mean 250 nucleotides; p(Silvestrol vs. Control)=7.6×10−12 using two-sample Kolmogorov-Smirnov) (FIG. 3D). On the other hand, the TE up group showed no significant difference in 5′UTR length (TE up: 265 nucleotides; p(Silvestrol vs. Control)=0.165) (FIG. 10I).

Known translation regulatory elements were sought. For example, TOP sequences (cytidine in pos. 2 followed by 4-14 pyrimidines) (Meyuhas, 2000), TOP-like sequences (cytidine in pos. 1-4 and >5 pyrimidines) (Thoreen et al., 2012), internal ribosome entry sites (IRES) (Pelletier and Sonenberg, 1988), and pyrimidine rich translational elements (PRTEs) (Meyuhas, 2000). Comparing TE down and the background lists no predilection was found for TOP, TOP-like, PRTE, or IRES elements (FIG. 3E). On the other hand, the TE up group showed a significant enrichment for IRES elements and this is consistent with the dual-luciferase reporter assay and previous characterization of IRES dependent translation (Bordeleau et al., 2006) (FIG. 10J; see also FIG. 2A, FIG. 9A).

Next it was sought to identify a sequence motif that might confer eIF4A dependence. The DREME algorithm was used to look for significantly enriched sequences in the TE down and TE up groups compared to the background list (as described in U.S. application Ser. No. 61/912,420, filed Dec. 5, 2013; and Wolfe et al., Nature. 2014 Sep. 4; 513(7516):65-70) (Bailey, 2011). No motif was found in the TE up group of mRNAs. However, the analysis revealed a 12-mer (GGC)₄ motif that was significantly over represented among the TE down transcripts and present in 94 out of 220 genes (p<2.2×10-16) (FIG. 3F, Table 3A). In addition, 14 shorter 9-mer motifs were found that were similarly enriched in the TE down group and occurred in 177 of 220 genes (p<4.2ex10-15) (Figure S3K, Table 3B). P-values were computed using a one-sided binomial test while accounting for the different 5′ UTR lengths. A significantly higher than expected number of motif occurrences than explained by the larger UTR lengths were found (p<2.2×10-16).

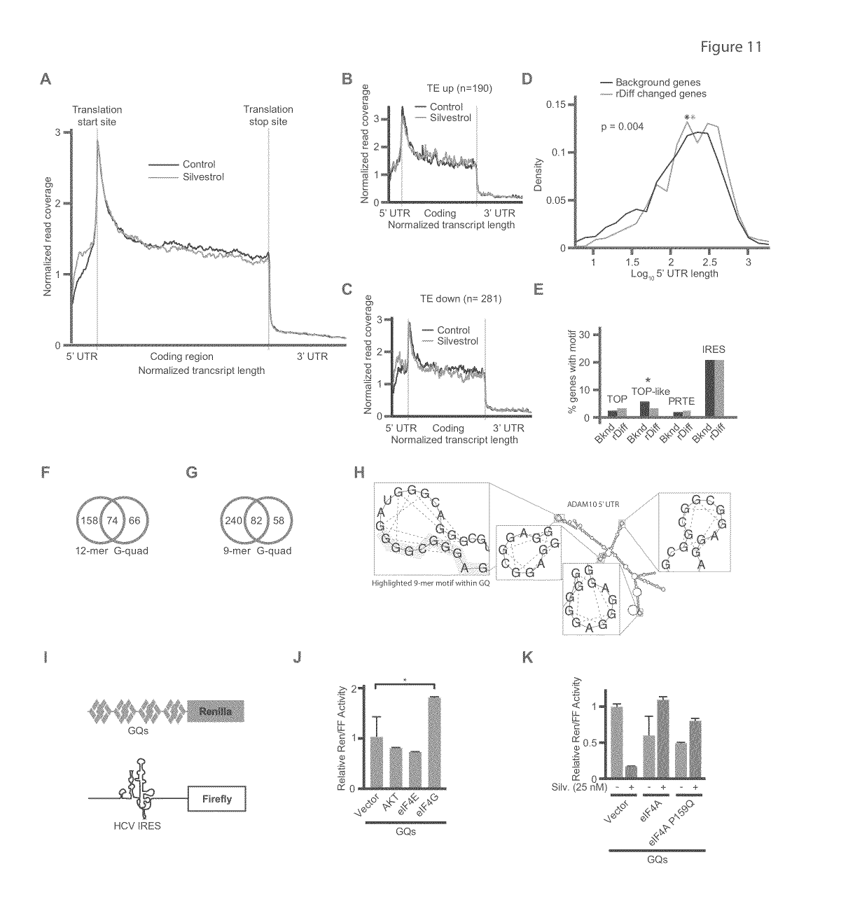

Whether silvestrol-sensitive mRNAs might have specific structural features that set them apart from less affected transcripts was considered. Using the program RNAfold (http://rna.tbi.univie.ac.at/cgi-bin/RNAfold.cgi) the background, TE up, and TE down genes were modeled and a striking enrichment was observed for G-quadruplex structures among the TE down genes (p=2×10-11) (FIG. 3G-I). Specifically, 79 of the 220 TE down transcripts with annotated 5′UTRs harbored at least one G-quadruplex. Moreover, in 48 out of 79 transcripts, G-quadruplex structures perfectly co-localized with the (GGC)4 12-mer sequence motif (FIG. 31, Table 3C). Briefly, G-quadruplex structures are based on non-Watson-Crick interactions between at least four paired guanine nucleotides that align in different planes and are connected by at least one linker nucleotide (FIG. 3F/G) (Bugaut and Balasubramanian, 2012). Most often two guanines were observed separated by an intervening cytosine and sometimes an adenine (FIG. 3F). The NDFIP1 5′UTR exemplifies the folding and typical pattern observed, with more than one G-quadruplex and one that directly matches the 12-mer (GGC)4 motif and a second larger structure that is formed by a longer nucleotide sequence including elements that are similar although not identical to the canonical (GGC)4 motif (FIG. 3J). While a 9-mer sequence is insufficient to form the structure, these motifs are found to be highly enriched within G-quadruplex structures. For example, the most common 9-mer motif overlapped with G-quadruplex structures in 45% (p=2.2×10-6), the second most common in 21% (p=1.4×10-10). In these instances, nucleotides adjacent to the motif completed the structure (Table 4, illustrated in FIG. 11H). Together, these analyses indicate that long 5′UTRs and a (GGC)4 motif or highly similar sequence patterns that can form G-quadruplex structures are the hallmarks of eIF4A-dependent and silvestrol-sensitive translation.

FIG. 11 shows the analysis of genes with differential ribosomal distribution (rDiff positive set). A) Representation of ribosome coverage for all 847 transcripts with significant changes in distribution between silvestrol (red) and vehicle (black); corresponding to the rDiff positive gene list. Both RF coverage and transcript length are normalized for comparison; translation start and stop sites are indicated by blue lines. B-C) Ribosomal distribution plots as in A showing how silvestrol affects ribosome distribution in all TE up genes (B) and all TE down genes (C). D) Length comparison of 5′UTRs of genes with significantly altered ribosomal distribution (rDiff positive: red) and background genes (black); *: mean value. E) Percentage of rDiff positive genes and background genes containing the indicated sequence motifs.* indicates p<0.05. F-G) Venn diagrams indicating overlap between genes containing 12-mers (F) or 9-mers (G) and G-quadruplexes in rDiff positive genes. H) Schematic of the ADAM10 5′UTR with G-quadruplexes and indicating an example of a 9-mer sequence contributing to the G-quadruplex. I) Diagram of Renilla luciferase expressed from four G-quadruplexes in tandem (GQs, red) and Firefly luciferase expressed from the HCV IRES (white). J) Relative amounts of Renilla luciferase expressed from the GQ construct in 3T3 cells and normalized to IRES/Firefly with either empty vector or the indicated genes (* p<0.05). K) Relative amounts of Renilla luciferase expressed from the GQ construct in 3T3 cells and normalized to IRES/Firefly with either empty vector or the indicated genes, treated with silvestrol (25 nM) for 24 hours.

### Example 5

### Silvestrol Causes an Accumulation of RFs in the 5′UTR of Sensitive Transcripts

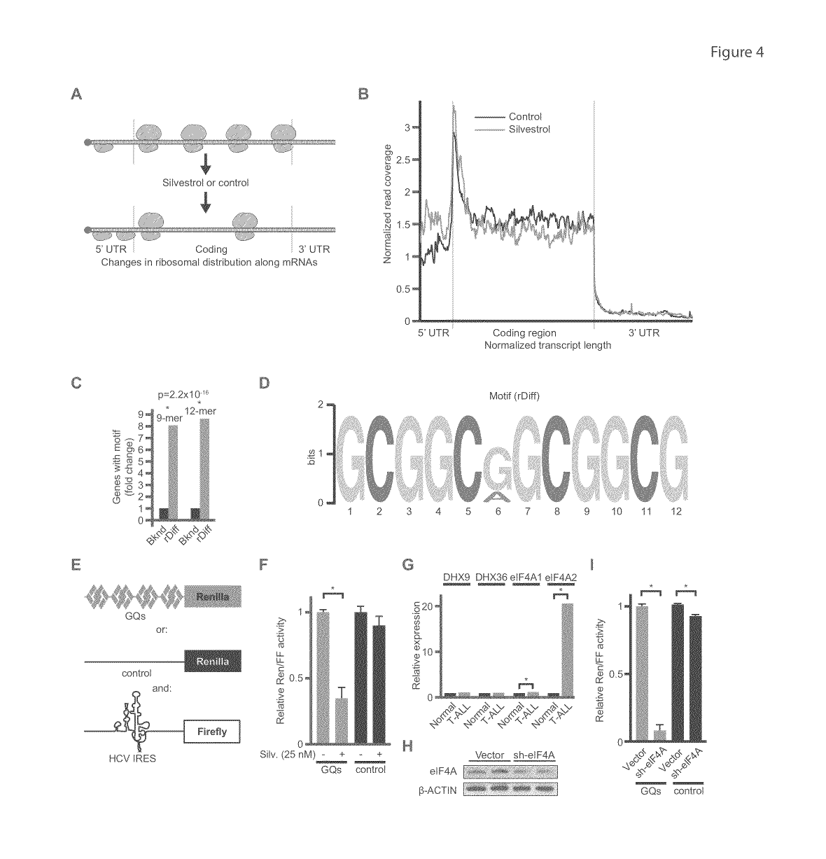

Next, the distribution of ribosomes was examined along the transcript as this might provide an additional indication of eIF4A sensitive translation (FIG. 4A). Note that the footprinting methodology provides exact sequence and positional information for each RF, and using the rDiff algorithm significant changes in read density were identified across the length of any given transcript (see method) (Drewe et al., 2013). A p-value cutoff of p<0.001 was used to identify a group (the rDiff positive set) of 847 protein-coding transcripts (641 with an annotated 5′UTR) that showed the most significant change in RF distribution (Table 5). These transcripts showed an accumulation in the 5′UTR and corresponding loss of coverage across the coding sequence. This silvestrol effect is most pronounced for the 62 genes that show decreased TE (TE down) and significant change in rDiff whereas it is absent in the TE up group (FIG. 4B, FIG. 11A-C, Table 6).

Similar to the TE down group an enrichment of longer 5′UTR in the rDiff positive set was found (rDiff pos.: n=641; mean length 271 nucleotides; Background (rDiff negative=no significant change): n=976, mean UTR length: 230 nucleotides; p=0.004) (Figure S4D). No significant enrichment for TOP, PRTE, or IRES elements was detected, however there was a small and significant drop in TOP-like sequences (Figure S4E). The DREME analysis for sequence motifs identified a significant enrichment for a 12-mer and three 9-mer motifs among rDiff positive genes (p=2.2×10-16) (FIG. 4C/D, Table 4, Table 7A/B). Among 641 genes in the rDiff group, the 12-mer motif occurred in 232, and an additional three 9-mer motifs were found in 322 genes. Notably, the motifs were nearly identical to the TE down motif (FIG. 3). Again, the 12-mer and 9-mer motifs co-localized to the majority of predicted G-quadruplexes observed in the rDiff positive gene set and this is illustrated with the ADAM10 5′UTR (FIG. 11F-H, Table 4, Table 7C). Hence, two different analyses—translation efficiency and RF distribution—point to the exact same patterns in eIF4A-sensitive transcripts: longer 5′UTRs with variations on the theme of a (GGC)4 sequence capable of G-quadruplex formation.

Next, directly testing the translational effect of the 12-mer sequence motif was sought. Briefly, a luciferase reporter system was constructed to directly compare four 12-mer motifs in tandem reflecting the common occurrence of multiple motifs in sensitive mRNAs (GQ construct) to a random sequence of equal length and GC content (control construct) and using an IRES-driven firefly luciferase as an internal control (FIG. 4E). First, treatment with silvestrol (25 nM) reduced the translation of the GQ construct and did not reduce the translation of the control luciferase. The RNA helicases DHX9 and DHX36 have been implicated in resolving G-quadruplex structures (Booy et al., 2012; Chakraborty and Grosse, 2011), however predominant expression was found of eIF4A in T-ALL (FIG. 4G) (Van Vlierberghe et al., 2011). Further direct testing was done of the effect of RNAi-mediated eIF4A knockdown in the same assay and a striking decrease in the translation from the GQ reporter observed, with little effect on the control sequence (FIG. 4H/I). Whether upstream activators or translation factors could enhance translation of the GQ construct was explored. It was found that only eIF4G could increase translation and that neither Akt, eIF4E, or eIF4A expression were sufficient (FIG. 11I/J). This is consistent with the notion that eIF4A levels are not limiting under physiological conditions and that additional factors (e.g. eIF4G) are needed for full eIF4A activation (Feoktistova et al., 2013; Oberer et al., 2005; Ozes et al., 2011). However, upon silvestrol treatment, it was observed that increased expression of wild type eIF4A or an RNA-binding site mutant protein (P159Q—homologous to S. cervesiae P147Q (Sadlish et al., 2013)) could render translation of the GQ reporter construct insensitive to silvestrol (FIG. 11K). Hence, pharmacologic and genetic evidence indicates that the 12-mer motif enriched in silvestrol sensitive transcripts requires eIF4A for translation.

FIG. 4 shows that silvestrol affects ribosome distribution in a subset of mRNAs. A) Diagram of differential ribosomal distribution along the length of a transcript. B) Representation of ribosome coverage for 62 TE down transcripts with changes in ribosomal distribution (rDiff positive); silvestrol (red), vehicle (black). RF coverage and transcript length are normalized for comparison, translation start and stop sites are indicated. C) rDiff positive genes were enriched for 9-mer and 12-mer motifs compared to background genes (* indicates p<0.05). D) The rDiff positive genes are enriched for the indicated 12-mer GC-rich consensus motif. E) Schematic of constructs expressing the indicated luciferase with 5′UTRs containing four 12-mer motifs in tandem (GQs, red), a random sequence matched for length and GC content (control, black), and the HCV IRES (white). F) Relative amounts of Renilla luciferase (normalized to Firefly) expressed from the GQs (red bars) or control construct (black bars), treated as indicated for 24 hours (* indicates p<0.05). G) Analysis of mRNA expression from (Van Vlierberghe et al., 2011) of the indicated RNA helicases in normal T-cells and T-ALL cells (* indicates p<0.05). H) Immunoblots of lysates from 3T3 cells with empty vector or sh-eIF4A and probed as indicated. I) Relative amounts of Renilla luciferase (normalized to Firefly) expressed from the GQs (red bars) or control construct (black bars), with empty vector or sh-eIF4A (* indicates p<0.05).

### Example 6

### Transcripts Affected by Silvestrol

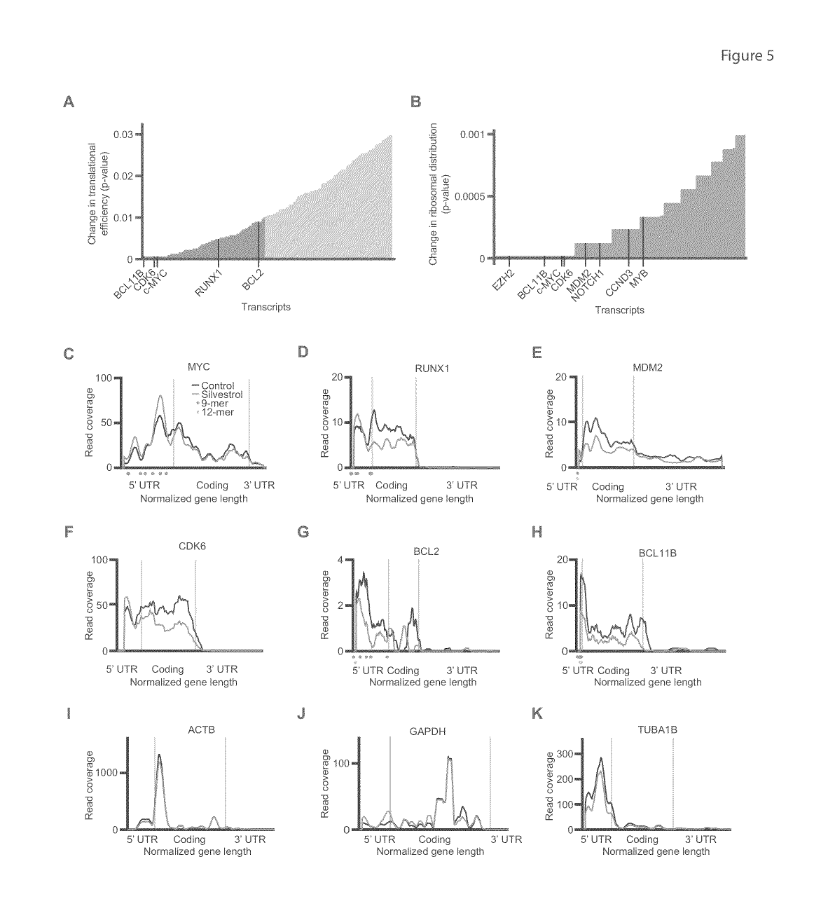

The most silvestrol sensitive transcripts in the TE down group and the rDiff positive set include many genes with known roles in T-ALL (FIG. 5A/B). Categorization by gene ontology reveals a preponderance of transcription factors, many oncogenes, but also potential tumor suppressors (Figure S5A/5B). Sub-grouping of TE down genes by 5′UTR features (12-mer, 9-mer motif, and G-quadruplex structures) illustrates how sometimes multiple features occur in the same transcripts (Figure S5C-E). Exploring individual RF distribution graphs (normalized for mean RF count and gene length) illustrates recurrent patterns and also variations. For example, the c-MYC transcript (TE: p=1.3×10-4; rDiff: p=3×10-8) harbors six 9-mer motifs in its 5′UTR which correspond to peaks in RF density (FIG. 5C). Similarly, MDM2 (TE: p=0.94; rDIff: p=4.9×10-7) and RUNX1 (TE: p=4×10-3; rDIff: p=5.2×10-3) harbor multiple motifs and show a 5′UTR RF accumulation and drop across the coding region (FIG. 5D/E). CDK6 (TE: p=4×10-8; rDIff: p=4.7×10-5) shows the same pattern, and while the DREME analysis did not retrieve the typical motif it might harbor an alternate element (FIG. 5F). BCL2 (TE: p=8.6×10-3; rDIff: p=6.0×10-1), and BCL11B (TE: p=5.4×10-10; rDIff: p=1.0×10-8) have multiple 9-mer and 12-mer motifs and these transcripts show a drop in 5′UTR RF counts and peak shifts in their exonic regions (FIG. 5G/H). Several housekeeping genes have no recognizable motif and in particular actin shows no detectable effect of silvestrol on RF patterns (FIG. 5I-K).

FIG. 5 shows that many cancer genes are differentially affected by silvestrol. A) TE down genes in silvestrol treated KOPT-K1 ranked by translational efficiency (red, up to p=0.01). B) rDiff positive genes ranked by changes in ribosome distribution (up to p=0.001). C-K) Distribution of ribosomal footprints for the indicated genes. Silvestrol: Red; Vehicle: black; purple dots: 9-mer motifs; blue dots 12-mer motif.

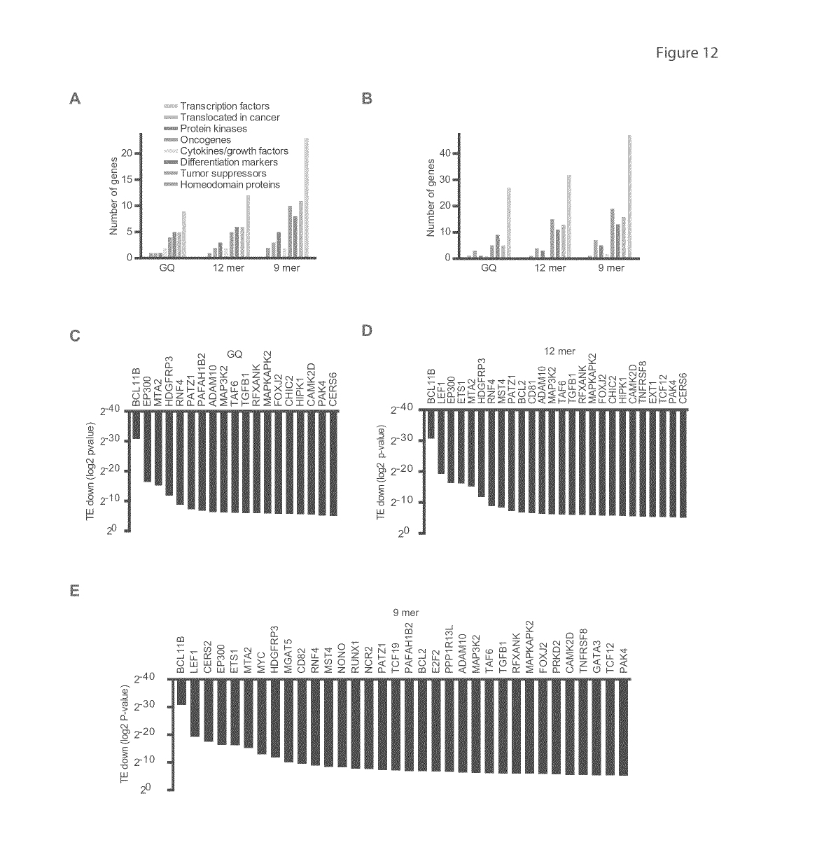

FIG. 12 shows that gene ontology analysis of silvestrol sensitive genes. A) Number of genes in TE down group with G-quadruplex, 12-mer and 9-mer motif in the indicated gene family classifications. B) Number of genes in rDiff positive group with G-quadruplex, 12-mer and 9-mer motif in the indicated gene family classifications. C-E) Representative transcription factors and oncogenes with G-quadruplex (C), 12-mer (D), and 9-mer (E) motif in TE down genes, ranked by significant changes in translational efficiency.

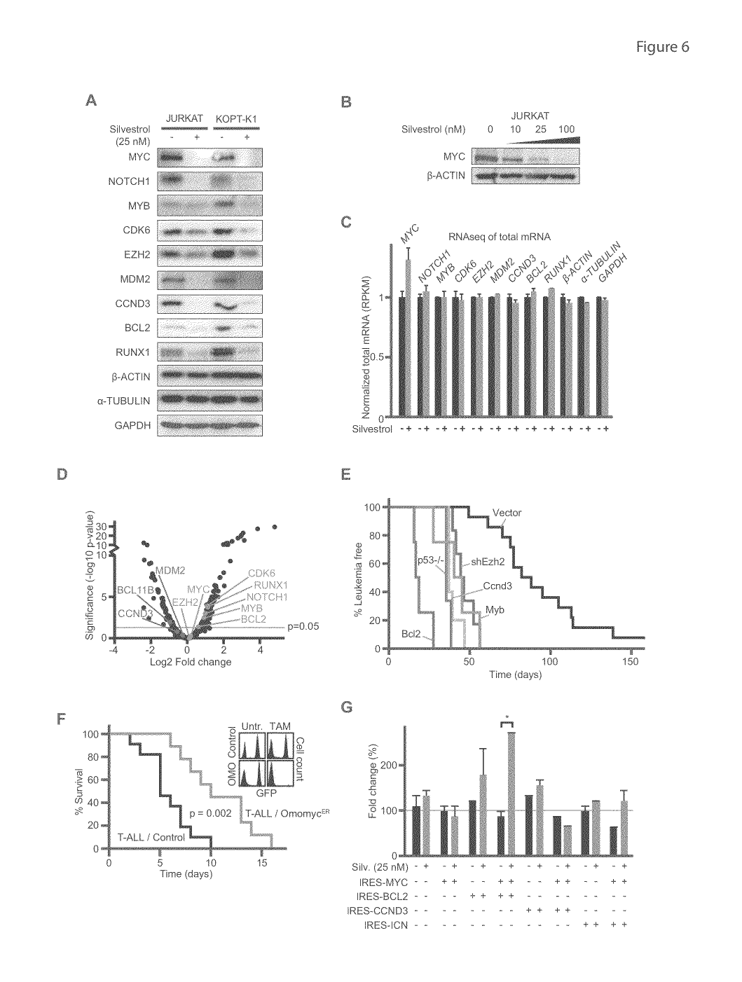

Given the complexity of the RF data analysis, it was important to directly confirm loss of expression for at least some of these proteins. Briefly, immunoblots on JURKAT and KOPT-K1 cells treated with silvestrol (25 nM) and loaded with equal amounts of total protein confirmed dramatic loss of MYC, NOTCH1, BCL2, and CCND3 proteins (FIG. 6A). The effect on MYC was especially striking, it was dose dependent (FIG. 6B), lasted for 48 h, and was also achieved in xenografts in vivo (Figure S6A-B). Others whose expression was somewhat less decreased included MYB, CDK6, EZH2, and RUNX1/AML1. As expected, ACTIN, TUBULIN, and GAPDH were not affected (FIG. 6A). The same result was confirmed with the silvestrol analogue (CR) (Figure S6C). The effects were indeed posttranscriptional and no decrease was observed in the corresponding mRNA expression (FIG. 6C). The small and significant increase in MYC mRNA levels might be consistent with prior reports of a MYC auto-regulatory mechanism (Penn et al., 1990a; Penn et al., 1990b) (FIG. 6C).

Genomic studies have implicated many silvestrol-sensitive genes in T-ALL and other cancers. For example oncogenic mutations of NOTCH (Weng et al., 2004), increased CDK6/CCND3 (Sawai et al., 2012), and amplifications of MYB (Lahortiga et al., 2007) have been reported in T-ALL. Similarly, a brief survey of mRNA expression using RNAseq on 9 primary T-ALL samples compared to 4 T-cell samples confirms increased expression of NOTCH, MYB, CDK6, and BCL2 in T-ALL (FIG. 6D). Increased MYC activity has also been implicated in T-ALL (Gutierrez et al., 2011a; Palomero et al., 2006), and it was observed abundant MYC protein expression in ˜70% of T-ALLs by immunohistochemistry (Figure S6D). A direct tested was conducted on some of these genes for their role in T-ALL using the same mouse model described above (FIG. 1A). Briefly, Myb (n=4, p<0.0001), a mutant Ccnd3 (T283A) (n=5; p<0.0001), Bcl2 (n=4, p<0.0001), and p53 loss (as a surrogate for Mdm2; n=3; p<0.0001) accelerated leukemogenesis in the Notch context in vivo (FIG. 6E). However, silvestrol also affected candidate tumor suppressors in T-ALL, for example BCL11b (Gutierrez et al., 2011b), RUNX1 (Della Gatta et al., 2012; Giambra et al., 2012), and EZH2 (Ntziachristos et al., 2012). Direct testing of the effect of EZH2 knockdown (n=6, p<0.0001) demonstrated that EZH2 indeed acts as a tumor suppressor in T-ALL in vivo (FIG. 6E).

Given the pleiotropic effects of eIF4A inhibition it was considered which of its target genes may account for the drug's anti-leukemia effect. The MYC oncogene is a first candidate, because of silvestrol's powerful effects on MYC levels and its known oncogenic role in this cancer (Gutierrez et al., 2011a; Palomero et al., 2006). Moreover, genetic MYC blockade using the tamoxifen-inducible OmomycER allele (Soucek et al., 2008) readily induces cell death and clears T-ALL cells from the marrow leading to an extended survival in leukemic animals (nOMO=9, ncontrol=10; p=0.002) (FIG. 6F and inset). However, IRES-driven expression of MYC alone was unable to protect murine T-ALL cells from silvestrol. Similarly, IRES-driven expression of additional oncogenes including BCL2, NOTCH1, CCND3 (T283A) was not protective. Instead, cells expressing both IRES-MYC and IRES-BCL2 were significantly selected upon silvestrol treatment (FIG. 6G). These results suggest that silvestrol acts by disrupting the production of multiple pro-oncogenic multiple factors that are required to maintain the leukemia.

FIG. 6 depicts validation of selected silvestrol targets. A) Immunoblots of lysates from human T-ALL lines treated with silvestrol (25 nM) and probed as indicated. B) Immunoblots of lysates from JURKAT cells treated with escalating doses of silvestrol and probed as indicated. C) mRNA levels for the corresponding genes treated with vehicle (DMSO, black) or silvestrol (red, 25 nM); D) Volcano plot of mRNA expression changes of all TE Down and rDiff genes in T-ALL samples (n=9) compared to normal thymocytes (n=4). E) Kaplan-Meier analysis showing time to leukemia development (as in FIG. 1B). Wild-type HPCs transduced with NOTCH1-ICN and empty vector (black, n=14), Myb (red, n=4), Ccnd3 T283A (orange, n=5), or shEzh2 (orange, n=10), vavP-Bcl2 (brown, n=4), p53−/− (green, n=3). F) Effect of OmoMYCER activation on survival of leukemic animals; (d=0 represents start of therapy/tamoxifen (TAM) administration). Inset: clearance of GFP expressing murine T-ALL cells from the marrow upon OmomycER activation (Untr: untreated, TAM: tamoxifen). G) Competition experiment (as in FIG. 1C/D) showing the percentage of each starting GFP positive population of murine T-ALL cells partially transduced with the indicated constructs and treated with silvestrol (* indicates p<0.05).

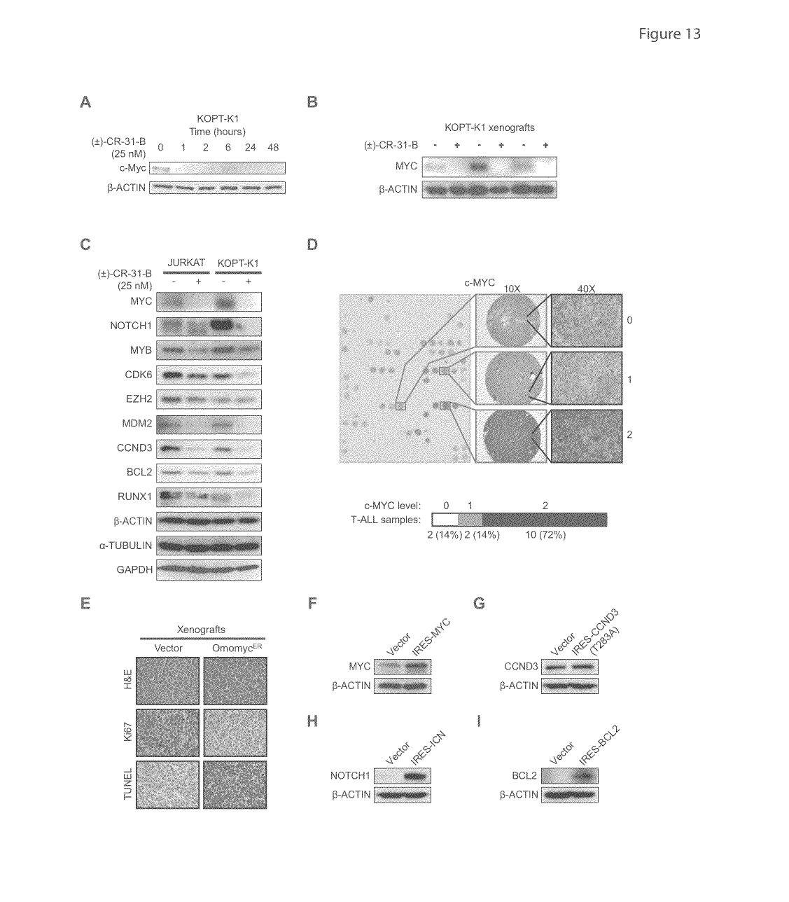

FIG. 13 depicts the relative contribution of MYC and other silvestrol targets in T-ALL. A) Time course analysis of protein expression in KOPT-K1 cells treated with CR (25 nM) for the indicated number of hours. B) Immunoblot on CR or vehicle treated KOPT-K1 xenografts, probed as indicated. C) Immunoblots of lysates from human T-ALL lines treated with CR (25 nM, 24H) and probed as indicated. D) Representative section of tissue microarrays (TMA) representing 14 human T-ALLs and stained for MYC (lower panel) Scoring of the T-ALL MYC TMA: ‘0’=0%-25% positive cells, ‘1’=25%-75% positive cells, ‘2’=75%-100% positive cells. Normal spleen, kidney, and lymph node negative controls were present on the same slides. E) Histology from tamoxifen treated (50 mg/kg) xenografted T-ALL tumors expressing a control vector or OmomycER and stained as indicated. F-I) Immunoblots of lysates from murine T-ALL cells expressing either vector control or IRES-MYC (F), IRES-CCND3 T283A (G), IRES-ICN (H), or IRES-BCL2 (I) and probed as indicated.

### Example 7

### Study of G-Quadruplex Unwinding Mechanisms

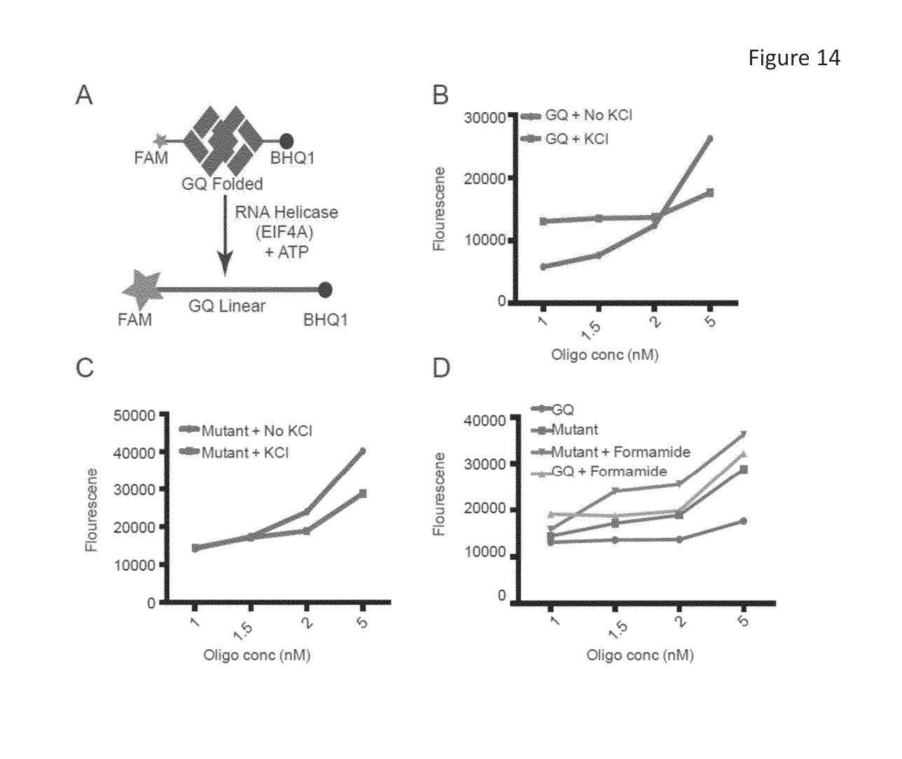

A FRET-based assay was set up for measuring the effect of RNA helicases on G-quadruplex unwinding, screening proteins that can unwind G-quadruplexes and identify small molecules that stabilize the G-quadruplex structure. An RNA oligonucleotide (1XTEDownMotif 5′-UAGAA ACUAC GGCGG CGGCG GAAUC GUAGA; SEQ ID NO:65) containing the G-quadruplex motif was labeled with fluorophore FAM on the 5′ end and quencher BHQ1 on the 3′end. When folded, the labeled GQ RNA oligonucleotide will exhibit minimum baseline fluorescence. Addition of specific RNA helicase such as EIF4A with ATP and/or small molecules would result in unwinding and increase in fluorescence signal measured in real time, as shown in FIG. 14A.

FIG. 14B shows the optimization of fluorescence quenching assay using labeled RNA G-quadruplex oligonucleotide. Fluorescence was measured as function of concentration using G-quadruplex RNA with or without KCl. Without KCl fluorescence intensity increases as a function of concentration while in the presence of KCl it remains stable, suggesting the formation of a stable G-quadruplex structure in the presence of KCl.

Fluorescence measured as function of concentration using a mutant RNA (1XMutant; 5′-UAGACCCUGCAACGUCAGCGUAGUCGUAGC; SEQ ID NO:66) with or without KCl is shown in FIG. 14C. Fluorescence intensity increase as a function of concentration irrespective of KCl suggesting no particular secondary structure present in the mutant RNA oligonucleotide.

In FIG. 14D, the G-quadruplex versus mutant RNA oligonucleotide were compared using the fluorescence quenching assay. The fluorescence intensity of the G-quadruplex RNA remains stable and lower compared to the mutant RNA oligonucleotide. Mutant RNA shows an increase in fluorescence intensity as a function of concentration. Chemical unwinding using formamide results in increase of fluorescence intensity of both G-quadruplex and mutant RNA oligonucleotide.

This assay can therefore be used for the aforementioned purpose as well as various other purposes such as but not limited to 1) measuring the effect of known RNA helicases such as eIF4A, DHX9 or DHX36 on G-quadruplex unwinding; 2) investigating the effect of other cofactors/inhibitors required for eIF4A activity; 3) a screening method to identify other proteins that can unwind G-quadruplexes; and 4) identifying and establishing the effect of small molecules that stabilize the G-quadruplex structure.

### Example 8

### Sensitivity of Cancers to Silvestrol

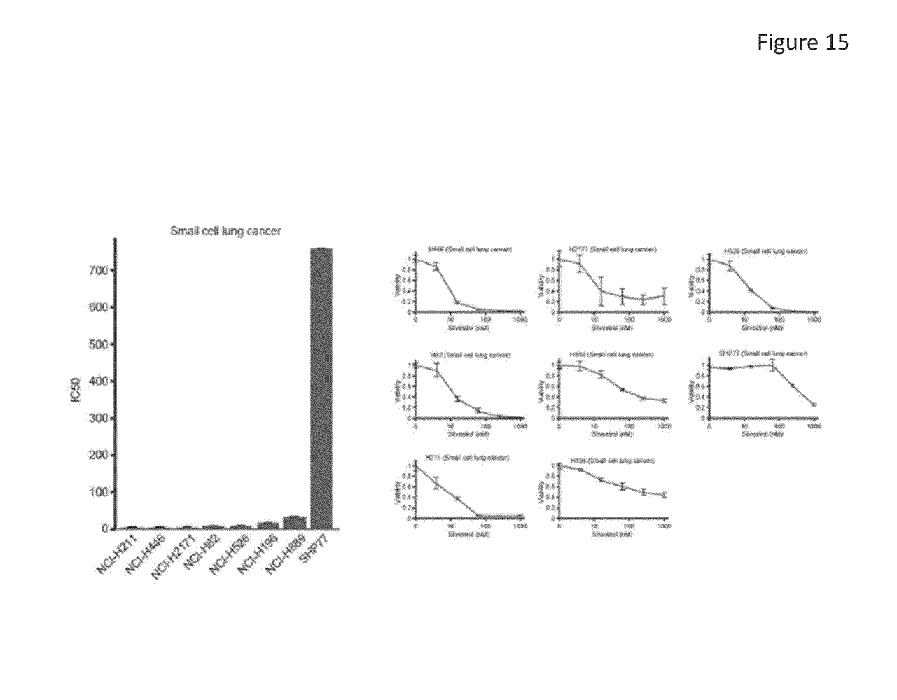

The IC50 of silvestrol in several small cell lung cancer lines was evaluated. As shown in FIG. 15, low IC50s were observed in cell lines NCI-H211, NCI-H446, NCI-H2171, NCI-H82, NCI-H526, NCI-H196 and NCI-H889, indicating high sensitivity to silvestrol. The IC50 values are shown in the left figure and the individual viability curves are shown at the right.

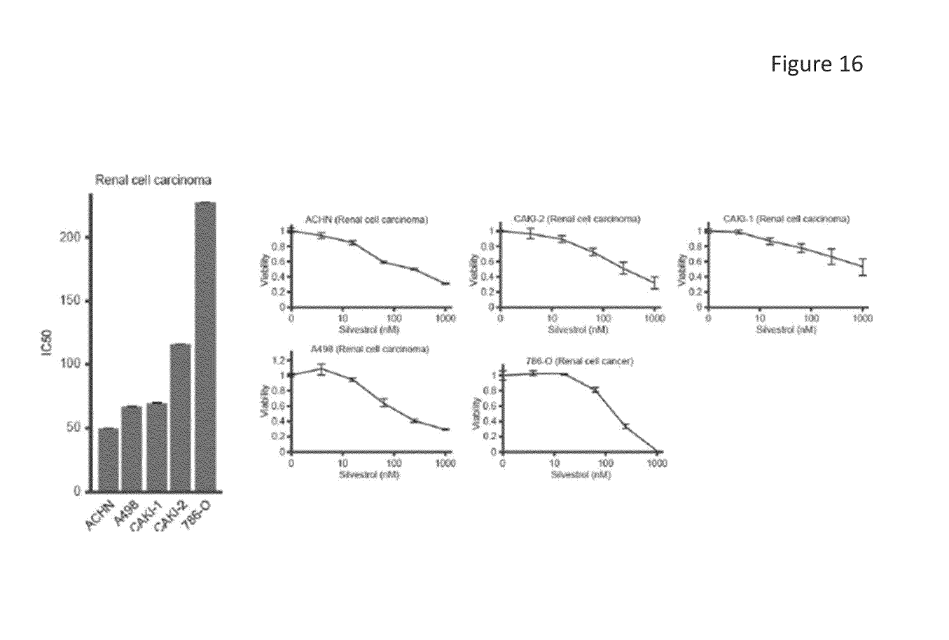

A range of sensitivities from renal carcinoma lines ACHN, A498, CAKI-1, CAKI-2 to 786-O was demonstrated, as shown in FIG. 16.

In addition, IC50s of 2 to 20 nM have been obtained with neuroblastoma cell lines SKNAS, CLBGA, IMR32 and N206. Pancreatic cancer line PANC-1 show sensitivity to 20 nM silvestrol and a loss of KRAS expression.

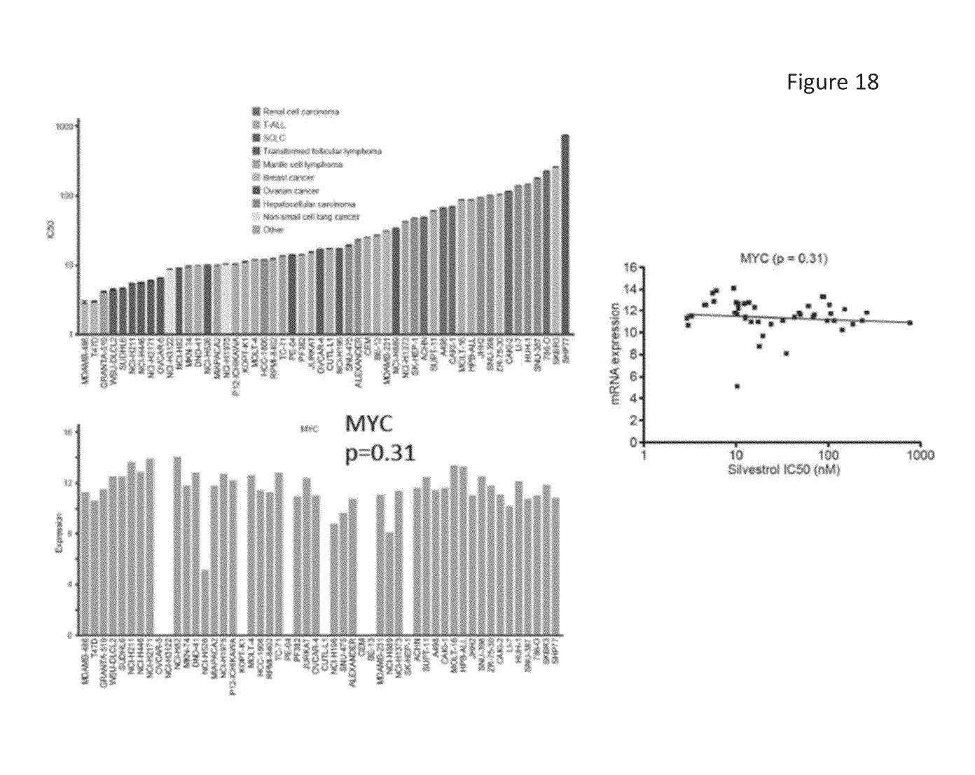

In addition to the renal cell carcinoma and small cell lung cancer lines mentioned above, about 60 cancer cell lines were evaluated for silvestrol sensitivity as shown in FIG. 17. Cancers including T-ALL, transformed follicular lymphoma, mantel cell lymphoma, breast cancer, ovarian cancer, hepatocellular carcinoma, and non-small cell lung cancer, as well as gastric cancer, pancreatic carcinoma, Ewing sarcoma and lung adenocarcinoma. FIG. 18, lower left panel, shows that MYC expression is not correlated with silvestrol sensitivity, indicating that MYC expression alone is not predictive of potential sensitivity of a tumor to silvestrol or other eIF4A inhibitor compounds as described herein, and indicates that the predictors of silvestrol sensitivity as described herein with the exclusion of MYC expression are useful for determining whether a patient's cancer will be sensitive to silvestrol.

### Example 9

### The Reporter Assay Determines Activity of Hippuristanol and Pateamine A

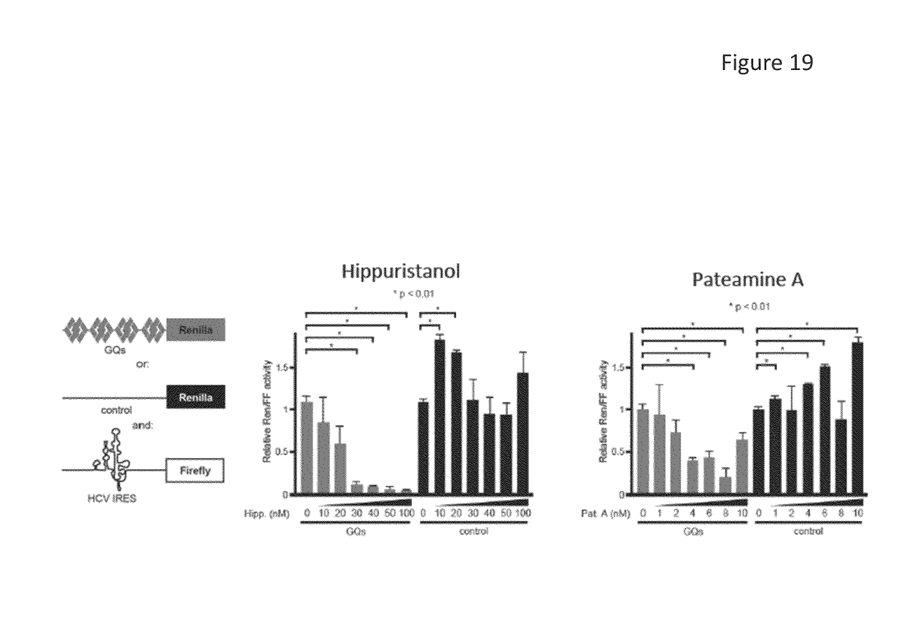

Using the dual-luciferase reporter assay described above, where renilla and firefly luciferase are either capped or under control of an internal ribosomal entry site (IRES) element, both hippuristanol and pateamine A were shown to preferentially block cap-dependent over IRES-dependent translation (FIG. 19).

### Example 10

### In Vitro Data on Silvestrol in Small Cell Lung Cancer

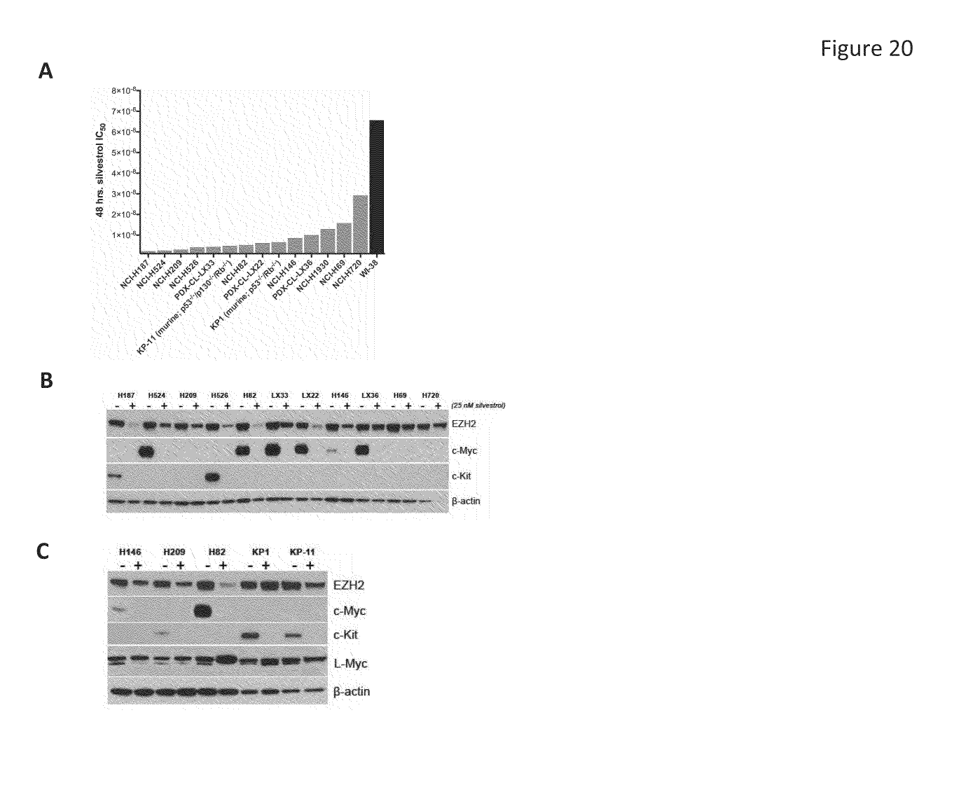

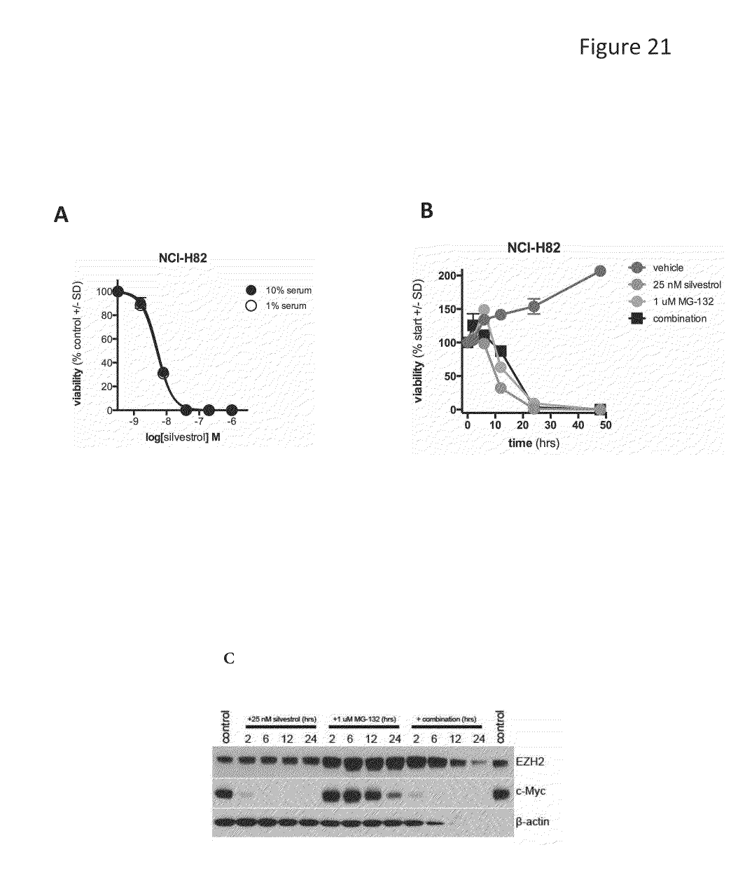

The in vitro activity of silvestrol was evaluated against a panel of small cell lung cancer cell lines, shown in FIG. 20A. In these cell lines, the effect of silvestrol on key target proteins MYC, EZH2 and cKit were determined (FIG. 20B, C). FIG. 21A shows the effect of silvestrol on H82 small cell lung cancer cells in the presence and absence of serum. FIG. 21B shows the effect of silvestrol alone and in combination with a proteasome inhibitor MG-132 against H82 small cell lung cancer cells and in FIG. 21C, the effects on key target proteins EZH2 and MYC.

### Example 11

### Effectiveness of Silvestrol in Neuroblastoma Cell Lines In Vitro

The effect of silvestrol on several neuroblastoma cell lines was evaluated. The results are shown in the table below.

| Cells | Silvestrol (IC50 nM) | Description |
|---|---|---|
| BE(1)N | 2.52 | N-MYC |
| IMR32 | 2.86 | N_MYC |
| SKNCM | 20.21 | |
| BE(2)C | 47.83 | N-MYC |

### Example 12

### Identification of G-Quadruplexes in KRAS

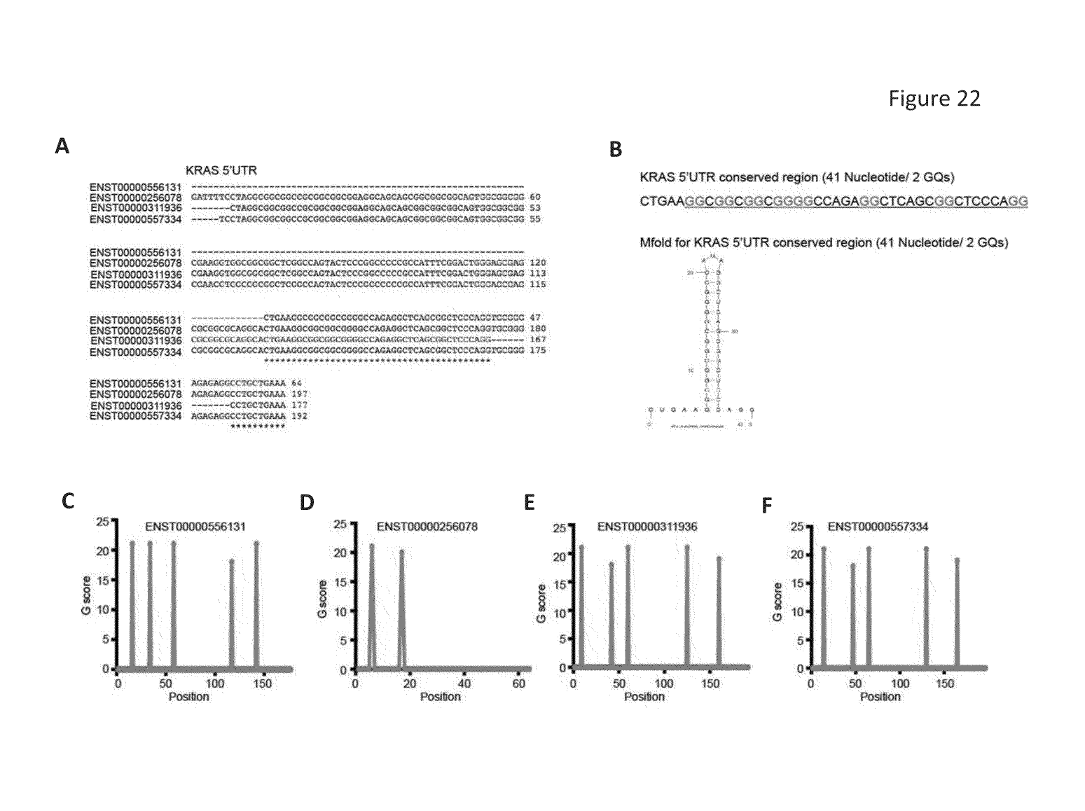

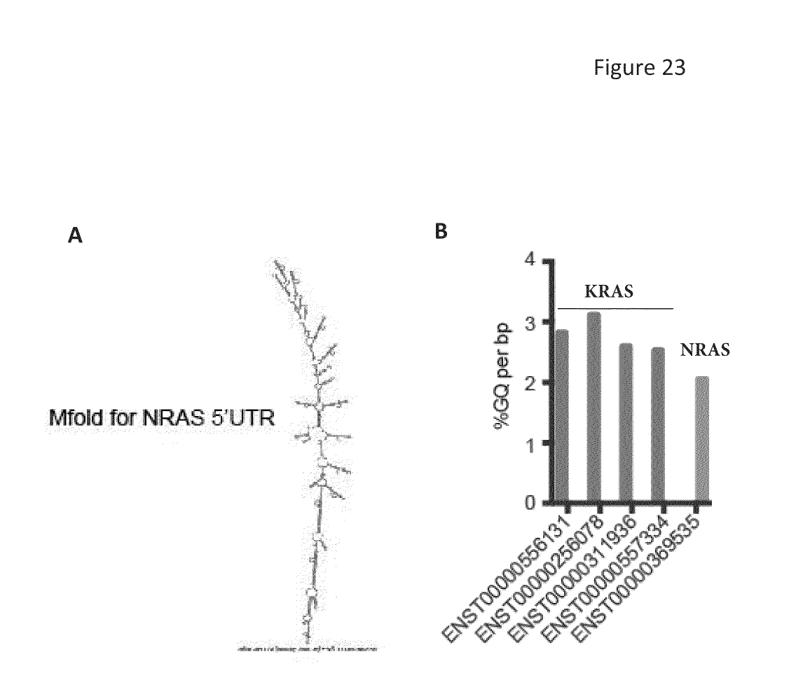

As shown in the four annotated transcripts of KRAS in FIG. 22A with different and overlapping 5′UTRs, the minimal/common region of the 5′UTR has two G-quadruplexes (FIG. 22B). They have at least two and up to five G-quadruplex structures (FIG. 22C-F). FIG. 23A shows the Mfold structure of the NRAS 5′UTR, and FIG. 23B compares the KRAS and NRAS transcripts showing a similar density of G-quadruplexes, identifying KRAS as a potential silvestrol target.

### Example 13

### Effect of Silvestrol on KRAS Protein Levels in PANC1 Cells In Vitro

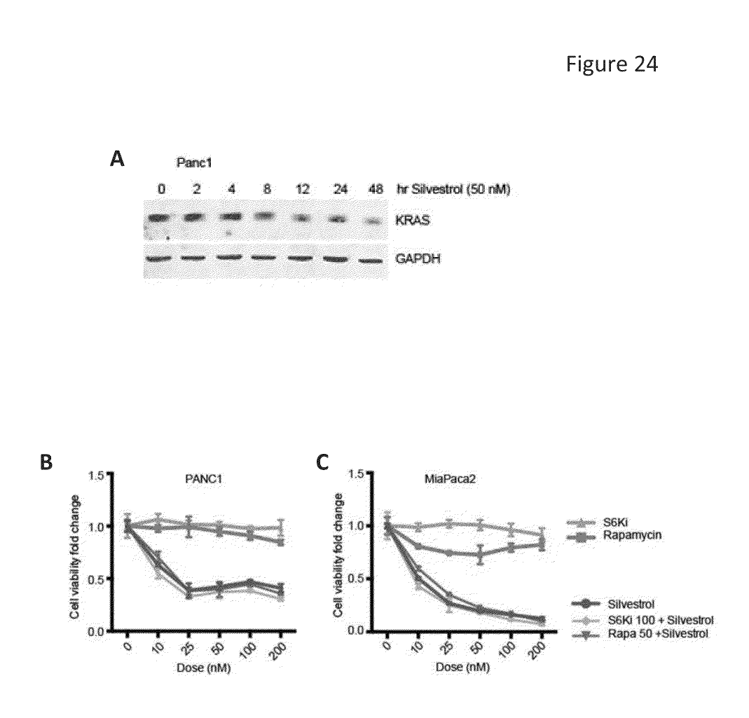

Similarly to that described in Example 8, silvestrol at 50 nM shows a loss of KRAS expression (FIG. 24A). KRAS has a long T_{1/2} compared to MYC, and therefore the effect is less pronounced. The in vitro activity of an S6 kinase inhibitor, rapamycin, silvestrol and combinations on PNAC1 and MiaPaca2 cells are shown in FIGS. 24B and 24C, respectively.

### Example 14

### In Vivo Activity of Silvestrol Against MiaPaca2 Xenografts

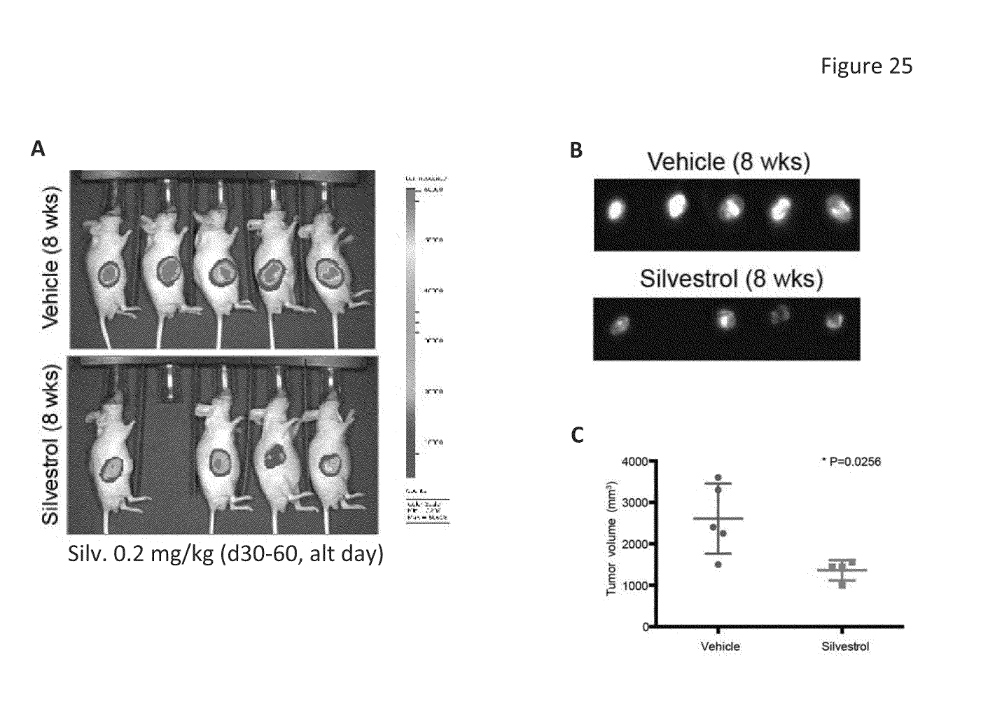

FIG. 25 shows the results in mice with MiaPaca2 xenografts when of 0.2 mg/kg silvestrol or vehicle was administered every other day from days 30-60 following tumor implantation. FIG. 25A shows the outcome using fluorescent imaging at 8 weeks; the excised tumors are shown in FIG. 25B; and the tumor volumes in FIG. 25C.

### Example 15

### In Vivo Activity of Silvestrol Against Small Cell Lung Cancer

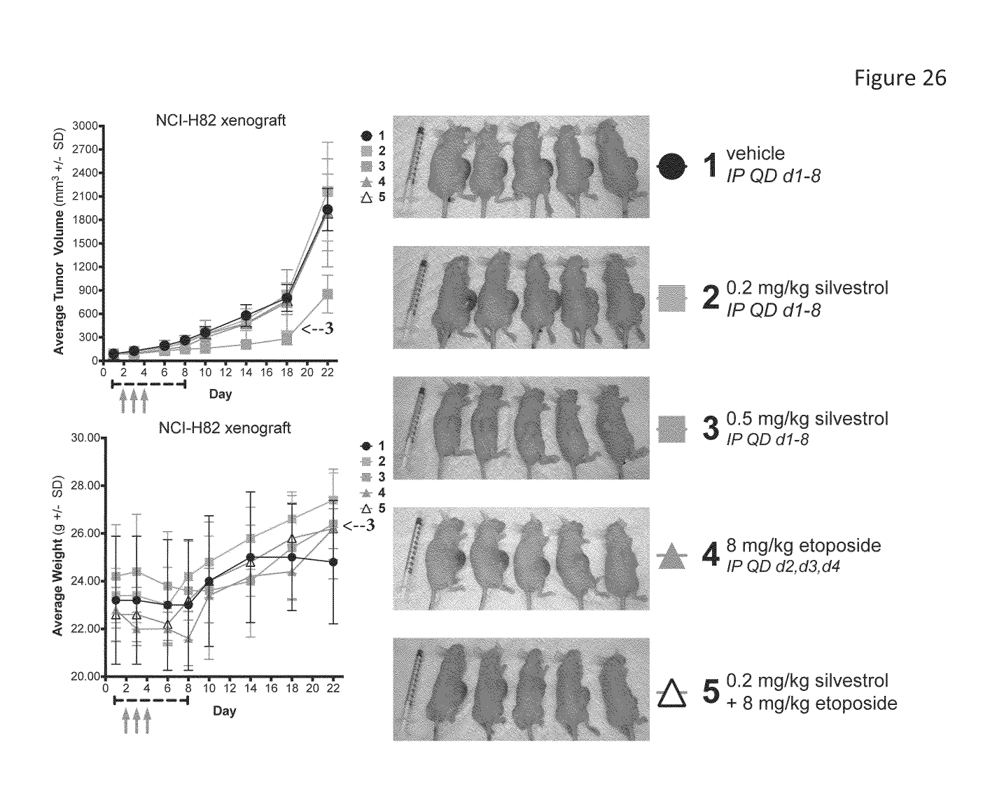

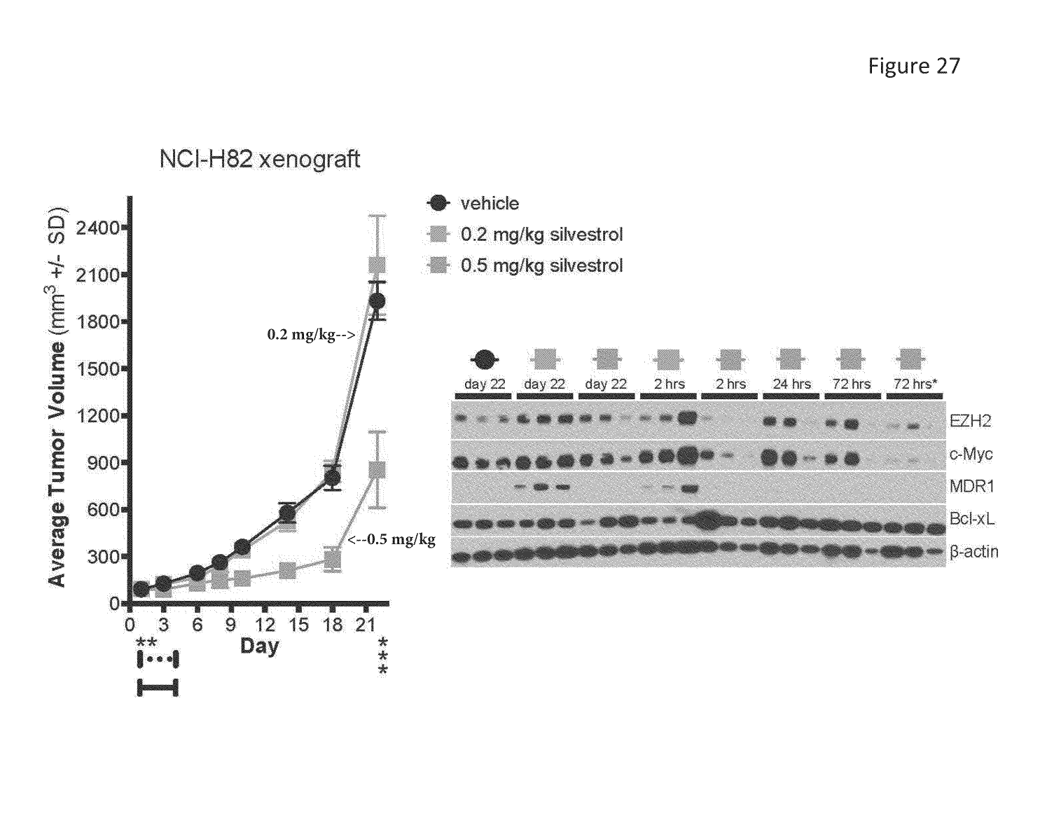

FIG. 26 shows the results of a xenograft experiment using small cell lung cancer line NCI-H82 and silvestrol administration. The average tumor volumes and animal weights are shown in the top left and bottom left graphs, respectively, and the data legends corresponding to the animal groups and treatments (dose level, dose regimen and compounds) in the photographs and their legends at the right. In FIG. 27, the results from the same experiment using 0.2 mg/kg and 0.5 mg/kg silvestrol are plotted in the left graph, and the effect on key target proteins at different time points on the right.

### REFERENCES

Alachkar, H., Santhanam, R., Harb, J. G., Lucas, D. M., Oaks, J. J., Hickey, C. J., Pan, L., Kinghorn, A. D., Caligiuri, M. A., Perrotti, D., et al. (2013). Silvestrol exhibits significant in vivo and in vitro antileukemic activities and inhibits FLT3 and miR-155 expressions in acute myeloid leukemia. Journal of hematology & oncology 6, 21.
Anders, S., Reyes, A., and Huber, W. (2012). Detecting differential usage of exons from RNA-seq data. Genome research 22, 2008-2017.
Bailey, T. L. (2011). DREME: motif discovery in transcription factor ChIP-seq data. Bioinformatics 27, 1653-1659.
Blagden, S. P., and Willis, A. E. (2011). The biological and therapeutic relevance of mRNA translation in cancer. Nature reviews Clinical oncology 8, 280-291.
Booy, E. P., Meier, M., Okun, N., Novakowski, S. K., Xiong, S., Stetefeld, J., and McKenna, S. A. (2012). The RNA helicase RHAU (DHX36) unwinds a G4-quadruplex in human telomerase RNA and promotes the formation of the P1 helix template boundary. Nucleic acids research 40, 4110-4124.
Bordeleau, M. E., Matthews, J., Wojnar, J. M., Lindqvist, L., Novac, O., Jankowsky, E., Sonenberg, N., Northcote, P., Teesdale-Spittle, P., and Pelletier, J. (2005). Stimulation of mammalian translation initiation factor eIF4A activity by a small molecule inhibitor of eukaryotic translation. Proceedings of the National Academy of Sciences of the United States of America 102, 10460-10465.
Bordeleau, M. E., Mori, A., Oberer, M., Lindqvist, L., Chard, L. S., Higa, T., Belsham, G. J., Wagner, G., Tanaka, J., and Pelletier, J. (2006). Functional characterization of IRESes by an inhibitor of the RNA helicase eIF4A. Nature chemical biology 2, 213-220.
Bordeleau, M. E., Robert, F., Gerard, B., Lindqvist, L., Chen, S. M., Wendel, H. G., Brem, B., Greger, H., Lowe, S. W., Porco, J. A., Jr., et al. (2008). Therapeutic suppression of translation initiation modulates chemosensitivity in a mouse lymphoma model. The Journal of clinical investigation 118, 2651-2660.
Bugaut, A., and Balasubramanian, S. (2012). 5′-UTR RNA G-quadruplexes: translation regulation and targeting. Nucleic acids research 40, 4727-4741.
Cencic, R. (2009). Antitumor activity and mechanism of action of the cyclopenta[b]benzofuran, silvestrol. PLoS ONE 4, e5223.
Cencic, R., Carrier, M., Trnkus, A., Porco, J. A., Jr., Minden, M., and Pelletier, J. (2010). Synergistic effect of inhibiting translation initiation in combination with cytotoxic agents in acute myelogenous leukemia cells. Leukemia research 34, 535-541.
Cencic, R., Hall, D. R., Robert, F., Du, Y., Min, J., Li, L., Qui, M., Lewis, I., Kurtkaya, S., Dingledine, R., et al. (2011). Reversing chemoresistance by small molecule inhibition of the translation initiation complex eIF4F. Proceedings of the National Academy of Sciences of the United States of America 108, 1046-1051.
Cencic, R., Robert, F., and Pelletier, J. (2007). Identifying small molecule inhibitors of eukaryotic translation initiation. Methods Enzymol 431, 269-302.
Chakraborty, P., and Grosse, F. (2011). Human DHX9 helicase preferentially unwinds RNA-containing displacement loops (R-loops) and G-quadruplexes. DNA repair 10, 654-665.
Choo, A. Y., Yoon, S. O., Kim, S. G., Roux, P. P., and Blenis, J. (2008). Rapamycin differentially inhibits S6Ks and 4E-BP1 to mediate cell-type-specific repression of mRNA translation. Proceedings of the National Academy of Sciences of the United States of America 105, 17414-17419.
D'Ambrogio, A., Nagaoka, K., and Richter, J. D. (2013). Translational control of cell growth and malignancy by the CPEBs. Nat Rev Cancer 13, 283-290.
De Keersmaecker, K., Atak, Z. K., Li, N., Vicente, C., Patchett, S., Girardi, T., Gianfelici, V., Geerdens, E., Clappier, E., Porcu, M., et al. (2013). Exome sequencing identifies mutation in CNOT3 and ribosomal genes RPL5 and RPL10 in T-cell acute lymphoblastic leukemia. Nature genetics 45, 186-190.
Della Gatta, G., Palomero, T., Perez-Garcia, A., Ambesi-Impiombato, A., Bansal, M., Carpenter, Z. W., De Keersmaecker, K., Sole, X., Xu, L., Paietta, E., et al. (2012). Reverse engineering of TLX oncogenic transcriptional networks identifies RUNX1 as tumor suppressor in T-ALL. Nature medicine 18, 436-440.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Drewe, P., Stegle, O., Hartmann, L., Kahles, A., Bohnert, R., Wachter, A., Borgwardt, K., and Ratsch, G. (2013). Accurate detection of differential RNA processing. Nucleic acids research 41, 5189-5198.
Feoktistova, K., Tuvshintogs, E., Do, A., and Fraser, C. S. (2013). Human eIF4E promotes mRNA restructuring by stimulating eIF4A helicase activity. Proceedings of the National Academy of Sciences of the United States of America 110, 13339-13344.
Furic, L., Rong, L., Larsson, O., Koumakpayi, I. H., Yoshida, K., Brueschke, A., Petroulakis, E., Robichaud, N., Pollak, M., Gaboury, L. A., et al. (2010). eIF4E phosphorylation promotes tumorigenesis and is associated with prostate cancer progression. Proceedings of the National Academy of Sciences of the United States of America 107, 14134-14139.
Giambra, V., Jenkins, C. R., Wang, H., Lam, S. H., Shevchuk, O. O., Nemirovsky, O., Wai, C., Gusscott, S., Chiang, M. Y., Aster, J. C., et al. (2012). NOTCH1 promotes T cell leukemia-initiating activity by RUNX-mediated regulation of PKC-theta and reactive oxygen species. Nature medicine 18, 1693-1698.
Guertin, D. A., and Sabatini, D. M. (2007). Defining the role of mTOR in cancer. Cancer cell 12, 9-22. Gutierrez, A., Grebliunaite, R., Feng, H., Kozakewich, E., Zhu, S., Guo, F., Payne, E., Mansour, M., Dahlberg, S. E., Neuberg, D. S., et al. (2011a). Pten mediates Myc oncogene dependence in a conditional zebrafish model of T cell acute lymphoblastic leukemia. The Journal of experimental medicine 208, 1595-1603.
Gutierrez, A., Kentsis, A., Sanda, T., Holmfeldt, L., Chen, S. C., Zhang, J., Protopopov, A., Chin, L., Dahlberg, S. E., Neuberg, D. S., et al. (2011b). The BCL11B tumor suppressor is mutated across the major molecular subtypes of T-cell acute lymphoblastic leukemia. Blood 118, 4169-4173.
Gutierrez, A., Sanda, T., Grebliunaite, R., Carracedo, A., Salmena, L., Ahn, Y., Dahlberg, S., Neuberg, D., Moreau, L. A., Winter, S. S., et al. (2009). High frequency of PTEN, PI3K, and AKT abnormalities in T-cell acute lymphoblastic leukemia. Blood 114, 647-650.
Guttman, M., Russell, P., Ingolia, N. T., Weissman, J. S., and Lander, E. S. (2013). Ribosome Profiling Provides Evidence that Large Noncoding RNAs Do Not Encode Proteins. Cell 154, 240-251.
Hay, N., and Sonenberg, N. (2004). Upstream and downstream of mTOR. Genes & development 18, 1926-1945.
Hsieh, A. C., Liu, Y., Edlind, M. P., Ingolia, N. T., Janes, M. R., Sher, A., Shi, E. Y., Stumpf, C. R., Christensen, C., Bonham, M. J., et al. (2012). The translational landscape of mTOR signalling steers cancer initiation and metastasis. Nature 485, 55-61.
Huppert, J. L., Bugaut, A., Kumari, S., and Balasubramanian, S. (2008). G-quadruplexes: the beginning and end of UTRs. Nucleic acids research 36, 6260-6268.
Ingolia, N. T., Ghaemmaghami, S., Newman, J. R., and Weissman, J. S. (2009). Genome-wide analysis in vivo of translation with nucleotide resolution using ribosome profiling. Science 324, 218-223.
Jackson, R. J., Hellen, C. U., and Pestova, T. V. (2010). The mechanism of eukaryotic translation initiation and principles of its regulation. Nature Rev Mol Cell Biol 11, 113-127.
Jean, G., Kahles, A., Sreedharan, V. T., De Bona, F., and Ratsch, G. (2010). RNA-Seq read alignments with PALMapper. Current protocols in bioinformatics/editoral board, Andreas D Baxevanis [et al] Chapter 11, Unit 11 16.
Jin, C., Rajabi, H., Rodrigo, C. M., Porco, J. A., Jr., and Kufe, D. (2012). Targeting the eIF4A RNA helicase blocks translation of the MUC1-C oncoprotein. Oncogene.
Kang, S. A., Pacold, M. E., Cervantes, C. L., Lim, D., Lou, H. J., Ottina, K., Gray, N. S., Turk, B. E., Yaffe, M. B., and Sabatini, D. M. (2013). mTORC1 phosphorylation sites encode their sensitivity to starvation and rapamycin. Science 341, 1236566.
Kentsis, A., Topisirovic, I., Culjkovic, B., Shao, L., and Borden, K. L. (2004). Ribavirin suppresses eIF4E-mediated oncogenic transformation by physical mimicry of the 7-methyl guanosine mRNA cap. Proceedings of the National Academy of Sciences of the United States of America 101, 18105-18110.
Kroczynska, B. (2009). Interferon-dependent engagement of eukaryotic initiation factor 4B via S6 kinase (S6K)- and ribosomal protein S6K-mediated signals. Mol Cell Biol 29, 2865-2875. Kumari, S., Bugaut, A., Huppert, J. L., and Balasubramanian, S. (2007). An RNA G-quadruplex in the 5′ UTR of the NRAS proto-oncogene modulates translation. Nat Chem Biol 3, 218-221.
Lahortiga, I., De Keersmaecker, K., Van Vlierberghe, P., Graux, C., Cauwelier, B., Lambert, F., Mentens, N., Beverloo, H. B., Pieters, R., Speleman, F., et al. (2007). Duplication of the MYB oncogene in T cell acute lymphoblastic leukemia. Nature genetics 39, 593-595.
Lazaris-Karatzas, A., Montine, K. S., and Sonenberg, N. (1990). Malignant transformation by a eukaryotic initiation factor subunit that binds to mRNA 5′ cap. Nature 345, 544-547. Li, H., Handsaker, B., Wysoker, A., Fennell, T., Ruan, J., Homer, N., Marth, G., Abecasis, G., Durbin, R., and Genome Project Data Processing, S. (2009a). The Sequence Alignment/Map format and SAMtools. Bioinformatics 25, 2078-2079.
Li, W., Dang, Y., Liu, J. O., and Yu, B. (2009b). Expeditious synthesis of hippuristanol and congeners with potent antiproliferative activities. Chemistry 15, 10356-10359.
Lindqvist, L., Oberer, M., Reibarkh, M., Cencic, R., Bordeleau, M. E., Vogt, E., Marintchev, A., Tanaka, J., Fagotto, F., Altmann, M., et al. (2008). Selective pharmacological targeting of a DEAD box RNA helicase. PLoS One 3, e1583.
Lucas, D. M., Edwards, R. B., Lozanski, G., West, D. A., Shin, J. D., Vargo, M. A., Davis, M. E., Rozewski, D. M., Johnson, A. J., Su, B. N., et al. (2009). The novel plant-derived agent silvestrol has B-cell selective activity in chronic lymphocytic leukemia and acute lymphoblastic leukemia in vitro and in vivo. Blood 113, 4656-4666.
Marintchev, A. (2009). Topology and regulation of the human eIF4A/4G/4H helicase complex in translation initiation. Cell 136, 447-460.
Marintchev, A. (2013). Roles of helicases in translation initiation: a mechanistic view. Biochim Biophys Acta 1829, 799-809.
Marintchev, A., Edmonds, K. A., Marintcheva, B., Hendrickson, E., Oberer, M., Suzuki, C., Herdy, B., Sonenberg, N., and Wagner, G. (2009). Topology and regulation of the human eIF4A/4G/4H helicase complex in translation initiation. Cell 136, 447-460.
Mavrakis, K. J., Van Der Meulen, J., Wolfe, A. L., Liu, X., Mets, E., Taghon, T., Khan, A. A., Setty, M., Rondou, P., Vandenberghe, P., et al. (2011). A cooperative microRNA-tumor suppressor gene network in acute T-cell lymphoblastic leukemia (T-ALL). Nat Genet 43, 673-678.
Meyuhas, O. (2000). Synthesis of the translational apparatus is regulated at the translational level. Eur J Biochem 267, 6321-6330.
Moerke, N. J., Aktas, H., Chen, H., Cantel, S., Reibarkh, M. Y., Fahmy, A., Gross, J. D., Degterev, A., Yuan, J., Chorev, M., et al. (2007). Small-molecule inhibition of the interaction between the translation initiation factors eIF4E and eIF4G. Cell 128, 257-267.
Morris, M. J., Negishi, Y., Pazsint, C., Schonhoft, J. D., and Basu, S. (2010). An RNA G-quadruplex is essential for cap-independent translation initiation in human VEGF IRES. J Am Chem Soc 132, 17831-17839.
Northcote, P. T., Blunt, J. W., and Munro, M. H. G. (1991). Pateamine: a potent cytotoxin from the New Zealand marine sponge, mycale sp. Tetrahedron Lett 32, 6411-6414.
Ntziachristos, P., Tsirigos, A., Van Vlierberghe, P., Nedjic, J., Trimarchi, T., Flaherty, M. S., Ferres-Marco, D., da Ros, V., Tang, Z., Siegle, J., et al. (2012). Genetic inactivation of the polycomb repressive complex 2 in T cell acute lymphoblastic leukemia. Nature medicine 18, 298-301.
O'Neil, J., Grim, J., Strack, P., Rao, S., Tibbitts, D., Winter, C., Hardwick, J., Welcker, M., Meijerink, J. P., Pieters, R., et al. (2007). FBW7 mutations in leukemic cells mediate NOTCH pathway activation and resistance to gamma-secretase inhibitors. The Journal of experimental medicine 204, 1813-1824.
O'Reilly, K. E., Rojo, F., She, Q. B., Solit, D., Mills, G. B., Smith, D., Lane, H., Hofmann, F., Hicklin, D. J., Ludwig, D. L., et al. (2006). mTOR inhibition induces upstream receptor tyrosine kinase signaling and activates Akt. Cancer research 66, 1500-1508.
Oberer, M., Marintchev, A., and Wagner, G. (2005). Structural basis for the enhancement of eIF4A helicase activity by eIF4G. Genes & development 19, 2212-2223.
Ozes, A. R., Feoktistova, K., Avanzino, B. C., and Fraser, C. S. (2011). Duplex unwinding and ATPase activities of the DEAD-box helicase eIF4A are coupled by eIF4G and eIF4B. Journal of molecular biology 412, 674-687.
Palomero, T., Lim, W. K., Odom, D. T., Sulis, M. L., Real, P. J., Margolin, A., Barnes, K. C., O'Neil, J., Neuberg, D., Weng, A. P., et al. (2006). NOTCH1 directly regulates c-MYC and activates a feed-forward-loop transcriptional network promoting leukemic cell growth. Proceedings of the National Academy of Sciences of the United States of America 103, 18261-18266.
Palomero, T., Sulis, M. L., Cortina, M., Real, P. J., Barnes, K., Ciofani, M., Caparros, E., Buteau, J., Brown, K., Perkins, S. L., et al. (2007). Mutational loss of PTEN induces resistance to NOTCH1 inhibition in T-cell leukemia. Nature medicine 13, 1203-1210.
Parsyan, A., Svitkin, Y., Shahbazian, D., Gkogkas, C., Lasko, P., Merrick, W. C., and Sonenberg, N. (2011). mRNA helicases: the tacticians of translational control. Nature reviews Molecular cell biology 12, 235-245.
Pear, W. S., Aster, J. C., Scott, M. L., Hasserjian, R. P., Soffer, B., Sklar, J., and Baltimore, D. (1996). Exclusive development of T cell neoplasms in mice transplanted with bone marrow expressing activated Notch alleles. The Journal of experimental medicine 183, 2283-2291.
Pelletier, J., and Sonenberg, N. (1988). Internal initiation of translation of eukaryotic mRNA directed by a sequence derived from poliovirus RNA. Nature 334, 320-325.
Penn, L. J., Brooks, M. W., Laufer, E. M., and Land, H. (1990a). Negative autoregulation of c-myc transcription. The EMBO journal 9, 1113-1121.
Penn, L. J., Brooks, M. W., Laufer, E. M., Littlewood, T. D., Morgenstern, J. P., Evan, G. I., Lee, W. M., and Land, H. (1990b). Domains of human c-myc protein required for autosuppression and cooperation with ras oncogenes are overlapping. Molecular and cellular biology 10, 4961-4966.
Real, P. J., Tosello, V., Palomero, T., Castillo, M., Hernando, E., de Stanchina, E., Sulis, M. L., Barnes, K., Sawai, C., Homminga, I., et al. (2009). Gamma-secretase inhibitors reverse glucocorticoid resistance in T cell acute lymphoblastic leukemia. Nature medicine 15, 50-58.
Rodrigo, C. M., Cencic, R., Roche, S. P., Pelletier, J., and Porco, J. A. (2012). Synthesis of rocaglamide hydroxamates and related compounds as eukaryotic translation inhibitors: synthetic and biological studies. Journal of medicinal chemistry 55, 558-562.
Rong, L., Livingstone, M., Sukarieh, R., Petroulakis, E., Gingras, A. C., Crosby, K., Smith, B., Polakiewicz, R. D., Pelletier, J., Ferraiuolo, M. A., et al. (2008). Control of eIF4E cellular localization by eIF4E-binding proteins, 4E-BPs. RNA 14, 1318-1327.
Rousseau, D., Gingras, A. C., Pause, A., and Sonenberg, N. (1996). The eIF4E-binding proteins 1 and 2 are negative regulators of cell growth. Oncogene 13, 2415-2420.
Rozovsky, N., Butterworth, A. C., and Moore, M. J. (2008). Interactions between eIF4AI and its accessory factors eIF4B and eIF4H. RNA 14, 2136-2148.
Ruggero, D., Montanaro, L., Ma, L., Xu, W., Londei, P., Cordon-Cardo, C., and Pandolfi, P. P. (2004). The translation factor eIF-4E promotes tumor formation and cooperates with c-Myc in lymphomagenesis. Nature medicine 10, 484-486.
Sadlish, H., Galicia-Vazquez, G., Paris, C. G., Aust, T., Bhullar, B., Chang, L., Helliwell, S. B., Hoepfner, D., Knapp, B., Riedl, R., et al. (2013). Evidence for a Functionally Relevant Rocaglamide Binding Site on the eIF4A-RNA Complex. ACS chemical biology.
Sawai, C. M., Freund, J., Oh, P., Ndiaye-Lobry, D., Bretz, J. C., Strikoudis, A., Genesca, L., Trimarchi, T., Kelliher, M. A., Clark, M., et al. (2012). Therapeutic targeting of the cyclin D3:CDK4/6 complex in T cell leukemia. Cancer cell 22, 452-465.
Schatz, J. H., Oricchio, E., Wolfe, A. L., Jiang, M., Linkov, I., Maragulia, J., Shi, W., Zhang, Z., Rajasekhar, V. K., Pagano, N. C., et al. (2011). Targeting cap-dependent translation blocks converging survival signals by AKT and PIM kinases in lymphoma. The Journal of experimental medicine 208, 1799-1807.
Shahbazian, D., Parsyan, A., Petroulakis, E., Topisirovic, I., Martineau, Y., Gibbs, B. F., Svitkin, Y., and Sonenberg, N. (2010). Control of cell survival and proliferation by mammalian eukaryotic initiation factor 4B. Molecular and cellular biology 30, 1478-1485.
Shahbazian, D., Roux, P. P., Mieulet, V., Cohen, M. S., Raught, B., Taunton, J., Hershey, J. W., Blenis, J., Pende, M., and Sonenberg, N. (2006). The mTOR/PI3K and MAPK pathways converge on eIF4B to control its phosphorylation and activity. Embo J 25, 2781-2791.
Shahid, R., Bugaut, A., and Balasubramanian, S. (2010). The BCL-2 5′ untranslated region contains an RNA G-quadruplex-forming motif that modulates protein expression. Biochemistry 49, 8300-8306.
Shima-Rich, E. A., Harden, A. M., McKeithan, T. W., Rowley, J. D., and Diaz, M. O. (1997). Molecular analysis of the t(8;14)(q24;q11) chromosomal breakpoint junctions in the T-cell leukemia line MOLT-16. Genes, chromosomes & cancer 20, 363-371.
Soucek, L., Whitfield, J., Martins, C. P., Finch, A. J., Murphy, D. J., Sodir, N. M., Karnezis, A. N., Swigart, L. B., Nasi, S., and Evan, G. I. (2008). Modelling Myc inhibition as a cancer therapy. Nature 455, 679-683.
Sun, S. Y., Rosenberg, L. M., Wang, X., Zhou, Z., Yue, P., Fu, H., and Khuri, F. R. (2005). Activation of Akt and eIF4E survival pathways by rapamycin-mediated mammalian target of rapamycin inhibition. Cancer research 65, 7052-7058.
Svitkin, Y. V. (2001). The requirement for eukaryotic initiation factor 4A (e1F4A) in translation is in direct proportion to the degree of mRNA 5[prime] secondary structure. RNA 7, 382-394.
Thoreen, C. C., Chantranupong, L., Keys, H. R., Wang, T., Gray, N. S., and Sabatini, D. M. (2012). A unifying model for mTORC1-mediated regulation of mRNA translation. Nature 485, 109-113.
Thoreen, C. C., Kang, S. A., Chang, J. W., Liu, Q., Zhang, J., Gao, Y., Reichling, L. J., Sim, T., Sabatini, D. M., and Gray, N. S. (2009). An ATP-competitive mammalian target of rapamycin inhibitor reveals rapamycin-resistant functions of mTORC1. The Journal of biological chemistry 284, 8023-8032.
Tsumuraya, T., Ishikawa, C., Machijima, Y., Nakachi, S., Senba, M., Tanaka, J., and Mori, N. (2011a). Effects of hippuristanol, an inhibitor of eIF4A, on adult T-cell leukemia. Biochemical pharmacology 81, 713-722.
Tsumuraya, T., Ishikawa, C., Machijima, Y., Nakachi, S., Senba, M., Tanaka, J., and Mori, N. (2011b). Effects of hippuristanol, an inhibitor of eIF4A, on adult T-cell leukemia. Biochemical pharmacology 81, 713-722.
Ueda, T., Watanabe-Fukunaga, R., Fukuyama, H., Nagata, S., and Fukunaga, R. (2004). Mnk2 and Mnk1 are essential for constitutive and inducible phosphorylation of eIF4E but not for cell growth or development. Mol Cell Biol 24, 6539-6549.
Van Vlierberghe, P., Ambesi-Impiombato, A., Perez-Garcia, A., Haydu, J. E., Rigo, I., Hadler, M., Tosello, V., Della Gatta, G., Paietta, E., Racevskis, J., et al. (2011). ETV6 mutations in early immature human T cell leukemias. The Journal of experimental medicine 208, 2571-2579.
Wan, X., Harkavy, B., Shen, N., Grohar, P., and Helman, L. J. (2007). Rapamycin induces feedback activation of Akt signaling through an IGF-1R-dependent mechanism. Oncogene 26, 1932-1940.
Weinstein, I. B. (2002). Cancer. Addiction to oncogenes—the Achilles heal of cancer. Science 297, 63-64.
Wendel, H. G., De Stanchina, E., Fridman, J. S., Malina, A., Ray, S., Kogan, S., Cordon-Cardo, C., Pelletier, J., and Lowe, S. W. (2004). Survival signalling by Akt and eIF4E in oncogenesis and cancer therapy. Nature 428, 332-337.
Wendel, H. G., Silva, R. L., Malina, A., Mills, J. R., Zhu, H., Ueda, T., Watanabe-Fukunaga, R., Fukunaga, R., Teruya-Feldstein, J., Pelletier, J., et al. (2007). Dissecting eIF4E action in tumorigenesis. Genes & development 21, 3232-3237.
Weng, A. P., Ferrando, A. A., Lee, W., Morris, J. P.t., Silverman, L. B., Sanchez-Irizarry, C., Blacklow, S. C., Look, A. T., and Aster, J. C. (2004). Activating mutations of NOTCH1 in human T cell acute lymphoblastic leukemia. Science 306, 269-271.
Weng, A. P., Millholland, J. M., Yashiro-Ohtani, Y., Arcangeli, M. L., Lau, A., Wai, C., Del Bianco, C., Rodriguez, C. G., Sai, H., Tobias, J., et al. (2006). c-Myc is an important direct target of Notchl in T-cell acute lymphoblastic leukemia/lymphoma. Genes & development 20, 2096-2109.
Yan, Y., Svitkin, Y., Lee, J. M., Bisaillon, M., and Pelletier, J. (2005). Ribavirin is not a functional mimic of the 7-methyl guanosine mRNA cap. RNA 11, 1238-1244.
Zenatti, P. P., Ribeiro, D., Li, W., Zuurbier, L., Silva, M. C., Paganin, M., Tritapoe, J., Hixon, J. A., Silveira, A. B., Cardoso, B. A., et al. (2011). Oncogenic IL7R gain-of-function mutations in childhood T-cell acute lymphoblastic leukemia. Nat Genet 43, 932-939.
Zhang, J., Ding, L., Holmfeldt, L., Wu, G., Heatley, S. L., Payne-Turner, D., Easton, J., Chen, X., Wang, J., Rusch, M., et al. (2012). The genetic basis of early T-cell precursor acute lymphoblastic leukaemia. Nature 481, 157-163.

TABLE 1
Detailed summary of T-ALL patient mutations

| | MYC | NOTCH1 mutation | | IL7R mutation | | | PTEN mutation + deletion | Genetic |
|---|---|---|---|---|---|---|---|---|
| ID | translocat | Nucleotide change Predicted prot. lesion | Domain | Nt chg. | Prot. chg. | Domain | Nucleotide change Predicted prot. lesion | subtype |
| 1 | t(8; 14) (q24; q11) | — | | — | | | heterozygous deletion | Unknown |
| 2 | — | c.7331_7332insCG (heterozygous) p.V2444fs*35 | PEST | — | | | c.[738delGTT; 737_741insCCCGG] | TAL/LMO (SIL-TAL) |
| | | | | | | | (heterozygous) p.P246fs*11 | |
| 3 | — | c.7393delC (heterozygous) p.L2465fs*13 | PEST | — | | | c.388C > T (homozygous) p.R130* | TAL/LMO (SIL-TAL) |
| | | c.[5023_5024delTT, 5022_025insCCC](het.) p.S1675fs*8 | HD | | | | | |
| 4 | — | c.[7372delC; 7372_7374insGGT] (het.) | PEST | — | | | heterozygous deletion exon 4-9 | TAL/LMO |
| | | p.L2458fs*21 | HD | | | | | (LMO2) |
| | | c.4802T > C (heterozygous) p.L1601P | | | | | | |
| 5 | — | c.4802T > C (heterozygous) p.L1601P | HD | — | | | — | HOXA (inv(7)) |
| 6 | — | c.7510C > T (heterozygous) p.Q2504* | PEST | — | | | — | TLX3 |
| 7 | — | C.5229C_5230GinsAGATTCCCTTATGGGACC (heterozygous) | JM | — | | | — | Unknown |
| 8 | — | C.4858_4864TACTACG > CCCGTCC (het.) | HD | — | | | — | TAL/LMO |
| | | p.1620_1622YYG > PDR | | | | | | (LMO2) |
| 9 | — | c.7504C > T (heterozygous) p.Q2502* | PEST | — | | | — | Unknown |
| | | c.5036T > C (heterozygous) p.L1679P | HD | | | | | |
| 10 | — | C.4748_4749insCCCCCCTTATAC (het.) | PY | — | | | — | Unknown |
| | | p.P1583_E1584insPPYT | | | | | | |
| 11 | — | c.[4749_4752delGGAG; 4748_4749insC] (het.) | HD | — | | | — | Unknown |
| | | p.E1584del | | | | | | |
| 12 | — | c.4732_4737delGTGGTG (heterozygous) | HD | — | | | — | TAL/LMO |
| | | p.V1578 _V1579del | PEST | | | | | (LMO2) |
| | | c.7462C > T (heterozygous) p.Q2488* | | | | | | |
| 13 | — | c. [7545_7548delTGAG; | PEST | — | | | — | HOXA (CALM-AF10 |
| | | 7545_7546insCTTT; 7552_7553insGAGC] (het.) | | | | | | |
| 14 | — | c.7318delG (heterozygous) p.P2439fs*39 | PEST | — | | | — | TLX3 |
| | | C.4728_4729insTTTCTCTTG (het.) | HD | | | | | |
| | | p.V1576_V1577insFLL | | | | | | |
| 15 | — | c.7563G > A (heterozygous) p.W2521* | PEST | — | | | — | TLX3 |
| 16 | — | c.7462C > T (heterozygous) p.Q2488* | PEST | — | | | — | TAL/LMO |
| | | c.5077T > A (heterozygous) p.C1693S | HD | | | | | (LMO2) |
| 17 | — | c.4821_4822insGGATTC (heterozygous) | HD | — | | | — | TAL/LMO |
| | | p.F1607 > LDS | | | | | | (LMO2) |
| 18 | — | c.4802T > C (heterozygous) p.L1601P | HD | — | | | — | TLX3 |
| 19 | — | c.4781T > C (heterozygous) p.L1594P | HD | — | | | — | TLX1 |
| 20 | — | c.4782_4783insGCC (heterozygous) | HD | — | | | — | TAL/LMO |
| | | p.L1594_R1595insP | HD | | | | | (SIL-TAL) |
| 21 | — | c.4802T > A (heterozygous) p.L1601Q | HD | — | | | — | TLX1 |
| 22 | — | — | | C.758T > G | p.V253G | TM | — | Unknown |
| 23 | — | — | | — | | | c.697C > T (heterozygous) p.R233* | TLX3 |
| | | | | | | | heterozygous deletion | |
| 24 | — | — | | | | | c.749_750insCCCCAGCTTGGATAG | TAL/LMO |
| | | | | | | | (heterozygous) p.G251fs*5 | (LMO2) |
| 25 | — | — | | — | | | c.295G > T (heterozygous) p.E99* | TAL/LMO |
| | | | | | | | | (SIL-TAL) |
| 26 | — | — | | — | | | heterozygous deletion exon 1-3 | TAL/LMO |
| | | | | | | | | (LMO2) |
| 27 | — | — | | — | | | — | HOXA (inv(7)) |
| 28 | — | — | | — | | | — | HOXA (MLL) |
| 29 | — | — | | — | | | — | HOXA (MLL) |
| 30 | — | — | | — | | | — | TAL/LMO |
| | | | | | | | | (SIL-TAL) |
| 31 | — | — | | — | | | — | TAL/LMO |
| | | | | | | | | (SIL-TAL) |
| 32 | — | — | | — | | | — | TAL/LMO |
| | | | | | | | | (SIL-TAL) |
| 33 | — | — | | — | | | — | TLX3 |
| 34 | — | — | | — | | | — | TLX1 |
| 35 | — | — | | — | | | — | TLX1 |
| 36 | — | — | | — | | | — | Unknown |

TABLE 2
In vivo toxicity study of (±)-CR-31-B
2A. Individual body and organ weights.

| | | Body Weight | Liver | Spleen | Left Kidney | Right Kidney |
|---|---|---|---|---|---|---|
| Group | Animal # | (gm) | (gm) | (gm) | (gm) | (gm) |
| Vehicle Female | 1 | 16.093 | 0.810 | 0.060 | 0.117 | 0.137 |
| Day 5 | 2 | 17.550 | 0.925 | 0.060 | 0.132 | 0.129 |
| | 3 | 18.866 | 0.965 | 0.059 | 0.176 | 0.192 |
| | 4 | 16.826 | 0.889 | 0.057 | 0.149 | 0.136 |
| | Mean | 17.334 | 0.897 | 0.059 | 0.144 | 0.149 |
| | Std Dev | 1.182 | 0.066 | 0.001 | 0.025 | 0.029 |
| | N | 4 | 4 | 4 | 4 | 4 |
| 0.2 mg/kg Female | 7 | 15.962 | 0.807 | 0.049 | 0.108 | 0.119 |
| Day 5 | 8 | 17.217 | 0.969 | 0.070 | 0.133 | 0.125 |
| | 9 | 17.463 | 0.914 | 0.086 | 0.138 | 0.135 |
| | 10 | 16.078 | 0.843 | 0.064 | 0.125 | 0.125 |
| | Mean | 16.680 | 0.883 | 0.067 | 0.126 | 0.126 |
| | Std Dev | 0.770 | 0.072 | 0.015 | 0.013 | 0.007 |
| | N | 4 | 4 | 4 | 4 | 4 |
| | % ChVh | −3.8 | −1.6 | 13.6 | −12.5 | −15.4 |
| Vehicle Female | 5 | 17.878 | 0.977 | 0.063 | 0.136 | 0.150 |
| Day 19 | 6 | 16.845 | 0.969 | 0.062 | 0.118 | 0.124 |
| | Mean | 17.362 | 0.973 | 0.063 | 0.127 | 0.137 |
| | Std Dev | 0.730 | 0.006 | 0.001 | 0.013 | 0.018 |
| | N | 2 | 2 | 2 | 2 | 2 |
| 0.2 mg/kg Female | 11 | 18.769 | 1.111 | 0.081 | 0.111 | 0.137 |
| Day 19 | 12 | 17.192 | 0.988 | 0.048 | 0.113 | 0.127 |
| | Mean | 17.981 | 1.050 | 0.065 | 0.112 | 0.132 |
| | Std Dev | 1.115 | 0.087 | 0.023 | 0.001 | 0.007 |
| | N | 2 | 2 | 2 | 2 | 2 |
| | % ChVh | 3.6 | 7.9 | 3.2 | −11.8 | −3.6 |

| | | Liver | Spleen | Left Kidney | Right Kidney |
|---|---|---|---|---|---|
| Group | Animal # | (% body wt) | (% body wt) | (% body wt) | (% body wt) |
| Vehicle Female | 1 | 5.03 | 0.37 | 0.85 | 0.85 |
| Day 5 | 2 | 5.27 | 0.34 | 0.75 | 0.74 |
| | 3 | 5.12 | 0.31 | 0.93 | 1.02 |
| | 4 | 5.28 | 0.34 | 0.89 | 0.81 |
| | Mean | 5.18 | 0.34 | 0.86 | 0.86 |
| | Std Dev | 0.12 | 0.02 | 0.08 | 0.12 |
| | N | 4 | 4 | 4 | 4 |
| 0.2 mg/kg Female | 7 | 5.06 | 0.31 | 0.75 | 0.75 |
| Day 5 | 8 | 5.63 | 0.41 | 0.77 | 0.73 |
| | 9 | 5.23 | 0.49 | 0.79 | 0.77 |
| | 10 | 5.24 | 0.40 | 0.78 | 0.78 |
| | Mean | 5.29 | 0.40 | 0.77 | 0.76 |
| | Std Dev | 0.24 | 0.07 | 0.02 | 0.02 |
| | N | 4 | 4 | 4 | 4 |
| | % ChVh | 2.1 | 17.6 | −10.5 | −11.6 |
| Vehicle Female | 5 | 5.46 | 0.35 | 0.84 | 0.84 |
| Day 19 | 6 | 5.75 | 0.37 | 0.70 | 0.74 |
| | Mean | 5.61 | 0.36 | 0.77 | 0.79 |
| | Std Dev | 0.21 | 0.01 | 0.10 | 0.07 |
| | N | 2 | 2 | 2 | 2 |
| 0.2 mg/kg Female | 11 | 5.92 | 0.43 | 0.73 | 0.73 |
| Day 19 | 12 | 5.75 | 0.28 | 0.66 | 0.74 |
| | Mean | 5.84 | 0.36 | 0.70 | 0.74 |
| | Std Dev | 0.12 | 0.11 | 0.05 | 0.01 |
| | N | 2 | 2 | 2 | 2 |
| | % ChVh | 4.1 | 0.0 | −9.1 | −6.3 |

2B. Individual hematology

| | | WBC | NEUT | LYM | MONO | EOS | BASO | | | | | |
|---|---|---|---|---|---|---|---|---|---|---|---|---|
| Group | Animal# | K/uL | K/uL | K/uL | K/uL | K/uL | K/uL | NEUT % | LYM % | MONO % | EOS % | BASO % |
| Vehicle Female | 1 | 7.24 | 0.65 | 6.33 | 0.24 | 0.01 | 0.01 | 8.92 | 87.47 | 3.38 | 0.17 | 0.07 |
| Day 5 | 2 | 7.44 | 1.00 | 6.30 | 0.10 | 0.02 | 0.01 | 13.43 | 84.72 | 1.41 | 0.27 | 0.17 |
| | 3 | 6.38 | 0.75 | 5.50 | 0.11 | 0.01 | 0.01 | 11.83 | 86.13 | 1.69 | 0.23 | 0.11 |
| | 4 | 4.52 | 0.72 | 3.76 | 0.03 | 0.00 | 0.00 | 15.88 | 83.26 | 0.68 | 0.11 | 0.07 |
| | Mean | 6.40 | 0.78 | 5.47 | 0.12 | 0.01 | 0.01 | 12.52 | 85.40 | 1.79 | 0.20 | 0.11 |
| | Std Dev | 1.33 | 0.15 | 1.20 | 0.09 | 0.01 | 0.01 | 2.92 | 1.81 | 1.14 | 0.07 | 0.05 |
| | N | 4 | 4 | 4 | 4 | 4 | 4 | 4 | 4 | 4 | 4 | 4 |
| 0.2 mg/kg | 7 | 4.78 | 1.05 | 3.64 | 0.08 | 0.00 | 0.00 | 21.94 | 76.21 | 1.77 | 0.01 | 0.07 |
| Female | 8 | 6.90 | 1.69 | 4.96 | 0.24 | 0.01 | 0.00 | 24.51 | 71.86 | 3.45 | 0.14 | 0.04 |
| Day 5 | 9 | 4.98 | 1.40 | 3.49 | 0.09 | 0.00 | 0.00 | 28.20 | 70.06 | 1.74 | 0.00 | 0.00 |
| | 10 | 6.96 | 1.70 | 5.13 | 0.12 | 0.01 | 0.00 | 24.44 | 73.76 | 1.70 | 0.10 | 0.00 |
| | Mean | 5.91 | 1.46 | 4.31 | 0.13 | 0.01 | 0.00 | 24.77 | 72.97 | 2.17 | 0.06 | 0.03 |
| | Std Dev | 1.19 | 0.31 | 0.86 | 0.07 | 0.01 | 0.00 | 2.58 | 2.63 | 0.86 | 0.07 | 0.03 |
| | N | 4 | 4 | 4 | 4 | 4 | 4 | 4 | 4 | 4 | 4 | 4 |
| | % ChVh | −7.7 | 87.2 | −21.2 | 8.3 | 0.0 | −100.0 | 97.8 | −14.6 | 21.2 | −70.0 | −72.7 |
| Vehicle Female | 5 | 5.44 | 0.79 | 4.51 | 0.10 | 0.04 | 0.01 | 14.44 | 82.94 | 1.76 | 0.76 | 0.10 |
| Day 19 | 6 | 8.68 | 1.12 | 7.43 | 0.09 | 0.03 | 0.01 | 12.85 | 85.63 | 1.06 | 0.34 | 0.11 |
| | Mean | 7.06 | 0.96 | 5.97 | 0.10 | 0.04 | 0.01 | 13.65 | 84.29 | 1.41 | 0.55 | 0.11 |
| | Std Dev | 2.29 | 0.23 | 2.06 | 0.01 | 0.01 | 0.00 | 1.12 | 1.90 | 0.49 | 0.30 | 0.01 |
| | N | 2 | 2 | 2 | 2 | 2 | 2 | 2 | 2 | 2 | 2 | 2 |
| 0.2 mg/kg Female | 11 | 3.36 | 0.51 | 2.80 | 0.03 | 0.02 | 0.00 | 15.03 | 83.42 | 0.91 | 0.50 | 0.14 |
| Day 19 | 12 | 2.50 | 0.33 | 2.12 | 0.02 | 0.02 | 0.00 | 13.29 | 84.96 | 0.95 | 0.66 | 0.13 |
| | Mean | 2.93 | 0.42 | 2.46 | 0.03 | 0.02 | 0.00 | 14.16 | 84.19 | 0.93 | 0.58 | 0.14 |
| | Std Dev | 0.61 | 0.13 | 0.48 | 0.01 | 0.00 | 0.00 | 1.23 | 1.09 | 0.03 | 0.11 | 0.01 |
| | N | 2 | 2 | 2 | 2 | 2 | 2 | 2 | 2 | 2 | 2 | 2 |
| | % ChVh | −58.5 | −56.3 | −58.8 | −70.0 | −50.0 | −100.0 | 3.7 | −0.1 | −34.0 | 5.5 | 27.3 |

| | | RBC | HGB | | MCV | MCH | MCHC | | PLT | MPV |
|---|---|---|---|---|---|---|---|---|---|---|
| Group | Animal# | M/uL | g/dL | HCT % | fL | Pg | g/dL | RDW % | K/uL | fL |
| Vehicle Female | 1 | 8.47 | 11.9 | 39.7 | 46.9 | 14.0 | 30.0 | 16.0 | 1343 | 4.6 |
| Day 5 | 2 | 9.19 | 13.8 | 46.9 | 51.0 | 15.0 | 29.4 | 15.8 | 1097 | 4.7 |
| | 3 | 8.51 | 13.1 | 44.3 | 52.0 | 15.4 | 29.6 | 16.3 | 839 | 4.4 |
| | 4 | 8.97 | 13.5 | 45.4 | 50.6 | 15.1 | 29.7 | 15.6 | 809 | 4.3 |
| | Mean | 8.79 | 13.1 | 44.1 | 50.1 | 14.9 | 29.7 | 15.9 | 1022.0 | 4.5 |
| | Std Dev | 0.35 | 0.83 | 3.11 | 2.23 | 0.61 | 0.25 | 0.30 | 250.02 | 0.18 |
| | N | 4 | 4 | 4 | 4 | 4 | 4 | 4 | 4 | 4 |
| 0.2 mg/kg | 7 | 9.31 | 14.3 | 48.3 | 51.9 | 15.4 | 29.6 | 15.6 | 872 | 4.3 |
| Female Day 5 | 8 | 8.95 | 13.8 | 45.1 | 50.4 | 15.4 | 30.6 | 16.1 | 673 | 4.3 |
| | 9 | 8.69 | 13.0 | 43.9 | 50.5 | 15.0 | 29.6 | 15.2 | 676 | 4.1 |
| | 10 | 8.89 | 13.5 | 45.0 | 50.6 | 15.2 | 30.0 | 16.4 | 682 | 4.3 |
| | Mean | 8.96 | 13.7 | 45.6 | 50.9 | 15.3 | 30.0 | 15.8 | 725.8 | 4.3 |
| | Std Dev | 0.26 | 0.54 | 1.90 | 0.70 | 0.19 | 0.47 | 0.53 | 97.57 | 0.10 |
| | N | 4 | 4 | 4 | 4 | 4 | 4 | 4 | 4 | 4 |
| | % ChVh | 1.9 | 4.4 | 3.4 | 1.4 | 2.5 | 0.9 | −0.6 | −29.0 | −5.6 |
| Vehicle Female | 5 | 9.02 | 14.0 | 43.8 | 48.6 | 15.5 | 32.0 | 16.3 | 1216 | 4.7 |
| Day 19 | 6 | 9.06 | 13.8 | 44.3 | 48.9 | 15.2 | 31.2 | 16.1 | 949 | 4.4 |
| | Mean | 9.04 | 13.9 | 44.1 | 48.8 | 15.4 | 31.6 | 16.2 | 1082.5 | 4.6 |
| | Std Dev | 0.03 | 0.14 | 0.35 | 0.21 | 0.21 | 0.57 | 0.14 | 188.80 | 0.21 |
| | N | 2 | 2 | 2 | 2 | 2 | 2 | 2 | 2 | 2 |
| 0.2 mg/kg Female | 11 | 9.33 | 13.4 | 45.9 | 49.2 | 14.4 | 29.2 | 16.4 | 990 | 4.6 |
| Day 19 | 12 | 8.70 | 13.0 | 42.5 | 48.8 | 14.9 | 30.6 | 16.2 | 1403 | 4.9 |
| | Mean | 9.02 | 13.2 | 44.2 | 49.0 | 14.7 | 29.9 | 16.3 | 1196.5 | 4.8 |
| | Std Dev | 0.45 | 0.28 | 2.40 | 0.28 | 0.35 | 0.99 | 0.14 | 292.04 | 0.21 |
| | N | 2 | 2 | 2 | 2 | 2 | 2 | 2 | 2 | 2 |
| | % ChVh | −0.2 | −5.0 | 0.2 | 0.4 | -4.5 | -5.4 | 0.6 | 10.5 | 4.3 |

2C. Bone marrow and spleen cytology
Bone Marrow

| | Mat | % mat | Pro | % Pro | Total | % T | Mat | % Mat | Prol | % Pro | Total | % T |
|---|---|---|---|---|---|---|---|---|---|---|---|---|
| Animal | myel | myel | myel | myel | myel | myel | ery | ery | ery | ery | Ery | ery |
| Vehicle #1 | 124 | 41.3 | 28 | 9.3 | 152 | 50.7 | 57 | 19.0 | 29 | 9.7 | 86 | 28.7 |
| Vehicle #2 | 121 | 40.3 | 30 | 10.0 | 151 | 50.3 | 64 | 21.3 | 24 | 8.0 | 88 | 29.3 |
| Vehicle #3 | 105 | 35.0 | 58 | 19.3 | 163 | 54.3 | 57 | 19.0 | 32 | 10.7 | 89 | 29.7 |
| Vehicle #4 | 115 | 38.3 | 46 | 15.3 | 161 | 53.7 | 47 | 15.7 | 26 | 8.7 | 73 | 24.3 |
| Treatment #7 | 122 | 40.7 | 34 | 11.3 | 156 | 52.0 | 56 | 18.7 | 24 | 8.0 | 80 | 26.7 |
| Treatment #8 | 88 | 29.3 | 64 | 21.3 | 152 | 50.7 | 62 | 20.7 | 26 | 8.7 | 88 | 29.3 |
| Treatment #9 | 120 | 40.0 | 52 | 17.3 | 172 | 57.3 | 45 | 15.0 | 22 | 7.3 | 67 | 22.3 |
| Treatment #10 | 121 | 40.3 | 72 | 24.0 | 193 | 64.3 | 38 | 12.7 | 25 | 8.3 | 63 | 21.0 |

Spleen

| Animal | % Lymph | % Macrophage | % Myeloid | % Erythroid | % Plasma | % Mesenchymal |
|---|---|---|---|---|---|---|
| Vehicle #1 | 92 | 6 | 1 | 1 | 0 | 0 |
| Vehicle #2 | 93 | 2 | 5 | 0 | 0 | 0 |
| Vehicle #3 | 96 | 1 | 1 | 0 | 0 | 2 |
| Vehicle #4 | 97 | 2 | 0 | 0 | 1 | 0 |
| Treatment #7 | 94 | 2 | 4 | 0 | 0 | 0 |
| Treatment #8 | 90 | 2 | 7 | 0 | 1 | 0 |
| Treatment #9 | 90 | 0 | 9 | 1 | 0 | 0 |
| Treatment #10 | 86 | 2 | 10 | 0 | 0 | 2 |

| | Mat | | | | | | | | | Total | |
|---|---|---|---|---|---|---|---|---|---|---|---|
| Animal | myel | Lymph | % Lymph | Plasma | % Plasma | Mast | % Mast | Mega* | Quality* | cell | M:E |
| Vehicle #1 | 124 | 62 | 20.7 | 0 | 0.0 | 0 | 0.0 | p | a | 300 | 1.8 |
| Vehicle #2 | 121 | 61 | 20.3 | 0 | 0.0 | 0 | 0.0 | p | a | 300 | 1.7 |
| Vehicle #3 | 105 | 48 | 16.0 | 0 | 0.0 | 0 | 0.0 | p | a | 300 | 1.8 |
| Vehicle #4 | 115 | 66 | 22.0 | 0 | 0.0 | 0 | 0.0 | P | a | 300 | 2.2 |
| | | | | | | 0.0 | 0.0 | | | | |
| Treatment #7 | 122 | 64 | 21.3 | 0 | 0.0 | 0 | 0.0 | P | a | 300 | 2.0 |
| Treatment #8 | 88 | 60 | 20.0 | 0 | 0.0 | 0 | 0.0 | p | a | 300 | 1.7 |
| Treatment #9 | 120 | 61 | 20.3 | 0 | 0.0 | 0 | 0.0 | p | a | 300 | 2.6 |
| Treatment #10 | 121 | 44 | 14.7 | 0 | 0.0 | 0 | 0.0 | | | 300 | 3.1 |

Spleen

| Animal | % Lymph | Comment | Total % |
|---|---|---|---|
| Vehicle #1 | 92 | | 100 |
| Vehicle #2 | 93 | | 100 |
| Vehicle #3 | 96 | | 100 |
| Vehicle #4 | 97 | large numbers of pleomorphic rod bacteria | 100 |
| | | in background of smear | |
| Treatment #7 | 94 | | 100 |
| Treatment #8 | 90 | | 100 |
| Treatment #9 | 90 | | 100 |
| Treatment #10 | 86 | | 100 |

2D. Individual chemistry

| | | ALP | ALT | AST | GGT | ALB | TP | GLOB | A/G | TBIL | BUN | CRTN |
|---|---|---|---|---|---|---|---|---|---|---|---|---|
| Group | Animal # | U/L | U/L | U/L | U/L | g/dL | g/dL | g/dL | Ratio | mg/d L | mg/d L | mg/dL |
| Vehicle Female | 1 | 125 | 22 | 39 | 0 | 2.8 | 4.5 | 1.7 | 1.6 | 0.2 | 24 | 0.1 |
| Day 5 | 2 | 115 | 14 | 31 | 0 | 3.1 | 4.9 | 1.8 | 1.7 | 0.2 | 28 | 0.2 |
| | 3 | 122 | 19 | 32 | 0 | 2.9 | 4.5 | 1.6 | 1.8 | 0.1 | 23 | 0.2 |
| | 4 | 133 | 22 | 34 | 0 | 3.0 | 4.7 | 1.7 | 1.8 | 0.2 | 31 | 0.2 |
| | Mean | 123.8 | 19.3 | 34.0 | 0.0 | 3.0 | 4.7 | 1.7 | 1.7 | 0.18 | 26.5 | 0.18 |
| | Std | 7.46 | 3.77 | 3.56 | 0.00 | 0.13 | 0.19 | 0.08 | 0.10 | 0.05 | 3.70 | 0.05 |
| | Dev | | | | | | | | | | | |
| | N | 4 | 4 | 4 | 4 | 4 | 4 | 4 | 4 | 4 | 4 | 4 |
| 0.2 mg/kg | 7 | 116 | 26 | 51 | 0 | 3.3 | 5.1 | 1.8 | 1.8 | 0.2 | 27 | 0.2 |
| Female | 8 | 114 | 20 | 35 | 0 | 3.0 | 4.8 | 1.8 | 1.7 | 0.2 | 26 | 0.2 |
| Day 5 | 9 | 123 | 34 | 44 | 0 | 2.9 | 4.8 | 1.9 | 1.5 | 0.1 | 27 | 0.2 |
| | 10 | 144 | 20 | 35 | 0 | 3.1 | 4.9 | 1.8 | 1.7 | 0.2 | 29 | 0.2 |
| | Mean | 124.3 | 25.0 | 41.3 | 0.0 | 3.1 | 4.9 | 1.8 | 1.7 | 0.18 | 27.3 | 0.20 |
| | Std | 13.72 | 6.63 | 7.76 | 0.00 | 0.17 | 0.14 | 0.05 | 0.13 | 0.05 | 1.26 | 0.00 |
| | Dev | | | | | | | | | | | |
| | N | 4 | 4 | 4 | 4 | 4 | 4 | 4 | 4 | 4 | 4 | 4 |
| | % ChVh | | | | | 3.3 | 4.3 | 5.9 | 0.0 | 0.0 | 3.0 | 11.1 |
| | FoldChVh | 1.0 | 1.3 | 1.2 | NC | | | | | | | |
| Vehicle Female | 5 | 164 | 20 | 46 | 0 | 3.2 | 5.1 | 1.9 | 1.7 | 0.1 | 31 | 0.2 |
| Day 19 | 6 | 193 | 40 | 78 | 0 | 2.9 | 4.6 | 1.7 | 1.7 | 0.0 | 34 | 0.2 |
| | Mean | 178.5 | 30.0 | 62.0 | 0.0 | 3.1 | 4.9 | 1.8 | 1.7 | 0.05 | 32.5 | 0.20 |
| | Std Dev | 20.51 | 14.14 | 22.63 | 0.00 | 0.21 | 0.35 | 0.14 | 0.00 | 0.07 | 2.12 | 0.00 |
| | N | 2 | 2 | 2 | 2 | 2 | 2 | 2 | 2 | 2 | 2 | 2 |
| 0.2 mg/kg Female | 11 | 144 | 20 | 33 | 0 | 2.9 | 4.7 | 1.8 | 1.6 | 0.1 | 29 | 0.2 |
| Day 19 | 12 | 160 | 22 | 38 | 0 | 2.9 | 4.7 | 1.8 | 1.6 | 0.1 | 35 | 0.2 |
| | Mean | 152.0 | 21.0 | 35.5 | 0.0 | 2.9 | 4.7 | 1.8 | 1.6 | 0.10 | 32.0 | 0.20 |
| | Std | 11.31 | 1.41 | 3.54 | 0.00 | 0.00 | 0.00 | 0.00 | 0.00 | 0.00 | 4.24 | 0.00 |
| | Dev | | | | | | | | | | | |
| | N | 2 | 2 | 2 | 2 | 2 | 2 | 2 | 2 | 2 | 2 | 2 |
| | % ChVh | | | | | -6.5 | -4.1 | 0.0 | -5.9 | 100.0 | -1.5 | 0.0 |
| | FoldChVh | 0.9 | 0.7 | 0.6 | NC | | | | | | | |

| | Animal | CHOL | GLUC | Ca | P | Cl | K | Na | Na/K |
|---|---|---|---|---|---|---|---|---|---|
| Group | # | mg/dL | mg/dL | mg/dL | mg/dL | mEq/L | mEq/L | mEq/L | Ratio |
| Vehicle Female | 1 | 62 | 301 | 7.1 | 8.1 | 113 | 5.7 | 148 | 26 |
| Day 5 | 2 | 78 | 287 | 9.8 | 7.3 | 113 | 3.8 | 150 | 39 |
| | 3 | 63 | 307 | 9.5 | 7.5 | 116 | 4.0 | 151 | 38 |
| | 4 | 61 | 273 | 9.2 | 9.8 | 111 | 4.0 | 148 | 37 |
| | Mean | 66.0 | 292.0 | 8.9 | 8.2 | 113.3 | 4.4 | 149.3 | 35.0 |
| | Std Dev | 8.04 | 15.19 | 1.22 | 1.14 | 2.06 | 0.89 | 1.50 | 6.06 |
| | N | 4 | 4 | 4 | 4 | 4 | 4 | 4 | 4 |
| | 7 | 68 | 270 | 8.9 | 6.3 | 116 | 4.6 | 152 | 33 |
| 0.2 mg/kg Female | 8 | 66 | 335 | 9.0 | 7.3 | 113 | 4.0 | 147 | 37 |
| Day 5 | 9 | 64 | 261 | 9.3 | 7.5 | 114 | 4.4 | 150 | 34 |
| | 10 | 66 | 323 | 9.3 | 7.0 | 113 | 3.9 | 147 | 38 |
| | Mean | 66.0 | 297.3 | 9.1 | 7.0 | 114.0 | 4.2 | 149.0 | 35.5 |
| | Std Dev | 1.63 | 37.17 | 0.21 | 0.53 | 1.41 | 0.33 | 2.45 | 2.38 |
| | N | 4 | 4 | 4 | 4 | 4 | 4 | 4 | 4 |
| | % ChVh | 0.0 | 1.8 | 2.2 | −14.6 | 0.6 | −4.5 | −0.2 | 1.4 |
| | FoldChVh | | | | | | | | |
| Vehicle Female | 5 | 74 | 261 | 9.4 | 6.0 | 116 | 4.3 | 148 | 34 |
| Day 19 | 6 | 59 | 261 | 9.5 | 7.5 | 116 | 4.1 | 148 | 36 |
| | Mean | 66.5 | 261.0 | 9.5 | 6.8 | 116.0 | 4.2 | 148.0 | 35.0 |
| | Std Dev | 10.61 | 0.00 | 0.07 | 1.06 | 0.00 | 0.14 | 0.00 | 1.41 |
| | N | 2 | 2 | 2 | 2 | 2 | 2 | 2 | 2 |
| 0.2 mg/kg Female | 11 | 71 | 254 | 9.0 | 9.6 | 112 | 3.5 | 147 | 42 |
| Day 19 | 12 | 59 | 227 | 9.4 | 10.8 | 117 | 4.0 | 149 | 37 |
| | Mean | 65.0 | 240.5 | 9.2 | 10.2 | 114.5 | 3.8 | 148.0 | 39.5 |
| | Std Dev | 8.49 | 19.09 | 0.28 | 0.85 | 3.54 | 0.35 | 1.41 | 3.54 |
| | N | 2 | 2 | 2 | 2 | 2 | 2 | 2 | 2 |
| | % ChVh | −2.3 | −7.9 | −3.2 | 50.0 | −1.3 | −9.5 | 0.0 | 12.9 |
| | FoldChVh | | | | | | | | |

*p = present a = adequate

TABLE 3A
Motifs and G-quadruplexes in TE down genes.

| Gene ID | Gene Name | Translational Efficiency (p-value) | log2 (Translational Efficiency) | rDiff (p-value) |
|---|---|---|---|---|
| ENSG00000204147 | ASAH2B | 0.015002357 | −1.322460998 | 1 |
| ENSG00000125827 | TMX4 | 0.004516727 | −1.251223509 | 1 |
| ENSG00000008710 | PKD1 | 0.016462045 | −1.049712325 | 1 |
| ENSG00000182197 | EXT1 | 0.024702029 | −0.999952768 | 1 |
| ENSG00000181027 | FKRP | 0.016390601 | −0.996001717 | 1 |
| ENSG00000056998 | GYG2 | 0.026501722 | −0.929650779 | 1 |
| ENSG00000124786 | SLC35B3 | 0.023814954 | −0.917840885 | 1 |
| ENSG00000164970 | FAM219A | 0.012381128 | −0.63799892 | 1 |
| ENSG00000065802 | ASB1 | 0.011120438 | −0.632831228 | 7.24E−02 |
| ENSG00000150995 | ITPR1 | 0.025529346 | −0.628260516 | 1 |
| ENSG00000130669 | PAK4 | 0.02752902 | −0.627975797 | 1 |
| ENSG00000166503 | HDGFRP3 | 0.000273828 | −0.608750084 | 1.19E−02 |
| ENSG00000180730 | SHISA2 | 2.85E−05 | −0.608613867 | 1.93E−02 |
| ENSG00000119844 | AFTPH | 0.008725735 | −0.591675727 | 1 |
| ENSG00000180035 | ZNF48 | 0.015918146 | −0.582772982 | 1 |
| ENSG00000133056 | PIK3C2B | 0.016052253 | −0.537952135 | 1 |
| ENSG00000109220 | CHIC2 | 0.018242688 | −0.521221308 | 1.70E−02 |
| ENSG00000127152 | BCL11B | 5.40E−10 | −0.517770746 | 1.00E−08 |
| ENSG00000140853 | NLRC5 | 0.009461003 | −0.515280789 | 1 |
| ENSG00000034677 | RNF19A | 0.025744017 | −0.483894067 | 1 |
| ENSG00000105321 | CCDC9 | 0.020946401 | −0.480466187 | 1 |
| ENSG00000151014 | CCRN4L | 0.007814184 | −0.480208055 | 7.50E−03 |
| ENSG00000123159 | GIPC1 | 0.00645275 | −0.478285755 | 6.90E−03 |
| ENSG00000171791 | BCL2 | 0.008656918 | −0.474172722 | 6.00E−01 |
| ENSG00000065970 | FOXJ2 | 0.017646559 | −0.4735107 | 1 |
| ENSG00000066933 | MYO9A | 0.008868511 | −0.473506657 | 1 |
| ENSG00000182150 | ERCC6L2 | 0.016924169 | −0.466789648 | 1 |
| ENSG00000100393 | EP300 | 1.18E−05 | −0.464539688 | 1.10E−03 |
| ENSG00000120949 | TNFRSF8 | 0.022812631 | −0.437286644 | 7.40E−03 |
| ENSG00000123575 | FAM199X | 0.029642368 | −0.431235143 | 2.08E−02 |
| ENSG00000179195 | ZNF664 | 0.005477293 | −0.42685597 | 1 |
| ENSG00000166024 | R3HCC1L | 0.024332543 | −0.416140766 | 1 |
| ENSG00000123066 | MED13L | 4.79E−05 | −0.415941737 | 2.20E−08 |
| ENSG00000145349 | CAMK2D | 0.021359574 | −0.408237368 | 7.82E−02 |
| ENSG00000110218 | PANX1 | 0.005542647 | −0.39874583 | 2.59E−02 |
| ENSG00000003402 | CFLAR | 0.024657096 | −0.397685039 | 4.71E−02 |
| ENSG00000164168 | TMEM184C | 0.015050183 | −0.397412646 | 2.50E−08 |
| ENSG00000169018 | FEM1B | 0.01910054 | −0.397020738 | 3.00E−04 |
| ENSG00000007168 | PAFAH1B1 | 0.000466042 | −0.383179082 | 9.80E−03 |
| ENSG00000169967 | MAP3K2 | 0.013056576 | −0.377048905 | 6.60E−03 |
| ENSG00000162889 | MAPKAPK2 | 0.016286083 | −0.352699883 | 5.49E−01 |
| ENSG00000063978 | RNF4 | 0.002157553 | −0.344201177 | 1.28E−02 |
| ENSG00000064490 | RFXANK | 0.015800837 | −0.324746409 | 3.00E−04 |
| ENSG00000100105 | PATZ1 | 0.00652701 | −0.324460116 | 3.10E−03 |
| ENSG00000103502 | CDIPT | 0.021406946 | −0.323954264 | 1.43E−01 |
| ENSG00000095380 | NANS | 0.027123309 | −0.321819229 | 5.39E−02 |
| ENSG00000160917 | CPSF4 | 0.016200762 | −0.319405125 | 1.70E−03 |
| ENSG00000153561 | RMND5A | 0.029110593 | −0.311729374 | 2.03E−01 |
| ENSG00000108510 | MED13 | 0.005034555 | −0.293870753 | 1.60E−03 |
| ENSG00000112531 | QKI | 0.00269178 | −0.29125342 | 8.40E−03 |
| ENSG00000163349 | HIPK1 | 0.020020123 | −0.282659771 | 2.00E−04 |
| ENSG00000111885 | MAN1A1 | 0.010654006 | −0.274580872 | 2.24E−02 |
| ENSG00000048405 | ZNF800 | 0.020652909 | −0.271097499 | 3.00E−08 |
| ENSG00000115419 | GLS | 0.000197719 | −0.269875671 | 4.00E−04 |
| ENSG00000182831 | C16orf72 | 0.00375696 | −0.255255837 | 1.07E−01 |
| ENSG00000131507 | NDF1P1 | 0.004173323 | −0.242895723 | 6.80E−03 |
| ENSG00000134602 | MST4 | 0.003080229 | −0.242407773 | 1.05E−02 |
| ENSG00000159692 | CTBP1 | 0.006057739 | −0.241648156 | 5.00E−03 |
| ENSG00000106609 | TMEM248 | 0.005316307 | −0.23621242 | 2.90E−07 |
| ENSG00000152684 | PELO | 0.01293572 | −0.236131973 | 7.82E−02 |
| ENSG00000134954 | ETS1 | 1.27E−05 | −0.232324455 | 5.70E−09 |
| ENSG00000140332 | TLE3 | 0.001343794 | −0.227829431 | 2.00E−08 |
| ENSG00000169905 | TOR1AIP2 | 0.025144824 | −0.223803399 | 7.00E−04 |
| ENSG00000149480 | MTA2 | 2.64E−05 | −0.22354576 | 6.00E−09 |
| ENSG00000105329 | TGFB1 | 0.015301045 | −0.221315351 | 2.00E−04 |
| ENSG00000131504 | DIAPH1 | 0.005406879 | −0.213577391 | 2.00E−04 |
| ENSG00000138795 | LEF1 | 1.60E−06 | −0.210659864 | 1.00E−09 |
| ENSG00000106290 | TAF6 | 0.014175182 | −0.210235711 | 5.00E−04 |
| ENSG00000137845 | ADAM10 | 0.012053048 | −0.208903322 | 9.00E−10 |
| ENSG00000136878 | USP20 | 0.019165529 | −0.206602358 | 2.00E−08 |
| ENSG00000172292 | CERS6 | 0.029552171 | −0.205124483 | 1.00E−09 |
| ENSG00000135932 | CAB39 | 0.019948395 | −0.200243436 | 1.00E−08 |
| ENSG00000118816 | CCNI | 0.001528498 | −0.189161037 | 6.90E−07 |
| ENSG00000151465 | CDC123 | 0.007301 | −0.184803611 | 3.23E−01 |
| ENSG00000140262 | TCF12 | 0.025334533 | −0.184064816 | 2.00E−10 |
| ENSG00000100796 | SMEK1 | 0.021404696 | −0.176469607 | 1.00E−11 |
| ENSG00000112306 | RPS12 | 0.029227861 | −0.173199482 | 1.13E−01 |
| ENSG00000105063 | PPP6R1 | 0.029786388 | −0.157573098 | 1.00E−10 |
| ENSG00000120727 | PAIP2 | 0.010801093 | −0.157320231 | 5.59E−01 |
| ENSG00000152601 | MBNL1 | 0.00777836 | −0.152181062 | 2.00E−11 |
| ENSG00000088325 | TPX2 | 0.000751758 | −0.147886462 | 4.00E−13 |
| ENSG00000171310 | CHST11 | 0.004536717 | −0.14604981 | 1.20E−07 |
| ENSG00000158985 | CDC42SE2 | 0.027991366 | −0.145160094 | 8.00E−08 |
| ENSG00000184007 | PTP4A2 | 0.00039459 | −0.142942918 | 1.50E−07 |
| ENSG00000153310 | FAM49B | 0.007506383 | −0.139159484 | 2.00E−04 |
| ENSG00000121083 | DYNLL2 | 0.02989098 | −0.137797441 | 1.00E−03 |
| ENSG00000078369 | GNB1 | 0.011642786 | −0.133797709 | 5.00E−04 |
| ENSG00000125743 | SNRPD2 | 0.024903253 | −0.131448444 | 2.38E−01 |
| ENSG00000110651 | CD81 | 0.010480682 | −0.130640591 | 1.00E−04 |
| ENSG00000077312 | SNRPA | 0.023537735 | −0.127819876 | 1.26E−01 |
| ENSG00000125970 | RALY | 0.004001428 | −0.112279678 | 3.64E−02 |
| ENSG00000169764 | UGP2 | 0.027598388 | −0.103615488 | 1.60E−03 |
| ENSG00000138668 | HNRNPD | 0.003261874 | −0.098862205 | 2.40E−07 |
| ENSG00000167978 | SRRM2 | 0.027585188 | −0.081656945 | 1.00E−04 |

TABLE 3B
TE Down genes with 9-mer GC-rich motif

| Gene ID | Gene Name | Translational Efficiency (p-value) | log2 (Translational Efficiency) | rDiff (p-value) |
|---|---|---|---|---|
| ENSG00000142530 | FAM71E1 | 0.007231579 | −13.06577528 | 1 |
| ENSG00000164877 | MICALL2 | 0.00343177 | −13.04340083 | 1 |
| ENSG00000205002 | AARD | 0.005983799 | −12.57393172 | 1 |
| ENSG00000096264 | NCR2 | 0.004901353 | −12.43253148 | 1 |
| ENSG00000104881 | PPP1R13L | 0.01002069 | −1.405330178 | 1 |
| ENSG00000154016 | GRAP | 0.010299775 | −1.388227224 | 1 |
| ENSG00000025434 | NR1H3 | 0.009825261 | −1.378487187 | 1 |
| ENSG00000204147 | ASAH2B | 0.015002357 | −1.322460998 | 1 |
| ENSG00000111664 | GNB3 | 0.016841552 | −1.309465795 | 1 |
| ENSG00000154783 | FGD5 | 0.010083869 | −1.258941532 | 1 |
| ENSG00000125827 | TMX4 | 0.004516727 | −1.251223509 | 1 |
| ENSG00000139112 | GABARAPL1 | 0.008844095 | −1.233522978 | 1 |
| ENSG00000162065 | TBC1D24 | 0.005127762 | −1.23071089 | 1 |
| ENSG00000102265 | TIMP1 | 0.019522742 | −1.114778075 | 1 |
| ENSG00000008710 | PKD1 | 0.016462045 | −1.049712325 | 1 |
| ENSG00000182986 | ZNF320 | 0.027309984 | −1.003743356 | 1 |
| ENSG00000106829 | TLE4 | 0.000832033 | −1.003696096 | 1 |
| ENSG00000182197 | EXT1 | 0.024702029 | −0.999952768 | 1 |
| ENSG00000181027 | FKRP | 0.016390601 | −0.996001717 | 1 |
| ENSG00000064687 | ABCA7 | 0.01073148 | −0.991801368 | 1 |
| ENSG00000056998 | GYG2 | 0.026501722 | −0.929650779 | 1 |
| ENSG00000124786 | SLC35B3 | 0.023814954 | −0.917840885 | 1 |
| ENSG00000075399 | VPS9D1 | 0.01039008 | −0.829631073 | 1 |
| ENSG00000172732 | MUS81 | 0.000200214 | −0.721332975 | 1.05E−01 |
| ENSG00000055208 | TAB2 | 0.005417389 | −0.647025741 | 1 |
| ENSG00000164970 | FAM219A | 0.012381128 | −0.63799892 | 1 |
| ENSG00000065802 | ASB1 | 0.011120438 | −0.632831228 | 7.24E−02 |
| ENSG00000150995 | ITPR1 | 0.025529346 | −0.628260516 | 1 |
| ENSG00000130669 | PAK4 | 0.02752902 | −0.627975797 | 1 |
| ENSG00000112394 | SLC16A10 | 0.018925329 | −0.615544597 | 1 |
| ENSG00000166503 | HDGFRP3 | 0.000273828 | −0.608750084 | 1.19E−02 |
| ENSG00000180730 | SHISA2 | 2.85E−05 | −0.608613867 | 1.93E−02 |
| ENSG00000176994 | SMCR8 | 0.003428912 | −0.604582332 | 1 |
| ENSG00000204348 | DOM3Z | 0.026501738 | −0.595853393 | 1 |
| ENSG00000152127 | MGAT5 | 0.000966439 | −0.592236096 | 1.04E−01 |
| ENSG00000119844 | AFTPH | 0.008725735 | −0.591675727 | 1 |
| ENSG00000180035 | ZNF48 | 0.015918146 | −0.582772982 | 1 |
| ENSG00000132879 | FBXO44 | 0.011714393 | −0.538058958 | 1.10E−01 |
| ENSG00000133056 | PIK3C2B | 0.016052253 | −0.537952135 | 1 |
| ENSG00000137822 | TUBGCP4 | 0.017824276 | −0.534568492 | 3.06E−02 |
| ENSG00000109220 | CHIC2 | 0.018242688 | −0.521221308 | 1.70E−02 |
| ENSG00000127152 | BCL11B | 5.40E−10 | −0.517770746 | 1.00E−08 |
| ENSG00000140853 | NLRC5 | 0.009461003 | −0.515280789 | 1 |
| ENSG00000135049 | AGTPBP1 | 0.005676581 | −0.500916234 | 1 |
| ENSG00000141873 | SLC39A3 | 0.00107034 | −0.49572766 | 1 |
| ENSG00000034677 | RNF19A | 0.025744017 | −0.483894067 | 1 |
| ENSG00000105321 | CCDC9 | 0.020946401 | −0.480466187 | 1 |
| ENSG00000151014 | CCRN4L | 0.007814184 | −0.480208055 | 7.50E−03 |
| ENSG00000123159 | GIPC1 | 0.00645275 | −0.478285755 | 6.90E−03 |
| ENSG00000102384 | CENPI | 0.021332262 | −0.475386617 | 5.97E−02 |
| ENSG00000171791 | BCL2 | 0.008656918 | −0.474172722 | 6.00E−01 |
| ENSG00000065970 | FOXJ2 | 0.017646559 | −0.4735107 | 1 |
| ENSG00000066933 | MYO9A | 0.008868511 | −0.473506657 | 1 |
| ENSG00000120709 | FAM53C | 0.016598125 | −0.471476024 | 2.15E−01 |
| ENSG00000182150 | ERCC6L2 | 0.016924169 | −0.466789648 | 1 |
| ENSG00000100393 | EP300 | 1.18E−05 | −0.464539688 | 1.10E−03 |
| ENSG00000143479 | DYRK3 | 0.013602392 | −0.462587869 | 3.58E−02 |
| ENSG00000136770 | DNAJC1 | 0.019563299 | −0.448209599 | 2.70E−03 |
| ENSG00000100354 | TNRC6B | 0.002839187 | −0.444181516 | 1 |
| ENSG00000120949 | TNFRSF8 | 0.022812631 | −0.437286644 | 7.40E−03 |
| ENSG00000154370 | TRIM11 | 0.010190424 | −0.431525912 | 3.01E−04 |
| ENSG00000111450 | STX2 | 0.024088299 | −0.4313432 | 3.52E−01 |
| ENSG00000123575 | FAM199X | 0.029642368 | −0.431235143 | 2.08E−02 |
| ENSG00000179195 | ZNF664 | 0.005477293 | −0.42685597 | 1 |
| ENSG00000165244 | ZNF367 | 0.002786549 | −0.420795786 | 5.50E−03 |
| ENSG00000166024 | R3HCC1L | 0.024332543 | −0.416140766 | 1 |
| ENSG00000123066 | MED13L | 4.79E−05 | −0.415941737 | 2.20E−08 |
| ENSG00000198924 | DCLRE1A | 0.011556077 | −0.415137858 | 1.40E−02 |
| ENSG00000143570 | SLC39A1 | 0.002068007 | −0.414472027 | 1.43E−01 |
| ENSG00000145349 | CAMK2D | 0.021359574 | −0.408237368 | 7.82E−02 |
| ENSG00000110218 | PANX1 | 0.005542647 | −0.39874583 | 2.59E−02 |
| ENSG00000003402 | CFLAR | 0.024657096 | −0.397685039 | 4.71E−02 |
| ENSG00000164168 | TMEM184C | 0.015050183 | −0.397412646 | 2.50E−08 |
| ENSG00000169018 | FEM1B | 0.01910054 | −0.397020738 | 3.00E−04 |
| ENSG00000168092 | PAFAH1B2 | 0.008647229 | −0.388340708 | 3.82E−02 |
| ENSG00000178209 | PLEC | 0.015088771 | −0.385965026 | 1 |
| ENSG00000007168 | PAFAH1B1 | 0.000466042 | −0.383179082 | 9.80E−03 |
| ENSG00000179912 | R3HDM2 | 0.018146798 | −0.377340675 | 9.00E−02 |
| ENSG00000169967 | MAP3K2 | 0.013056576 | −0.377048905 | 6.60E−03 |
| ENSG00000157600 | TMEM164 | 0.010364528 | −0.374375608 | 3.56E−01 |
| ENSG00000213654 | GPSM3 | 0.018261413 | −0.371438487 | 1.02E−01 |
| ENSG00000137310 | TCF19 | 0.006852109 | −0.371039482 | 2.27E−02 |
| ENSG00000126215 | XRCC3 | 0.022817606 | −0.355664276 | 1.50E−03 |
| ENSG00000033170 | FUT8 | 0.006226232 | −0.355628717 | 6.00E−04 |
| ENSG00000162889 | MAPKAPK2 | 0.016286083 | −0.352699883 | 5.49E−01 |
| ENSG00000063978 | RNF4 | 0.002157553 | −0.344201177 | 1.28E−02 |
| ENSG00000177542 | SLC25A22 | 0.013143394 | −0.338628583 | 1.17E−01 |
| ENSG00000132388 | UBE2G1 | 0.000234936 | −0.336746882 | 6.21E−03 |
| ENSG00000170340 | B3GNT2 | 0.004125239 | −0.332264603 | 2.00E−02 |
| ENSG00000064490 | RFXANK | 0.015800837 | −0.324746409 | 3.00E−04 |
| ENSG00000100105 | PATZ1 | 0.00652701 | −0.324460116 | 3.10E−03 |
| ENSG00000103502 | CDIPT | 0.021406946 | −0.323954264 | 1.43E−01 |
| ENSG00000095380 | NANS | 0.027123309 | −0.321819229 | 5.39E−02 |
| ENSG00000160917 | CPSF4 | 0.016200762 | −0.319405125 | 1.70E−03 |
| ENSG00000158435 | CNOT11 | 0.001841931 | −0.314941827 | 8.69E−02 |
| ENSG00000153561 | RMND5A | 0.029110593 | −0.311729374 | 2.03E−01 |
| ENSG00000102858 | MGRN1 | 0.02977034 | −0.309624822 | 1.40E−02 |
| ENSG00000058668 | ATP2B4 | 0.000680955 | −0.302809666 | 1.00E−09 |
| ENSG00000143418 | CERS2 | 5.25E−06 | −0.30206333 | 2.77E−01 |
| ENSG00000089009 | RPL6 | 0.004810696 | −0.297683768 | 3.87E−01 |
| ENSG00000196155 | PLEKHG4 | 0.015595222 | −0.295432913 | 1.00E−04 |
| ENSG00000108510 | MED13 | 0.005034555 | −0.293870753 | 1.60E−03 |
| ENSG00000112531 | QKI | 0.00269178 | −0.29125342 | 8.40E−03 |
| ENSG00000053770 | AP5M1 | 0.011896453 | −0.283753407 | 1.00E−02 |
| ENSG00000163349 | HIPK1 | 0.020020123 | −0.282659771 | 2.00E−04 |
| ENSG00000122257 | RBBP6 | 0.000891329 | −0.280964053 | 1.25E−09 |
| ENSG00000017483 | SLC38A5 | 0.015966238 | −0.280122734 | 2.30E−03 |
| ENSG00000171522 | PTGER4 | 0.002288634 | −0.274827847 | 2.40E−07 |
| ENSG00000111885 | MAN1A1 | 0.010654006 | −0.274580872 | 2.24E−02 |
| ENSG00000048405 | ZNF800 | 0.020652909 | −0.271097499 | 3.00E−08 |
| ENSG00000115419 | GLS | 0.000197719 | −0.269875671 | 4.00E−04 |
| ENSG00000112851 | E RBB2IP | 0.005962767 | −0.26900197 | 3.00E−04 |
| ENSG00000105287 | PRKD2 | 0.018773736 | −0.262403451 | 1.25E−02 |
| ENSG00000182831 | C16or172 | 0.00375696 | −0.255255837 | 1.07E−01 |
| ENSG00000007968 | E2F2 | 0.009479782 | −0.24865366 | 4.20E−03 |
| ENSG00000100225 | FBXO7 | 0.00882935 | −0.246942196 | 1.08E−02 |
| ENSG00000171552 | BCL2L1 | 0.00522943 | −0.245325394 | 6.32E−02 |
| ENSG00000131507 | NDFIP1 | 0.004173323 | −0.242895723 | 6.80E−03 |
| ENSG00000090621 | PABPC4 | 0.000282201 | −0.242834811 | 1.71E−02 |
| ENSG00000134602 | MST4 | 0.003080229 | −0.242407773 | 1.05E−02 |
| ENSG00000159692 | CTBP1 | 0.006057739 | −0.241648156 | 5.00E−03 |
| ENSG00000133657 | ATP13A3 | 0.021301072 | −0.237052311 | 1.00E−10 |
| ENSG00000106609 | TMEM248 | 0.005316307 | −0.23621242 | 2.90E−07 |
| ENSG00000152684 | PELO | 0.01293572 | −0.236131973 | 7.82E−02 |
| ENSG00000104325 | DECR1 | 0.01371001 | −0.232829844 | 1.19E−02 |
| ENSG00000134954 | ETS1 | 1.27E−05 | −0.232324455 | 5.70E−09 |
| ENSG00000140332 | TLE3 | 0.001343794 | −0.227829431 | 2.00E−08 |
| ENSG00000147140 | NONO | 0.003389405 | −0.227308156 | 3.57E−02 |
| ENSG00000169905 | TOR1AIP2 | 0.025144824 | −0.223803399 | 7.00E−04 |
| ENSG00000149480 | MTA2 | 2.64E−05 | −0.22354576 | 6.00E−09 |
| ENSG00000136997 | MYC | 0.000130485 | −0.222358961 | 3.00E−08 |
| ENSG00000105329 | TGFB1 | 0.015301045 | −0.221315351 | 2.00E−04 |
| ENSG00000107485 | GATA3 | 0.025270056 | −0.218676008 | 1.34E−02 |
| ENSG00000171858 | RPS21 | 0.00024084 | −0.216003759 | 5.41E−03 |
| ENSG00000131504 | DIAPH1 | 0.005406879 | −0.213577391 | 2.00E−04 |
| ENSG00000138795 | LEF1 | 1.60E−06 | −0.210659864 | 1.00E−09 |
| ENSG00000106290 | TAF6 | 0.014175182 | −0.210235711 | 5.00E−04 |
| ENSG00000137845 | ADAM10 | 0.012053048 | −0.208903322 | 9.00E−10 |
| ENSG00000136878 | USP20 | 0.019165529 | −0.206602358 | 2.00E−08 |
| ENSG00000174579 | MSL2 | 0.027763257 | −0.205275001 | 5.00E−04 |
| ENSG00000172292 | CERS6 | 0.029552171 | −0.205124483 | 1.00E−09 |
| ENSG00000125691 | RPL23 | 0.004476603 | −0.201854357 | 1.43E−02 |
| ENSG00000135932 | CAB39 | 0.019948395 | −0.200243436 | 1.00E−08 |
| ENSG00000155508 | CNOT8 | 0.026465866 | −0.200101165 | 6.78E−02 |
| ENSG00000108578 | BLMH | 0.011959202 | −0.198123991 | 1.66E−01 |
| ENSG00000118816 | CCNI | 0.001528498 | −0.189161037 | 6.90E−07 |
| ENSG00000101972 | STAG2 | 0.001047325 | −0.187270211 | 7.00E−04 |
| ENSG00000151465 | CDC123 | 0.007301 | −0.184803611 | 3.23E−01 |
| ENSG00000140262 | TCF12 | 0.025334533 | −0.184064816 | 2.00E−10 |
| ENSG00000159216 | RUNX1 | 0.004534671 | −0.177621274 | 5.20E−03 |
| ENSG00000100796 | SMEK1 | 0.021404696 | −0.176469607 | 1.00E−11 |
| ENSG00000143889 | HNRPLL | 0.02829111 | −0.174975752 | 1.60E−03 |
| ENSG00000112306 | RPS12 | 0.029227861 | −0.173199482 | 1.13E−01 |
| ENSG00000108424 | KPNB1 | 8.66E−05 | −0.171777065 | 2.00E−11 |
| ENSG00000085117 | CD82 | 0.001401537 | −0.168060459 | 2.70E−03 |
| ENSG00000111371 | SLC38A1 | 0.006852608 | −0.165827185 | 3.43E−02 |
| ENSG00000099800 | TIMM13 | 0.01109652 | −0.165570746 | 8.64E−02 |
| ENSG00000105063 | PPP6R1 | 0.029786388 | −0.157573098 | 1.00E−10 |
| ENSG00000120727 | PAIP2 | 0.010801093 | −0.157320231 | 5.59E−01 |
| ENSG00000109685 | WHSC1 | 0.0222009 | −0.153361985 | 1.00E−11 |
| ENSG00000152601 | MBNL1 | 0.00777836 | −0.152181062 | 2.00E−11 |
| ENSG00000197771 | MCMBP | 0.0158736 | −0.149779012 | 5.90E−03 |
| ENSG00000088325 | TPX2 | 0.000751758 | −0.147886462 | 4.00E−13 |
| ENSG00000171310 | CHST11 | 0.004536717 | −0.14604981 | 1.20E−07 |
| ENSG00000158985 | CDC42SE2 | 0.027991366 | −0.145160094 | 8.00E−08 |
| ENSG00000184007 | PTP4A2 | 0.00039459 | −0.142942918 | 1.50E−07 |
| ENSG00000153310 | FAM49B | 0.007506383 | −0.139159484 | 2.00E−04 |
| ENSG00000121083 | DYNLL2 | 0.02989098 | −0.137797441 | 1.00E−03 |
| ENSG00000078369 | GNB1 | 0.011642786 | −0.133797709 | 5.00E−04 |
| ENSG00000125743 | SNRPD2 | 0.024903253 | −0.131448444 | 2.38E−01 |
| ENSG00000110651 | CD81 | 0.010480682 | −0.130640591 | 1.00E−04 |
| ENSG00000077312 | SNRPA | 0.023537735 | −0.127819876 | 1.26E−01 |
| ENSG00000125970 | RALY | 0.004001428 | −0.112279678 | 3.64E−02 |
| ENSG00000186468 | RPS23 | 0.008328741 | −0.104395342 | 5.03E−01 |
| ENSG00000169764 | UGP2 | 0.027598388 | −0.103615488 | 1.60E−03 |
| ENSG00000138668 | HNRNPD | 0.003261874 | −0.098862205 | 2.40E−07 |
| ENSG00000167978 | SRRM2 | 0.027585188 | −0.081656945 | 1.00E−04 |

TABLE 3C
TE down genes with G-Quadruplex structure

| Gene ID | Gene Name | Translational Efficiency (p-value) | log2(Translational Efficiency) | rDiff (p-value) |
|---|---|---|---|---|
| ENSG00000127152 | BCL11B | 5.40E−10 | −0.517770746 | 1.00E−08 |
| ENSG00000100393 | EP300 | 1.18E−05 | −0.464539688 | 1.10E−03 |
| ENSG00000149480 | MTA2 | 2.64E−05 | −0.22354576 | 6.00E−09 |
| ENSG00000180730 | SHISA2 | 2.85E−05 | −0.608613867 | 1.93E−02 |
| ENSG00000123066 | MED13L | 4.79E−05 | −0.415941737 | 2.20E−08 |
| ENSG00000132388 | UBE2G1 | 0.000234936 | −0.336746882 | 6.21E−03 |
| ENSG00000166503 | HDGFRP3 | 0.000273828 | −0.608750084 | 1.19E−02 |
| ENSG00000090621 | PABPC4 | 0.000282201 | −0.242834811 | 1.71E−02 |
| ENSG00000184007 | PTP4A2 | 0.00039459 | −0.142942918 | 1.50E−07 |
| ENSG00000007168 | PAFAH1B1 | 0.000466042 | −0.383179082 | 9.80E−03 |
| ENSG00000058668 | ATP2B4 | 0.000680955 | −0.302809666 | 1.00E−09 |
| ENSG00000101972 | STAG2 | 0.001047325 | −0.187270211 | 7.00E−04 |
| ENSG00000109654 | TRIM2 | 0.001320775 | −2.146623909 | 1 |
| ENSG00000140332 | TLE3 | 0.001343794 | −0.227829431 | 2.00E−08 |
| ENSG00000063978 | RNF4 | 0.002157553 | −0.344201177 | 1.28E−02 |
| ENSG00000171522 | PTGER4 | 0.002288634 | −0.274827847 | 2.40E−07 |
| ENSG00000112531 | QKI | 0.00269178 | −0.29125342 | 8.40E−03 |
| ENSG00000100354 | TNRC6B | 0.002839187 | −0.444181516 | 1 |
| ENSG00000182831 | C16orf72 | 0.00375696 | −0.255255837 | 1.07E−01 |
| ENSG00000131507 | NDFIP1 | 0.004173323 | −0.242895723 | 6.80E−03 |
| ENSG00000171310 | CHST11 | 0.004536717 | −0.14604981 | 1.20E−07 |
| ENSG00000108510 | MED13 | 0.005034555 | −0.293870753 | 1.60E−03 |
| ENSG00000162065 | TBC1D24 | 0.005127762 | −1.23071089 | 1 |
| ENSG00000106609 | TMEM248 | 0.005316307 | −0.23621242 | 2.90E−07 |
| ENSG00000131504 | DIAPH1 | 0.005406879 | −0.213577391 | 2.00E−04 |
| ENSG00000110218 | PANX1 | 0.005542647 | −0.39874583 | 2.59E−02 |
| ENSG00000123159 | GIPC1 | 0.00645275 | −0.478285755 | 6.90E−03 |
| ENSG00000100105 | PATZ1 | 0.00652701 | −0.324460116 | 3.10E−03 |
| ENSG00000153310 | FAM49B | 0.007506383 | −0.139159484 | 2.00E−04 |
| ENSG00000152601 | MBNL1 | 0.00777836 | −0.152181062 | 2.00E−11 |
| ENSG00000151014 | CCRN4L | 0.007814184 | −0.480208055 | 7.50E−03 |
| ENSG00000168092 | PAFAH1B2 | 0.008647229 | −0.388340708 | 3.82E−02 |
| ENSG00000119844 | AFTPH | 0.008725735 | −0.591675727 | 1 |
| ENSG00000139112 | GABARAPL1 | 0.008844095 | −1.233522978 | 1 |
| ENSG00000066933 | MYO9A | 0.008868511 | −0.473506657 | 1 |
| ENSG00000120727 | PAIP2 | 0.010801093 | −0.157320231 | 5.59E−01 |
| ENSG00000078369 | GNB1 | 0.011642786 | −0.133797709 | 5.00E−04 |
| ENSG00000137845 | ADAM10 | 0.012053048 | −0.208903322 | 9.00E−10 |
| ENSG00000169967 | MAP3K2 | 0.013056576 | −0.377048905 | 6.60E−03 |
| ENSG00000177542 | SLC25A22 | 0.013143394 | −0.338628583 | 1.17E−01 |
| ENSG00000106290 | TAF6 | 0.014175182 | −0.210235711 | 5.00E−04 |
| ENSG00000105329 | TGFB1 | 0.015301045 | −0.221315351 | 2.00E−04 |
| ENSG00000064490 | RFXANK | 0.015800837 | −0.324746409 | 3.00E−04 |
| ENSG00000180035 | ZNF48 | 0.015918146 | −0.582772982 | 1 |
| ENSG00000160917 | CPSF4 | 0.016200762 | −0.319405125 | 1.70E−03 |
| ENSG00000162889 | MAPKAPK2 | 0.016286083 | −0.352699883 | 5.49E−01 |
| ENSG00000181027 | FKRP | 0.016390601 | −0.996001717 | 1 |
| ENSG00000065970 | FOXJ2 | 0.017646559 | −0.4735107 | 1 |
| ENSG00000137822 | TUBGCP4 | 0.017824276 | −0.534568492 | 3.06E−02 |
| ENSG00000109220 | CHIC2 | 0.018242688 | −0.521221308 | 1.70E−02 |
| ENSG00000136878 | USP20 | 0.019165529 | −0.206602358 | 2.00E−08 |
| ENSG00000163349 | HIPK1 | 0.020020123 | −0.282659771 | 2.00E−04 |
| ENSG00000048405 | ZNF800 | 0.020652909 | −0.271097499 | 3.00E−08 |
| ENSG00000145349 | CAMK2D | 0.021359574 | −0.408237368 | 7.82E−02 |
| ENSG00000100796 | SMEK1 | 0.021404696 | −0.176469607 | 1.00E−11 |
| ENSG00000103502 | CDIPT | 0.021406946 | −0.323954264 | 1.43E−01 |
| ENSG00000124786 | SLC35B3 | 0.023814954 | −0.917840885 | 1 |
| ENSG00000150995 | ITPR1 | 0.025529346 | −0.628260516 | 1 |
| ENSG00000034677 | RNF19A | 0.025744017 | −0.483894067 | 1 |
| ENSG00000056998 | GYG2 | 0.026501722 | −0.929650779 | 1 |
| ENSG00000204348 | DOM3Z | 0.026501738 | −0.595853393 | 1 |
| ENSG00000095380 | NANS | 0.027123309 | −0.321819229 | 5.39E−02 |
| ENSG00000130669 | PAK4 | 0.02752902 | −0.627975797 | 1 |
| ENSG00000167978 | SRRM2 | 0.027585188 | −0.081656945 | 1.00E−04 |
| ENSG00000112306 | RPS12 | 0.029227861 | −0.173199482 | 1.13E−01 |
| ENSG00000172292 | CERS6 | 0.029552171 | −0.205124483 | 1.00E−09 |
| ENSG00000102858 | MGRN1 | 0.02977034 | −0.309624822 | 1.40E−02 |
| ENSG00000105063 | PPP6R1 | 0.029786388 | −0.157573098 | 1.00E−10 |
| ENSG00000121083 | DYNLL2 | 0.02989098 | −0.137797441 | 1.00E−03 |

TABLE 4
rDiff positive genes have significant changes in ribosome footprint distribution

| Gene ID | Gene Name | rDiff (p-value) | Translational Efficiency (p-value) | log2(Translational Efficiency) |
|---|---|---|---|---|
| ENSG00000002822 | MAD1L1 | 1.70E−07 | 0.758278428 | −0.03032717 |
| ENSG00000003056 | M6PR | 8.05E−04 | 0.341786644 | −0.06800052 |
| ENSG00000004700 | RECQL | 1.00E−08 | 0.598214663 | −0.04903859 |
| ENSG00000004779 | NDUFAB1 | 5.26E−06 | 0.902185935 | 0.006429888 |
| ENSG00000005007 | UPF1 | 3.00E−04 | 0.109682217 | −0.088407059 |
| ENSG00000005955 | GGNBP2 | 6.00E−04 | 0.626526855 | −0.042670122 |
| ENSG00000006114 | SYNRG | 2.00E−09 | 0.411165702 | 0.079008553 |
| ENSG00000008952 | SEC62 | 9.90E−07 | 0.412197652 | 0.074389117 |
| ENSG00000009307 | CSDE1 | 5.60E−07 | 0.282559234 | −0.043586952 |
| ENSG00000009335 | UBE3C | 7.00E−08 | 0.96703173 | 0.005178317 |
| ENSG00000009790 | TRAF3IP3 | 2.00E−04 | 0.932693262 | 0.007653676 |
| ENSG00000009954 | BAZ1B | 1.00E−12 | 0.813245824 | 0.011597992 |
| ENSG00000010810 | FYN | 4.02E−04 | 0.194054982 | −0.146493062 |
| ENSG00000011295 | TTC19 | 3.00E−04 | 0.726452954 | 0.048391976 |
| ENSG00000011376 | LARS2 | 6.02E−04 | 0.269847002 | −0.142543886 |
| ENSG00000013810 | TACC3 | 2.00E−09 | 0.886452126 | −0.009226853 |
| ENSG00000018699 | TTC27 | 3.00E−04 | 0.640010253 | −0.057075791 |
| ENSG00000021355 | SERPINB1 | 4.00E−04 | 0.866004942 | −0.014203345 |
| ENSG00000021762 | OSBPL5 | 7.01E−04 | 0.905417327 | −0.030769757 |
| ENSG00000027697 | IFNGR1 | 4.00E−04 | 0.409322091 | 0.099560346 |
| ENSG00000030066 | NUP160 | 1.00E−11 | 0.645030987 | −0.030045128 |
| ENSG00000030419 | IKZF2 | 3.00E−04 | 0.413365119 | −0.055123312 |
| ENSG00000031698 | SARS | 2.00E−04 | 0.872730663 | 0.011917 |
| ENSG00000033030 | ZCCHC8 | 6.10E−08 | 0.867990166 | −0.021717099 |
| ENSG00000033170 | FUT8 | 6.00E−04 | 0.006226232 | −0.355628717 |
| ENSG00000033178 | UBA6 | 4.00E−04 | 0.07871533 | 0.184240847 |
| ENSG00000033800 | PIAS1 | 6.01E−04 | 0.942720076 | 0.005741934 |
| ENSG00000036257 | CUL3 | 3.00E−04 | 0.568119382 | 0.048886832 |
| ENSG00000038210 | PI4K2B | 1.01E−04 | 0.689067203 | 0.060060784 |
| ENSG00000038219 | BOD1L1 | 1.00E−04 | 0.003376816 | 0.278967432 |
| ENSG00000038358 | EDC4 | 3.00E−04 | 0.449660549 | 0.070646944 |
| ENSG00000039123 | SKIV2L2 | 5.00E−08 | 0.397584419 | 0.067295718 |
| ENSG00000043462 | LCP2 | 1.00E−04 | 0.236691265 | −0.074243034 |
| ENSG00000047315 | POLR2B | 9.04E−04 | 0.311086732 | 0.072452266 |
| ENSG00000047410 | TPR | 2.00E−11 | 0.045762118 | 0.103472955 |
| ENSG00000048405 | ZNF800 | 3.00E−08 | 0.020652909 | −0.271097499 |
| ENSG00000048740 | CELF2 | 8.00E−04 | 0.039583811 | 0.136650125 |
| ENSG00000049618 | ARID1B | 1.00E−04 | 0.385309666 | 0.127414641 |
| ENSG00000051523 | CYBA | 1.00E−04 | 0.816434248 | 0.0204381 |
| ENSG00000052841 | TTC17 | 9.01E−04 | 0.972969728 | 0.004383509 |
| ENSG00000054654 | SYNE2 | 9.00E−09 | 0.143319349 | −0.17547751 |
| ENSG00000055044 | NOP58 | 1.00E−11 | 0.905915474 | −0.006508419 |
| ENSG00000055130 | CUL1 | 2.00E−04 | 0.24134893 | −0.116033963 |
| ENSG00000055163 | CYFIP2 | 6.00E−13 | 0.757974081 | −0.02427969 |
| ENSG00000055483 | USP36 | 2.00E−10 | 0.857880476 | 0.014047197 |
| ENSG00000058063 | ATP11B | 5.00E−04 | 0.340893448 | 0.106997948 |
| ENSG00000058668 | ATP2B4 | 1.00E−09 | 0.000680955 | −0.302809666 |
| ENSG00000058729 | RIOK2 | 1.00E−09 | 0.452540408 | 0.12495794 |
| ENSG00000059573 | ALDH18A1 | 4.00E−08 | 0.254939161 | −0.109183666 |
| ENSG00000060069 | CTDP1 | 1.00E−04 | 0.606500761 | −0.103959418 |
| ENSG00000060237 | WNK1 | 8.00E−04 | 0.614924113 | −0.029766546 |
| ENSG00000060339 | CCAR1 | 1.00E−04 | 0.526971388 | 0.044049323 |
| ENSG00000060491 | OGFR | 6.00E−04 | 0.881572577 | −0.018437371 |
| ENSG00000062650 | WAPAL | 8.00E−04 | 0.019004475 | −0.211592796 |
| ENSG00000062822 | POLD1 | 1.00E−04 | 0.881523419 | −0.010752676 |
| ENSG00000063245 | EPN1 | 6.00E−04 | 0.095672127 | −0.260058118 |
| ENSG00000064115 | TM7SF3 | 3.00E−04 | 0.921247137 | 0.007233323 |
| ENSG00000064419 | TNPO3 | 1.00E−04 | 0.030219393 | −0.182349237 |
| ENSG00000064490 | RFXANK | 3.00E−04 | 0.015800837 | −0.324746409 |
| ENSG00000065150 | IPO5 | 1.00E−04 | 0.241050243 | 0.063914843 |
| ENSG00000065328 | MCM10 | 3.00E−08 | 0.796034459 | −0.019990672 |
| ENSG00000065357 | DGKA | 7.00E−04 | 0.601907834 | −0.04235016 |
| ENSG00000065526 | SPEN | 1.00E−04 | 0.859973887 | −0.020485515 |
| ENSG00000065613 | SLK | 2.00E−08 | 0.412051605 | −0.115711529 |
| ENSG00000066084 | DIP2B | 2.20E−08 | 0.728581353 | 0.041530278 |
| ENSG00000066279 | ASPM | 2.00E−09 | 0.013635649 | 0.181712013 |
| ENSG00000066654 | THUMPD1 | 1.00E−04 | 0.066318449 | 0.218332022 |
| ENSG00000067082 | KLF6 | 6.00E−04 | 0.873448029 | 0.015089776 |
| ENSG00000067167 | TRAM1 | 1.80E−07 | 0.785985329 | −0.016953438 |
| ENSG00000067225 | PKM | 6.00E−04 | 0.089243304 | 0.079429696 |
| ENSG00000067596 | DHX8 | 2.00E−08 | 0.577229448 | 0.056415372 |
| ENSG00000068024 | HDAC4 | 1.00E−08 | 0.08742941 | −0.17058278 |
| ENSG00000068796 | KIF2A | 1.00E−04 | 0.056041279 | 0.123499472 |
| ENSG00000070756 | PABPC1 | 1.00E−07 | 0.067231582 | −0.133311245 |
| ENSG00000071054 | MAP4K4 | 3.00E−10 | 0.191479972 | −0.114359829 |
| ENSG00000071127 | WDR1 | 1.00E−08 | 0.661342679 | −0.016553317 |
| ENSG00000071564 | TCF3 | 1.70E−07 | 0.851040343 | −0.020991025 |
| ENSG00000071626 | DAZAP1 | 5.20E−07 | 0.060128417 | −0.110195121 |
| ENSG00000071894 | CPSF1 | 1.00E−04 | 0.292670446 | −0.087303379 |
| ENSG00000072062 | PRKACA | 5.00E−04 | 0.823686793 | −0.022935317 |
| ENSG00000072310 | SREBF1 | 1.00E−04 | 0.744899078 | −0.026352209 |
| ENSG00000072364 | AFF4 | 4.01E−04 | 0.18444246 | −0.147821651 |
| ENSG00000072778 | ACADVL | 2.01E−04 | 0.634381953 | 0.052306846 |
| ENSG00000073060 | SCARB1 | 4.00E−04 | 0.444106259 | −0.124760329 |
| ENSG00000073614 | KDM5A | 7.00E−04 | 0.292729244 | −0.104574461 |
| ENSG00000074370 | ATP2A3 | 1.00E−04 | 0.51081004 | −0.034487857 |
| ENSG00000074603 | DPP8 | 1.09E−06 | 0.371935225 | −0.109929668 |
| ENSG00000074695 | LMAN1 | 6.00E−04 | 0.136972912 | 0.109603097 |
| ENSG00000074755 | ZZEF1 | 4.01E−04 | 0.145270305 | 0.179883886 |
| ENSG00000075415 | SLC25A3 | 3.30E−07 | 0.062332659 | −0.064744845 |
| ENSG00000075539 | FRYL | 1.00E−04 | 0.128443961 | 0.156722872 |
| ENSG00000075975 | MKRN2 | 6.01E−04 | 0.98382419 | 0.004421651 |
| ENSG00000076108 | BAZ2A | 1.00E−04 | 0.708000473 | −0.042895235 |
| ENSG00000076770 | MBNL3 | 2.60E−07 | 0.135662004 | −0.159547551 |
| ENSG00000077097 | TOP2B | 1.00E−04 | 0.84957972 | 0.01045839 |
| ENSG00000077232 | DNAJC10 | 2.00E−04 | 0.043053807 | −0.322931196 |
| ENSG00000078369 | GNB1 | 5.00E−04 | 0.011642786 | −0.133797709 |
| ENSG00000078618 | NRD1 | 5.00E−04 | 0.441231318 | −0.057082496 |
| ENSG00000078674 | PCM1 | 5.00E−11 | 0.600752059 | −0.058335335 |
| ENSG00000078687 | TNRC6C | 3.00E−04 | 0.332794972 | −0.148534844 |
| ENSG00000079313 | REXO1 | 7.00E−04 | 0.723402497 | −0.050607046 |
| ENSG00000079432 | CIC | 8.00E−04 | 0.820970963 | −0.041443313 |
| ENSG00000079805 | DNM2 | 1.53E−06 | 0.594275253 | −0.034637408 |
| ENSG00000080345 | RIF1 | 3.00E−11 | 0.722609171 | 0.022548991 |
| ENSG00000080815 | PSEN1 | 1.50E−07 | 0.468385762 | −0.105188191 |
| ENSG00000080986 | NDC80 | 4.00E−04 | 0.202768868 | 0.097858575 |
| ENSG00000081019 | RSBN1 | 8.00E−04 | 0.864427786 | −0.025477267 |
| ENSG00000081237 | PTPRC | 7.00E−04 | 0.015733814 | 0.122576702 |
| ENSG00000081791 | KIAA0141 | 9.00E−04 | 0.905406058 | −0.017374448 |
| ENSG00000082212 | ME2 | 8.00E−04 | 0.795777072 | −0.028699073 |
| ENSG00000082516 | GEMIN5 | 1.00E−04 | 0.382720363 | 0.09923243 |
| ENSG00000082641 | NFE2L1 | 1.00E−09 | 0.267959196 | −0.205302853 |
| ENSG00000082898 | XPO1 | 1.00E−11 | 0.026533538 | −0.106116515 |
| ENSG00000083312 | TNPO1 | 8.00E−04 | 0.96323084 | 0.002633087 |
| ENSG00000083642 | PDS5B | 6.00E−04 | 0.586150567 | 0.042212365 |
| ENSG00000083845 | RPS5 | 3.02E−04 | 0.290962665 | −0.089607269 |
| ENSG00000083857 | FAT1 | 2.07E−09 | 0.602892898 | −0.038812678 |
| ENSG00000084093 | REST | 1.00E−09 | 0.221405653 | −0.118069779 |
| ENSG00000084207 | GSTP1 | 8.00E−04 | 0.028975686 | 0.078499851 |
| ENSG00000084733 | RAB10 | 7.00E−08 | 0.078220422 | −0.136343032 |
| ENSG00000084774 | CAD | 2.00E−08 | 0.242515439 | 0.087577807 |
| ENSG00000086102 | NFX1 | 7.00E−04 | 0.176154723 | 0.162273705 |
| ENSG00000086504 | MRPL28 | 1.00E−04 | 0.056394 | −0.151444666 |
| ENSG00000086758 | HUWE1 | 2.40E−11 | 0.464534104 | −0.039863394 |
| ENSG00000087087 | SRRT | 1.00E−10 | 0.966692349 | 0.001824104 |
| ENSG00000087365 | SF3B2 | 1.00E−04 | 0.108200543 | 0.071832895 |
| ENSG00000087460 | GNAS | 3.00E−10 | 0.461136397 | −0.032870857 |
| ENSG00000088247 | KHSRP | 2.00E−04 | 0.673716802 | −0.021332247 |
| ENSG00000088325 | TPX2 | 4.00E−13 | 0.000751758 | −0.147886462 |
| ENSG00000088930 | XRN2 | 1.00E−04 | 0.054328641 | 0.120769979 |
| ENSG00000089053 | ANAPC5 | 2.00E−04 | 0.71210468 | −0.020762022 |
| ENSG00000089094 | KDM2B | 4.00E−08 | 0.242750733 | −0.121485992 |
| ENSG00000089154 | GCN1L1 | 3.00E−09 | 0.913083626 | −0.007259602 |
| ENSG00000089234 | BRAP | 2.00E−04 | 0.93786126 | −0.010542696 |
| ENSG00000090061 | CCNK | 8.00E−08 | 0.870725186 | −0.014115514 |
| ENSG00000090372 | STRN4 | 6.00E−04 | 0.942223216 | 0.00735298 |
| ENSG00000090520 | DNAJB11 | 1.00E−04 | 0.403800964 | −0.067101447 |
| ENSG00000090861 | AARS | 6.00E−04 | 0.574152586 | 0.035132718 |
| ENSG00000091127 | PUS7 | 9.00E−04 | 0.204909104 | −0.155033013 |
| ENSG00000091164 | TXNL1 | 1.00E−07 | 0.371475872 | −0.0727392 |
| ENSG00000091317 | CMTM6 | 2.00E−08 | 0.443439517 | 0.064753558 |
| ENSG00000092094 | OSGEP | 2.00E−08 | 0.977762307 | −0.002519015 |
| ENSG00000092853 | CLSPN | 1.00E−10 | 0.062944195 | 0.125340878 |
| ENSG00000092964 | DPYSL2 | 1.72E−06 | 0.18869348 | −0.144294954 |
| ENSG00000093009 | CDC45 | 4.00E−04 | 0.918341518 | 0.007977992 |
| ENSG00000093167 | LRRFIP2 | 1.00E−04 | 0.814819674 | −0.032970227 |
| ENSG00000095319 | NUP188 | 5.00E−04 | 0.409660705 | −0.062866839 |
| ENSG00000096401 | CDC5L | 4.00E−11 | 0.958393737 | −0.003388653 |
| ENSG00000097046 | CDC7 | 3.00E−04 | 0.543156786 | −0.062966344 |
| ENSG00000099331 | MYO9B | 6.00E−04 | 0.792985514 | 0.024635401 |
| ENSG00000099381 | SETD1A | 1.00E−08 | 0.334979113 | 0.098318494 |
| ENSG00000099991 | CABIN1 | 1.00E−08 | 0.104911155 | −0.208959207 |
| ENSG00000100029 | PES1 | 3.00E−04 | 0.805834098 | −0.019606907 |
| ENSG00000100138 | NHP2L1 | 1.00E−04 | 0.451814068 | −0.053140436 |
| ENSG00000100147 | CCDC134 | 6.00E−08 | 0.397335351 | −0.115525081 |
| ENSG00000100242 | SUN2 | 4.00E−04 | 0.443353969 | 0.107423956 |
| ENSG00000100258 | LMF2 | 3.01E−04 | 0.446590324 | −0.096455839 |
| ENSG00000100280 | AP1B1 | 1.00E−04 | 0.155202288 | −0.113131916 |
| ENSG00000100296 | THOC5 | 1.00E−04 | 0.777068501 | 0.04404948 |
| ENSG00000100345 | MYH9 | 2.10E−13 | 0.195171025 | −0.051020259 |
| ENSG00000100350 | FOXRED2 | 1.00E−04 | 0.939313293 | −0.00792074 |
| ENSG00000100401 | RANGAP1 | 1.00E−04 | 0.59275986 | −0.053239149 |
| ENSG00000100403 | ZC3H7B | 4.00E−04 | 0.241293577 | −0.127436934 |
| ENSG00000100422 | CERK | 5.01E−04 | 0.767879843 | −0.038668 |
| ENSG00000100461 | RBM23 | 2.00E−04 | 0.126115054 | −0.188774943 |
| ENSG00000100528 | CNIH | 2.00E−11 | 0.396062577 | 0.115843844 |
| ENSG00000100554 | ATP6V1D | 1.00E−09 | 0.428461734 | −0.082721884 |
| ENSG00000100697 | DICER1 | 4.00E−04 | 0.937569952 | 0.007626111 |
| ENSG00000100714 | MTHFD1 | 5.00E−04 | 0.068239627 | 0.121426205 |
| ENSG00000100796 | SMEK1 | 1.00E−11 | 0.021404696 | −0.176469607 |
| ENSG00000100813 | ACIN1 | 1.00E−04 | 0.513552164 | −0.041074263 |
| ENSG00000100888 | CHD8 | 5.00E−09 | 0.833778017 | 0.019607725 |
| ENSG00000100911 | PSME2 | 6.11E−04 | 0.954296798 | −0.00949908 |
| ENSG00000100994 | PYGB | 2.02E−04 | 0.741200463 | 0.05467102 |
| ENSG00000100997 | ABHD12 | 1.00E−08 | 0.489735178 | −0.117139463 |
| ENSG00000101161 | PRPF6 | 5.00E−04 | 0.452014829 | 0.060790849 |
| ENSG00000101182 | PSMA7 | 5.01E−04 | 0.980181485 | 0.001199919 |
| ENSG00000101191 | DIDO1 | 9.00E−09 | 0.846657226 | −0.024545447 |
| ENSG00000101224 | CDC25B | 2.00E−09 | 0.25893922 | 0.059946483 |
| ENSG00000101294 | HM13 | 4.00E−04 | 0.788344267 | −0.016820212 |
| ENSG00000101310 | SEC23B | 2.00E−08 | 0.242275151 | 0.116563376 |
| ENSG00000101343 | CRNKL1 | 2.00E−04 | 0.587849423 | 0.05818265 |
| ENSG00000101464 | PIGU | 7.02E−04 | 0.68308036 | −0.052876011 |
| ENSG00000101596 | SMCHD1 | 1.00E−09 | 0.434566245 | −0.059009881 |
| ENSG00000101868 | POLA1 | 2.00E−04 | 0.520751395 | 0.053293939 |
| ENSG00000101972 | STAG2 | 7.00E−04 | 0.001047325 | −0.187270211 |
| ENSG00000102054 | RBBP7 | 1.60E−12 | 0.003411029 | −0.129303881 |
| ENSG00000102125 | TAZ | 7.01E−04 | 0.35021839 | 0.152681248 |
| ENSG00000102189 | EEA1 | 8.02E−04 | 0.70653248 | −0.043342085 |
| ENSG00000102245 | CD40LG | 1.00E−04 | 0.514830532 | 0.081808759 |
| ENSG00000102606 | ARHGEF7 | 6.00E−08 | 0.279523802 | −0.128921833 |
| ENSG00000102908 | NFAT5 | 5.01E−04 | 0.225277986 | −0.178477519 |
| ENSG00000102974 | CTCF | 1.00E−09 | 0.919312546 | −0.009349348 |
| ENSG00000103222 | ABCC1 | 2.00E−04 | 0.969797812 | 0.002188323 |
| ENSG00000103415 | HMOX2 | 1.00E−09 | 0.569411146 | 0.05835055 |
| ENSG00000103479 | RBL2 | 1.00E−04 | 0.773913697 | −0.038025558 |
| ENSG00000103495 | MAZ | 1.30E−07 | 0.642227894 | 0.032737594 |
| ENSG00000103544 | C16orf62 | 3.01E−04 | 0.742406188 | 0.038231542 |
| ENSG00000103591 | AAGAB | 9.00E−04 | 0.205123038 | 0.124804927 |
| ENSG00000104177 | MYEF2 | 7.01E−04 | 0.774370445 | −0.035966517 |
| ENSG00000104365 | IKBKB | 4.00E−04 | 0.474002406 | 0.087889302 |
| ENSG00000104472 | CHRAC1 | 8.00E−04 | 0.353904047 | 0.105337178 |
| ENSG00000104517 | UBR5 | 3.00E−10 | 0.744729033 | 0.031234126 |
| ENSG00000104518 | GSDMD | 5.00E−04 | 0.437398468 | −0.076674502 |
| ENSG00000104549 | SQLE | 1.30E−07 | 0.043864022 | −0.265964104 |
| ENSG00000104613 | INTS10 | 1.00E−04 | 0.796914737 | 0.022823898 |
| ENSG00000104695 | PPP2CB | 7.00E−04 | 0.032333256 | −0.321922476 |
| ENSG00000104738 | MCM4 | 1.00E−10 | 0.915393017 | 0.003107424 |
| ENSG00000104824 | HNRNPL | 3.60E−07 | 0.95648967 | 0.003621772 |
| ENSG00000104825 | NFKBIB | 1.01E−04 | 0.438223723 | 0.114649527 |
| ENSG00000104852 | SNRNP70 | 9.00E−04 | 0.594216034 | −0.038334929 |
| ENSG00000104886 | PLEKHJ1 | 6.00E−08 | 0.445334658 | 0.068851577 |
| ENSG00000105063 | PPP6R1 | 1.00E−10 | 0.029786388 | −0.157573098 |
| ENSG00000105221 | AKT2 | 2.00E−04 | 0.381664023 | −0.141483695 |
| ENSG00000105248 | CCDC94 | 3.00E−04 | 0.324134308 | −0.124113502 |
| ENSG00000105281 | SLC1A5 | 2.90E−10 | 0.106631749 | −0.153858078 |
| ENSG00000105329 | TGFB1 | 2.00E−04 | 0.015301045 | −0.221315351 |
| ENSG00000105374 | NKG7 | 9.00E−04 | 0.272175864 | −0.097254231 |
| ENSG00000105401 | CDC37 | 2.02E−04 | 0.182664767 | 0.098508161 |
| ENSG00000105486 | LIG1 | 9.00E−04 | 0.865181674 | −0.011170383 |
| ENSG00000105618 | PRPF31 | 4.00E−04 | 0.189221467 | 0.102321971 |
| ENSG00000105676 | ARMC6 | 8.00E−04 | 0.830466125 | −0.018392253 |
| ENSG00000105677 | TMEM147 | 5.00E−08 | 0.824197085 | −0.020622451 |
| ENSG00000105810 | CDK6 | 4.00E−08 | 4.76E−05 | −0.145254993 |
| ENSG00000105939 | ZC3HAV1 | 4.00E−04 | 0.868091195 | 0.01093142 |
| ENSG00000105953 | OGDH | 1.20E−08 | 0.404842964 | −0.086222511 |
| ENSG00000106263 | EIF3B | 1.00E−11 | 0.448608969 | −0.028252846 |
| ENSG00000106268 | NUDT1 | 5.77E−06 | 0.36073421 | 0.053967036 |
| ENSG00000106290 | TAF6 | 5.00E−04 | 0.014175182 | −0.210235711 |
| ENSG00000106443 | PHF14 | 6.00E−04 | 0.445863703 | −0.062088645 |
| ENSG00000106459 | NRF1 | 1.01E−04 | 0.323046456 | −0.119510858 |
| ENSG00000106462 | EZH2 | 3.20E−11 | 0.08121275 | 0.130806426 |
| ENSG00000106609 | TMEM248 | 2.90E−07 | 0.005316307 | −0.23621242 |
| ENSG00000106624 | AEBP1 | 1.10E−07 | 0.158176685 | −0.063824438 |
| ENSG00000106628 | POLD2 | 1.00E−08 | 0.658612976 | −0.035385479 |
| ENSG00000106948 | AKNA | 1.00E−04 | 0.251174368 | −0.100775455 |
| ENSG00000107099 | DOCK8 | 9.00E−10 | 0.488970714 | −0.040530824 |
| ENSG00000107164 | FUBP3 | 3.00E−04 | 0.891753365 | −0.016568849 |
| ENSG00000107223 | EDF1 | 1.00E−09 | 0.174777133 | 0.108777319 |
| ENSG00000107672 | NSMCE4A | 8.00E−04 | 0.421951683 | −0.076069575 |
| ENSG00000107854 | TNKS2 | 1.00E−04 | 0.358901659 | 0.113182695 |
| ENSG00000107937 | GTPBP4 | 3.00E−04 | 0.193844537 | 0.095836593 |
| ENSG00000108021 | FAM208B | 1.70E−08 | 0.236407718 | −0.130316925 |
| ENSG00000108094 | CUL2 | 4.00E−08 | 0.617398064 | −0.046747644 |
| ENSG00000108175 | ZMIZ1 | 2.00E−04 | 0.05690215 | −0.222817452 |
| ENSG00000108256 | NUFIP2 | 5.00E−11 | 0.429062757 | 0.11100338 |
| ENSG00000108270 | AATF | 1.00E−04 | 0.359806561 | −0.064247245 |
| ENSG00000108384 | RAD51C | 1.00E−09 | 0.010774857 | 0.189709393 |
| ENSG00000108424 | KPNB1 | 2.00E−11 | 8.66E−05 | −0.171777065 |
| ENSG00000108439 | PNPO | 3.00E−08 | 0.034630355 | 0.181670952 |
| ENSG00000108479 | GALK1 | 1.00E−04 | 0.608240213 | −0.053110682 |
| ENSG00000108506 | INTS2 | 8.01E−04 | 0.295086648 | 0.109973837 |
| ENSG00000108679 | LGALS3BP | 2.00E−08 | 0.973226652 | 0.000565498 |
| ENSG00000108848 | LUC7L3 | 2.60E−07 | 0.135191506 | 0.089311012 |
| ENSG00000109062 | SLC9A3R1 | 6.01E−04 | 0.122301958 | −0.064055136 |
| ENSG00000109111 | SUPT6H | 2.00E−10 | 0.994134615 | 0.001145357 |
| ENSG00000109332 | UBE2D3 | 1.00E−08 | 0.601564183 | 0.04567695 |
| ENSG00000109445 | ZNF330 | 8.00E−04 | 0.392762523 | −0.09281569 |
| ENSG00000109606 | DHX15 | 2.00E−04 | 0.687578939 | 0.016706661 |
| ENSG00000109685 | WHSC1 | 1.00E−11 | 0.0222009 | −0.153361985 |
| ENSG00000109805 | NCAPG | 9.00E−04 | 0.002277256 | 0.259598117 |
| ENSG00000110047 | EHD1 | 9.00E−04 | 0.373381573 | −0.084500637 |
| ENSG00000110075 | PPP6R3 | 4.00E−08 | 0.652181808 | −0.029536425 |
| ENSG00000110108 | TMEM109 | 1.00E−07 | 0.375160957 | −0.055219962 |
| ENSG00000110321 | EIF4G2 | 2.00E−08 | 0.4789335 | −0.030452093 |
| ENSG00000110367 | DDX6 | 6.00E−08 | 0.469603134 | −0.047598358 |
| ENSG00000110497 | AMBRA1 | 5.02E−04 | 0.151399742 | −0.200325064 |
| ENSG00000110619 | CARS | 2.00E−04 | 0.186855283 | 0.157414334 |
| ENSG00000110651 | CD81 | 1.00E−04 | 0.010480682 | −0.130640591 |
| ENSG00000110713 | NUP98 | 1.00E−10 | 0.656641011 | −0.027832725 |
| ENSG00000110955 | ATP5B | 4.00E−04 | 0.002969053 | 0.076326726 |
| ENSG00000111335 | OAS2 | 2.00E−04 | 0.677929702 | 0.048737643 |
| ENSG00000111348 | ARHGDIB | 1.50E−07 | 0.037505737 | −0.051698045 |
| ENSG00000111602 | TIMELESS | 4.00E−04 | 0.702100642 | 0.024415465 |
| ENSG00000111640 | GAPDH | 6.14E−04 | 0.68110279 | 0.024257691 |
| ENSG00000111641 | NOP2 | 1.00E−04 | 0.785381859 | 0.025711262 |
| ENSG00000111642 | CHD4 | 1.00E−04 | 0.289163376 | 0.048737019 |
| ENSG00000111670 | GNPTAB | 5.01E−04 | 0.585766626 | 0.059976694 |
| ENSG00000111726 | CMAS | 2.01E−04 | 0.649986681 | 0.073406219 |
| ENSG00000111737 | RAB35 | 7.01E−04 | 0.267070813 | −0.097986528 |
| ENSG00000111906 | HDDC2 | 2.00E−04 | 0.810276479 | −0.022306759 |
| ENSG00000112029 | FBXO5 | 2.00E−08 | 0.13434145 | 0.116650476 |
| ENSG00000112159 | MDN1 | 7.00E−08 | 0.503352158 | 0.068423753 |
| ENSG00000112200 | ZNF451 | 5.00E−04 | 0.487557618 | 0.072986258 |
| ENSG00000112308 | C6orf62 | 3.00E−04 | 0.221892591 | −0.102283924 |
| ENSG00000112576 | CCND3 | 2.00E−04 | 0.995647363 | −0.00039872 |
| ENSG00000112667 | DNPH1 | 3.00E−08 | 0.220468437 | 0.223545108 |
| ENSG00000112851 | ERBB2IP | 3.00E−04 | 0.005962767 | −0.26900197 |
| ENSG00000112972 | HMGCS1 | 1.00E−10 | 0.049358853 | 0.122227347 |
| ENSG00000112984 | KIF20A | 1.00E−08 | 0.934731349 | −0.011172951 |
| ENSG00000113369 | ARRDC3 | 8.03E−04 | 0.778199879 | 0.045698203 |
| ENSG00000113522 | RAD50 | 4.00E−08 | 0.22034331 | 0.108849135 |
| ENSG00000113580 | NR3C1 | 2.01E−04 | 0.497681416 | 0.120970261 |
| ENSG00000113649 | TCERG1 | 5.00E−04 | 0.001091576 | 0.213551854 |
| ENSG00000113810 | SMC4 | 1.00E−11 | 0.586987805 | −0.030468428 |
| ENSG00000114023 | FAM162A | 1.00E−04 | 0.569698046 | −0.05039296 |
| ENSG00000114030 | KPNA1 | 3.90E−07 | 0.001820335 | −0.311253874 |
| ENSG00000114126 | TFDP2 | 7.00E−09 | 0.567162468 | −0.026000796 |
| ENSG00000114200 | BCHE | 6.02E−04 | 0.422143365 | 0.11979247 |
| ENSG00000114416 | FXR1 | 6.00E−04 | 0.827823905 | 0.018508158 |
| ENSG00000114735 | HEMK1 | 1.01E−04 | 0.020519619 | −0.432698852 |
| ENSG00000114737 | CISH | 2.01E−04 | 0.710188905 | −0.018282662 |
| ENSG00000114867 | EIF4G1 | 4.00E−04 | 0.49755475 | 0.028286796 |
| ENSG00000115020 | PIKFYVE | 7.00E−04 | 0.874274951 | −0.045055571 |
| ENSG00000115053 | NCL | 5.50E−07 | 0.060545427 | 0.042909924 |
| ENSG00000115232 | ITGA4 | 3.00E−09 | 0.73709161 | −0.017356009 |
| ENSG00000115306 | SPTBN1 | 3.00E−14 | 0.008372115 | −0.348514727 |
| ENSG00000115419 | GLS | 4.00E−04 | 0.000197719 | −0.269875671 |
| ENSG00000115457 | IGFBP2 | 3.00E−04 | 0.641399806 | 0.036552753 |
| ENSG00000115464 | USP34 | 1.00E−04 | 0.352056564 | −0.122781815 |
| ENSG00000115524 | SF3B1 | 1.00E−04 | 0.157426391 | 0.074199855 |
| ENSG00000115526 | CHST10 | 6.00E−04 | 0.038600005 | −0.351592853 |
| ENSG00000115548 | KDM3A | 5.00E−04 | 0.40321263 | 0.088419558 |
| ENSG00000115694 | STK25 | 4.00E−04 | 0.063817944 | −0.233874856 |
| ENSG00000115760 | BIRC6 | 1.00E−04 | 0.297237048 | 0.088149326 |
| ENSG00000115761 | NOL10 | 9.00E−04 | 0.442727268 | 0.090675848 |
| ENSG00000115806 | GORASP2 | 4.00E−08 | 0.107877983 | −0.140430182 |
| ENSG00000115866 | DARS | 2.00E−08 | 0.047519868 | −0.122885656 |
| ENSG00000116120 | FARSB | 4.30E−07 | 0.448807369 | 0.078225185 |
| ENSG00000116133 | DHCR24 | 7.00E−04 | 0.242618057 | −0.119340931 |
| ENSG00000116213 | WRAP73 | 5.00E−08 | 0.033620725 | −0.365648037 |
| ENSG00000116406 | EDEM3 | 1.00E−04 | 0.160515088 | 0.161467793 |
| ENSG00000116698 | SMG7 | 3.00E−08 | 0.886915303 | −0.013938554 |
| ENSG00000116830 | TTF2 | 1.00E−04 | 0.605327274 | −0.05123716 |
| ENSG00000116863 | ADPRHL2 | 6.00E−04 | 0.363129878 | 0.185539492 |
| ENSG00000116984 | MTR | 3.00E−04 | 0.692634133 | −0.048662075 |
| ENSG00000117318 | ID3 | 4.00E−04 | 0.306098706 | 0.13190299 |
| ENSG00000117523 | PRRC2C | 1.00E−11 | 0.24687917 | 0.051514482 |
| ENSG00000117632 | STMN1 | 4.01E−04 | 0.46870306 | 0.016853098 |
| ENSG00000117713 | ARID1A | 2.10E−09 | 0.944760522 | 0.009363654 |
| ENSG00000117724 | CENPF | 6.00E−04 | 0.178526649 | 0.061700857 |
| ENSG00000117906 | RCN2 | 3.00E−08 | 0.060079639 | 0.177995705 |
| ENSG00000118007 | STAG1 | 2.04E−04 | 0.802913841 | −0.02813744 |
| ENSG00000118058 | MLL | 3.50E−07 | 0.817626816 | −0.021608686 |
| ENSG00000118193 | KIF14 | 2.00E−09 | 0.302902759 | 0.10403347 |
| ENSG00000118482 | PHF3 | 1.00E−08 | 0.873633816 | 0.012763981 |
| ENSG00000118513 | MYB | 2.00E−04 | 0.964892671 | −0.004218901 |
| ENSG00000118816 | CCNI | 6.90E−07 | 0.001528498 | −0.189161037 |
| ENSG00000119041 | GTF3C3 | 1.00E−04 | 0.862861391 | 0.021723507 |
| ENSG00000119397 | CNTRL | 5.00E−08 | 0.240770456 | −0.130222735 |
| ENSG00000119403 | PHF19 | 2.01E−04 | 0.072821269 | 0.168059289 |
| ENSG00000119596 | YLPM1 | 4.00E−09 | 0.122997252 | 0.126832643 |
| ENSG00000119638 | NEK9 | 4.00E−04 | 0.69912267 | −0.04034303 |
| ENSG00000119669 | IRF2BPL | 3.02E−04 | 0.737023088 | −0.046311272 |
| ENSG00000119912 | IDE | 1.00E−04 | 0.846898243 | −0.022139638 |
| ENSG00000120071 | KANSL1 | 5.00E−09 | 0.455238668 | −0.080812302 |
| ENSG00000120254 | MTHFD1L | 2.00E−04 | 0.953621846 | 0.005118686 |
| ENSG00000120690 | ELF1 | 1.00E−04 | 0.003793641 | −0.220693753 |
| ENSG00000120699 | EXOSC8 | 6.00E−04 | 0.446432736 | 0.059033278 |
| ENSG00000120733 | KDM3B | 8.00E−09 | 0.056284307 | −0.188410685 |
| ENSG00000120738 | EGR1 | 3.01E−04 | 0.317981925 | −0.167567968 |
| ENSG00000120800 | UTP20 | 1.80E−07 | 0.730921404 | 0.032446721 |
| ENSG00000120910 | PPP3CC | 7.00E−04 | 0.20233537 | −0.200494687 |
| ENSG00000121152 | NCAPH | 1.00E−04 | 0.569736027 | 0.064203664 |
| ENSG00000121621 | KIF18A | 6.01E−04 | 0.278023728 | −0.157166557 |
| ENSG00000121691 | CAT | 5.00E−04 | 0.059201981 | −0.151859968 |
| ENSG00000121864 | ZNF639 | 5.00E−04 | 0.468232115 | −0.0841423 |
| ENSG00000121892 | PDS5A | 2.00E−10 | 0.26251314 | −0.063220359 |
| ENSG00000122257 | RBBP6 | 1.25E−09 | 0.000891329 | −0.280964053 |
| ENSG00000122862 | SRGN | 6.00E−04 | 0.307943894 | −0.179460425 |
| ENSG00000122882 | ECD | 1.00E−04 | 0.79331662 | 0.025252457 |
| ENSG00000122966 | CIT | 9.00E−04 | 0.886264235 | −0.011615594 |
| ENSG00000123066 | MED13L | 2.20E−08 | 4.79E−05 | −0.415941737 |
| ENSG00000123144 | C19orf43 | 6.00E−04 | 0.121848164 | 0.139094396 |
| ENSG00000123213 | NLN | 7.02E−04 | 0.499519365 | 0.090955024 |
| ENSG00000123338 | NCKAP1L | 1.20E−11 | 0.299683199 | 0.062171215 |
| ENSG00000123473 | STIL | 8.01E−04 | 0.246311874 | −0.148306759 |
| ENSG00000123485 | HJURP | 2.00E−08 | 0.006526616 | 0.216805783 |
| ENSG00000123983 | ACSL3 | 7.01E−04 | 0.236015207 | 0.104976406 |
| ENSG00000124177 | CHD6 | 7.01E−04 | 0.577660003 | −0.071504202 |
| ENSG00000124181 | PLCG1 | 1.00E−04 | 0.95873485 | −0.001417988 |
| ENSG00000124193 | SRSF6 | 3.00E−04 | 0.119574817 | 0.116472581 |
| ENSG00000124228 | DDX27 | 5.00E−08 | 0.834023162 | 0.015972497 |
| ENSG00000124541 | RRP36 | 2.00E−10 | 0.604769753 | 0.044529012 |
| ENSG00000124575 | HIST1H1D | 1.00E−15 | 0.457298328 | 0.015260005 |
| ENSG00000124641 | MED20 | 1.00E−04 | 0.941332678 | −0.01659058 |
| ENSG00000124693 | HIST1H3B | 2.23E−12 | 0.254240878 | 0.040459312 |
| ENSG00000124789 | NUP153 | 3.20E−10 | 0.677271772 | −0.045251116 |
| ENSG00000125304 | TM9SF2 | 7.00E−04 | 0.431186651 | −0.044738316 |
| ENSG00000125484 | GTF3C4 | 1.01E−04 | 0.265749952 | −0.128337377 |
| ENSG00000125651 | GTF2F1 | 1.10E−08 | 0.031215966 | −0.168644284 |
| ENSG00000125686 | MED1 | 1.00E−08 | 0.18711587 | −0.124662129 |
| ENSG00000125755 | SYMPK | 1.00E−09 | 0.552114085 | −0.054422769 |
| ENSG00000125826 | RBCK1 | 1.01E−04 | 0.817834713 | −0.030877268 |
| ENSG00000125885 | MCM8 | 1.20E−07 | 0.740692289 | −0.028115948 |
| ENSG00000125971 | DYNLRB1 | 2.10E−07 | 0.436287925 | −0.07746413 |
| ENSG00000126001 | CEP250 | 7.10E−09 | 0.148680909 | 0.141831107 |
| ENSG00000126804 | ZBTB1 | 1.00E−04 | 0.453845337 | −0.079720916 |
| ENSG00000126883 | NUP214 | 1.00E−04 | 0.847640341 | −0.020473757 |
| ENSG00000127152 | BCL11B | 1.00E−08 | 5.40E−10 | −0.517770746 |
| ENSG00000127184 | COX7C | 3.00E−04 | 0.392077126 | −0.082208185 |
| ENSG00000127616 | SMARCA4 | 3.00E−12 | 0.813848874 | −0.012039575 |
| ENSG00000128191 | DGCR8 | 2.00E−08 | 0.071123809 | −0.242947421 |
| ENSG00000128829 | EIF2AK4 | 9.00E−04 | 0.627581021 | −0.077669277 |
| ENSG00000129317 | PUS7L | 2.00E−04 | 0.653028133 | −0.054700064 |
| ENSG00000129351 | ILF3 | 1.30E−07 | 0.030039666 | −0.082482869 |
| ENSG00000129355 | CDKN2D | 3.01E−04 | 0.933543847 | −0.011782808 |
| ENSG00000130175 | PRKCSH | 3.00E−08 | 0.492538567 | −0.034477239 |
| ENSG00000130255 | RPL36 | 1.40E−06 | 0.000331082 | −0.237194765 |
| ENSG00000130311 | DDA1 | 2.01E−04 | 0.855575658 | −0.028673083 |
| ENSG00000130402 | ACTN4 | 3.01E−04 | 0.05655675 | −0.168897398 |
| ENSG00000130640 | TUBGCP2 | 2.00E−04 | 0.083088036 | −0.263652098 |
| ENSG00000130724 | CHMP2A | 2.00E−11 | 0.770037849 | 0.028723436 |
| ENSG00000130726 | TRIM28 | 1.00E−11 | 0.034098412 | −0.092503983 |
| ENSG00000130816 | DNMT1 | 5.00E−04 | 0.595705453 | −0.027738247 |
| ENSG00000131148 | EMC8 | 5.00E−11 | 0.89007858 | −0.019540666 |
| ENSG00000131174 | COX7B | 5.00E−04 | 0.280235251 | −0.066677874 |
| ENSG00000131446 | MGAT1 | 5.01E−04 | 0.513530158 | −0.076217655 |
| ENSG00000131467 | PSME3 | 3.01E−04 | 0.03715923 | −0.116973165 |
| ENSG00000131504 | DIAPH1 | 2.00E−04 | 0.005406879 | −0.213577391 |
| ENSG00000132142 | ACACA | 1.00E−04 | 0.384701339 | −0.088267863 |
| ENSG00000132155 | RAF1 | 1.90E−07 | 0.727868161 | 0.035824977 |
| ENSG00000132182 | NUP210 | 4.00E−11 | 0.396376427 | −0.053178121 |
| ENSG00000132294 | EFR3A | 6.00E−04 | 0.101176956 | −0.138753026 |
| ENSG00000132305 | IMMT | 2.00E−04 | 0.911802156 | −0.008862008 |
| ENSG00000132383 | RPA1 | 1.00E−04 | 0.536237887 | 0.027348564 |
| ENSG00000132436 | FIGNL1 | 4.00E−04 | 0.90689028 | 0.012761853 |
| ENSG00000132463 | GRSF1 | 3.01E−04 | 0.003456732 | 0.144799456 |
| ENSG00000132466 | ANKRD17 | 6.00E−04 | 0.746590296 | 0.023300227 |
| ENSG00000132612 | VPS4A | 3.05E−06 | 0.187740524 | −0.117542241 |
| ENSG00000132646 | PCNA | 4.00E−09 | 0.449582299 | 0.020688405 |
| ENSG00000132680 | KIAA0907 | 7.00E−04 | 0.406235984 | −0.089721055 |
| ENSG00000132842 | AP3B1 | 5.00E−04 | 0.894277559 | 0.020029663 |
| ENSG00000132953 | XPO4 | 4.00E−04 | 0.386914625 | −0.098355739 |
| ENSG00000133026 | MYH10 | 1.00E−04 | 0.734506152 | 0.038657519 |
| ENSG00000133454 | MYO18B | 6.00E−11 | 0.694394285 | −0.024618977 |
| ENSG00000133639 | BTG1 | 7.00E−04 | 0.111632247 | −0.307842825 |
| ENSG00000133657 | ATP13A3 | 1.00E−10 | 0.021301072 | −0.237052311 |
| ENSG00000133706 | LARS | 4.00E−04 | 0.597497941 | −0.035619738 |
| ENSG00000133961 | NUMB | 9.01E−04 | 0.646567716 | 0.059486807 |
| ENSG00000134313 | KIDINS220 | 1.00E−08 | 0.857549373 | 0.021963698 |
| ENSG00000134371 | CDC73 | 3.00E−04 | 0.078009542 | −0.179882162 |
| ENSG00000134480 | CCNH | 6.00E−04 | 0.669625945 | 0.058777209 |
| ENSG00000134516 | DOCK2 | 6.00E−04 | 0.505754891 | 0.037062588 |
| ENSG00000134644 | PUM1 | 2.00E−04 | 0.207254614 | −0.119306565 |
| ENSG00000134686 | PHC2 | 8.90E−07 | 0.974560029 | 0.001948917 |
| ENSG00000134697 | GNL2 | 2.00E−08 | 0.31879467 | 0.080020877 |
| ENSG00000134759 | ELP2 | 2.00E−04 | 0.297463108 | 0.107815275 |
| ENSG00000134910 | STT3A | 2.00E−11 | 0.248910784 | 0.068871835 |
| ENSG00000134954 | ETS1 | 5.70E−09 | 1.27E−05 | −0.232324455 |
| ENSG00000134987 | WDR36 | 3.00E−04 | 0.028738034 | 0.151716239 |
| ENSG00000135090 | TAOK3 | 2.00E−04 | 0.163787333 | −0.139600964 |
| ENSG00000135316 | SYNCRIP | 5.00E−08 | 0.141605449 | −0.082838833 |
| ENSG00000135439 | AGAP2 | 6.00E−04 | 0.408030001 | −0.104437193 |
| ENSG00000135521 | LTV1 | 9.00E−04 | 0.577086029 | 0.047229663 |
| ENSG00000135679 | MDM2 | 4.90E−07 | 0.948348125 | 0.012834459 |
| ENSG00000135763 | URB2 | 4.01E−04 | 0.668047421 | 0.04463194 |
| ENSG00000135837 | CEP350 | 1.00E−08 | 0.152171166 | −0.140217831 |
| ENSG00000135905 | DOCK10 | 1.00E−08 | 0.717332641 | 0.040604232 |
| ENSG00000135932 | CAB39 | 1.00E−08 | 0.019948395 | −0.200243436 |
| ENSG00000135940 | COX5B | 4.00E−04 | 0.629646454 | −0.025317297 |
| ENSG00000136051 | KIAA1033 | 2.00E−04 | 0.30132225 | 0.128371731 |
| ENSG00000136068 | FLNB | 2.90E−12 | 0.062111584 | −0.409977879 |
| ENSG00000136104 | RNASEH2B | 4.20E−07 | 0.392137768 | 0.057232924 |
| ENSG00000136146 | MED4 | 8.00E−04 | 0.169595348 | −0.110732537 |
| ENSG00000136167 | LCP1 | 2.00E−04 | 0.489381124 | −0.018010751 |
| ENSG00000136271 | DDX56 | 1.10E−07 | 0.710493262 | −0.028033209 |
| ENSG00000136286 | MYO1G | 4.00E−08 | 0.20294865 | 0.071258537 |
| ENSG00000136381 | IREB2 | 3.00E−04 | 0.349257048 | 0.088289702 |
| ENSG00000136492 | BRIP1 | 3.00E−10 | 0.419920204 | −0.069475773 |
| ENSG00000136527 | TRA2B | 1.00E−04 | 0.403669503 | −0.031939853 |
| ENSG00000136536 | MARCH7 | 1.00E−10 | 0.683034436 | 0.035756577 |
| ENSG00000136628 | EPRS | 1.00E−04 | 0.75764814 | 0.018770161 |
| ENSG00000136653 | RASSF5 | 1.00E−04 | 0.545754108 | −0.05276974 |
| ENSG00000136709 | WDR33 | 9.00E−04 | 0.304508163 | −0.081701638 |
| ENSG00000136738 | STAM | 3.00E−04 | 0.366470963 | 0.094693082 |
| ENSG00000136754 | ABI1 | 2.00E−04 | 0.893753832 | −0.015741049 |
| ENSG00000136758 | YME1L1 | 1.00E−04 | 0.090002669 | −0.100978955 |
| ENSG00000136824 | SMC2 | 8.00E−04 | 0.373245909 | 0.054833376 |
| ENSG00000136827 | TOR1A | 1.00E−08 | 0.306962116 | 0.113750173 |
| ENSG00000136878 | USP20 | 2.00E−08 | 0.019165529 | −0.206602358 |
| ENSG00000136997 | MYC | 3.00E−08 | 0.000130485 | −0.222358961 |
| ENSG00000137076 | TLN1 | 2.00E−09 | 0.114582751 | −0.09941367 |
| ENSG00000137106 | GRHPR | 3.00E−04 | 0.415762699 | −0.09172823 |
| ENSG00000137770 | CTDSPL2 | 5.06E−04 | 0.972005432 | −0.008284378 |
| ENSG00000137776 | SLTM | 2.00E−04 | 0.100597307 | 0.119688243 |
| ENSG00000137812 | CASC5 | 9.01E−04 | 0.164406778 | 0.150771957 |
| ENSG00000137818 | RPLP1 | 3.04E−04 | 0.002330818 | −0.514464579 |
| ENSG00000137845 | ADAM10 | 9.00E−10 | 0.012053048 | −0.208903322 |
| ENSG00000138081 | FBXO11 | 3.00E−04 | 0.421443356 | −0.098168828 |
| ENSG00000138095 | LRPPRC | 3.00E−04 | 0.234050395 | −0.065104179 |
| ENSG00000138107 | ACTR1A | 3.01E−04 | 0.37194789 | −0.072733569 |
| ENSG00000138182 | KIF20B | 1.00E−10 | 0.00406547 | 0.242659079 |
| ENSG00000138231 | DBR1 | 8.00E−04 | 0.870540124 | 0.016538189 |
| ENSG00000138442 | WDR12 | 6.00E−08 | 0.190351341 | −0.142989262 |
| ENSG00000138496 | PARP9 | 1.00E−04 | 0.385276066 | 0.103537296 |
| ENSG00000138592 | USP8 | 7.06E−04 | 0.313673018 | −0.181127726 |
| ENSG00000138668 | HNRNPD | 2.40E−07 | 0.003261874 | −0.098862205 |
| ENSG00000138698 | RAP1GDS1 | 5.00E−04 | 0.715002624 | −0.035260421 |
| ENSG00000138778 | CENPE | 2.00E−10 | 0.214004921 | 0.115990377 |
| ENSG00000138795 | LEF1 | 1.00E−09 | 1.60E−06 | −0.210659864 |
| ENSG00000138802 | SEC24B | 1.00E−09 | 0.515439824 | −0.072741233 |
| ENSG00000139154 | AEBP2 | 2.00E−04 | 0.48380984 | 0.088405889 |
| ENSG00000139197 | PEX5 | 9.02E−04 | 0.510476835 | 0.078299498 |
| ENSG00000139218 | SCAF11 | 1.00E−08 | 0.50709776 | −0.04074801 |
| ENSG00000139350 | NEDD1 | 1.00E−04 | 0.725169134 | 0.037847451 |
| ENSG00000139505 | MTMR6 | 4.00E−08 | 0.747037745 | −0.038766186 |
| ENSG00000139613 | SMARCC2 | 1.00E−12 | 0.334491125 | 0.087836839 |
| ENSG00000139620 | KANSL2 | 4.00E−04 | 0.2859956 | 0.116502184 |
| ENSG00000139641 | ESYT1 | 6.00E−04 | 0.115537694 | 0.094065695 |
| ENSG00000139687 | RB1 | 4.00E−11 | 0.227456544 | 0.062123731 |
| ENSG00000139842 | CUL4A | 8.01E−04 | 0.735833147 | −0.028312464 |
| ENSG00000139946 | PELI2 | 8.01E−04 | 0.84869402 | −0.030488571 |
| ENSG00000140259 | MFAP1 | 4.00E−04 | 0.286171294 | 0.110298043 |
| ENSG00000140262 | TCF12 | 2.00E−10 | 0.025334533 | −0.184064816 |
| ENSG00000140299 | BNIP2 | 9.00E−08 | 0.453644947 | 0.060595449 |
| ENSG00000140332 | TLE3 | 2.00E−08 | 0.001343794 | −0.227829431 |
| ENSG00000140525 | FANCI | 2.00E−04 | 0.688024573 | 0.029180218 |
| ENSG00000140829 | DHX38 | 8.00E−08 | 0.404193545 | −0.083781852 |
| ENSG00000140943 | MBTPS1 | 4.00E−04 | 0.905572549 | 0.017038735 |
| ENSG00000141027 | NCOR1 | 1.00E−04 | 0.129071822 | −0.136116164 |
| ENSG00000141252 | VPS53 | 2.00E−04 | 0.91838956 | 0.011174255 |
| ENSG00000141367 | CLTC | 2.00E−15 | 0.808942699 | −0.009782716 |
| ENSG00000141378 | PTRH2 | 1.00E−04 | 0.917590739 | −0.014270194 |
| ENSG00000141456 | PELP1 | 2.00E−04 | 0.806300676 | 0.019902797 |
| ENSG00000141551 | CSNK1D | 5.00E−04 | 0.119201328 | −0.129522549 |
| ENSG00000141556 | TBCD | 1.00E−04 | 0.591494459 | −0.026526545 |
| ENSG00000142002 | DPP9 | 1.00E−04 | 0.734190324 | −0.049637386 |
| ENSG00000142453 | CARM1 | 3.00E−04 | 0.397417148 | −0.08333644 |
| ENSG00000143106 | PSMA5 | 4.00E−08 | 0.382567623 | 0.045346574 |
| ENSG00000143401 | ANP32E | 1.00E−04 | 0.915890957 | 0.004516508 |
| ENSG00000143442 | POGZ | 2.00E−09 | 0.037681202 | −0.207363006 |
| ENSG00000143476 | DTL | 3.01E−04 | 0.748549723 | −0.021380096 |
| ENSG00000143514 | TP53BP2 | 7.00E−04 | 0.283581835 | −0.117348193 |
| ENSG00000143624 | INTS3 | 7.01E−04 | 0.265735445 | −0.146742464 |
| ENSG00000143870 | PDIA6 | 1.00E−04 | 0.042049362 | 0.102641235 |
| ENSG00000143924 | EML4 | 7.01E−04 | 0.028666301 | −0.26184158 |
| ENSG00000144028 | SNRNP200 | 8.00E−14 | 0.023390105 | 0.101854033 |
| ENSG00000144554 | FANCD2 | 2.00E−04 | 0.730926111 | 0.036291397 |
| ENSG00000144559 | TAMM41 | 5.00E−04 | 0.851527782 | 0.02836682 |
| ENSG00000144580 | RQCD1 | 2.00E−08 | 0.506022372 | −0.045037202 |
| ENSG00000144895 | EIF2A | 9.00E−04 | 0.746023779 | −0.024414611 |
| ENSG00000145041 | VPRBP | 4.00E−04 | 0.348933992 | −0.094987498 |
| ENSG00000145375 | SPATA5 | 1.00E−04 | 0.784103808 | −0.049246739 |
| ENSG00000145604 | SKP2 | 1.00E−04 | 0.979279396 | 0.003747348 |
| ENSG00000145675 | PIK3R1 | 4.02E−04 | 0.168588299 | −0.102576778 |
| ENSG00000145741 | BTF3 | 5.00E−04 | 0.339388798 | −0.072822964 |
| ENSG00000145833 | DDX46 | 2.00E−04 | 0.23392151 | 0.072701228 |
| ENSG00000146457 | WTAP | 3.00E−04 | 0.883760976 | 0.015398065 |
| ENSG00000146918 | NCAPG2 | 1.50E−07 | 0.276197103 | 0.072778556 |
| ENSG00000147130 | ZMYM3 | 2.00E−04 | 0.059490308 | −0.195960662 |
| ENSG00000147650 | LRP12 | 2.00E−04 | 0.590549253 | 0.057987792 |
| ENSG00000147677 | EIF3H | 4.01E−04 | 0.507259945 | −0.031197371 |
| ENSG00000148175 | STOM | 5.00E−04 | 0.202025121 | −0.203176311 |
| ENSG00000148229 | POLE3 | 6.06E−04 | 0.60752674 | −0.042630647 |
| ENSG00000148334 | PTGES2 | 1.04E−04 | 0.626929001 | −0.068481398 |
| ENSG00000148337 | CIZ1 | 3.00E−04 | 0.65773939 | −0.053627564 |
| ENSG00000148396 | SEC16A | 1.00E−04 | 0.806810949 | 0.025438251 |
| ENSG00000148400 | NOTCH1 | 1.00E−04 | 0.276344103 | −0.101708517 |
| ENSG00000148773 | MKI67 | 1.00E−10 | 0.123777629 | 0.063404366 |
| ENSG00000148840 | PPRC1 | 5.00E−04 | 0.85534363 | −0.028695475 |
| ENSG00000148843 | PDCD11 | 5.00E−04 | 0.56753983 | −0.05144919 |
| ENSG00000149262 | INTS4 | 4.00E−04 | 0.697964901 | 0.097540919 |
| ENSG00000149273 | RPS3 | 2.35E−05 | 0.448604972 | −0.041417003 |
| ENSG00000149308 | NPAT | 2.00E−04 | 0.81995279 | 0.029267153 |
| ENSG00000149480 | MTA2 | 6.00E−09 | 2.64E−05 | −0.22354576 |
| ENSG00000149554 | CHEK1 | 3.00E−08 | 0.029907425 | 0.142987957 |
| ENSG00000149806 | FAU | 3.02E−04 | 0.009610398 | 0.1703503 |
| ENSG00000149925 | ALDOA | 3.02E−04 | 0.183371478 | −0.061277735 |
| ENSG00000150990 | DHX37 | 2.00E−04 | 0.614661587 | −0.044689634 |
| ENSG00000151131 | C12orf45 | 8.00E−04 | 0.485034329 | −0.088403349 |
| ENSG00000151366 | NDUFC2 | 2.00E−09 | 0.361312276 | −0.043896664 |
| ENSG00000151502 | VPS26B | 2.00E−04 | 0.277634848 | 0.100319458 |
| ENSG00000151503 | NCAPD3 | 3.00E−04 | 0.100416068 | 0.106975594 |
| ENSG00000151694 | ADAM17 | 7.30E−07 | 0.446223538 | −0.1096626 |
| ENSG00000151702 | FLI1 | 1.00E−04 | 0.221034285 | −0.138078723 |
| ENSG00000151835 | SACS | 2.00E−04 | 0.575717819 | 0.054319675 |
| ENSG00000152082 | MZT2B | 1.30E−08 | 0.784207016 | −0.02186646 |
| ENSG00000152147 | GEMIN6 | 8.00E−04 | 0.112633984 | 0.186427654 |
| ENSG00000152601 | MBNL1 | 2.00E−11 | 0.00777836 | −0.152181062 |
| ENSG00000152818 | UTRN | 1.00E−04 | 0.61246869 | −0.063349119 |
| ENSG00000153187 | HNRNPU | 3.00E−04 | 0.973831029 | −0.000601876 |
| ENSG00000153283 | CD96 | 2.00E−04 | 0.267330877 | −0.089143298 |
| ENSG00000153310 | FAM49B | 2.00E−04 | 0.007506383 | −0.139159484 |
| ENSG00000153827 | TRIP12 | 2.80E−09 | 0.04819259 | −0.171340571 |
| ENSG00000153922 | CHD1 | 3.00E−09 | 0.997926928 | −0.000811209 |
| ENSG00000154370 | TRIM11 | 3.01E−04 | 0.010190424 | −0.431525912 |
| ENSG00000155097 | ATP6V1C1 | 4.00E−04 | 0.714750549 | −0.035031119 |
| ENSG00000155561 | NUP205 | 1.00E−10 | 0.810978988 | −0.015620768 |
| ENSG00000155827 | RNF20 | 1.00E−08 | 0.674024322 | 0.06246891 |
| ENSG00000156273 | BACH1 | 6.00E−04 | 0.916576258 | −0.031050024 |
| ENSG00000156858 | PRR14 | 1.10E−07 | 0.581714014 | −0.069166264 |
| ENSG00000156875 | HIAT1 | 1.00E−04 | 0.380713082 | −0.10910263 |
| ENSG00000156970 | BUB1B | 4.00E−10 | 0.947953681 | 0.006781777 |
| ENSG00000156983 | BRPF1 | 4.00E−04 | 0.933570558 | −0.010817335 |
| ENSG00000157540 | DYRK1A | 2.00E−09 | 0.276566574 | −0.107710063 |
| ENSG00000157593 | SLC35B2 | 1.00E−04 | 0.149940449 | −0.2407324 |
| ENSG00000158290 | CUL4B | 1.00E−04 | 0.931575328 | −0.009597839 |
| ENSG00000158373 | HIST1H2BD | 2.00E−08 | 0.665459706 | −0.010325486 |
| ENSG00000158406 | HIST1H4H | 5.50E−09 | 0.616647851 | 0.034929668 |
| ENSG00000158526 | TSR2 | 1.00E−04 | 0.033164157 | 0.270897208 |
| ENSG00000158623 | COPG2 | 2.00E−04 | 0.583240481 | −0.080548482 |
| ENSG00000158864 | NDUFS2 | 1.40E−07 | 0.692802138 | 0.037508569 |
| ENSG00000158985 | CDC42SE2 | 8.00E−08 | 0.027991366 | −0.145160094 |
| ENSG00000159131 | GART | 7.00E−04 | 0.231639477 | −0.068059954 |
| ENSG00000159140 | SON | 1.20E−12 | 0.122942939 | 0.067823184 |
| ENSG00000159314 | ARHGAP27 | 2.00E−04 | 0.839973686 | −0.028088528 |
| ENSG00000159720 | ATP6V0D1 | 5.00E−04 | 0.616798782 | 0.050867945 |
| ENSG00000160294 | MCM3AP | 2.00E−04 | 0.271306147 | −0.134887854 |
| ENSG00000160710 | ADAR | 2.00E−04 | 0.080829161 | −0.088103689 |
| ENSG00000160796 | NBEAL2 | 1.00E−04 | 0.231202003 | 0.128523719 |
| ENSG00000160877 | NACC1 | 2.00E−04 | 0.286689117 | −0.119046325 |
| ENSG00000160949 | TONSL | 1.00E−04 | 0.196335035 | −0.17410121 |
| ENSG00000161618 | ALDH16A1 | 2.02E−04 | 0.73604894 | −0.075930576 |
| ENSG00000161980 | POLR3K | 3.00E−04 | 0.780264569 | 0.030710578 |
| ENSG00000162434 | JAK1 | 3.00E−04 | 0.381361282 | −0.087910305 |
| ENSG00000162607 | USP1 | 5.00E−11 | 0.111472525 | −0.094654587 |
| ENSG00000162642 | C1orf52 | 7.06E−04 | 0.242610098 | 0.178613459 |
| ENSG00000162664 | ZNF326 | 9.00E−04 | 0.113292053 | 0.1523951 |
| ENSG00000163104 | SMARCAD1 | 1.00E−07 | 0.928265836 | −0.008266525 |
| ENSG00000163349 | HIPK1 | 2.00E−04 | 0.020020123 | −0.282659771 |
| ENSG00000163466 | ARPC2 | 1.00E−08 | 0.883580647 | 0.005900615 |
| ENSG00000163607 | GTPBP8 | 1.00E−04 | 0.426296869 | −0.158064045 |
| ENSG00000163655 | GMPS | 2.00E−11 | 0.775716157 | 0.020676052 |
| ENSG00000163808 | KIF15 | 1.00E−04 | 0.593535319 | 0.070077932 |
| ENSG00000163902 | RPN1 | 3.00E−04 | 0.036709758 | 0.092942803 |
| ENSG00000163904 | SENP2 | 2.00E−04 | 0.045589218 | −0.287466993 |
| ENSG00000163939 | PBRM1 | 2.00E−04 | 0.686874801 | −0.037496 |
| ENSG00000163946 | FAM208A | 1.00E−08 | 0.438939056 | −0.067851035 |
| ENSG00000164134 | NAA15 | 4.00E−09 | 0.476257503 | −0.043457933 |
| ENSG00000164168 | TMEM184C | 2.50E−08 | 0.015050183 | −0.397412646 |
| ENSG00000164190 | NIPBL | 2.00E−09 | 0.303068767 | −0.09710725 |
| ENSG00000164209 | SLC25A46 | 2.00E−04 | 0.530752611 | 0.054652482 |
| ENSG00000164754 | RAD21 | 1.00E−08 | 0.756335554 | −0.013371036 |
| ENSG00000164978 | NUDT2 | 1.01E−04 | 0.680662594 | −0.053435289 |
| ENSG00000164985 | PSIP1 | 1.00E−04 | 0.469682012 | 0.047128687 |
| ENSG00000165209 | STRBP | 7.00E−04 | 0.030348179 | −0.279083308 |
| ENSG00000165271 | NOL6 | 2.00E−08 | 0.335093224 | −0.094631942 |
| ENSG00000165304 | MELK | 2.00E−04 | 0.351342781 | 0.077213211 |
| ENSG00000165417 | GTF2A1 | 7.00E−11 | 0.844760071 | −0.015019218 |
| ENSG00000165480 | SKA3 | 2.00E−04 | 0.96112422 | 0.004310634 |
| ENSG00000165494 | PCF11 | 2.00E−09 | 0.168402482 | −0.149826279 |
| ENSG00000165502 | RPL36AL | 5.00E−08 | 0.586754886 | −0.050125431 |
| ENSG00000165527 | ARF6 | 1.00E−10 | 0.156807609 | −0.122434848 |
| ENSG00000165678 | GHITM | 2.00E−09 | 0.40434721 | −0.044927385 |
| ENSG00000165782 | TMEM55B | 1.00E−04 | 0.742872155 | 0.097568043 |
| ENSG00000165916 | PSMC3 | 2.00E−04 | 0.327136153 | 0.067066757 |
| ENSG00000166037 | CEP57 | 1.00E−04 | 0.563570438 | −0.056277887 |
| ENSG00000166226 | CCT2 | 2.00E−04 | 0.424973179 | 0.028289737 |
| ENSG00000166747 | AP1G1 | 1.00E−04 | 0.033859721 | −0.180376252 |
| ENSG00000166888 | STAT6 | 1.00E−04 | 0.047894287 | −0.21884119 |
| ENSG00000166963 | MAP1A | 1.00E−09 | 0.569547165 | 0.03924313 |
| ENSG00000166986 | MARS | 9.00E−08 | 0.040009981 | −0.120193487 |
| ENSG00000167258 | CDK12 | 5.00E−04 | 0.030045657 | −0.185190158 |
| ENSG00000167323 | STIM1 | 5.00E−04 | 0.977475163 | 0.002228555 |
| ENSG00000167468 | GPX4 | 7.00E−04 | 0.056273288 | 0.118694036 |
| ENSG00000167470 | MIDN | 3.00E−04 | 0.037975203 | −0.404562969 |
| ENSG00000167491 | GATAD2A | 3.00E−04 | 0.460886475 | −0.052425426 |
| ENSG00000167522 | ANKRD11 | 1.00E−08 | 0.255387498 | 0.10026816 |
| ENSG00000167548 | MLL2 | 7.70E−09 | 0.196266437 | −0.106714093 |
| ENSG00000167658 | EEF2 | 8.00E−09 | 0.147519118 | −0.034288496 |
| ENSG00000167670 | CHAF1A | 1.00E−08 | 0.388610811 | −0.064999638 |
| ENSG00000167747 | C19orf48 | 1.80E−07 | 0.222151707 | −0.136113219 |
| ENSG00000167775 | CD320 | 6.01E−04 | 0.822695325 | 0.022736769 |
| ENSG00000167978 | SRRM2 | 1.00E−04 | 0.027585188 | −0.081656945 |
| ENSG00000168159 | RNF187 | 5.00E−04 | 0.806336099 | 0.019371782 |
| ENSG00000168264 | IRF2BP2 | 7.60E−07 | 0.400567441 | −0.098542726 |
| ENSG00000168298 | HIST1H1E | 8.00E−12 | 0.575494886 | 0.00693283 |
| ENSG00000168374 | ARF4 | 2.00E−04 | 0.706405122 | −0.070945614 |
| ENSG00000168476 | REEP4 | 8.00E−04 | 0.228797357 | −0.127937767 |
| ENSG00000168575 | SLC20A2 | 4.02E−04 | 0.354909308 | −0.119617959 |
| ENSG00000168906 | MAT2A | 3.00E−04 | 0.308184492 | −0.065364304 |
| ENSG00000168918 | INPP5D | 3.00E−04 | 0.934564983 | 0.00569134 |
| ENSG00000169018 | FEM1B | 3.00E−04 | 0.01910054 | −0.397020738 |
| ENSG00000169221 | TBC1D10B | 8.00E−04 | 0.845261963 | 0.037666933 |
| ENSG00000169251 | NMD3 | 3.03E−04 | 0.950093056 | −0.00374493 |
| ENSG00000169375 | SIN3A | 3.00E−04 | 0.134711475 | −0.125528448 |
| ENSG00000169710 | FASN | 1.10E−11 | 0.30709726 | −0.046236104 |
| ENSG00000169813 | HNRNPF | 2.00E−04 | 0.261636371 | −0.045390453 |
| ENSG00000169905 | TOR1AIP2 | 7.00E−04 | 0.025144824 | −0.223803399 |
| ENSG00000169994 | MYO7B | 3.30E−14 | 0.067830781 | −0.06063889 |
| ENSG00000170004 | CHD3 | 1.00E−08 | 0.982259628 | 0.001839786 |
| ENSG00000170242 | USP47 | 2.00E−09 | 0.545269342 | −0.072546866 |
| ENSG00000170430 | MGMT | 4.00E−04 | 0.64248843 | 0.054326376 |
| ENSG00000171202 | TMEM126A | 8.07E−04 | 0.731670543 | 0.060950022 |
| ENSG00000171298 | GAA | 1.10E−08 | 0.092998845 | −0.248293341 |
| ENSG00000171310 | CHST11 | 1.20E−07 | 0.004536717 | −0.14604981 |
| ENSG00000171522 | PTGER4 | 2.40E−07 | 0.002288634 | −0.274827847 |
| ENSG00000171608 | PIK3CD | 2.00E−08 | 0.621359547 | −0.054808961 |
| ENSG00000171681 | ATF7IP | 3.00E−10 | 0.209720012 | −0.150308959 |
| ENSG00000171861 | RNMTL1 | 8.04E−04 | 0.07761122 | 0.249083661 |
| ENSG00000172046 | USP19 | 5.00E−04 | 0.245610178 | −0.156637412 |
| ENSG00000172053 | QARS | 5.00E−04 | 0.867300993 | 0.013028643 |
| ENSG00000172292 | CERS6 | 1.00E−09 | 0.029552171 | −0.205124483 |
| ENSG00000172534 | HCFC1 | 2.87E−12 | 6.58E−09 | −0.313512877 |
| ENSG00000172590 | MRPL52 | 2.10E−06 | 0.325139446 | −0.06780364 |
| ENSG00000172716 | SLFN11 | 2.00E−04 | 0.664664806 | 0.04271628 |
| ENSG00000172725 | CORO1B | 6.00E−04 | 0.292853887 | −0.118190498 |
| ENSG00000172775 | FAM192A | 1.00E−04 | 0.366617379 | −0.209977577 |
| ENSG00000172795 | DCP2 | 4.00E−04 | 0.294839777 | 0.094471533 |
| ENSG00000172893 | DHCR7 | 8.00E−08 | 0.073439676 | −0.22022124 |
| ENSG00000172939 | OXSR1 | 2.01E−04 | 0.260117099 | −0.092965672 |
| ENSG00000172995 | ARPP21 | 7.01E−04 | 0.648766475 | −0.073812799 |
| ENSG00000173020 | ADRBK1 | 1.00E−08 | 0.708537254 | −0.02554542 |
| ENSG00000173141 | MRP63 | 1.00E−11 | 0.385791511 | 0.081117583 |
| ENSG00000173163 | COMMD1 | 7.00E−04 | 0.313197556 | 0.20935172 |
| ENSG00000173442 | EHBP1L1 | 3.00E−04 | 0.648913241 | 0.061932902 |
| ENSG00000173585 | CCR9 | 4.00E−04 | 0.004934791 | −0.343972085 |
| ENSG00000173598 | NUDT4 | 4.01E−04 | 0.997345883 | −0.001074841 |
| ENSG00000173674 | EIF1AX | 8.00E−04 | 0.780846703 | 0.072635112 |
| ENSG00000173692 | PSMD1 | 2.00E−09 | 0.749735343 | 0.022709414 |
| ENSG00000173821 | RNF213 | 1.00E−10 | 0.78788961 | 0.011822428 |
| ENSG00000174010 | KLHL15 | 8.01E−04 | 0.770357983 | −0.039693382 |
| ENSG00000174173 | TRMT10C | 2.00E−04 | 0.047069196 | 0.19484858 |
| ENSG00000174197 | MGA | 2.30E−08 | 0.926221975 | 0.009158715 |
| ENSG00000174231 | PRPF8 | 1.00E−10 | 0.595739886 | 0.019539188 |
| ENSG00000174238 | PITPNA | 3.00E−04 | 0.470422902 | −0.066848619 |
| ENSG00000174579 | MSL2 | 5.00E−04 | 0.027763257 | −0.205275001 |
| ENSG00000174851 | YIF1A | 4.00E−04 | 0.662843111 | 0.058226495 |
| ENSG00000175216 | CKAP5 | 1.50E−08 | 0.03814885 | 0.117491174 |
| ENSG00000175221 | MED16 | 3.82E−06 | 0.907545434 | −0.022207491 |
| ENSG00000175467 | SART1 | 1.00E−04 | 0.126941788 | −0.10443742 |
| ENSG00000175931 | UBE2O | 3.00E−04 | 0.447757287 | 0.068133312 |
| ENSG00000176619 | LMNB2 | 3.00E−04 | 0.831805089 | 0.019798531 |
| ENSG00000176890 | TYMS | 4.00E−04 | 0.016419414 | 0.072462812 |
| ENSG00000177084 | POLE | 1.00E−11 | 0.256661974 | 0.078668239 |
| ENSG00000177156 | TALDO1 | 8.00E−04 | 0.975031972 | −0.001808282 |
| ENSG00000177370 | TIMM22 | 9.00E−04 | 0.455177571 | 0.122153476 |
| ENSG00000177731 | FLII | 2.00E−04 | 0.139734019 | 0.11682969 |
| ENSG00000177733 | HNRNPA0 | 2.00E−04 | 0.789027425 | 0.010338975 |
| ENSG00000177885 | GRB2 | 1.00E−08 | 0.474905024 | −0.037372741 |
| ENSG00000178202 | KDELC2 | 5.00E−04 | 0.480571508 | 0.092686527 |
| ENSG00000178252 | WDR6 | 1.00E−04 | 0.9305271 | 0.005862339 |
| ENSG00000178921 | PFAS | 5.00E−04 | 0.196160647 | 0.110574411 |
| ENSG00000179085 | DPM3 | 4.00E−04 | 0.434227322 | 0.195823923 |
| ENSG00000179091 | CYC1 | 6.02E−04 | 0.55486733 | 0.052028776 |
| ENSG00000179262 | RAD23A | 5.10E−07 | 0.674786887 | 0.038029829 |
| ENSG00000179409 | GEMIN4 | 7.20E−07 | 0.143223537 | −0.152149612 |
| ENSG00000180104 | EXOC3 | 9.00E−04 | 0.538380165 | −0.070376673 |
| ENSG00000180573 | HIST1H2AC | 7.04E−04 | 0.896293947 | −0.002455334 |
| ENSG00000181090 | EHMT1 | 9.00E−04 | 0.697892305 | 0.047799788 |
| ENSG00000181192 | DHTKD1 | 5.06E−04 | 0.791904623 | −0.080578455 |
| ENSG00000181222 | POLR2A | 1.00E−12 | 0.038830003 | −0.102840994 |
| ENSG00000181555 | SETD2 | 3.00E−04 | 0.918631253 | 0.007282024 |
| ENSG00000181789 | COPG1 | 3.00E−04 | 0.241621066 | 0.077715176 |
| ENSG00000182473 | EXOC7 | 6.00E−04 | 0.343587671 | −0.074730044 |
| ENSG00000182481 | KPNA2 | 3.00E−04 | 0.644150482 | −0.032122311 |
| ENSG00000182551 | ADI1 | 9.00E−04 | 0.372377154 | 0.072506123 |
| ENSG00000182827 | ACBD3 | 3.00E−08 | 0.404828614 | 0.130448872 |
| ENSG00000183495 | EP400 | 6.00E−04 | 0.637303595 | 0.055214724 |
| ENSG00000183918 | SH2D1A | 7.01E−04 | 0.283846866 | 0.067991179 |
| ENSG00000184007 | PTP4A2 | 1.50E−07 | 0.00039459 | −0.142942918 |
| ENSG00000184009 | ACTG1 | 9.00E−09 | 0.153012011 | 0.060571337 |
| ENSG00000184357 | HIST1H1B | 3.00E−14 | 0.101743718 | 0.056835835 |
| ENSG00000184432 | COPB2 | 1.00E−04 | 0.99225242 | 0.000643147 |
| ENSG00000184445 | KNTC1 | 3.00E−04 | 0.290700324 | 0.117936173 |
| ENSG00000184634 | MED12 | 2.00E−09 | 0.441484568 | 0.08225596 |
| ENSG00000184661 | CDCA2 | 6.00E−04 | 0.129977922 | −0.116602274 |
| ENSG00000184719 | RNLS | 9.00E−04 | 0.124096231 | −0.160207128 |
| ENSG00000184825 | HIST1H2AH | 1.50E−08 | 0.236178337 | 0.035788181 |
| ENSG00000184990 | SIVA1 | 5.01E−04 | 0.001360314 | 0.174493974 |
| ENSG00000185000 | DGAT1 | 3.01E−04 | 0.711930638 | −0.055676496 |
| ENSG00000185104 | FAF1 | 1.00E−04 | 0.186505473 | −0.111825327 |
| ENSG00000185163 | DDX51 | 4.22E−06 | 0.69311416 | −0.05606435 |
| ENSG00000185236 | RAB11B | 9.00E−04 | 0.250648111 | −0.08681802 |
| ENSG00000185262 | UBALD2 | 5.00E−04 | 0.504061449 | 0.054009121 |
| ENSG00000185344 | ATP6V0A2 | 2.03E−04 | 0.449082903 | 0.078626222 |
| ENSG00000186106 | ANKRD46 | 1.00E−08 | 0.938464567 | 0.008636835 |
| ENSG00000186298 | PPP1CC | 9.02E−04 | 0.246006029 | −0.058739406 |
| ENSG00000186395 | KRT10 | 1.00E−04 | 0.616095519 | 0.04261803 |
| ENSG00000186480 | INSIG1 | 3.08E−06 | 0.573173068 | −0.037858571 |
| ENSG00000186517 | ARHGAP30 | 1.00E−04 | 0.332792401 | 0.068602551 |
| ENSG00000186566 | GPATCH8 | 9.00E−04 | 0.836769737 | −0.022374401 |
| ENSG00000186575 | NF2 | 5.00E−04 | 0.511808 | 0.06843328 |
| ENSG00000186716 | BCR | 4.02E−04 | 0.28449305 | −0.206113331 |
| ENSG00000187257 | RSBN1L | 2.10E−07 | 0.682200483 | 0.045962809 |
| ENSG00000187531 | SIRT7 | 4.01E−04 | 0.591150723 | −0.160016903 |
| ENSG00000187764 | SEMA4D | 4.00E−04 | 0.413891066 | −0.073148377 |
| ENSG00000187837 | HIST1H1C | 3.00E−09 | 0.000257571 | 0.053651622 |
| ENSG00000188229 | TUBB4B | 6.00E−08 | 0.176644347 | 0.091849122 |
| ENSG00000188486 | H2AFX | 2.64E−06 | 0.075491176 | 0.09777223 |
| ENSG00000188987 | HIST1H4D | 1.00E−13 | 0.875362395 | 0.003774928 |
| ENSG00000196155 | PLEKHG4 | 1.00E−04 | 0.015595222 | −0.295432913 |
| ENSG00000196230 | TUBB | 2.40E−07 | 0.534866787 | 0.035871882 |
| ENSG00000196235 | SUPT5H | 2.00E−04 | 0.195564051 | −0.152021425 |
| ENSG00000196305 | IARS | 3.00E−04 | 0.401865475 | 0.075934866 |
| ENSG00000196367 | TRRAP | 3.00E−10 | 0.181318964 | −0.088383498 |
| ENSG00000196396 | PTPN1 | 2.00E−04 | 0.634603291 | 0.040420571 |
| ENSG00000196498 | NCOR2 | 6.00E−04 | 0.072769487 | −0.155938346 |
| ENSG00000196504 | PRPF40A | 1.00E−09 | 0.142009995 | 0.08561315 |
| ENSG00000196535 | MYO18A | 6.00E−04 | 0.972106963 | −0.002927619 |
| ENSG00000196683 | TOMM7 | 3.00E−08 | 0.90908379 | −0.015495382 |
| ENSG00000196700 | ZNF512B | 2.09E−04 | 0.699101376 | 0.063580592 |
| ENSG00000196787 | HIST1H2AG | 5.50E−08 | 0.747550904 | −0.019583582 |
| ENSG00000196924 | FLNA | 1.00E−12 | 0.169192017 | −0.078099713 |
| ENSG00000197061 | HIST1H4C | 1.00E−09 | 0.897532466 | 0.002378482 |
| ENSG00000197081 | IGF2R | 4.10E−09 | 0.531193218 | 0.066454289 |
| ENSG00000197102 | DYNC1H1 | 5.00E−10 | 0.149889643 | 0.128641432 |
| ENSG00000197153 | HIST1H3J | 7.00E−11 | 0.888297027 | −0.007272098 |
| ENSG00000197157 | SND1 | 1.01E−04 | 0.257912014 | 0.060457678 |
| ENSG00000197312 | DDI2 | 6.01E−04 | 0.856181146 | −0.025215686 |
| ENSG00000197323 | TRIM33 | 6.00E−04 | 0.267737397 | −0.112414388 |
| ENSG00000197409 | HIST1H3D | 3.00E−12 | 0.512483364 | −0.019287888 |
| ENSG00000197601 | FAR1 | 3.30E−08 | 0.659187685 | −0.041267074 |
| ENSG00000197694 | SPTAN1 | 2.00E−10 | 0.081327953 | −0.18679623 |
| ENSG00000197697 | HIST1H2BE | 9.00E−08 | 0.493425798 | 0.028198489 |
| ENSG00000197746 | PSAP | 2.00E−10 | 0.088797396 | 0.046479701 |
| ENSG00000197903 | HIST1H2BK | 2.00E−04 | 0.781823807 | −0.022923109 |
| ENSG00000197930 | ERO1L | 6.50E−07 | 0.973303548 | −0.003430597 |
| ENSG00000198015 | MRPL42 | 2.00E−04 | 0.883226659 | −0.031389992 |
| ENSG00000198087 | CD2AP | 4.00E−04 | 0.957505459 | −0.006300497 |
| ENSG00000198231 | DDX42 | 6.00E−11 | 0.824728919 | −0.015904689 |
| ENSG00000198276 | UCKL1 | 3.30E−07 | 0.304042297 | −0.145350623 |
| ENSG00000198327 | HIST1H4F | 3.00E−09 | 0.208678387 | −0.022044471 |
| ENSG00000198339 | HIST1H4I | 2.00E−08 | 0.495872312 | 0.011398728 |
| ENSG00000198374 | HIST1H2AL | 2.05E−06 | 0.072591854 | 0.039969658 |
| ENSG00000198380 | GFPT1 | 2.00E−04 | 0.636068909 | 0.045606566 |
| ENSG00000198520 | C1orf228 | 7.80E−07 | 0.473771444 | −0.055437679 |
| ENSG00000198563 | DDX39B | 8.00E−04 | 0.271266603 | −0.112598519 |
| ENSG00000198604 | BAZ1A | 5.00E−10 | 0.704916782 | −0.030835374 |
| ENSG00000198646 | NCOA6 | 4.01E−04 | 0.116549501 | −0.173636683 |
| ENSG00000198648 | STK39 | 5.01E−04 | 0.740902509 | 0.034721562 |
| ENSG00000198728 | LDB1 | 6.00E−04 | 0.333004346 | −0.147406224 |
| ENSG00000198730 | CTR9 | 1.00E−09 | 0.11470634 | −0.142355213 |
| ENSG00000198786 | MT-ND5 | 2.00E−08 | 0.038698438 | −0.086785336 |
| ENSG00000198824 | CHAMP1 | 4.00E−04 | 0.249023725 | −0.097569413 |
| ENSG00000198911 | SREBF2 | 1.00E−04 | 0.382129128 | −0.081310955 |
| ENSG00000198917 | C9orf114 | 8.03E−04 | 0.004093641 | 0.394051515 |
| ENSG00000198952 | SMG5 | 3.00E−04 | 0.489633279 | 0.073022366 |
| ENSG00000203813 | HIST1H3H | 1.00E−04 | 0.178969463 | −0.025149763 |
| ENSG00000204138 | PHACTR4 | 6.00E−08 | 0.121133788 | −0.222449915 |
| ENSG00000204178 | TMEM57 | 3.00E−04 | 0.010544799 | −0.345157048 |
| ENSG00000204227 | RING1 | 1.00E−04 | 0.123205251 | −0.184910275 |
| ENSG00000204256 | BRD2 | 1.00E−08 | 0.232280315 | −0.080114261 |
| ENSG00000204371 | EHMT2 | 1.00E−08 | 0.297240081 | 0.090410757 |
| ENSG00000204394 | VARS | 5.00E−04 | 0.304208452 | −0.069883333 |
| ENSG00000204469 | PRRC2A | 2.00E−09 | 0.549464658 | 0.030387491 |
| ENSG00000204713 | TRIM27 | 8.00E−04 | 0.790207031 | −0.026814299 |
| ENSG00000205268 | PDE7A | 3.00E−04 | 0.558620633 | −0.048541326 |
| ENSG00000205336 | GPR56 | 8.01E−04 | 0.228558377 | −0.136323001 |
| ENSG00000205629 | LCMT1 | 1.00E−04 | 0.552673909 | −0.089560402 |
| ENSG00000205744 | DENND1C | 8.04E−04 | 0.849269508 | −0.026567467 |
| ENSG00000213064 | SFT2D2 | 8.00E−04 | 0.081132453 | −0.203416362 |
| ENSG00000214078 | CPNE1 | 3.00E−04 | 0.910315945 | −0.012942408 |
| ENSG00000215301 | DDX3X | 2.00E−04 | 0.924710307 | −0.00796857 |
| ENSG00000216490 | IFI30 | 4.13E−04 | 0.518872644 | −0.117960608 |
| ENSG00000221829 | FANCG | 1.00E−04 | 0.38011695 | 0.102243151 |
| ENSG00000227057 | WDR46 | 1.00E−10 | 0.24341875 | 0.098260531 |
| ENSG00000231925 | TAPBP | 2.00E−04 | 0.970644204 | −0.003684474 |
| ENSG00000233224 | HIST1H2AM | 1.00E−11 | 0.547023548 | −0.003832791 |
| ENSG00000234127 | TRIM26 | 9.02E−04 | 0.038240212 | −0.218304118 |
| ENSG00000241978 | AKAP2 | 1.00E−08 | 0.863267602 | 0.030178646 |
| ENSG00000253729 | PRKDC | 1.00E−12 | 0.427584653 | 0.050339925 |
| ENSG00000254870 | ATP6V1G2-DDX39B | 7.01E−04 | 0.57599435 | 0.036177813 |
| ENSG00000257103 | LSM14A | 2.00E−04 | 0.836442459 | −0.017136198 |
| ENSG00000261661 | RP11-31I10.4 | 1.27E−13 | NaN | NaN |
| ENSG00000267740 | AC024592.12 | 4.00E−04 | 0.536893222 | −0.044671372 |

TABLE 5
Genes that are both TE down and rDiff positive

| Gene ID | Gene Name | Translational Efficiency (p-value) | log2(Translational Efficiency) | rDiff (p-value) |
|---|---|---|---|---|
| ENSG00000137845.9 | ADAM10 | 0.012053048 | −0.208903322 | 8.99999E−10 |
| ENSG00000133657.10 | ATP13A3 | 0.021301072 | −0.237052311 | 9.99999E−11 |
| ENSG00000058668.10 | ATP2B4 | 0.000680955 | −0.302809666 | 9.9999E−10 |
| ENSG00000127152.13 | BCL11B | 5.40428E−10 | −0.517770746 | 9.999E−09 |
| ENSG00000135932.6 | CAB39 | 0.019948395 | −0.200243436 | 9.999E−09 |
| ENSG00000118816.5 | CCNI | 0.001528498 | −0.189161037 | 6.89931E−07 |
| ENSG00000173585.11 | CCR9 | 0.004934791 | −0.343972085 | 0.00040005 |
| ENSG00000110651.6 | CD81 | 0.010480682 | −0.130640591 | 0.00010002 |
| ENSG00000158985.9 | CDC42SE2 | 0.027991366 | −0.145160094 | 7.9992E−08 |
| ENSG00000105810.5 | CDK6 | 4.75568E−05 | −0.145254993 | 3.9996E−08 |
| ENSG00000172292.10 | CERS6 | 0.029552171 | −0.205124483 | 9.9999E−10 |
| ENSG00000171310.6 | CHST11 | 0.004536717 | −0.14604981 | 1.19988E−07 |
| ENSG00000131504.11 | DIAPH1 | 0.005406879 | −0.213577391 | 0.0002 |
| ENSG00000120690.9 | ELF1 | 0.003793641 | −0.220693753 | 0.0001 |
| ENSG00000143924.14 | EML4 | 0.028666301 | −0.26184158 | 0.00070056 |
| ENSG00000112851.10 | ERBB2IP | 0.005962767 | −0.26900197 | 0.00030017 |
| ENSG00000134954.9 | ETS1 | 1.26966E−05 | −0.232324455 | 5.69999E−09 |
| ENSG00000153310.13 | FAM49B | 0.007506383 | −0.139159484 | 0.0002 |
| ENSG00000169018.5 | FEM1B | 0.01910054 | −0.397020738 | 0.00030007 |
| ENSG00000033170.12 | FUT8 | 0.006226232 | −0.355628717 | 0.00060004 |
| ENSG00000115419.8 | GLS | 0.000197719 | −0.269875671 | 0.00040019 |
| ENSG00000078369.10 | GNB1 | 0.011642786 | −0.133797709 | 0.00050004 |
| ENSG00000172534.9 | HCFC1 | 6.57909E−09 | −0.313512877 | 2.87E−12 |
| ENSG00000114735.5 | HEMK1 | 0.020519619 | −0.432698852 | 0.00010083 |
| ENSG00000163349.15 | HIPK1 | 0.020020123 | −0.282659771 | 0.00020003 |
| ENSG00000138668.14 | HNRNPD | 0.003261874 | −0.098862205 | 2.39976E−07 |
| ENSG00000114030.8 | KPNA1 | 0.001820335 | −0.311253874 | 3.89961E−07 |
| ENSG00000108424.5 | KPNB1 | 8.66259E−05 | −0.171777065 | 2E−11 |
| ENSG00000138795.5 | LEF1 | 1.59715E−06 | −0.210659864 | 9.9999E−10 |
| ENSG00000152601.13 | MBNL1 | 0.00777836 | −0.152181062 | 2E−11 |
| ENSG00000123066.3 | MED13L | 4.78765E−05 | −0.415941737 | 2.19998E−08 |
| ENSG00000174579.3 | MSL2 | 0.027763257 | −0.205275001 | 0.0005 |
| ENSG00000149480.2 | MTA2 | 2.63948E−05 | −0.22354576 | 5.99994E−09 |
| ENSG00000136997.10 | MYC | 0.000130485 | −0.222358961 | 2.9997E−08 |
| ENSG00000196155.8 | PLEKHG4 | 0.015595222 | −0.295432913 | 0.00010019 |
| ENSG00000105063.14 | PPP6R1 | 0.029786388 | −0.157573098 | 9.99999E−11 |
| ENSG00000171522.5 | PTGER4 | 0.002288634 | −0.274827847 | 2.39976E−07 |
| ENSG00000184007.11 | PTP4A2 | 0.00039459 | −0.142942918 | 1.49985E−07 |
| ENSG00000122257.14 | RBBP6 | 0.000891329 | −0.280964053 | 1.25E−09 |
| ENSG00000102054.12 | RBBP7 | 0.003411029 | −0.129303881 | 1.6E−12 |
| ENSG00000064490.7 | RFXANK | 0.015800837 | −0.324746409 | 0.00030015 |
| ENSG00000130255.8 | RPL36 | 0.000331082 | −0.237194765 | 1.39986E−06 |
| ENSG00000137818.7 | RPLP1 | 0.002330818 | −0.514464579 | 0.00030353 |
| ENSG00000100796.13 | SMEK1 | 0.021404696 | −0.176469607 | 1E−11 |
| ENSG00000115306.10 | SPTBN1 | 0.008372115 | −0.348514727 | 3E−14 |
| ENSG00000167978.11 | SRRM2 | 0.027585188 | −0.081656945 | 0.0001 |
| ENSG00000101972.14 | STAG2 | 0.001047325 | −0.187270211 | 0.0007 |
| ENSG00000106290.10 | TAF6 | 0.014175182 | −0.210235711 | 0.00050041 |
| ENSG00000140262.13 | TCF12 | 0.025334533 | −0.184064816 | 2E−10 |
| ENSG00000105329.4 | TGFB1 | 0.015301045 | −0.221315351 | 0.0002 |
| ENSG00000140332.11 | TLE3 | 0.001343794 | −0.227829431 | 1.9998E−08 |
| ENSG00000164168.3 | TMEM184C | 0.015050183 | −0.397412646 | 2.49998E−08 |
| ENSG00000106609.11 | TMEM248 | 0.005316307 | −0.23621242 | 2.89971E−07 |
| ENSG00000204178.5 | TMEM57 | 0.010544799 | −0.345157048 | 0.00030006 |
| ENSG00000169905.7 | TOR1AIP2 | 0.025144824 | −0.223803399 | 0.00070005 |
| ENSG00000088325.11 | TPX2 | 0.000751758 | −0.147886462 | 4E−13 |
| ENSG00000154370.8 | TRIM11 | 0.010190424 | −0.431525912 | 0.00030064 |
| ENSG00000136878.7 | USP20 | 0.019165529 | −0.206602358 | 1.9998E−08 |
| ENSG00000062650.12 | WAPAL | 0.019004475 | −0.211592796 | 0.00080001 |
| ENSG00000109685.13 | WHSC1 | 0.0222009 | −0.153361985 | 1E−11 |
| ENSG00000082898.12 | XPO1 | 0.026533538 | −0.106116515 | 1E−11 |
| ENSG00000048405.5 | ZNF800 | 0.020652909 | −0.271097499 | 2.9997E−08 |

TABLE 6
Motifs and G-quadruplexes in rDiff positive

| | | | | |
|---|---|---|---|---|
| Gene ID | Gene Name | rDiff (p-value) | Translational Efficiency (p-value) | log2(Translational Efficiency) |

Table 6A. rDiff genes with 12-mer motif

| | | | | |
|---|---|---|---|---|
| ENSG00000088325 | TPX2 | 4.00E−13 | 0.000751758 | −0.147886462 |
| ENSG00000055163 | CYFIP2 | 6.00E−13 | 0.757974081 | −0.02427969 |
| ENSG00000009954 | BAZ1B | 1.00E−12 | 0.813245824 | 0.011597992 |
| ENSG00000139613 | SMARCC2 | 1.00E−12 | 0.334491125 | 0.087836839 |
| ENSG00000181222 | POLR2A | 1.00E−12 | 0.038830003 | −0.102840994 |
| ENSG00000136068 | FLNB | 2.90E−12 | 0.062111584 | −0.409977879 |
| ENSG00000127616 | SMARCA4 | 3.00E−12 | 0.813848874 | −0.012039575 |
| ENSG00000100796 | SMEK1 | 1.00E−11 | 0.021404696 | −0.176469607 |
| ENSG00000130726 | TRIM28 | 1.00E−11 | 0.034098412 | −0.092503983 |
| ENSG00000130724 | CHMP2A | 2.00E−11 | 0.770037849 | 0.028723436 |
| ENSG00000152601 | MBNL1 | 2.00E−11 | 0.00777836 | −0.152181062 |
| ENSG00000163655 | GMPS | 2.00E−11 | 0.775716157 | 0.020676052 |
| ENSG00000086758 | HUWE1 | 2.40E−11 | 0.464534104 | −0.039863394 |
| ENSG00000080345 | RIF1 | 3.00E−11 | 0.722609171 | 0.022548991 |
| ENSG00000078674 | PCM1 | 5.00E−11 | 0.600752059 | −0.058335335 |
| ENSG00000131148 | EMC8 | 5.00E−11 | 0.89007858 | −0.019540666 |
| ENSG00000198231 | DDX42 | 6.00E−11 | 0.824728919 | −0.015904689 |
| ENSG00000165417 | GTF2A1 | 7.00E−11 | 0.844760071 | −0.015019218 |
| ENSG00000104738 | MCM4 | 1.00E−10 | 0.915393017 | 0.003107424 |
| ENSG00000105063 | PPP6R1 | 1.00E−10 | 0.029786388 | −0.157573098 |
| ENSG00000110713 | NUP98 | 1.00E−10 | 0.656641011 | −0.027832725 |
| ENSG00000148773 | MKI67 | 1.00E−10 | 0.123777629 | 0.063404366 |
| ENSG00000055483 | USP36 | 2.00E−10 | 0.857880476 | 0.014047197 |
| ENSG00000109111 | SUPT6H | 2.00E−10 | 0.994134615 | 0.001145357 |
| ENSG00000197694 | SPTAN1 | 2.00E−10 | 0.081327953 | −0.18679623 |
| ENSG00000087460 | GNAS | 3.00E−10 | 0.461136397 | −0.032870857 |
| ENSG00000104517 | UBR5 | 3.00E−10 | 0.744729033 | 0.031234126 |
| ENSG00000171681 | ATF7IP | 3.00E−10 | 0.209720012 | −0.150308959 |
| ENSG00000124789 | NUP153 | 3.20E−10 | 0.677271772 | −0.045251116 |
| ENSG00000137845 | ADAM10 | 9.00E−10 | 0.012053048 | −0.208903322 |
| ENSG00000082641 | NFE2L1 | 1.00E−09 | 0.267959196 | −0.205302853 |
| ENSG00000084093 | REST | 1.00E−09 | 0.221405653 | −0.118069779 |
| ENSG00000101596 | SMCHD1 | 1.00E−09 | 0.434566245 | −0.059009881 |
| ENSG00000125755 | SYMPK | 1.00E−09 | 0.552114085 | −0.054422769 |
| ENSG00000138795 | LEF1 | 1.00E−09 | 1.60E−06 | −0.210659864 |
| ENSG00000172292 | CERS6 | 1.00E−09 | 0.029552171 | −0.205124483 |
| ENSG00000198730 | CTR9 | 1.00E−09 | 0.11470634 | −0.142355213 |
| ENSG00000013810 | TACC3 | 2.00E−09 | 0.886452126 | −0.009226853 |
| ENSG00000101224 | CDC25B | 2.00E−09 | 0.25893922 | 0.059946483 |
| ENSG00000137076 | TLN1 | 2.00E−09 | 0.114582751 | −0.09941367 |
| ENSG00000143442 | POGZ | 2.00E−09 | 0.037681202 | −0.207363006 |
| ENSG00000157540 | DYRK1A | 2.00E−09 | 0.276566574 | −0.107710063 |
| ENSG00000164190 | NIPBL | 2.00E−09 | 0.303068767 | −0.09710725 |
| ENSG00000165494 | PCF11 | 2.00E−09 | 0.168402482 | −0.149826279 |
| ENSG00000117713 | ARID1A | 2.10E−09 | 0.944760522 | 0.009363654 |
| ENSG00000153827 | TRIP12 | 2.80E−09 | 0.04819259 | −0.171340571 |
| ENSG00000132646 | PCNA | 4.00E−09 | 0.449582299 | 0.020688405 |
| ENSG00000164134 | NAA15 | 4.00E−09 | 0.476257503 | −0.043457933 |
| ENSG00000197081 | IGF2R | 4.10E−09 | 0.531193218 | 0.066454289 |
| ENSG00000134954 | ETS1 | 5.70E−09 | 1.27E−05 | −0.232324455 |
| ENSG00000149480 | MTA2 | 6.00E−09 | 2.64E−05 | −0.22354576 |
| ENSG00000114126 | TFDP2 | 7.00E−09 | 0.567162468 | −0.026000796 |
| ENSG00000120733 | KDM3B | 8.00E−09 | 0.056284307 | −0.188410685 |
| ENSG00000054654 | SYNE2 | 9.00E−09 | 0.143319349 | −0.17547751 |
| ENSG00000101191 | DIDO1 | 9.00E−09 | 0.846657226 | −0.024545447 |
| ENSG00000184009 | ACTG1 | 9.00E−09 | 0.153012011 | 0.060571337 |
| ENSG00000068024 | HDAC4 | 1.00E−08 | 0.08742941 | −0.17058278 |
| ENSG00000099381 | SETD1A | 1.00E−08 | 0.334979113 | 0.098318494 |
| ENSG00000118482 | PHF3 | 1.00E−08 | 0.873633816 | 0.012763981 |
| ENSG00000125686 | MED1 | 1.00E−08 | 0.18711587 | −0.124662129 |
| ENSG00000127152 | BCL11B | 1.00E−08 | 5.40E−10 | −0.517770746 |
| ENSG00000135905 | DOCK10 | 1.00E−08 | 0.717332641 | 0.040604232 |
| ENSG00000135932 | CAB39 | 1.00E−08 | 0.019948395 | −0.200243436 |
| ENSG00000139218 | SCAF11 | 1.00E−08 | 0.50709776 | −0.04074801 |
| ENSG00000163466 | ARPC2 | 1.00E−08 | 0.883580647 | 0.005900615 |
| ENSG00000167522 | ANKRD11 | 1.00E−08 | 0.255387498 | 0.10026816 |
| ENSG00000167670 | CHAF1A | 1.00E−08 | 0.388610811 | −0.064999638 |
| ENSG00000173020 | ADRBK1 | 1.00E−08 | 0.708537254 | −0.02554542 |
| ENSG00000125651 | GTF2F1 | 1.10E−08 | 0.031215966 | −0.168644284 |
| ENSG00000171298 | GAA | 1.10E−08 | 0.092998845 | −0.248293341 |
| ENSG00000108021 | FAM208B | 1.70E−08 | 0.236407718 | −0.130316925 |
| ENSG00000065613 | SLK | 2.00E−08 | 0.412051605 | −0.115711529 |
| ENSG00000110321 | EIF4G2 | 2.00E−08 | 0.4789335 | −0.030452093 |
| ENSG00000128191 | DGCR8 | 2.00E−08 | 0.071123809 | −0.242947421 |
| ENSG00000136878 | USP20 | 2.00E−08 | 0.019165529 | −0.206602358 |
| ENSG00000140332 | TLE3 | 2.00E−08 | 0.001343794 | −0.227829431 |
| ENSG00000144580 | RQCD1 | 2.00E−08 | 0.506022372 | −0.045037202 |
| ENSG00000123066 | MED13L | 2.20E−08 | 4.79E−05 | −0.415941737 |
| ENSG00000048405 | ZNF800 | 3.00E−08 | 0.020652909 | −0.271097499 |
| ENSG00000116698 | SMG7 | 3.00E−08 | 0.886915303 | −0.013938554 |
| ENSG00000113522 | RAD50 | 4.00E−08 | 0.22034331 | 0.108849135 |
| ENSG00000115806 | GORASP2 | 4.00E−08 | 0.107877983 | −0.140430182 |
| ENSG00000104886 | PLEKHJ1 | 6.00E−08 | 0.445334658 | 0.068851577 |
| ENSG00000110367 | DDX6 | 6.00E−08 | 0.469603134 | −0.047598358 |
| ENSG00000084733 | RAB10 | 7.00E−08 | 0.078220422 | −0.136343032 |
| ENSG00000140829 | DHX38 | 8.00E−08 | 0.404193545 | −0.083781852 |
| ENSG00000158985 | CDC42SE2 | 8.00E−08 | 0.027991366 | −0.145160094 |
| ENSG00000070756 | PABPC1 | 1.00E−07 | 0.067231582 | −0.133311245 |
| ENSG00000171310 | CHST11 | 1.20E−07 | 0.004536717 | −0.14604981 |
| ENSG00000103495 | MAZ | 1.30E−07 | 0.642227894 | 0.032737594 |
| ENSG00000080815 | PSEN1 | 1.50E−07 | 0.468385762 | −0.105188191 |
| ENSG00000184007 | PTP4A2 | 1.50E−07 | 0.00039459 | −0.142942918 |
| ENSG00000002822 | MAD1L1 | 1.70E−07 | 0.758278428 | −0.03032717 |
| ENSG00000071564 | TCF3 | 1.70E−07 | 0.851040343 | −0.020991025 |
| ENSG00000138668 | HNRNPD | 2.40E−07 | 0.003261874 | −0.098862205 |
| ENSG00000171522 | PTGER4 | 2.40E−07 | 0.002288634 | −0.274827847 |
| ENSG00000106609 | TMEM248 | 2.90E−07 | 0.005316307 | −0.23621242 |
| ENSG00000136104 | RNASEH2B | 4.20E−07 | 0.392137768 | 0.057232924 |
| ENSG00000135679 | MDM2 | 4.90E−07 | 0.948348125 | 0.012834459 |
| ENSG00000179262 | RAD23A | 5.10E−07 | 0.674786887 | 0.038029829 |
| ENSG00000071626 | DAZAP1 | 5.20E−07 | 0.060128417 | −0.110195121 |
| ENSG00000197930 | ERO1L | 6.50E−07 | 0.973303548 | −0.003430597 |
| ENSG00000118816 | CCNI | 6.90E−07 | 0.001528498 | −0.189161037 |
| ENSG00000179409 | GEMIN4 | 7.20E−07 | 0.143223537 | −0.152149612 |
| ENSG00000074603 | DPP8 | 1.09E−06 | 0.371935225 | −0.109929668 |
| ENSG00000079805 | DNM2 | 1.53E−06 | 0.594275253 | −0.034637408 |
| ENSG00000064419 | TNPO3 | 1.00E−04 | 0.030219393 | −0.182349237 |
| ENSG00000068796 | KIF2A | 1.00E−04 | 0.056041279 | 0.123499472 |
| ENSG00000077097 | TOP2B | 1.00E−04 | 0.84957972 | 0.01045839 |
| ENSG00000100401 | RANGAP1 | 1.00E−04 | 0.59275986 | −0.053239149 |
| ENSG00000104613 | INTS10 | 1.00E−04 | 0.796914737 | 0.022823898 |
| ENSG00000107854 | TNKS2 | 1.00E−04 | 0.358901659 | 0.113182695 |
| ENSG00000110651 | CD81 | 1.00E−04 | 0.010480682 | −0.130640591 |
| ENSG00000111642 | CHD4 | 1.00E−04 | 0.289163376 | 0.048737019 |
| ENSG00000124181 | PLCG1 | 1.00E−04 | 0.95873485 | −0.001417988 |
| ENSG00000136653 | RASSF5 | 1.00E−04 | 0.545754108 | −0.05276974 |
| ENSG00000138496 | PARP9 | 1.00E−04 | 0.385276066 | 0.103537296 |
| ENSG00000139350 | NEDD1 | 1.00E−04 | 0.725169134 | 0.037847451 |
| ENSG00000141027 | NCOR1 | 1.00E−04 | 0.129071822 | −0.136116164 |
| ENSG00000141556 | TBCD | 1.00E−04 | 0.591494459 | −0.026526545 |
| ENSG00000143870 | PDIA6 | 1.00E−04 | 0.042049362 | 0.102641235 |
| ENSG00000151702 | FLI1 | 1.00E−04 | 0.221034285 | −0.138078723 |
| ENSG00000156875 | HIAT1 | 1.00E−04 | 0.380713082 | −0.10910263 |
| ENSG00000157593 | SLC35B2 | 1.00E−04 | 0.149940449 | −0.2407324 |
| ENSG00000160796 | NBEAL2 | 1.00E−04 | 0.231202003 | 0.128523719 |
| ENSG00000166747 | AP1G1 | 1.00E−04 | 0.033859721 | −0.180376252 |
| ENSG00000167978 | SRRM2 | 1.00E−04 | 0.027585188 | −0.081656945 |
| ENSG00000198911 | SREBF2 | 1.00E−04 | 0.382129128 | −0.081310955 |
| ENSG00000204227 | RING1 | 1.00E−04 | 0.123205251 | −0.184910275 |
| ENSG00000205629 | LCMT1 | 1.00E−04 | 0.552673909 | −0.089560402 |
| ENSG00000104825 | NFKBIB | 1.01E−04 | 0.438223723 | 0.114649527 |
| ENSG00000125484 | GTF3C4 | 1.01E−04 | 0.265749952 | −0.128337377 |
| ENSG00000148334 | PTGES2 | 1.04E−04 | 0.626929001 | −0.068481398 |
| ENSG00000055130 | CUL1 | 2.00E−04 | 0.24134893 | −0.116033963 |
| ENSG00000077232 | DNAJC10 | 2.00E−04 | 0.043053807 | −0.322931196 |
| ENSG00000089234 | BRAP | 2.00E−04 | 0.93786126 | −0.010542696 |
| ENSG00000103222 | ABCC1 | 2.00E−04 | 0.969797812 | 0.002188323 |
| ENSG00000108175 | ZMIZ1 | 2.00E−04 | 0.05690215 | −0.222817452 |
| ENSG00000110619 | CARS | 2.00E−04 | 0.186855283 | 0.157414334 |
| ENSG00000131504 | DIAPH1 | 2.00E−04 | 0.005406879 | −0.213577391 |
| ENSG00000135090 | TAOK3 | 2.00E−04 | 0.163787333 | −0.139600964 |
| ENSG00000140525 | FANCI | 2.00E−04 | 0.688024573 | 0.029180218 |
| ENSG00000144554 | FANCD2 | 2.00E−04 | 0.730926111 | 0.036291397 |
| ENSG00000147650 | LRP12 | 2.00E−04 | 0.590549253 | 0.057987792 |
| ENSG00000151502 | VPS26B | 2.00E−04 | 0.277634848 | 0.100319458 |
| ENSG00000153310 | FAM49B | 2.00E−04 | 0.007506383 | −0.139159484 |
| ENSG00000160877 | NACC1 | 2.00E−04 | 0.286689117 | −0.119046325 |
| ENSG00000163349 | HIPK1 | 2.00E−04 | 0.020020123 | −0.282659771 |
| ENSG00000163904 | SENP2 | 2.00E−04 | 0.045589218 | −0.287466993 |
| ENSG00000177731 | FLII | 2.00E−04 | 0.139734019 | 0.11682969 |
| ENSG00000196396 | PTPN1 | 2.00E−04 | 0.634603291 | 0.040420571 |
| ENSG00000257103 | LSM14A | 2.00E−04 | 0.836442459 | −0.017136198 |
| ENSG00000072778 | ACADVL | 2.01E−04 | 0.634381953 | 0.052306846 |
| ENSG00000113580 | NR3C1 | 2.01E−04 | 0.497681416 | 0.120970261 |
| ENSG00000130311 | DDA1 | 2.01E−04 | 0.855575658 | −0.028673083 |
| ENSG00000185344 | ATP6V0A2 | 2.03E−04 | 0.449082903 | 0.078626222 |
| ENSG00000005007 | UPF1 | 3.00E−04 | 0.109682217 | −0.088407059 |
| ENSG00000011295 | TTC19 | 3.00E−04 | 0.726452954 | 0.048391976 |
| ENSG00000036257 | CUL3 | 3.00E−04 | 0.568119382 | 0.048886832 |
| ENSG00000064115 | TM7SF3 | 3.00E−04 | 0.921247137 | 0.007233323 |
| ENSG00000107164 | FUBP3 | 3.00E−04 | 0.891753365 | −0.016568849 |
| ENSG00000112308 | C6orf62 | 3.00E−04 | 0.221892591 | −0.102283924 |
| ENSG00000134371 | CDC73 | 3.00E−04 | 0.078009542 | −0.179882162 |
| ENSG00000142453 | CARM1 | 3.00E−04 | 0.397417148 | −0.08333644 |
| ENSG00000167470 | MIDN | 3.00E−04 | 0.037975203 | −0.404562969 |
| ENSG00000167491 | GATAD2A | 3.00E−04 | 0.460886475 | −0.052425426 |
| ENSG00000169018 | FEM1B | 3.00E−04 | 0.01910054 | −0.397020738 |
| ENSG00000173442 | EHBP1L1 | 3.00E−04 | 0.648913241 | 0.061932902 |
| ENSG00000174238 | PITPNA | 3.00E−04 | 0.470422902 | −0.066848619 |
| ENSG00000176619 | LMNB2 | 3.00E−04 | 0.831805089 | 0.019798531 |
| ENSG00000198952 | SMG5 | 3.00E−04 | 0.489633279 | 0.073022366 |
| ENSG00000205268 | PDE7A | 3.00E−04 | 0.558620633 | −0.048541326 |
| ENSG00000214078 | CPNE1 | 3.00E−04 | 0.910315945 | −0.012942408 |
| ENSG00000120738 | EGR1 | 3.01E−04 | 0.317981925 | −0.167567968 |
| ENSG00000129355 | CDKN2D | 3.01E−04 | 0.933543847 | −0.011782808 |
| ENSG00000130402 | ACTN4 | 3.01E−04 | 0.05655675 | −0.168897398 |
| ENSG00000073060 | SCARB1 | 4.00E−04 | 0.444106259 | −0.124760329 |
| ENSG00000100242 | SUN2 | 4.00E−04 | 0.443353969 | 0.107423956 |
| ENSG00000100697 | DICER1 | 4.00E−04 | 0.937569952 | 0.007626111 |
| ENSG00000115694 | STK25 | 4.00E−04 | 0.063817944 | −0.233874856 |
| ENSG00000119638 | NEK9 | 4.00E−04 | 0.69912267 | −0.04034303 |
| ENSG00000140943 | MBTPS1 | 4.00E−04 | 0.905572549 | 0.017038735 |
| ENSG00000156983 | BRPF1 | 4.00E−04 | 0.933570558 | −0.010817335 |
| ENSG00000198087 | CD2AP | 4.00E−04 | 0.957505459 | −0.006300497 |
| ENSG00000072364 | AFF4 | 4.01E−04 | 0.18444246 | −0.147821651 |
| ENSG00000198646 | NCOA6 | 4.01E−04 | 0.116549501 | −0.173636683 |
| ENSG00000186716 | BCR | 4.02E−04 | 0.28449305 | −0.206113331 |
| ENSG00000058063 | ATP11B | 5.00E−04 | 0.340893448 | 0.106997948 |
| ENSG00000078369 | GNB1 | 5.00E−04 | 0.011642786 | −0.133797709 |
| ENSG00000078618 | NRD1 | 5.00E−04 | 0.441231318 | −0.057082496 |
| ENSG00000106290 | TAF6 | 5.00E−04 | 0.014175182 | −0.210235711 |
| ENSG00000112200 | ZNF451 | 5.00E−04 | 0.487557618 | 0.072986258 |
| ENSG00000115548 | KDM3A | 5.00E−04 | 0.40321263 | 0.088419558 |
| ENSG00000130816 | DNMT1 | 5.00E−04 | 0.595705453 | −0.027738247 |
| ENSG00000167323 | STIM1 | 5.00E−04 | 0.977475163 | 0.002228555 |
| ENSG00000185262 | UBALD2 | 5.00E−04 | 0.504061449 | 0.054009121 |
| ENSG00000100422 | CERK | 5.01E−04 | 0.767879843 | −0.038668 |
| ENSG00000102908 | NFAT5 | 5.01E−04 | 0.225277986 | −0.178477519 |
| ENSG00000005955 | GGNBP2 | 6.00E−04 | 0.626526855 | −0.042670122 |
| ENSG00000033170 | FUT8 | 6.00E−04 | 0.006226232 | −0.355628717 |
| ENSG00000063245 | EPN1 | 6.00E−04 | 0.095672127 | −0.260058118 |
| ENSG00000067225 | PKM | 6.00E−04 | 0.089243304 | 0.079429696 |
| ENSG00000115526 | CHST10 | 6.00E−04 | 0.038600005 | −0.351592853 |
| ENSG00000132466 | ANKRD17 | 6.00E−04 | 0.746590296 | 0.023300227 |
| ENSG00000184661 | CDCA2 | 6.00E−04 | 0.129977922 | −0.116602274 |
| ENSG00000197323 | TRIM33 | 6.00E−04 | 0.267737397 | −0.112414388 |
| ENSG00000198728 | LDB1 | 6.00E−04 | 0.333004346 | −0.147406224 |
| ENSG00000033800 | PIAS1 | 6.01E−04 | 0.942720076 | 0.005741934 |
| ENSG00000075975 | MKRN2 | 6.01E−04 | 0.98382419 | 0.004421651 |
| ENSG00000104695 | PPP2CB | 7.00E−04 | 0.032333256 | −0.321922476 |
| ENSG00000115020 | PIKFYVE | 7.00E−04 | 0.874274951 | −0.045055571 |
| ENSG00000120910 | PPP3CC | 7.00E−04 | 0.20233537 | −0.200494687 |
| ENSG00000133639 | BTG1 | 7.00E−04 | 0.111632247 | −0.307842825 |
| ENSG00000143514 | TP53BP2 | 7.00E−04 | 0.283581835 | −0.117348193 |
| ENSG00000165209 | STRBP | 7.00E−04 | 0.030348179 | −0.279083308 |
| ENSG00000169905 | TOR1AIP2 | 7.00E−04 | 0.025144824 | −0.223803399 |
| ENSG00000102125 | TAZ | 7.01E−04 | 0.35021839 | 0.152681248 |
| ENSG00000111737 | RAB35 | 7.01E−04 | 0.267070813 | −0.097986528 |
| ENSG00000123983 | ACSL3 | 7.01E−04 | 0.236015207 | 0.104976406 |
| ENSG00000060237 | WNK1 | 8.00E−04 | 0.614924113 | −0.029766546 |
| ENSG00000083312 | TNPO1 | 8.00E−04 | 0.96323084 | 0.002633087 |
| ENSG00000104472 | CHRAC1 | 8.00E−04 | 0.353904047 | 0.105337178 |
| ENSG00000105676 | ARMC6 | 8.00E−04 | 0.830466125 | −0.018392253 |
| ENSG00000136824 | SMC2 | 8.00E−04 | 0.373245909 | 0.054833376 |
| ENSG00000168476 | REEP4 | 8.00E−04 | 0.228797357 | −0.127937767 |
| ENSG00000173674 | EIF1AX | 8.00E−04 | 0.780846703 | 0.072635112 |
| ENSG00000104852 | SNRNP70 | 9.00E−04 | 0.594216034 | −0.038334929 |
| ENSG00000105486 | LIG1 | 9.00E−04 | 0.865181674 | −0.011170383 |
| ENSG00000135521 | LTV1 | 9.00E−04 | 0.577086029 | 0.047229663 |
| ENSG00000180104 | EXOC3 | 9.00E−04 | 0.538380165 | −0.070376673 |
| ENSG00000185236 | RAB11B | 9.00E−04 | 0.250648111 | −0.08681802 |
| ENSG00000133961 | NUMB | 9.01E−04 | 0.646567716 | 0.059486807 |

Table 6B. rDiff genes with 9-mer motif

| | | | | |
|---|---|---|---|---|
| ENSG00000088325 | TPX2 | 4.00E−13 | 0.000751758 | −0.147886462 |
| ENSG00000055163 | CYFIP2 | 6.00E−13 | 0.757974081 | −0.02427969 |
| ENSG00000009954 | BAZ1B | 1.00E−12 | 0.813245824 | 0.011597992 |
| ENSG00000139613 | SMARCC2 | 1.00E−12 | 0.334491125 | 0.087836839 |
| ENSG00000181222 | POLR2A | 1.00E−12 | 0.038830003 | −0.102840994 |
| ENSG00000136068 | FLNB | 2.90E−12 | 0.062111584 | −0.409977879 |
| ENSG00000127616 | SMARCA4 | 3.00E−12 | 0.813848874 | −0.012039575 |
| ENSG00000055044 | NOP58 | 1.00E−11 | 0.905915474 | −0.006508419 |
| ENSG00000100796 | SMEK1 | 1.00E−11 | 0.021404696 | −0.176469607 |
| ENSG00000130726 | TRIM28 | 1.00E−11 | 0.034098412 | −0.092503983 |
| ENSG00000108424 | KPNB1 | 2.00E−11 | 8.66E−05 | −0.171777065 |
| ENSG00000130724 | CHMP2A | 2.00E−11 | 0.770037849 | 0.028723436 |
| ENSG00000152601 | MBNL1 | 2.00E−11 | 0.00777836 | −0.152181062 |
| ENSG00000163655 | GMPS | 2.00E−11 | 0.775716157 | 0.020676052 |
| ENSG00000086758 | HUWE1 | 2.40E−11 | 0.464534104 | −0.039863394 |
| ENSG00000080345 | RIF1 | 3.00E−11 | 0.722609171 | 0.022548991 |
| ENSG00000139687 | RB1 | 4.00E−11 | 0.227456544 | 0.062123731 |
| ENSG00000078674 | PCM1 | 5.00E−11 | 0.600752059 | −0.058335335 |
| ENSG00000131148 | EMC8 | 5.00E−11 | 0.89007858 | −0.019540666 |
| ENSG00000162607 | USP1 | 5.00E−11 | 0.111472525 | −0.094654587 |
| ENSG00000198231 | DDX42 | 6.00E−11 | 0.824728919 | −0.015904689 |
| ENSG00000165417 | GTF2A1 | 7.00E−11 | 0.844760071 | −0.015019218 |
| ENSG00000087087 | SRRT | 1.00E−10 | 0.966692349 | 0.001824104 |
| ENSG00000104738 | MCM4 | 1.00E−10 | 0.915393017 | 0.003107424 |
| ENSG00000105063 | PPP6R1 | 1.00E−10 | 0.029786388 | −0.157573098 |
| ENSG00000110713 | NUP98 | 1.00E−10 | 0.656641011 | −0.027832725 |
| ENSG00000148773 | MKI67 | 1.00E−10 | 0.123777629 | 0.063404366 |
| ENSG00000174231 | PRPF8 | 1.00E−10 | 0.595739886 | 0.019539188 |
| ENSG00000109111 | SUPT6H | 2.00E−10 | 0.994134615 | 0.001145357 |
| ENSG00000140262 | TCF12 | 2.00E−10 | 0.025334533 | −0.184064816 |
| ENSG00000197694 | SPTAN1 | 2.00E−10 | 0.081327953 | −0.18679623 |
| ENSG00000087460 | GNAS | 3.00E−10 | 0.461136397 | −0.032870857 |
| ENSG00000104517 | UBR5 | 3.00E−10 | 0.744729033 | 0.031234126 |
| ENSG00000171681 | ATF7IP | 3.00E−10 | 0.209720012 | −0.150308959 |
| ENSG00000124789 | NUP153 | 3.20E−10 | 0.677271772 | −0.045251116 |
| ENSG00000137845 | ADAM10 | 9.00E−10 | 0.012053048 | −0.208903322 |
| ENSG00000082641 | NFE2L1 | 1.00E−09 | 0.267959196 | −0.205302853 |
| ENSG00000084093 | REST | 1.00E−09 | 0.221405653 | −0.118069779 |
| ENSG00000100554 | ATP6V1D | 1.00E−09 | 0.428461734 | −0.082721884 |
| ENSG00000101596 | SMCHD1 | 1.00E−09 | 0.434566245 | −0.059009881 |
| ENSG00000125755 | SYMPK | 1.00E−09 | 0.552114085 | −0.054422769 |
| ENSG00000138795 | LEF1 | 1.00E−09 | 1.60E−06 | −0.210659864 |
| ENSG00000172292 | CERS6 | 1.00E−09 | 0.029552171 | −0.205124483 |
| ENSG00000198730 | CTR9 | 1.00E−09 | 0.11470634 | −0.142355213 |
| ENSG00000013810 | TACC3 | 2.00E−09 | 0.886452126 | −0.009226853 |
| ENSG00000066279 | ASPM | 2.00E−09 | 0.013635649 | 0.181712013 |
| ENSG00000118193 | KIF14 | 2.00E−09 | 0.302902759 | 0.10403347 |
| ENSG00000137076 | TLN1 | 2.00E−09 | 0.114582751 | −0.09941367 |
| ENSG00000143442 | POGZ | 2.00E−09 | 0.037681202 | −0.207363006 |
| ENSG00000151366 | NDUFC2 | 2.00E−09 | 0.361312276 | −0.043896664 |
| ENSG00000164190 | NIPBL | 2.00E−09 | 0.303068767 | −0.09710725 |
| ENSG00000165494 | PCF11 | 2.00E−09 | 0.168402482 | −0.149826279 |
| ENSG00000204469 | PRRC2A | 2.00E−09 | 0.549464658 | 0.030387491 |
| ENSG00000117713 | ARID1A | 2.10E−09 | 0.944760522 | 0.009363654 |
| ENSG00000153827 | TRIP12 | 2.80E−09 | 0.04819259 | −0.171340571 |
| ENSG00000132646 | PCNA | 4.00E−09 | 0.449582299 | 0.020688405 |
| ENSG00000164134 | NAA15 | 4.00E−09 | 0.476257503 | −0.043457933 |
| ENSG00000197081 | IGF2R | 4.10E−09 | 0.531193218 | 0.066454289 |
| ENSG00000134954 | ETS1 | 5.70E−09 | 1.27E−05 | −0.232324455 |
| ENSG00000149480 | MTA2 | 6.00E−09 | 2.64E−05 | −0.22354576 |
| ENSG00000114126 | TFDP2 | 7.00E−09 | 0.567162468 | −0.026000796 |
| ENSG00000120733 | KDM3B | 8.00E−09 | 0.056284307 | −0.188410685 |
| ENSG00000054654 | SYNE2 | 9.00E−09 | 0.143319349 | −0.17547751 |
| ENSG00000101191 | DIDO1 | 9.00E−09 | 0.846657226 | −0.024545447 |
| ENSG00000184009 | ACTG1 | 9.00E−09 | 0.153012011 | 0.060571337 |
| ENSG00000068024 | HDAC4 | 1.00E−08 | 0.08742941 | −0.17058278 |
| ENSG00000099381 | SETD1A | 1.00E−08 | 0.334979113 | 0.098318494 |
| ENSG00000099991 | CABIN1 | 1.00E−08 | 0.104911155 | −0.208959207 |
| ENSG00000109332 | UBE2D3 | 1.00E−08 | 0.601564183 | 0.04567695 |
| ENSG00000118482 | PHF3 | 1.00E−08 | 0.873633816 | 0.012763981 |
| ENSG00000125686 | MED1 | 1.00E−08 | 0.18711587 | −0.124662129 |
| ENSG00000127152 | BCL11B | 1.00E−08 | 5.40E−10 | −0.517770746 |
| ENSG00000134313 | KIDINS220 | 1.00E−08 | 0.857549373 | 0.021963698 |
| ENSG00000135905 | DOCK10 | 1.00E−08 | 0.717332641 | 0.040604232 |
| ENSG00000135932 | CAB39 | 1.00E−08 | 0.019948395 | −0.200243436 |
| ENSG00000139218 | SCAF11 | 1.00E−08 | 0.50709776 | −0.04074801 |
| ENSG00000155827 | RNF20 | 1.00E−08 | 0.674024322 | 0.06246891 |
| ENSG00000163466 | ARPC2 | 1.00E−08 | 0.883580647 | 0.005900615 |
| ENSG00000167522 | ANKRD11 | 1.00E−08 | 0.255387498 | 0.10026816 |
| ENSG00000167670 | CHAF1A | 1.00E−08 | 0.388610811 | −0.064999638 |
| ENSG00000173020 | ADRBK1 | 1.00E−08 | 0.708537254 | −0.02554542 |
| ENSG00000125651 | GTF2F1 | 1.10E−08 | 0.031215966 | −0.168644284 |
| ENSG00000171298 | GAA | 1.10E−08 | 0.092998845 | −0.248293341 |
| ENSG00000175216 | CKAP5 | 1.50E−08 | 0.03814885 | 0.117491174 |
| ENSG00000108021 | FAM208B | 1.70E−08 | 0.236407718 | −0.130316925 |
| ENSG00000065613 | SLK | 2.00E−08 | 0.412051605 | −0.115711529 |
| ENSG00000084774 | CAD | 2.00E−08 | 0.242515439 | 0.087577807 |
| ENSG00000110321 | EIF4G2 | 2.00E−08 | 0.4789335 | −0.030452093 |
| ENSG00000128191 | DGCR8 | 2.00E−08 | 0.071123809 | −0.242947421 |
| ENSG00000136878 | USP20 | 2.00E−08 | 0.019165529 | −0.206602358 |
| ENSG00000140332 | TLE3 | 2.00E−08 | 0.001343794 | −0.227829431 |
| ENSG00000144580 | RQCD1 | 2.00E−08 | 0.506022372 | −0.045037202 |
| ENSG00000171608 | PIK3CD | 2.00E−08 | 0.621359547 | −0.054808961 |
| ENSG00000123066 | MED13L | 2.20E−08 | 4.79E−05 | −0.415941737 |
| ENSG00000164168 | TMEM184C | 2.50E−08 | 0.015050183 | −0.397412646 |
| ENSG00000048405 | ZNF800 | 3.00E−08 | 0.020652909 | −0.271097499 |
| ENSG00000108439 | PNPO | 3.00E−08 | 0.034630355 | 0.181670952 |
| ENSG00000116698 | SMG7 | 3.00E−08 | 0.886915303 | −0.013938554 |
| ENSG00000117906 | RCN2 | 3.00E−08 | 0.060079639 | 0.177995705 |
| ENSG00000130175 | PRKCSH | 3.00E−08 | 0.492538567 | −0.034477239 |
| ENSG00000136997 | MYC | 3.00E−08 | 0.000130485 | −0.222358961 |
| ENSG00000113522 | RAD50 | 4.00E−08 | 0.22034331 | 0.108849135 |
| ENSG00000115806 | GORASP2 | 4.00E−08 | 0.107877983 | −0.140430182 |
| ENSG00000105677 | TMEM147 | 5.00E−08 | 0.824197085 | −0.020622451 |
| ENSG00000135316 | SYNCRIP | 5.00E−08 | 0.141605449 | −0.082838833 |
| ENSG00000104886 | PLEKHJ1 | 6.00E−08 | 0.445334658 | 0.068851577 |
| ENSG00000110367 | DDX6 | 6.00E−08 | 0.469603134 | −0.047598358 |
| ENSG00000188229 | TUBB4B | 6.00E−0S | 0.176644347 | 0.091849122 |
| ENSG00000084733 | RAB10 | 7.00E−08 | 0.078220422 | −0.136343032 |
| ENSG00000140829 | DHX38 | S.00E−08 | 0.404193545 | −0.083781852 |
| ENSG00000158985 | CDC42SE2 | 8.00E−08 | 0.027991366 | −0.145160094 |
| ENSG00000166986 | MARS | 9.00E−08 | 0.040009981 | −0.120193487 |
| ENSG00000070756 | PA8PC1 | 1.00E−07 | 0.067231582 | −0.13331124S |
| ENSG00000171310 | CHST11 | 1.20E−07 | 0.004536717 | −0.14604981 |
| ENSG00000103495 | MAZ | 1.30E−07 | 0.642227894 | 0.032737594 |
| ENSG00000080815 | PSEN1 | 1.50E−07 | 0.468385762 | −0.105188191 |
| ENSG00000184007 | PTP4A2 | 1.50E−07 | 0.00039459 | −0.142942918 |
| ENSG00000002822 | MAD1L1 | 1.70E−07 | 0.758278428 | −0.03032717 |
| ENSG00000071564 | TCF3 | 1.70E−07 | 0.851040343 | −0.020991025 |
| ENSG00000120800 | UTP20 | 1.80E−07 | 0.730921404 | 0.032446721 |
| ENSG00000167747 | C19orf48 | 1.80E−07 | 0.222151707 | −0.136113219 |
| ENSG00000132155 | RAF1 | 1.90E−07 | 0.727868161 | 0.035824977 |
| ENSG00000138668 | HNRNPD | 2.40E−07 | 0.003261874 | −0.098862205 |
| ENSG00000171522 | PTGER4 | 2.40E−07 | 0.002288634 | −0.274827847 |
| ENSG00000196230 | TUBB | 2.40E−07 | 0.534866787 | 0.035871882 |
| ENSG00000106609 | TMEM248 | 2.90E−07 | 0.005316307 | −0.23621242 |
| ENSG00000198276 | UCKL1 | 3.30E−07 | 0.304042297 | −0.145350623 |
| ENSG00000136104 | RNASEH2B | 4.20E−07 | 0.392137768 | 0.057232924 |
| ENSG00000135679 | MDM2 | 4.90E−07 | 0.948348125 | 0.012834459 |
| ENSG00000179262 | RAD23A | 5.10E−07 | 0.674786887 | 0.038029829 |
| ENSG00000071626 | DAZAP1 | 5.20E−07 | 0.060128417 | −0.110195121 |
| ENSG00000115053 | NCL | 5.50E−07 | 0.060545427 | 0.042909924 |
| ENSG00000197930 | ERO1L | 6.50E−07 | 0.973303548 | −0.003430597 |
| ENSG00000118816 | CCNI | 6.90E−07 | 0.001528498 | −0.189161037 |
| ENSG00300179409 | GEMIN4 | 7.20E−07 | 0.143223537 | −0.152149612 |
| ENSG00000151694 | ADAM17 | 7.30E−07 | 0.446223538 | −0.1096626 |
| ENSG00000074603 | DPP8 | 1.09E−06 | 0.371935225 | −0.109929668 |
| ENSG00000079805 | DNM2 | 1.53E−06 | 0.594275253 | −0.034637408 |
| ENSG00000132612 | VPS4A | 3.05E−06 | 0.187740524 | −0.117542241 |
| ENSG00000186480 | INSIG1 | 3.08E−06 | 0.573173068 | −0.037858571 |
| ENSG00000149273 | RPS3 | 2.35E−05 | 0.448604972 | −0.041417003 |
| ENSG00000038219 | BOD1L1 | 1.00E−04 | 0.003376816 | 0.278967432 |
| ENSG00000051523 | CYBA | 1.00E−04 | 0.816434248 | 0.0204381 |
| ENSG00000068796 | KIF2A | 1.00E−04 | 0.056041279 | 0.123499472 |
| ENSG00000072310 | SREBF1 | 1.00E−04 | 0.744899078 | −0.026352209 |
| ENSG00000077097 | TOP2B | 1.00E−04 | 0.84957972 | 0.01045839 |
| ENSG00000100401 | RANGAP1 | 1.00E−04 | 0.59275986 | −0.053239149 |
| ENSG00000104613 | INTS10 | 1.00E−04 | 0.796914737 | 0.022823898 |
| ENSG00000107854 | TNKS2 | 1.00E−04 | 0.358901659 | 0.113182695 |
| ENSG00000110651 | CD81 | 1.00E−04 | 0.010480682 | −0.130640591 |
| ENSG00000111642 | CHD4 | 1.00E−04 | 0.289163376 | 0.048737019 |
| ENSG00000119041 | GTF3C3 | 1.00E−04 | 0.862861391 | 0.021723507 |
| ENSG00000124181 | PLCG1 | 1.00E−04 | 0.95873485 | −0.001417988 |
| ENSG00000136653 | RASSF5 | 1.00E−04 | 0.545754108 | −0.05276974 |
| ENSG00000136758 | YME1L1 | 1.00E−04 | 0.090002669 | −0.100978955 |
| ENSG00000139350 | NEDD1 | 1.00E−04 | 0.725169134 | 0.037847451 |
| ENSG00000141027 | NCOR1 | 1.00E−04 | 0.129071822 | −0.136116164 |
| ENSG00000141556 | TBCD | 1.00E−04 | 0.591494459 | −0.026526545 |
| ENSG00000142002 | DPP9 | 1.00E−04 | 0.734190324 | −0.049637386 |
| ENSG00000143870 | PDIA6 | 1.00E−04 | 0.042049362 | 0.102641235 |
| ENSG00000156875 | HIAT1 | 1.00E−04 | 0.380713082 | −0.10910263 |
| ENSG00000157593 | SLC35B2 | 1.00E−04 | 0.149940449 | −0.2407324 |
| ENSG00000160796 | NBEAL2 | 1.00E−04 | 0.231202003 | 0.128523719 |
| ENSG00000167978 | SRRM2 | 1.00E−04 | 0.027585188 | −0.081656945 |
| ENSG00000172775 | FAM192A | 1.00E−04 | 0.366617379 | −0.209977577 |
| ENSG00000178252 | WDR6 | 1.00E−04 | 0.9305271 | 0.005862339 |
| ENSG00000184432 | COPB2 | 1.00E−04 | 0.99225242 | 0.000643147 |
| ENSG00000198911 | SREBF2 | 1.00E−04 | 0.382129128 | −0.081310955 |
| ENSG00000204227 | RING1 | 1.00E−04 | 0.123205251 | −0.184910275 |
| ENSG00000205629 | LCMT1 | 1.00E−04 | 0.552673909 | −0.089560402 |
| ENSG00000221829 | FANCG | 1.00E−04 | 0.38011695 | 0.102243151 |
| ENSG00000038210 | PI4K2B | 1.01E−04 | 0.689067203 | 0.060060784 |
| ENSG00000104825 | NFKBIB | 1.01E−04 | 0.438223723 | 0.114649527 |
| ENSG00000106459 | NRF1 | 1.01E−04 | 0.323046456 | −0.119510858 |
| ENSG00000125484 | GTF3C4 | 1.01E−04 | 0.265749952 | −0.128337377 |
| ENSG00000148334 | PTGES2 | 1.04E−04 | 0.626929001 | −0.068481398 |
| ENSG00000055130 | CUL1 | 2.00E−04 | 0.24134893 | −0.116033963 |
| ENSG00000077232 | DNAJC10 | 2.00E−04 | 0.043053807 | −0.322931196 |
| ENSG00000088247 | KHSRP | 2.00E−04 | 0.673716802 | −0.021332247 |
| ENSG00000089053 | ANAPC5 | 2.00E−04 | 0.71210468 | −0.020762022 |
| ENSG00000089234 | BRAP | 2.00E−04 | 0.93786126 | −0.010542696 |
| ENSG00000103222 | ABCC1 | 2.00E−04 | 0.969797812 | 0.002188323 |
| ENSG00000105221 | AKT2 | 2.00E−04 | 0.381664023 | −0.141483695 |
| ENSG00000105329 | TGFB1 | 2.00E−04 | 0.015301045 | −0.221315351 |
| ENSG00000108175 | ZMIZ1 | 2.00E−04 | 0.05690215 | −0.222817452 |
| ENSG00000111906 | HDDC2 | 2.00E−04 | 0.810276479 | −0.022306759 |
| ENSG00000131504 | DIAPH1 | 2.00E−04 | 0.005406879 | −0.213577391 |
| ENSG00000135090 | TAOK3 | 2.00E−04 | 0.163787333 | −0.139600964 |
| ENSG00000144554 | FANCD2 | 2.00E−04 | 0.730926111 | 0.036291397 |
| ENSG00000145833 | DDX46 | 2.00E−04 | 0.23392151 | 0.072701228 |
| ENSG00000147650 | LRP12 | 2.00E−04 | 0.590549253 | 0.057987792 |
| ENSG00000151502 | VPS26B | 2.00E−04 | 0.277634848 | 0.100319458 |
| ENSG00000153310 | FAM49B | 2.00E−04 | 0.007506383 | −0.139159484 |
| ENSG00000160877 | NACC1 | 2.00E−04 | 0.286689117 | −0.119046325 |
| ENSG00000163349 | HIPK1 | 2.00E−04 | 0.020020123 | −0.282659771 |
| ENSG00000163904 | SENP2 | 2.00E−04 | 0.045589218 | −0.287466993 |
| ENSG00000144431 | FLII | 2.00E−04 | 0.139734019 | 0.11682969 |
| ENSG00000196396 | PTPN1 | 2.00E−04 | 0.634603291 | 0.040420571 |
| ENSG00000257103 | LSM14A | 2.00E−04 | 0.836442459 | −0.017136198 |
| ENSG00000111726 | CMAS | 2.01E−04 | 0.649986681 | 0.073406219 |
| ENSG00000113580 | NR3C1 | 2.01E−04 | 0.497681416 | 0.120970261 |
| ENSG00000119403 | PHF19 | 2.01E−04 | 0.072821269 | 0.168059289 |
| ENSG00000130311 | DDA1 | 2.01E−04 | 0.855575658 | −0.028673083 |
| ENSG00000100994 | PTGB | 2.02E−04 | 0.741200463 | 0.05467102 |
| ENSG00000105401 | CDC37 | 2.02E−04 | 0.182664767 | 0.098508161 |
| ENSG00000185344 | ATP6V0A2 | 2.03E−04 | 0.449082903 | 0.078626222 |
| ENSG00000118007 | STAG1 | 2.04E−04 | 0.802913741 | −0.02813744 |
| ENSG00000196700 | ZNF512B | 2.09E−04 | 0.699101376 | 0.063580592 |
| ENSG00000005007 | UPF1 | 3.00E−04 | 0.109682217 | −0.088407059 |
| ENSG00000011295 | TTC19 | 3.00E−04 | 0.726452954 | 0.048391976 |
| ENSG00000036257 | CUL3 | 3.00E−04 | 0.568119382 | 0.048886832 |
| ENSG00000064115 | TM7SF3 | 3.00E−04 | 0.921247137 | 0.007233323 |
| ENSG00000064490 | RFXANK | 3.00E−04 | 0.015800837 | −0.324746409 |
| ENSG00000107164 | FUBP3 | 3.00E−04 | 0.891753365 | −0.016568849 |
| ENSG00000112308 | C6orf62 | 3.00E−04 | 0.221892591 | −0.102283924 |
| ENSG00000124193 | SRSF6 | 3.00E−04 | 0.119574817 | 0.116472581 |
| ENSG00000134371 | CDC73 | 3.00E−04 | 0.078009542 | −0.179882162 |
| ENSG00000137106 | GRHPR | 3.00E−04 | 0.415762699 | −0.09172823 |
| ENSG00000138081 | FBXO11 | 3.00E−04 | 0.421443356 | −0.098168828 |
| ENSG00000142453 | CARM1 | 3.00E−04 | 0.397417148 | −0.08333644 |
| ENSG00000167470 | MIDN | 3.00E−04 | 0.037975203 | −0.404562969 |
| ENSG00000167491 | GATAD2A | 3.00E−04 | 0.460886475 | −0.052425426 |
| ENSG00000169018 | FEM1B | 3.00E−04 | 0.01910054 | −0.397020738 |
| ENSG00000173442 | EHBP1L1 | 3.00E−04 | 0.648913241 | 0.061932902 |
| ENSG00000174238 | PITPNA | 3.00E−04 | 0.470422902 | −0.066848619 |
| ENSG00000175931 | UBE2O | 3.00E−04 | 0.447757287 | 0.068133312 |
| ENSG00000176619 | LMNB2 | 3.00E−04 | 0.831805089 | 0.019798531 |
| ENSG00000198952 | SMG5 | 3.00E−04 | 0.489633279 | 0.073022366 |
| ENSG00000205268 | PDE7A | 3.00E−04 | 0.558620633 | −0.048541326 |
| ENSG00000214078 | CPNE1 | 3.00E−04 | 0.910315945 | −0.012942408 |
| ENSG00000120738 | EGR1 | 3.01E−04 | 0.317981925 | −0.167567968 |
| ENSG00000129355 | CDKN2D | 3.01E−04 | 0.933543847 | −0.011782808 |
| ENSG00000130402 | ACTN4 | 3.01E−04 | 0.05655675 | −0.168897398 |
| ENSG00000131467 | PSME3 | 3.01E−04 | 0.03715923 | −0.116973165 |
| ENSG00000154370 | TRIM11 | 3.01E−04 | 0.010190424 | −0.431525912 |
| ENSG00000073060 | SCARB1 | 4.00E−04 | 0.44106259 | −0.124760329 |
| ENSG00000093009 | CDC45 | 4.00E−04 | 0.918341518 | 0.007977992 |
| ENSG00000100242 | SUN2 | 4.00E−04 | 0.443353969 | 0.107423956 |
| ENSG00000100697 | DICER1 | 4.00E−04 | 0.937569952 | 0.007626111 |
| ENSG00000104365 | IKBKB | 4.00E−04 | 0.474002406 | 0.087889302 |
| ENSG00000105939 | ZC3HAV1 | 4.00E−04 | 0.868091195 | 0.01093142 |
| ENSG00000114867 | EIF4G1 | 4.00E−04 | 0.49755475 | 0.028286796 |
| ENSG00000115419 | GLS | 4.00E−04 | 0.000197719 | −0.269875671 |
| ENSG00000115694 | STK25 | 4.00E−04 | 0.063817944 | −0.233874856 |
| ENSG00000119638 | NEK9 | 4.00E−04 | 0.69912267 | −0.04034303 |
| ENSG00000140943 | MBTPS1 | 4.00E−04 | 0.905572549 | 0.017038735 |
| ENSG00000156983 | BRPF1 | 4.00E−04 | 0.933570558 | −0.010817335 |
| ENSG00000172795 | DCP2 | 4.00E−04 | 0.294839777 | 0.094471533 |
| ENSG00000198087 | CD2AP | 4.00E−04 | 0.957505459 | −0.006300497 |
| ENSG00000072364 | AFF4 | 4.01E−04 | 0.18444246 | −0.147821651 |
| ENSG00000135763 | URB2 | 4.01E−04 | 0.668047421 | 0.04463194 |
| ENSG00000198646 | NCOA6 | 4.01E−04 | 0.116549501 | −0.173636683 |
| ENSG00000186716 | BCR | 4.02E−04 | 0.28449305 | −0.206113331 |
| ENSG00000216490 | IFI30 | 4.13E−04 | 0.518872644 | −0.117960608 |
| ENSG00000058063 | ATP11B | 5.00E−04 | 0.340893448 | 0.106997948 |
| ENSG00000078369 | GNB1 | 5.00E−04 | 0.011642786 | −0.133797709 |
| ENSG00000078618 | NRD1 | 5.00E−04 | 0.441231318 | −0.057082496 |
| ENSG00000106290 | TAF6 | 5.00E−04 | 0.014175182 | −0.210235711 |
| ENSG00000112200 | ZNF451 | 5.00E−04 | 0.487557618 | 0.072986258 |
| ENSG00000115548 | KDM3A | 5.00E−04 | 0.40321263 | 0.088419558 |
| ENSG00000130816 | DNMT1 | 5.00E−04 | 0.595705453 | −0.027738247 |
| ENSG00000132842 | AP3B1 | 5.00E−04 | 0.894277559 | 0.020029663 |
| ENSG00000138698 | RAP1GDS1 | 5.00E−04 | 0.715002624 | −0.035260421 |
| ENSG00000167323 | STIM1 | 5.00E−04 | 0.977475163 | 0.002228555 |
| ENSG00000174579 | MSL2 | 5.00E−04 | 0.027763257 | −0.205275001 |
| ENSG00000185262 | UBALD2 | 5.00E−04 | 0.504061449 | 0.054009121 |
| ENSG00000186575 | NF2 | 5.00E−04 | 0.511808 | 0.06843328 |
| ENSG00000100422 | CERK | 5.01E−04 | 0.767879843 | −0.038668 |
| ENSG00000102908 | NFAT5 | 5.01E−04 | 0.225277986 | −0.178477519 |
| ENSG00000005955 | GGNBP2 | 6.00E−04 | 0.626526855 | −0.042670122 |
| ENSG00000033170 | FUT8 | 6.00E−04 | 0.006226232 | −0.355628717 |
| ENSG00000060491 | OGFR | 6.00E−04 | 0.881572577 | −0.018437371 |
| ENSG00000063245 | EPN1 | 6.00E−04 | 0.095672127 | −0.260058118 |
| ENSG00000067225 | PKM | 6.00E−04 | 0.089243304 | 0.079429696 |
| ENSG00000090372 | STRN4 | 6.00E−04 | 0.942223216 | 0.00735298 |
| ENSG00000115526 | CHST10 | 6.00E−04 | 0.038600005 | −0.351592853 |
| ENSG00000132466 | ANKRD17 | 6.00E−04 | 0.746590296 | 0.023300227 |
| ENSG00000197323 | TRIM33 | 6.00E−04 | 0.267737397 | −0.112414388 |
| ENSG00000198728 | LDB1 | 6.00E−04 | 0.333004346 | −0.147406224 |
| ENSG00000033800 | PIAS1 | 6.01E−04 | 0.942720076 | 0.005741934 |
| ENSG00000075975 | MKRN2 | 6.01E−04 | 0.98382419 | 0.004421651 |
| ENSG00000109062 | SLC9A3R1 | 6.01E−04 | 0.122301958 | −0.064055136 |
| ENSG00000167775 | CD320 | 6.01E−04 | 0.822695325 | 0.022736769 |
| ENSG00000197312 | DDI2 | 6.01E−04 | 0.856181146 | −0.025215686 |
| ENSG00000011376 | LARS2 | 6.02E−04 | 0.269847002 | −0.142543886 |
| ENSG00000104695 | PPP2CB | 7.00E−04 | 0.032333256 | −0.321922476 |
| ENSG00000115020 | PIKFYVE | 7.00E−04 | 0.874274951 | −0.045055571 |
| ENSG00000116133 | DHCR24 | 7.00E−04 | 0.242618057 | −0.119340931 |
| ENSG00000120910 | PPP3CC | 7.00E−04 | 0.20233537 | −0.200494687 |
| ENSG00000133639 | BTG1 | 7.00E−04 | 0.111632247 | −0.307842825 |
| ENSG00000143514 | TP53BP2 | 7.00E−04 | 0.283581835 | −0.117348193 |
| ENSG00000165209 | STRBP | 7.00E−04 | 0.030348179 | −0.279083308 |
| ENSG00000169905 | TOR1AIP2 | 7.00E−04 | 0.025144824 | −0.223803399 |
| ENSG00000021762 | OSBPL5 | 7.01E−04 | 0.905417327 | −0.030769757 |
| ENSG00000102125 | TAZ | 7.01E−04 | 0.35021839 | 0.152681248 |
| ENSG00000111737 | RAB35 | 7.01E−04 | 0.267070813 | −0.097986528 |
| ENSG00000123983 | ACSL3 | 7.01E−04 | 0.236015207 | 0.104976406 |
| ENSG00000123213 | NLN | 7.02E−04 | 0.499519365 | 0.090955024 |
| ENSG00000060237 | WNK1 | 8.00E−04 | 0.614924113 | −0.029766546 |
| ENSG00000082212 | ME2 | 8.00E−04 | 0.795777072 | −0.028699073 |
| ENSG00000083312 | TNPO1 | 8.00E−04 | 0.96323084 | 0.002633087 |
| ENSG00000104472 | CHRAC1 | 8.00E−04 | 0.353904047 | 0.105337178 |
| ENSG00000105676 | ARMC6 | 8.00E−04 | 0.830466125 | −0.018392253 |
| ENSG00000138231 | DBR1 | 8.00E−04 | 0.870540124 | 0.016538189 |
| ENSG00000168476 | REEP4 | 8.00E−04 | 0.228797357 | −0.127937767 |
| ENSG00000169221 | TBC1D10B | 8.00E−04 | 0.845261963 | 0.037666933 |
| ENSG00000173674 | EIF1AX | 8.00E−04 | 0.780846703 | 0.072635112 |
| ENSG00000177156 | TALDO1 | 8.00E−04 | 0.975031972 | −0.001808282 |
| ENSG00000204713 | TR1M27 | 8.00E−04 | 0.790207031 | −0.026814299 |
| ENSG00000139946 | PELI2 | 8.01E−04 | 0.84869402 | −0.030488571 |
| ENSG00000174010 | KLHL15 | 8.01E−04 | 0.770357983 | −0.039693382 |
| ENSG00000171861 | RNMTL1 | 8.04E−04 | 0.07761122 | 0.249083661 |
| ENSG00000171202 | TMEM126A | 8.07E−04 | 0.731670543 | 0.060950022 |
| ENSG00000081791 | KIAA0141 | 9.00E−04 | 0.905406058 | −0.017374448 |
| ENSG00000104852 | SNRNP70 | 9.00E−04 | 0.594216034 | −0.038334929 |
| ENSG00000105486 | LIG1 | 9.00E−04 | 0.865181674 | −0.011170383 |
| ENSG00000115761 | NOL10 | 9.00E−04 | 0.442727268 | 0.090675848 |
| ENSG00000136709 | WDR33 | 9.00E−04 | 0.304508163 | −0.081701638 |
| ENSG00000180104 | EXOC3 | 9.00E−04 | 0.538380165 | −0.070376673 |
| ENSG00000184719 | RNLS | 9.00E−04 | 0.124096231 | −0.160207128 |
| ENSG00000185236 | RAB11B | 9.00E−04 | 0.250648111 | −0.08681802 |
| ENSG00000133961 | NUMB | 9.01E−04 | 0.646567716 | 0.059486807 |

Table 6C. rDiff genes with G-Quadruplex structure

| | | | | |
|---|---|---|---|---|
| ENSG00000009954 | BAZ1B | 1.00E−12 | 0.813245824 | .011597992 |
| ENSG00000139613 | SMARCC2 | 1.00E−12 | 0.334491125 | 0.087836839 |
| ENSG00000127616 | SMARCA4 | 3.00E−12 | 0.813848874 | −.012039575 |
| ENSG00000100796 | SMEK1 | 1.00E−11 | 0.021404696 | −.176469607 |
| ENSG00000130726 | TRIM28 | 1.00E−11 | 0.034098412 | −.092503983 |
| ENSG00000130724 | CHMP2A | 2.00E−11 | 0.770037849 | 0.028723436 |
| ENSG00000152601 | MBNL1 | 2.00E−11 | 0.00777836 | −.152181062 |
| ENSG00000163655 | GMPS | 2.00E−11 | 0.775716157 | 0.020676052 |
| ENSG00000198231 | DDX42 | 6.00E−11 | 0.824728919 | −.015904689 |
| ENSG00000105063 | PPP6R1 | 1.00E−10 | 0.029786388 | −0.157573098 |
| ENSG00000109111 | SUPT6H | 2.00E−10 | 0.994134615 | 0.001145357 |
| ENSG00000104517 | UBR5 | 3.00E−10 | 0.744729033 | 0.031234126 |
| ENSG00000171681 | ATF7IP | 3.00E−10 | 0.209720012 | −0.150308959 |
| ENSG00000137845 | ADAM10 | 9.00E−10 | 0.012053048 | −0.208903322 |
| ENSG00000058668 | ATP2B4 | 1.00E−09 | 0.000680955 | −0.302809666 |
| ENSG00000082641 | NFE2L1 | 1.00E−09 | 0.267959196 | −0.205302853 |
| ENSG00000125755 | SYMPK | 1.00E−09 | 0.552114085 | −0.054422769 |
| ENSG00000172292 | CERS6 | 1.00E−09 | 0.029552171 | −0.205124483 |
| ENSG00000013810 | TACC3 | 2.00E−09 | 0.886452126 | −0.009226853 |
| ENSG00000066279 | ASPM | 2.00E−09 | 0.013635649 | 0.181712013 |
| ENSG00000164190 | NIPBL | 2.00E−09 | 0.303068767 | −0.09710725 |
| ENSG00000117713 | ARID1A | 2.10E−09 | 0.944760522 | 0.009363654 |
| ENSG00000153827 | TRIP12 | 2.80E−09 | 0.04819259 | −0.171340571 |
| ENSG00000164134 | NAA15 | 4.00E−09 | 0.476257503 | −0.043457933 |
| ENSG00000149480 | MTA2 | 6.00E−09 | 2.64E−05 | −0.22354576 |
| ENSG00000120733 | KDM3B | 8.00E−09 | 0.056284307 | −0.188410685 |
| ENSG00000184009 | ACTG1 | 9.00E−09 | 0.153012011 | 0.060571337 |
| ENSG00000068024 | HDAC4 | 1.00E−08 | 0.08742941 | −0.17058278 |
| ENSG00000106628 | POLD2 | 1.00E−08 | 0.658612976 | −0.035385479 |
| ENSG00000118482 | PHF3 | 1.00E−08 | 0.873633816 | 0.012763981 |
| ENSG00000127152 | BCL11B | 1.00E−08 | 5.40E−10 | −0.517770746 |
| ENSG00000163466 | ARPC2 | 1.00E−08 | 0.883580647 | 0.005900615 |
| ENSG00000173020 | ADRBK1 | 1.00E−08 | 0.708537254 | −0.02554542 |
| ENSG00000108021 | FAM208B | 1.70E−08 | 0.236407718 | −0.130316925 |
| ENSG00000064613 | SLK | 2.00E−08 | 0.412051605 | −0.115711529 |
| ENSG00000093094 | OSGEP | 2.00E−08 | 0.977762307 | −0.002519015 |
| ENSG00000136878 | USP20 | 2.00E−08 | 0.019165529 | −0.206602358 |
| ENSG00000140332 | TLE3 | 2.00E−08 | 0.001343794 | −0.227829431 |
| ENSG00000171608 | PIK3CD | 2.00E−08 | 0.621359547 | −0.054808961 |
| ENSG00000123066 | MED13L | 2.20E−08 | 4.79E−05 | −0.415941737 |
| ENSG00000048405 | ZNF800 | 3.00E−08 | 0.020652909 | −0.271097499 |
| ENSG00000116698 | SMG7 | 6.00E−08 | 0.886915303 | −0.013938554 |
| ENSG00000102606 | ARHGEF7 | 6.00E−08 | 0.279523802 | −0.128921833 |
| ENSG00000125885 | MCM8 | 1.20E−07 | 0.740692289 | −0.028115948 |
| ENSG00000171310 | CHST11 | 1.20E−07 | 0.004536717 | −0.14604981 |
| ENSG00000184007 | PTP4A2 | 1.50E−07 | 0.00039459 | −0.142942918 |
| ENSG00000071564 | TCF3 | 1.70E−07 | 0.851040343 | −0.020991025 |
| ENSG00000171522 | PTGER4 | 2.40E−07 | 0.002288634 | −0.274827847 |
| ENSG00000106609 | TMEM248 | 2.90E−07 | 0.005316307 | −0.23621242 |
| ENSG00000079805 | DNM2 | 1.53E−06 | 0.594275253 | −0.034637408 |
| ENSG00000051523 | CYBA | 1.00E−04 | 0.816434248 | 0.0204381 |
| ENSG00000086504 | MRPL28 | 1.00E−04 | 0.056394 | −0.151444666 |
| ENSG00000104613 | INTS10 | 1.00E−04 | 0.796914737 | 0.022823898 |
| ENSG00000122882 | ECD | 1.00E−04 | 0.79331662 | 0.025252457 |
| ENSG00000136653 | RASSF5 | 1.00E−04 | 0.545754108 | −0.05276974 |
| ENSG00000141027 | NCOR1 | 1.00E−04 | 0.129071822 | −0.136116164 |
| ENSG00000143401 | ANP32E | 1.00E−04 | 0.915890957 | 0.004516508 |
| ENSG00000143870 | PDIA6 | 1.00E−04 | 0.042049362 | 0.102641235 |
| ENSG00000157593 | SLC35B2 | 1.00E−04 | 0.149940449 | −0.2407324 |
| ENSG00000160796 | NBEAL2 | 1.00E−04 | 0.231202003 | 0.128523719 |
| ENSG00000163808 | KIF15 | 1.00E−04 | 0.593535319 | 0.070077932 |
| ENSG00000166888 | STAT6 | 1.00E−04 | 0.047894287 | −0.21884119 |
| ENSG00000167978 | SRRM2 | 1.00E−04 | 0.027585188 | −0.081656945 |
| ENSG00000198911 | SREBF2 | 1.00E−04 | 0.382129128 | −0.081310955 |
| ENSG00000204227 | RING1 | 1.00E−04 | 0.123205251 | −0.184910275 |
| ENSG00000104825 | NFKBIB | 1.01E−04 | 0.438223723 | 0.114649527 |
| ENSG00000125484 | GTF3C4 | 1.01E−04 | 0.265749952 | −0.128337377 |
| ENSG00000089234 | BRAP | 2.00E−04 | 0.93786126 | −0.010542696 |
| ENSG00000105329 | TGFB1 | 2.00E−04 | 0.015301045 | −0.221315351 |
| ENSG00000108175 | ZMIZ1 | 2.00E−04 | 0.05690215 | −0.222817452 |
| ENSG00000129317 | PUS7L | 2.00E−04 | 0.653028133 | −0.054700064 |
| ENSG00000131504 | DIAPH1 | 2.00E−04 | 0.005406879 | −0.213577391 |
| ENSG00000135090 | TAOK3 | 2.00E−04 | 0.163787333 | −0.139600964 |
| ENSG00000147650 | LRP12 | 2.00E−04 | 0.590549253 | 0.057987792 |
| ENSG00000153310 | FAM49B | 2.00E−04 | 0.007506383 | −0.139159484 |
| ENSG00000160877 | NACC1 | 2.00E−04 | 0.586689117 | −0.119046325 |
| ENSG00000163349 | HIPK1 | 2.00E−04 | 0.020020123 | −0.282659771 |
| ENSG00000163904 | SENP2 | 2.00E−04 | 0.045589218 | −0.287466993 |
| ENSG00000177731 | FLII | 2.00E−04 | 0.139734019 | 0.11682969 |
| ENSG00000257103 | LSM14A | 2.00E−04 | 0.836442459 | −0.017136198 |
| ENSG00000111726 | CMAS | 2.01E−04 | 0.649986681 | 0.073406219 |
| ENSG00000113580 | NR3C1 | 2.01E−04 | 0.497681416 | 0.120970261 |
| ENSG00000130311 | DDA1 | 2.01E−04 | 0.855575658 | −0.028673083 |
| ENSG00000005007 | UPF1 | 3.00E−04 | 0.109682217 | −0.088407059 |
| ENSG00000064490 | RFXANK | 3.00E−04 | 0.015800837 | −0.324746409 |
| ENSG00000100029 | PES1 | 3.00E−04 | 0.805834098 | −0.019606907 |
| ENSG00000107164 | FUBP3 | 3.00E−04 | 0.891753365 | −0.016568849 |
| ENSG00000112308 | C6orf62 | 3.00E−04 | 0.221892591 | −0.102283924 |
| ENSG00000134371 | CDC73 | 3.00E−04 | 0.078009542 | −0.179882162 |
| ENSG00000142453 | CARM1 | 3.00E−04 | 0.397417148 | −0.08333644 |
| ENSG00000173442 | EHBP1L1 | 3.00E−04 | 0.648913241 | 0.064932902 |
| ENSG00000176619 | LMNB2 | 3.00E−04 | 0.831805089 | 0.019798531 |
| ENSG00000198952 | SMG5 | 3.00E−04 | 0.489633279 | 0.073022366 |
| ENSG00000214078 | CPNE1 | 3.00E−04 | 0.910315945 | −0.012942408 |
| ENSG00000129355 | CDKN2D | 3.01E−04 | 0.933543847 | −0.011782808 |
| ENSG00000100697 | DICER1 | 4.00E−04 | 0.937569952 | 0.007626111 |
| ENSG00000114867 | EIF4G1 | 4.00E−04 | 0.49755475 | 0.028286796 |
| ENSG00000115694 | STK25 | 4.00E−04 | 0.063817944 | −0.233874856 |
| ENSG00000198087 | CD2AP | 4.00E−04 | 0.957505459 | −0.006300497 |
| ENSG00000186716 | BCR | 4.02E−04 | 0.28449305 | −0.206113331 |
| ENSG00000058063 | ATP11B | 5.00E−04 | 0.340893448 | 0.106997948 |
| ENSG00000078369 | GMB1 | 5.00E−04 | 0.011642786 | −0.133797709 |
| ENSG00000078618 | NRD1 | 5.00E−04 | 0.441231318 | −0.057082496 |
| ENSG00000106290 | TAF6 | 5.00E−04 | 0.014175182 | −0.210235711 |
| ENSG00000115548 | KDM3A | 5.00E−04 | 0.40321263 | 0.088419558 |
| ENSG00000167323 | STIM1 | 5.00E−04 | 0.977475163 | 0.002228555 |
| ENSG00000185262 | UBALD2 | 5.00E−04 | 0.504061449 | 0.054009121 |
| ENSG00000102908 | NFAT5 | 5.01E−04 | 0.225277986 | −0.178477519 |
| ENSG00000005955 | GGNBP2 | 6.00E−04 | 0.626526855 | −0.042670122 |
| ENSG00000067225 | PKM | 6.00E−04 | 0.089243304 | 0.079429696 |
| ENSG00000114416 | FXR1 | 6.00E−04 | 0.827823905 | 0.018508158 |
| ENSG00000132466 | ANKRD17 | 6.00E−04 | 0.746590296 | 0.023300227 |
| ENSG00000184661 | CDCA2 | 6.00E−04 | 0.129977922 | −0.116602274 |
| ENSG00000197323 | TRIM33 | 6.00E−04 | 0.267737397 | −0.112414388 |
| ENSG00000198728 | LDB1 | 6.00E−04 | 0.333004346 | −0.147406224 |
| ENSG00000033800 | PIAS1 | 6.01E−04 | 0.942720076 | 0.005741934 |
| ENSG00000075975 | MKRN2 | 6.01E−04 | 0.98382419 | 0.004421651 |
| ENSG00000100911 | PSME2 | 6.11E−04 | 0.954296798 | −0.00949908 |
| ENSG00000101972 | STAG2 | 7.00E−04 | 0.001047325 | −0.187270211 |
| ENSG00000104695 | PPP2CB | 7.00E−04 | 0.032333256 | −0.321922476 |
| ENSG00000165209 | STRBP | 7.00E−04 | 0.030348179 | −0.279083308 |
| ENSG00000111737 | RAB35 | 7.01E−04 | 0.267070813 | −0.097986528 |
| ENSG00000060237 | WNK1 | 8.00E−04 | 0.614924113 | −0.029766546 |
| ENSG00000171202 | TMEM126A | 8.07E−04 | 0.731670543 | 0.060950022 |
| ENSG00000180104 | EXOC3 | 9.00E−04 | 0.538380165 | −0.070376673 |

Table 7A-C. Motifs and G-quadruplexes in rDiff positive genes.

TABLE 7A
rDiff genes with 12-mer motif

| Gene ID | Gene Name | Diff (p-value) | Translational Efficiency (p-value) | log2(Translational Efficiency) |
|---|---|---|---|---|
| ENSG00000088325 | TPX2 | 4.00E−13 | 0.000751758 | −0.147886462 |
| ENSG00000055163 | CYFIP2 | 6.00E−13 | 0.757974081 | −0.02427969 |
| ENSG00000009954 | BAZ1B | 1.00E−12 | 0.813245824 | 0.011597992 |
| ENSG00000139613 | SMARCC2 | 1.00E−12 | 0.334491125 | 0.087836839 |
| ENSG00000181222 | POLR2A | 1.00E−12 | 0.038830003 | −0.102840994 |
| ENSG00000136068 | FLNB | 2.90E−12 | 0.062111584 | −0.409977879 |
| ENSG00000127616 | SMARCA4 | 3.00E−12 | 0.813848874 | −0.012039575 |
| ENSG00000100796 | SMEK1 | 1.00E−11 | 0.021404696 | −0.176469607 |
| ENSG00000130726 | TRIM28 | 1.00E−11 | 0.034098412 | −0.092503983 |
| ENSG00000130724 | CHMP2A | 2.00E−11 | 0.770037849 | 0.028723436 |
| ENSG00000152601 | MBNL1 | 2.00E−11 | 0.00777836 | −0.152181062 |
| ENSG00000163655 | GM PS | 2.00E−11 | 0.775716157 | 0.020676052 |
| ENSG00000086758 | HUWE1 | 2.40E−11 | 0.464534104 | −0.039863394 |
| ENSG00000080345 | RIF1 | 3.00E−11 | 0.722609171 | 0.022548991 |
| ENSG00000078674 | PCM1 | 5.00E−11 | 0.600752059 | −0.058335335 |
| ENSG00000131148 | EMC8 | 5.00E−11 | 0.89007858 | −0.019540666 |
| ENSG00000198231 | DDX42 | 6.00E−11 | 0.824728919 | −0.015904689 |
| ENSG00000165417 | GTF2A1 | 7.00E−11 | 0.844760071 | −0.015019218 |
| ENSG00000104738 | MCM4 | 1.00E−10 | 0.915393017 | 0.003107424 |
| ENSG00000105063 | PPP6R1 | 1.00E−10 | 0.029786388 | −0.157573098 |
| ENSG00000109111 | NUP98 | 1.00E−10 | 0.656641011 | −0.027832725 |
| ENSG00000148773 | MK167 | 1.00E−10 | 0.123777629 | 0.063404366 |
| ENSG00000055483 | USP36 | 2.00E−10 | 0.857880476 | 0.014047197 |
| ENSG00000114126 | TFDP2 | 7.00E−09 | 0.567162468 | −0.026000796 |
| ENSG00000120733 | KDM3B | 8.00E−09 | 0.056284307 | −0.188410685 |
| ENSG00000054654 | SYNE2 | 9.00E−09 | 0.143319349 | −0.17547751 |
| ENSG00000101191 | DIDO1 | 9.00E−09 | 0.846657226 | −0.024545447 |
| ENSG00000184009 | ACTG1 | 9.00E−09 | 0.153012011 | 0.060571337 |
| ENSG00000068024 | HDAC4 | 1.00E−08 | 0.08742941 | −0.17058278 |
| ENSG00000099381 | SETD1A | 1.00E−08 | 0.334979113 | 0.098318494 |
| ENSG00000118482 | PHF3 | 1.00E−08 | 0.873633816 | 0.012763981 |
| ENSG00000125686 | MED1 | 1.00E−08 | 0.18711587 | −0.124662129 |
| ENSG00000127152 | BCL11B | 1.00E−08 | 5.40E−10 | −0.517770746 |
| ENSG00000135905 | DOCK10 | 1.00E−08 | 0.717332641 | 0.040604232 |
| ENSG00000135932 | CAB39 | 1.00E−08 | 0.019948395 | −0.200243436 |
| ENSG00000139218 | SCAF11 | 1.00E−08 | 0.50709776 | −0.04074801 |
| ENSG00000163466 | ARPC2 | 1.00E−08 | 0.883580647 | 0.005900615 |
| ENSG00000167522 | ANKRD11 | 1.00E−08 | 0.255387498 | 0.10026816 |
| ENSG00000167670 | CHAF1A | 1.00E−08 | 0.388610811 | −0.064999638 |
| ENSG00000173020 | ADRBK1 | 1.00E−08 | 0.708537254 | −0.02554542 |
| ENSG00000125651 | GTF2F1 | 1.10E−08 | 0.031215966 | −0.168644284 |
| ENSG00000171298 | GAA | 1.10E−08 | 0.092998845 | −0.248293341 |
| ENSG00000108021 | FAM208B | 1.70E−08 | 0.236407718 | −0.130316925 |
| ENSG00000065613 | SLK | 2.00E−08 | 0.412051605 | −0.115711529 |
| ENSG00000110321 | EIF4G2 | 2.00E−08 | 0.4789335 | −0.030452093 |
| ENSG00000128191 | DGCR8 | 2.00E−08 | 0.071123809 | −0.242947421 |
| ENSG00000136878 | USP20 | 2.00E−08 | 0.019165529 | −0.206602358 |
| ENSG00000140332 | TLE3 | 2.00E−08 | 0.001343794 | −0.227829431 |
| ENSG00000144580 | RQCD1 | 2.00E−08 | 0.506022372 | −0.045037202 |
| ENSG00000123066 | MED13L | 2.20E−08 | 4.79E−05 | −0.415941737 |
| ENSG00000048405 | ZNF800 | 3.00E−08 | 0.020652909 | −0.271097499 |
| ENSG00000116698 | SMG7 | 3.00E−08 | 0.886915303 | −0.013938554 |
| ENSG00000113522 | RAD50 | 4.00E−08 | 0.22034331 | 0.108849135 |
| ENSG00000115806 | GORASP2 | 4.00E−08 | 0.107877983 | −0.140430182 |
| ENSG00000104886 | PLEKHJ1 | 6.00E−08 | 0.445334658 | 0.068851577 |
| ENSG00000110367 | DDX6 | 6.00E−08 | 0.469603134 | −0.047598358 |
| ENSG00000084733 | RAB10 | 7.00E−08 | 0.078220422 | −0.136343032 |
| ENSG00000140829 | DHX38 | 8.00E−08 | 0.404193545 | −0.083781852 |
| ENSG00000158985 | CDC42SE2 | 8.00E−08 | 0.027991366 | −0.145160094 |
| ENSG00000070756 | PABPC1 | 1.00E−07 | 0.067231582 | −0.133311245 |
| ENSG00000171310 | CHST11 | 1.20E−07 | 0.004536717 | −0.14604981 |
| ENSG00000103495 | MAZ | 1.30E−07 | 0.642227894 | 0.032737594 |
| ENSG00000080815 | PSEN1 | 1.50E−07 | 0.468385762 | −0.105188191 |
| ENSG00000184007 | PTP4A2 | 1.50E−07 | 0.00039459 | −0.142942918 |
| ENSG00000002822 | MAD1L1 | 1.70E−07 | 0.758278428 | −0.03032717 |
| ENSG00000071564 | TCF3 | 1.70E−07 | 0.851040343 | −0.020991025 |
| ENSG00000138668 | HNRNPD | 2.40E−07 | 0.003261874 | −0.098862205 |
| ENSG00000171522 | PTGER4 | 2.40E−07 | 0.002288634 | −0.274827847 |
| ENSG00000106609 | TMEM248 | 2.90E−07 | 0.005316307 | −0.23621242 |
| ENSG00000136104 | RNASEH2B | 4.20E−07 | 0.392137768 | 0.057232924 |
| ENSG00000135679 | MDM2 | 4.90E−07 | 0.948348125 | 0.012834459 |
| ENSG00000179262 | RAD23A | 5.10E−07 | 0.674786887 | 0.038029829 |
| ENSG00000071626 | DAZAP1 | 5.20E−07 | 0.060128417 | −0.110195121 |
| ENSG00000197930 | ERO1L | 6.50E−07 | 0.973303548 | −0.003430597 |
| ENSG00000118816 | CCNI | 6.90E−07 | 0.001528498 | −0.189161037 |
| ENSG00000179409 | GEMIN4 | 7.20E−07 | 0.143223537 | −0.152149612 |
| ENSG00000074603 | DPP8 | 1.09E−06 | 0.371935225 | −0.109929668 |
| ENSG00000079805 | DNM2 | 1.53E−06 | 0.594275253 | −0.034637408 |
| ENSG00000064419 | TNPO3 | 1.00E−04 | 0.030219393 | −0.182349237 |
| ENSG00000068796 | KIF2A | 1.00E−04 | 0.056041279 | 0.123499472 |
| ENSG00000077097 | TOP2B | 1.00E−04 | 0.84957972 | 0.01045839 |
| ENSG00000100401 | RANGAP1 | 1.00E−04 | 0.59275986 | −0.053239149 |
| ENSG00000104613 | INTS10 | 1.00E−04 | 0.796914737 | 0.022823898 |
| ENSG00000107854 | TNKS2 | 1.00E−04 | 0.358901659 | 0.113182695 |
| ENSG00000110651 | CD81 | 1.00E−04 | 0.010480682 | −0.130640591 |
| ENSG00000111642 | CHD4 | 1.00E−04 | 0.289163376 | 0.048737019 |
| ENSG00000124181 | PLCG1 | 1.00E−04 | 0.95873485 | −0.001417988 |
| ENSG00000136653 | RASSF5 | 1.00E−04 | 0.545754108 | −0.05276974 |
| ENSG00000138496 | PARP9 | 1.00E−04 | 0.385276066 | 0.103537296 |
| ENSG00000139350 | NEDD1 | 1.00E−04 | 0.725169134 | 0.037847451 |
| ENSG00000141027 | NCOR1 | 1.00E−04 | 0.129071822 | −0.136116164 |
| ENSG00000141556 | TBCD | 1.00E−04 | 0.591494459 | −0.026526545 |
| ENSG00000143870 | PDIA6 | 1.00E−04 | 0.042049362 | 0.102641235 |
| ENSG00000151702 | FLI1 | 1.00E−04 | 0.221034285 | −0.138078723 |
| ENSG00000156875 | HIAT1 | 1.00E−04 | 0.380713082 | −0.10910263 |
| ENSG00000157593 | SLC35B2 | 1.00E−04 | 0.149940449 | −0.2407324 |
| ENSG00000160796 | NBEAL2 | 1.00E−04 | 0.231202003 | 0.128523719 |
| ENSG00000166747 | AP1G1 | 1.00E−04 | 0.033859721 | −0.180376252 |
| ENSG00000167978 | SRRM2 | 1.00E−04 | 0.027585188 | −0.081656945 |
| ENSG00000198911 | SREBF2 | 1.00E−04 | 0.382129128 | −0.081310955 |
| ENSG00000204227 | RING1 | 1.00E−04 | 0.123205251 | −0.184910275 |
| ENSG00000205629 | LCMT1 | 1.00E−04 | 0.552673909 | −0.089560402 |
| ENSG00000104825 | NFKBIB | 1.01E−04 | 0.438223723 | 0.114649527 |
| ENSG00000125484 | GTF3C4 | 1.01E−04 | 0.265749952 | −0.128337377 |
| ENSG00000148334 | PTGES2 | 1.04E−04 | 0.626929001 | −0.068481398 |
| ENSG00000055130 | CUL1 | 2.00E−04 | 0.24134893 | −0.116033963 |
| ENSG00000077232 | DNAJC10 | 2.00E−04 | 0.043053807 | −0.322931196 |
| ENSG00000089234 | BRAP | 2.00E−04 | 0.93786126 | −0.010542696 |
| ENSG00000103222 | ABCC1 | 2.00E−04 | 0.969797812 | 0.002188323 |
| ENSG00000108175 | ZMIZ1 | 2.00E−04 | 0.05690215 | −0.222817452 |
| ENSG00000110619 | CARS | 2.00E−04 | 0.186855283 | 0.157414334 |
| ENSG00000131504 | DIAPH1 | 2.00E−04 | 0.005406879 | −0.213577391 |
| ENSG00000135090 | TAOK3 | 2.00E−04 | 0.163787333 | −0.139600964 |
| ENSG00000140525 | FANCI | 2.00E−04 | 0.688024573 | 0.029180218 |
| ENSG00000144554 | FANCD2 | 2.00E−04 | 0.730926111 | 0.036291397 |
| ENSG00000147650 | LRP12 | 2.00E−04 | 0.590549253 | 0.057987792 |
| ENSG00000151502 | VPS26B | 2.00E−04 | 0.277634848 | 0.100319458 |
| ENSG00000153310 | FAM49B | 2.00E−04 | 0.007506383 | −0.139159484 |
| ENSG00000160877 | NACC1 | 2.00E−04 | 0.286689117 | −0.119046325 |
| ENSG00000163349 | HIPK1 | 2.00E−04 | 0.020020123 | −0.282659771 |
| ENSG00000163904 | SENP2 | 2.00E−04 | 0.045589218 | −0.287466993 |
| ENSG00000177731 | FLIT | 2.00E−04 | 0.139734019 | 0.11682969 |
| ENSG00000196396 | PTPN1 | 2.00E−04 | 0.634603291 | 0.040420571 |
| ENSG00000257103 | LSM14A | 2.00E−04 | 0.836442459 | −0.017136198 |
| ENSG00000072778 | ACADVL | 2.01E−04 | 0.634381953 | 0.052306846 |
| ENSG00000113580 | NR3C1 | 2.01E−04 | 0.497681416 | 0.120970261 |
| ENSG00000130311 | DDA1 | 2.01E−04 | 0.855575658 | −0.028673083 |
| ENSG00000185344 | ATP6V0A2 | 2.03E−04 | 0.449082903 | 0.078626222 |
| ENSG00000005007 | UPF1 | 3.00E−04 | 0.109682217 | −0.088407059 |
| ENSG00000011295 | TTC19 | 3.00E−04 | 0.726452954 | 0.048391976 |
| ENSG00000036257 | CUL3 | 3.00E−04 | 0.568119382 | 0.048886832 |
| ENSG00000064115 | TM7SF3 | 3.00E−04 | 0.921247137 | 0.007233323 |
| ENSG00000107164 | FUBP3 | 3.00E−04 | 0.891753365 | −0.016568849 |
| ENSG00000112308 | C6orf62 | 3.00E−04 | 0.221892591 | −0.102283924 |
| ENSG00000134371 | CDC73 | 3.00E−04 | 0.078009542 | −0.179882162 |
| ENSG00000142453 | CARM1 | 3.00E−04 | 0.397417148 | −0.08333644 |
| ENSG00000167470 | MIDN | 3.00E−04 | 0.037975203 | −0.404562969 |
| ENSG00000167491 | GATAD2A | 3.00E−04 | 0.460886475 | −0.052425426 |
| ENSG00000169018 | FEM1B | 3.00E−04 | 0.01910054 | −0.397020738 |
| ENSG00000173442 | EHBP1L1 | 3.00E−04 | 0.648913241 | 0.061932902 |
| ENSG00000174238 | PITPNA | 3.00E−04 | 0.470422902 | −0.066848619 |
| ENSG00000176619 | LMNB2 | 3.00E−04 | 0.831805089 | 0.019798531 |
| ENSG00000198952 | SMGS | 3.00E−04 | 0.489633279 | 0.073022366 |
| ENSG00000205268 | PDE7A | 3.00E−04 | 0.558620633 | −0.048541326 |
| ENSG00000214078 | CPNE1 | 3.00E−04 | 0.910315945 | −0.012942408 |
| ENSG00000120738 | EGR1 | 3.01E−04 | 0.317981925 | −0.167567968 |
| ENSG00000129355 | CDKN2D | 3.01E−04 | 0.933543847 | −0.011782808 |
| ENSG00000130402 | ACTN4 | 3.01E−04 | 0.05655675 | −0.168897398 |
| ENSG00000073060 | SCARB1 | 4.00E−04 | 0.444106259 | −0.124760329 |
| ENSG00000100242 | SUN2 | 4.00E−04 | 0.443353969 | 0.107423956 |
| ENSG00000100697 | DICER1 | 4.00E−04 | 0.937569952 | 0.007626111 |
| ENSG00000115694 | STK25 | 4.00E−04 | 0.063817944 | −0.233874856 |
| ENSG00000119638 | NEK9 | 4.00E−04 | 0.69912267 | −0.04034303 |
| ENSG00000140943 | MBTPS1 | 4.00E−04 | 0.905572549 | 0.017038735 |
| ENSG00000156983 | BRPF1 | 4.00E−04 | 0.933570558 | −0.010817335 |
| ENSG00000198087 | CD2AP | 4.00E−04 | 0.957505459 | −0.006300497 |
| ENSG00000072364 | AFF4 | 4.01E−04 | 0.18444246 | −0.147821651 |
| ENSG00000198646 | NCOA6 | 4.01E−04 | 0.116549501 | −0.173636683 |
| ENSG00000186716 | BCR | 4.02E−04 | 0.28449305 | −0.206113331 |
| ENSG00000058063 | ATP11B | 5.00E−04 | 0.340893448 | 0.106997948 |
| ENSG00000078369 | GNB1 | 5.00E−04 | 0.011642786 | −0.133797709 |
| ENSG00000078618 | NRD1 | 5.00E−04 | 0.441231318 | −0.057082496 |
| ENSG00000106290 | TAF6 | 5.00E−04 | 0.014175182 | −0.210235711 |
| ENSG00000112200 | ZNF451 | 5.00E−04 | 0.487557618 | 0.072986258 |
| ENSG00000115548 | KDM3A | 5.00E−04 | 0.40321263 | 0.088419558 |
| ENSG00000130816 | DNMT1 | 5.00E−04 | 0.595705453 | −0.027738247 |
| ENSG00000167323 | STIM1 | 5.00E−04 | 0.977475163 | 0.002228555 |
| ENSG00000185262 | UBALD2 | 5.00E−04 | 0.504061449 | 0.054009121 |
| ENSG00000100422 | CERK | 5.01E−04 | 0.767879843 | −0.038668 |
| ENSG00000102908 | NFAT5 | 5.01E−04 | 0.225277986 | −0.178477519 |
| ENSG00000005955 | GGNBP2 | 6.00E−04 | 0.626526855 | −0.042670122 |
| ENSG00000033170 | FUT8 | 6.00E−04 | 0.006226232 | −0.355628717 |
| ENSG00000063245 | EPN1 | 6.00E−04 | 0.095672127 | −0.260058118 |
| ENSG00000067225 | PKM | 6.00E−04 | 0.089243304 | 0.079429696 |
| ENSG00000115526 | CHST10 | 6.00E−04 | 0.038600005 | −0.351592853 |
| ENSG00000132466 | ANKRD17 | 6.00E−04 | 0.746590296 | 0.023300227 |
| ENSG00000184661 | CDCA2 | 6.00E−04 | 0.129977922 | −0.116602274 |
| ENSG00000197323 | TRIM33 | 6.00E−04 | 0.267737397 | −0.112414388 |
| ENSG00000198728 | LDB1 | 6.00E−04 | 0.333004346 | −0.147406224 |
| ENSG00000033800 | PIAS1 | 6.01E−04 | 0.942720076 | 0.005741934 |
| ENSG00000075975 | MKRN2 | 6.01E−04 | 0.98382419 | 0.004421651 |
| ENSG00000104695 | PPP2CB | 7.00E−04 | 0.032333256 | −0.321922476 |
| ENSG00000115020 | PIKFYVE | 7.00E−04 | 0.874274951 | −0.045055571 |
| ENSG00000120910 | PPP3CC | 7.00E−04 | 0.20233537 | −0.200494687 |
| ENSG00000133639 | BTG1 | 7.00E−04 | 0.111632247 | −0.307842825 |
| ENSG00000143514 | TP53BP2 | 7.00E−04 | 0.283581835 | −0.117348193 |
| ENSG00000165209 | STRBP | 7.00E−04 | 0.030348179 | −0.279083308 |
| ENSG00000169905 | TOR1AIP2 | 7.00E−04 | 0.025144824 | −0.223803399 |
| ENSG00000102125 | TAZ | 7.01E−04 | 0.35021839 | 0.152681248 |
| ENSG00000111737 | RAB35 | 7.01E−04 | 0.267070813 | −0.097986528 |
| ENSG00000123983 | ACSL3 | 7.01E−04 | 0.236015207 | 0.104976406 |
| ENSG00000060237 | WNK1 | 8.00E−04 | 0.614924113 | −0.029766546 |
| ENSG00000083312 | TNPO1 | 8.00E−04 | 0.96323084 | 0.002633087 |
| ENSG00000104472 | CHRAC1 | 8.00E−04 | 0.353904047 | 0.105337178 |
| ENSG00000105676 | ARMC6 | 8.00E−04 | 0.830466125 | −0.018392253 |
| ENSG00000136824 | SMC2 | 8.00E−04 | 0.373245909 | 0.054833376 |
| ENSG00000168476 | REEP4 | 8.00E−04 | 0.228797357 | −0.127937767 |
| ENSG00000173674 | EIF1AX | 8.00E−04 | 0.780846703 | 0.072635112 |
| ENSG00000104852 | SNRNP70 | 9.00E−04 | 0.594216034 | −0.038334929 |
| ENSG00000105486 | LIG1 | 9.00E−04 | 0.865181674 | −0.011170383 |
| ENSG00000135521 | LTV1 | 9.00E−04 | 0.577086029 | 0.047229663 |
| ENSG00000180104 | EXOC3 | 9.00E−04 | 0.538380165 | −0.070376673 |
| ENSG00000185236 | RAB11B | 9.00E−04 | 0.250648111 | −0.08681802 |

### ENSG00000133961 NUMB 9.01E-04 0.646567716 0.059486807

TABLE 7B
rDiff genes with 9-mer motif

| Gene ID | Gene Name | rDiff (p-value) | Translational Efficiency (p-value) | log2(Translational efficiency) |
|---|---|---|---|---|
| ENSG00000088325 | TPX2 | 4.00E−13 | 0.000751758 | −.147886462 |
| ENSG00000055163 | CYFIP2 | 6.00E−13 | 0.757974081 | −0.02427969 |
| ENSG00000009954 | BAZ1B | 1.00E−12 | 0.813245824 | 0.011597992 |
| ENSG00000139613 | SMARCC2 | 1.00E−12 | 0.334491125 | 0.087836839 |
| ENSG00000181222 | POLR2A | 1.00E−12 | 0.038830003 | −.102840994 |
| ENSG00000136068 | FLNB | 2.90E−12 | 0.062111584 | −.409977879 |
| ENSG00000127616 | SMARCA4 | 3.00E−12 | 0.813848874 | −.012039575 |
| ENSG00000055044 | NOP58 | 1.00E−11 | 0.905915474 | −.006508419 |
| ENSG00000100796 | SMEK1 | 1.00E−11 | 0.021404696 | −.176469607 |
| ENSG00000130726 | TRIM28 | 1.00E−11 | 0.034098412 | −.092503983 |
| ENSG00000108424 | KPNB1 | 2.00E−11 | 8.66E−05 | −.171777065 |
| ENSG00000130724 | CHMP2A | 2.00E−11 | 0.770037849 | 0.028723436 |
| ENSG00000152601 | MBNL1 | 2.00E−11 | 0.00777836 | −0.152181062 |
| ENSG00000163655 | GMPS | 2.00E−11 | 0.775716157 | 0.020676052 |
| ENSG00000086758 | HUWE1 | 2.40E−11 | 0.464534104 | −0.039863394 |
| ENSG00000080345 | RIF1 | 3.00E−11 | 0.722609171 | 0.022548991 |
| ENSG00000139687 | RB1 | 4.00E−11 | 0.227456544 | 0.062123731 |
| ENSG00000078674 | PCM1 | 5.00E−11 | 0.600752059 | −0.058335335 |
| ENSG00000131148 | EMC8 | 5.00E−11 | 0.89007858 | −0.019540666 |
| ENSG00000162607 | USP1 | 5.00E−11 | 0.111472525 | −0.094654587 |
| ENSG00000198231 | DDX42 | 6.00E−11 | 0.824728919 | −0.015904689 |
| ENSG00000165417 | GTF2A1 | 7.00E−11 | 0.844760071 | −0.015019218 |
| ENSG00000087087 | SRRT | 1.00E−10 | 0.966692349 | 0.001824104 |
| ENSG00000104738 | MCM4 | 1.00E−10 | 0.915393017 | 0.003107424 |
| ENSG00000105063 | PPP6R1 | 1.00E−10 | 0.029786388 | −0.157573098 |
| ENSG00000110713 | NUP98 | 1.00E−10 | 0.656641011 | −0.027832725 |
| ENSG00000148773 | MK167 | 1.00E−10 | 0.123777629 | 0.063404366 |
| ENSG00000174231 | PRPF8 | 1.00E−10 | 0.595739886 | 0.019539188 |
| ENSG00000109111 | SUPT6H | 2.00E−10 | 0.994134615 | 0.001145357 |
| ENSG00000140262 | TCF12 | 2.00E−10 | 0.025334533 | −0.184064816 |
| ENSG00000197694 | SPTAN1 | 2.00E−10 | 0.081327953 | −0.18679623 |
| ENSG00000087460 | GNAS | 3.00E−10 | 0.461136397 | −0.032870857 |
| ENSG00000104517 | UBRS | 3.00E−10 | 0.744729033 | 0.031234126 |
| ENSG00000171681 | ATF7IP | 3.00E−10 | 0.209720012 | −0.150308959 |
| ENSG00000124789 | NUP153 | 3.20E−10 | 0.677271772 | −0.045251116 |
| ENSG00000137845 | ADAM10 | 9.00E−10 | 0.012053048 | −0.208903322 |
| ENSG00000082641 | NFE2L1 | 1.00E−09 | 0.267959196 | −0.205302853 |
| ENSG00000084093 | REST | 1.00E−09 | 0.221405653 | −0.118069779 |
| ENSG00000100554 | ATP6V1D | 1.00E−09 | 0.428461734 | −0.082721884 |
| ENSG00000101596 | SMCHD1 | 1.00E−09 | 0.434566245 | −0.059009881 |
| ENSG00000125755 | SYMPK | 1.00E−09 | 0.552114085 | −0.054422769 |
| ENSG00000138795 | LEF1 | 1.00E−09 | 1.60E−06 | −0.210659864 |
| ENSG00000172292 | CERS6 | 1.00E−09 | 0.029552171 | −0.205124483 |
| ENSG00000198730 | CTR9 | 1.00E−09 | 0.11470634 | −0.142355213 |
| ENSG00000013810 | TACC3 | 2.00E−09 | 0.886452126 | −0.009226853 |
| ENSG00000066279 | ASPM | 2.00E−09 | 0.013635649 | 0.181712013 |
| ENSG00000118193 | KIF14 | 2.00E−09 | 0.302902759 | 0.10403347 |
| ENSG00000137076 | TLN1 | 2.00E−09 | 0.114582751 | −0.09941367 |
| ENSG00000143442 | POGZ | 2.00E−09 | 0.037681202 | −0.207363006 |
| ENSG00000151366 | NDUFC2 | 2.00E−09 | 0.361312276 | −0.043896664 |
| ENSG00000164190 | NIPBL | 2.00E−09 | 0.303068767 | −0.09710725 |
| ENSG00000165494 | PCF11 | 2.00E−09 | 0.168402482 | −0.149826279 |
| ENSG00000204469 | PRRC2A | 2.00E−09 | 0.549464658 | 0.030387491 |
| ENSG00000117713 | ARID1A | 2.10E−09 | 0.944760522 | 0.009363654 |
| ENSG00000153827 | TRIP12 | 2.80E−09 | 0.04819259 | −0.171340571 |
| ENSG00000132646 | PCNA | 4.00E−09 | 0.449582299 | 0.020688405 |
| ENSG00000164134 | NAA15 | 4.00E−09 | 0.476257503 | −0.043457933 |
| ENSG00000197081 | IGF2R | 4.10E−09 | 0.531193218 | 0.066454289 |
| ENSG00000134954 | ETS1 | 5.70E−09 | 1.27E−05 | −0.232324455 |
| ENSG00000149480 | MTA2 | 6.00E−09 | 2.64E−05 | −0.22354576 |
| ENSG00000114126 | TFDP2 | 7.00E−09 | 0.567162468 | −0.026000796 |
| ENSG00000120733 | KDM3B | 8.00E−09 | 0.056284307 | −0.188410685 |
| ENSG00000054654 | SYNE2 | 9.00E−09 | 0.143319349 | −0.17547751 |
| ENSG00000101191 | DIDO1 | 9.00E−09 | 0.846657226 | −0.024545447 |
| ENSG00000184009 | ACTG1 | 9.00E−09 | 0.153012011 | 0.060571337 |
| ENSG00000068024 | HDAC4 | 1.00E−08 | 0.08742941 | −0.17058278 |
| ENSG00000099381 | SETD1A | 1.00E−08 | 0.334979113 | 0.098318494 |
| ENSG00000099991 | CABIN1 | 1.00E−08 | 0.104911155 | −0.208959207 |
| ENSG00000109332 | UBE2D3 | 1.00E−08 | 0.601564183 | 0.04567695 |
| ENSG00000118482 | PHF3 | 1.00E−08 | 0.873633816 | 0.012763981 |
| ENSG00000125686 | MED1 | 1.00E−08 | 0.18711587 | −0.124662129 |
| ENSG00000127152 | BCL11B | 1.00E−08 | 5.40E−10 | −0.517770746 |
| ENSG00000134313 | KIDINS220 | 1.00E−08 | 0.857549373 | 0.021963698 |
| ENSG00000135905 | DOCK10 | 1.00E−08 | 0.717332641 | 0.040604232 |
| ENSG00000135932 | CAB39 | 1.00E−08 | 0.019948395 | −0.200243436 |
| ENSG00000139218 | SCAF11 | 1.00E−08 | 0.50709776 | −0.04074801 |
| ENSG00000155827 | RNF20 | 1.00E−08 | 0.674024322 | 0.06246891 |
| ENSG00000163466 | ARPC2 | 1.00E−08 | 0.883580647 | 0.005900615 |
| ENSG00000167522 | ANKRD11 | 1.00E−08 | 0.255387498 | 0.10026816 |
| ENSG00000167670 | CHAF1A | 1.00E−08 | 0.388610811 | −0.064999638 |
| ENSG00000173020 | ADRBK1 | 1.00E−08 | 0.708537254 | −0.02554542 |
| ENSG00000125651 | GTF2F1 | 1.10E−08 | 0.031215966 | −0.168644284 |
| ENSG00000171298 | GAA | 1.10E−08 | 0.092998845 | −0.248293341 |
| ENSG00000175216 | CKAP5 | 1.50E−08 | 0.03814885 | 0.117491174 |
| ENSG00000108021 | FAM208B | 1.70E−08 | 0.236407718 | −0.130316925 |
| ENSG00000065613 | SLK | 2.00E−08 | 0.412051605 | −0.115711529 |
| ENSG00000084774 | CAD | 2.00E−08 | 0.242515439 | 0.087577807 |
| ENSG00000110321 | EIF4G2 | 2.00E−08 | 0.4789335 | −0.030452093 |
| ENSG00000128191 | DGCR8 | 2.00E−08 | 0.071123809 | −0.242947421 |
| ENSG00000136878 | USP20 | 2.00E−08 | 0.019165529 | −0.206602358 |
| ENSG00000140332 | TLE3 | 2.00E−08 | 0.001343794 | −0.227829431 |
| ENSG00000144580 | RQCD1 | 2.00E−08 | 0.506022372 | −0.045037202 |
| ENSG00000171608 | PIK3CD | 2.00E−08 | 0.621359547 | −0.054808961 |
| ENSG00000123066 | MED13L | 2.20E−08 | 4.79E−05 | −0.415941737 |
| ENSG00000164168 | TMEM184C | 2.50E−08 | 0.015050183 | −0.397412646 |
| ENSG00000048405 | ZNF800 | 3.00E−08 | 0.020652909 | −0.271097499 |
| ENSG00000108439 | PNPO | 3.00E−08 | 0.034630355 | 0.181670952 |
| ENSG00000116698 | SMG7 | 3.00E−08 | 0.886915303 | −0.013938554 |
| ENSG00000117906 | RCN2 | 3.00E−08 | 0.060079639 | 0.177995705 |
| ENSG00000130175 | PRKCSH | 3.00E−08 | 0.492538567 | −0.034477239 |
| ENSG00000136997 | MYC | 3.00E−08 | 0.000130485 | −0.222358961 |
| ENSG00000113522 | RAD50 | 4.00E−08 | 0.22034331 | 0.108849135 |
| ENSG00000115806 | GORASP2 | 4.00E−08 | 0.107877983 | −0.140430182 |
| ENSG00000105677 | TMEM147 | 5.00E−08 | 0.824197085 | −0.020622451 |
| ENSG00000135316 | SYNCRIP | 5.00E−08 | 0.141605449 | −0.082838833 |
| ENSG00000104886 | PLEKHJ1 | 6.00E−08 | 0.445334658 | 0.068851577 |
| ENSG00000110367 | DDX6 | 6.00E−08 | 0.469603134 | −0.047598358 |
| ENSG00000188229 | TUBB4B | 6.00E−08 | 0.176644347 | 0.091849122 |
| ENSG00000084733 | RAB10 | 7.00E−08 | 0.078220422 | −0.136343032 |
| ENSG00000140829 | DHX38 | 8.00E−08 | 0.404193545 | −0.083781852 |
| ENSG00000158985 | CDC42SE2 | 8.00E−08 | 0.027991366 | −0.145160094 |
| ENSG00000166986 | MARS | 9.00E−08 | 0.040009981 | −0.120193487 |
| ENSG00000070756 | PABPC1 | 1.00E−07 | 0.067231582 | −0.133311245 |
| ENSG00000171310 | CHST11 | 1.20E−07 | 0.004536717 | −0.14604981 |
| ENSG00000103495 | MAZ | 1.30E−07 | 0.642227894 | 0.032737594 |
| ENSG00000080815 | PSEN1 | 1.50E−07 | 0.468385762 | −0.105188191 |
| ENSG00000184007 | PTP4A2 | 1.50E−07 | 0.00039459 | −0.142942918 |
| ENSG00000002822 | MAD1L1 | 1.70E−07 | 0.758278428 | −0.03032717 |
| ENSG00000071564 | TCF3 | 1.70E−07 | 0.851040343 | −0.020991025 |
| ENSG00000120800 | UTP20 | 1.80E−07 | 0.730921404 | 0.032446721 |
| ENSG00000167747 | C19orf48 | 1.80E−07 | 0.222151707 | −0.136113219 |
| ENSG00000132155 | RAF1 | 1.90E−07 | 0.727868161 | 0.035824977 |
| ENSG00000138668 | HNRNPD | 2.40E−07 | 0.003261874 | −0.098862205 |
| ENSG00000171522 | PTGER4 | 2.40E−07 | 0.002288634 | −0.274827847 |
| ENSG00000196230 | TUBB | 2.40E−07 | 0.534866787 | 0.035871882 |
| ENSG00000106609 | TMEM248 | 2.90E−07 | 0.005316307 | −0.23621242 |
| ENSG00000198276 | UCKL1 | 3.30E−07 | 0.304042297 | −0.145350623 |
| ENSG00000136104 | RNASEH2B | 4.20E−07 | 0.392137768 | 0.057232924 |
| ENSG00000135679 | MDM2 | 4.90E−07 | 0.948348125 | 0.012834459 |
| ENSG00000179262 | RAD23A | 5.10E−07 | 0.674786887 | 0.038029829 |
| ENSG00000071626 | DAZAP1 | 5.20E−07 | 0.060128417 | −0.110195121 |
| ENSG00000115053 | NCL | 5.50E−07 | 0.060545427 | 0.042909924 |
| ENSG00000197930 | ERO1L | 6.50E−07 | 0.973303548 | −0.003430597 |
| ENSG00000118816 | CCNI | 6.90E−07 | 0.001528498 | −0.189161037 |
| ENSG00000179409 | GEMIN4 | 7.20E−07 | 0.143223537 | −0.152149612 |
| ENSG00000151694 | ADAM17 | 7.30E−07 | 0.446223538 | −0.1096626 |
| ENSG00000074603 | DPP8 | 1.09E−06 | 0.371935225 | −0.109929668 |
| ENSG00000079805 | DNM2 | 1.53E−06 | 0.594275253 | −0.034637408 |
| ENSG00000132612 | VPS4A | 3.05E−06 | 0.187740524 | −0.117542241 |
| ENSG00000186480 | INSIG1 | 3.08E−06 | 0.573173068 | −0.037858571 |
| ENSG00000149273 | RPS3 | 2.35E−05 | 0.448604972 | −0.041417003 |
| ENSG00000038219 | BOD1L1 | 1.00E−04 | 0.003376816 | 0.278967432 |
| ENSG00000051523 | CYBA | 1.00E−04 | 0.816434248 | 0.0204381 |
| ENSG00000068796 | KIF2A | 1.00E−04 | 0.056041279 | 0.123499472 |
| ENSG00000072310 | SREBF1 | 1.00E−04 | 0.744899078 | −0.026352209 |
| ENSG00000077097 | TOP2B | 1.00E−04 | 0.84957972 | 0.01045839 |
| ENSG00000100401 | RANGAP1 | 1.00E−04 | 0.59275986 | −0.053239149 |
| ENSG00000104613 | INTS10 | 1.00E−04 | 0.796914737 | 0.022823898 |
| ENSG00000107854 | TNKS2 | 1.00E−04 | 0.358901659 | 0.113182695 |
| ENSG00000110651 | CD81 | 1.00E−04 | 0.010480682 | −0.130640591 |
| ENSG00000111642 | CHD4 | 1.00E−04 | 0.289163376 | 0.048737019 |
| ENSG00000119041 | GTF3C3 | 1.00E−04 | 0.862861391 | 0.021723507 |
| ENSG00000124181 | PLCG1 | 1.00E−04 | 0.95873485 | −0.001417988 |
| ENSG00000136653 | RASSF5 | 1.00E−04 | 0.545754108 | −0.05276974 |
| ENSG00000136758 | YME1L1 | 1.00E−04 | 0.090002669 | −0.100978955 |
| ENSG00000139350 | NEDD1 | 1.00E−04 | 0.725169134 | 0.037847451 |
| ENSG00000141027 | NCOR1 | 1.00E−04 | 0.129071822 | −0.136116164 |
| ENSG00000141556 | TBCD | 1.00E−04 | 0.591494459 | −0.026526545 |
| ENSG00000142002 | DPP9 | 1.00E−04 | 0.734190324 | −0.049637386 |
| ENSG00000143870 | PDIA6 | 1.00E−04 | 0.042049362 | 0.102641235 |
| ENSG00000156875 | HIAT1 | 1.00E−04 | 0.380713082 | −0.10910263 |
| ENSG00000157593 | SLC35B2 | 1.00E−04 | 0.149940449 | −0.2407324 |
| ENSG00000160796 | NBEAL2 | 1.00E−04 | 0.231202003 | 0.128523719 |
| ENSG00000167978 | SRRM2 | 1.00E−04 | 0.027585188 | −0.081656945 |
| ENSG00000172775 | FAM192A | 1.00E−04 | 0.366617379 | −0.209977577 |
| ENSG00000178252 | WDR6 | 1.00E−04 | 0.9305271 | 0.005862339 |
| ENSG00000184432 | COPB2 | 1.00E−04 | 0.99225242 | 0.000643147 |
| ENSG00000198911 | SREBF2 | 1.00E−04 | 0.382129128 | −0.081310955 |
| ENSG00000204227 | RING1 | 1.00E−04 | 0.123205251 | −0.184910275 |
| ENSG00000205629 | LCMT1 | 1.00E−04 | 0.552673909 | −0.089560402 |
| ENSG00000221829 | FANCG | 1.00E−04 | 0.38011695 | 0.102243151 |
| ENSG00000038210 | P14K2B | 1.01E−04 | 0.689067203 | 0.060060784 |
| ENSG00000104825 | NFKBIB | 1.01E−04 | 0.438223723 | 0.114649527 |
| ENSG00000106459 | NRF1 | 1.01E−04 | 0.323046456 | −0.119510858 |
| ENSG00000125484 | GTF3C4 | 1.01E−04 | 0.265749952 | −0.128337377 |
| ENSG00000148334 | PTGES2 | 1.04E−04 | 0.626929001 | −0.068481398 |
| ENSG00000055130 | CUL1 | 2.00E−04 | 0.24134893 | −0.116033963 |
| ENSG00000077232 | DNAJC10 | 2.00E−04 | 0.043053807 | −0.322931196 |
| ENSG00000088247 | KHSRP | 2.00E−04 | 0.673716802 | −0.021332247 |
| ENSG00000089053 | ANAPCS | 2.00E−04 | 0.71210468 | −0.020762022 |
| ENSG00000089234 | BRAP | 2.00E−04 | 0.93786126 | −0.010542696 |
| ENSG00000103222 | ABCC1 | 2.00E−04 | 0.969797812 | 0.002188323 |
| ENSG00000105221 | AKT2 | 2.00E−04 | 0.381664023 | −0.141483695 |
| ENSG00000105329 | TGFB1 | 2.00E−04 | 0.015301045 | −0.221315351 |
| ENSG00000108175 | ZMIZ1 | 2.00E−04 | 0.05690215 | −0.222817452 |
| ENSG00000111906 | HDDC2 | 2.00E−04 | 0.810276479 | −0.022306759 |
| ENSG00000131504 | DIAPH1 | 2.00E−04 | 0.005406879 | −0.213577391 |
| ENSG00000135090 | TAOK3 | 2.00E−04 | 0.163787333 | −0.139600964 |
| ENSG00000144554 | FANCD2 | 2.00E−04 | 0.730926111 | 0.036291397 |
| ENSG00000145833 | DDX46 | 2.00E−04 | 0.23392151 | 0.072701228 |
| ENSG00000147650 | LRP12 | 2.00E−04 | 0.590549253 | 0.057987792 |
| ENSG00000151502 | VPS26B | 2.00E−04 | 0.277634848 | 0.100319458 |
| ENSG00000153310 | FAM49B | 2.00E−04 | 0.007506383 | −0.139159484 |
| ENSG00000160877 | NACC1 | 2.00E−04 | 0.286689117 | −0.119046325 |
| ENSG00000163349 | HIPK1 | 2.00E−04 | 0.020020123 | −0.282659771 |
| ENSG00000163904 | SENP2 | 2.00E−04 | 0.045589218 | −0.287466993 |
| ENSG00000177731 | FLI I | 2.00E−04 | 0.139734019 | 0.11682969 |
| ENSG00000196396 | PTPN1 | 2.00E−04 | 0.634603291 | 0.040420571 |
| ENSG00000257103 | LSM14A | 2.00E−04 | 0.836442459 | −0.017136198 |
| ENSG00000111726 | CMAS | 2.01E−04 | 0.649986681 | 0.073406219 |
| ENSG00000113580 | NR3C1 | 2.01E−04 | 0.497681416 | 0.120970261 |
| ENSG00000119403 | PHF19 | 2.01E−04 | 0.072821269 | 0.168059289 |
| ENSG00000130311 | DDA1 | 2.01E−04 | 0.855575658 | −0.028673083 |
| ENSG00000100994 | PYGB | 2.02E−04 | 0.741200463 | 0.05467102 |
| ENSG00000105401 | CDC37 | 2.02E−04 | 0.182664767 | 0.098508161 |
| ENSG00000185344 | ATP6VOA2 | 2.03E−04 | 0.449082903 | 0.078626222 |
| ENSG00000118007 | STAG1 | 2.04E−04 | 0.802913841 | −0.02813744 |
| ENSG00000196700 | ZNF512B | 2.09E−04 | 0.699101376 | 0.063580592 |
| ENSG00000005007 | UPF1 | 3.00E−04 | 0.109682217 | −0.088407059 |
| ENSG00000011295 | TTC19 | 3.00E−04 | 0.726452954 | 0.048391976 |
| ENSG00000036257 | CUL3 | 3.00E−04 | 0.568119382 | 0.048886832 |
| ENSG00000064115 | TM7SF3 | 3.00E−04 | 0.921247137 | 0.007233323 |
| ENSG00000064490 | RFXANK | 3.00E−04 | 0.015800837 | −0.324746409 |
| ENSG00000107164 | FUBP3 | 3.00E−04 | 0.891753365 | −0.016568849 |
| ENSG00000112308 | C6orf62 | 3.00E−04 | 0.221892591 | −0.102283924 |
| ENSG00000124193 | SRSF6 | 3.00E−04 | 0.119574817 | 0.116472581 |
| ENSG00000134371 | CDC73 | 3.00E−04 | 0.078009542 | −0.179882162 |
| ENSG00000137106 | GRHPR | 3.00E−04 | 0.415762699 | −0.09172823 |
| ENSG00000138081 | FBXO11 | 3.00E−04 | 0.421443356 | −0.098168828 |
| ENSG00000142453 | CARM1 | 3.00E−04 | 0.397417148 | −0.08333644 |
| ENSG00000167470 | MIDN | 3.00E−04 | 0.037975203 | −0.404562969 |
| ENSG00000167491 | GATAD2A | 3.00E−04 | 0.460886475 | −0.052425426 |
| ENSG00000169018 | FEM1B | 3.00E−04 | 0.01910054 | −0.397020738 |
| ENSG00000173442 | EHBP1L1 | 3.00E−04 | 0.648913241 | 0.061932902 |
| ENSG00000174238 | PITPNA | 3.00E−04 | 0.470422902 | −0.066848619 |
| ENSG00000175931 | UBE2O | 3.00E−04 | 0.447757287 | 0.068133312 |
| ENSG00000176619 | LMNB2 | 3.00E−04 | 0.831805089 | 0.019798531 |
| ENSG00000198952 | SMGS | 3.00E−04 | 0.489633279 | 0.073022366 |
| ENSG00000205268 | PDE7A | 3.00E−04 | 0.558620633 | −0.048541326 |
| ENSG00000214078 | CPNE1 | 3.00E−04 | 0.910315945 | −0.012942408 |
| ENSG00000120738 | EGR1 | 3.01E−04 | 0.317981925 | −0.167567968 |
| ENSG00000129355 | CDKN2D | 3.01E−04 | 0.933543847 | −0.011782808 |
| ENSG00000130402 | ACTN4 | 3.01E−04 | 0.05655675 | −0.168897398 |
| ENSG00000131467 | PSME3 | 3.01E−04 | 0.03715923 | −0.116973165 |
| ENSG00000154370 | TRIM11 | 3.01E−04 | 0.010190424 | −0.431525912 |
| ENSG00000073060 | SCARB1 | 4.00E−04 | 0.444106259 | −0.124760329 |
| ENSG00000093009 | CDC45 | 4.00E−04 | 0.918341518 | 0.007977992 |
| ENSG00000100242 | SUN2 | 4.00E−04 | 0.443353969 | 0.107423956 |
| ENSG00000100697 | DICER1 | 4.00E−04 | 0.937569952 | 0.007626111 |
| ENSG00000104365 | IKBKB | 4.00E−04 | 0.474002406 | 0.087889302 |
| ENSG00000105939 | ZC3HAV1 | 4.00E−04 | 0.868091195 | 0.01093142 |
| ENSG00000114867 | EIF4G1 | 4.00E−04 | 0.49755475 | 0.028286796 |
| ENSG00000115419 | GLS | 4.00E−04 | 0.000197719 | −0.269875671 |
| ENSG00000115694 | STK25 | 4.00E−04 | 0.063817944 | −0.233874856 |
| ENSG00000119638 | NEK9 | 4.00E−04 | 0.69912267 | −0.04034303 |
| ENSG00000140943 | MBTPS1 | 4.00E−04 | 0.905572549 | 0.017038735 |
| ENSG00000156983 | BRPF1 | 4.00E−04 | 0.933570558 | −0.010817335 |
| ENSG00000172795 | DCP2 | 4.00E−04 | 0.294839777 | 0.094471533 |
| ENSG00000198087 | CD2AP | 4.00E−04 | 0.957505459 | −0.006300497 |
| ENSG00000072364 | AFF4 | 4.01E−04 | 0.18444246 | −0.147821651 |
| ENSG00000135763 | URB2 | 4.01E−04 | 0.668047421 | 0.04463194 |
| ENSG00000198646 | NCOA6 | 4.01E−04 | 0.116549501 | −0.173636683 |
| ENSG00000186716 | BCR | 4.02E−04 | 0.28449305 | −0.206113331 |
| ENSG00000216490 | IFI30 | 4.13E−04 | 0.518872644 | −0.117960608 |
| ENSG00000058063 | ATP11B | 5.00E−04 | 0.340893448 | 0.106997948 |
| ENSG00000078369 | GNB1 | 5.00E−04 | 0.011642786 | −0.133797709 |
| ENSG00000078618 | NRD1 | 5.00E−04 | 0.441231318 | −0.057082496 |
| ENSG00000106290 | TAF6 | 5.00E−04 | 0.014175182 | −0.210235711 |
| ENSG00000112200 | ZNF451 | 5.00E−04 | 0.487557618 | 0.072986258 |
| ENSG00000115548 | KDM3A | 5.00E−04 | 0.40321263 | 0.088419558 |
| ENSG00000130816 | DNMT1 | 5.00E−04 | 0.595705453 | −0.027738247 |
| ENSG00000132842 | AP3B1 | 5.00E−04 | 0.894277559 | 0.020029663 |
| ENSG00000138698 | RAP1GDS1 | 5.00E−04 | 0.715002624 | −0.035260421 |
| ENSG00000167323 | STIM1 | 5.00E−04 | 0.977475163 | 0.002228555 |
| ENSG00000174579 | MSL2 | 5.00E−04 | 0.027763257 | −0.205275001 |
| ENSG00000185262 | UBALD2 | 5.00E−04 | 0.504061449 | 0.054009121 |
| ENSG00000186575 | NF2 | 5.00E−04 | 0.511808 | 0.06843328 |
| ENSG00000100422 | CERK | 5.01E−04 | 0.767879843 | −0.038668 |
| ENSG00000102908 | NFAT5 | 5.01E−04 | 0.225277986 | −0.178477519 |
| ENSG00000005955 | GGNBP2 | 6.00E−04 | 0.626526855 | −0.042670122 |
| ENSG00000033170 | FUT8 | 6.00E−04 | 0.006226232 | −0.355628717 |
| ENSG00000060491 | OGFR | 6.00E−04 | 0.881572577 | −0.018437371 |
| ENSG00000063245 | EPN1 | 6.00E−04 | 0.095672127 | −0.260058118 |
| ENSG00000067225 | PKM | 6.00E−04 | 0.089243304 | 0.079429696 |
| ENSG00000090372 | STRN4 | 6.00E−04 | 0.942223216 | 0.00735298 |
| ENSG00000115526 | CHST10 | 6.00E−04 | 0.038600005 | −0.351592853 |
| ENSG00000132466 | ANKRD17 | 6.00E−04 | 0.746590296 | 0.023300227 |
| ENSG00000197323 | TRIM33 | 6.00E−04 | 0.267737397 | −0.112414388 |
| ENSG00000198728 | LDB1 | 6.00E−04 | 0.333004346 | −0.147406224 |
| ENSG00000033800 | PIAS1 | 6.01E−04 | 0.942720076 | 0.005741934 |
| ENSG00000075975 | MKRN2 | 6.01E−04 | 0.98382419 | 0.004421651 |
| ENSG00000109062 | SLC9A3R1 | 6.01E−04 | 0.122301958 | −0.064055136 |
| ENSG00000167775 | CD320 | 6.01E−04 | 0.822695325 | 0.022736769 |
| ENSG00000197312 | DDI2 | 6.01E−04 | 0.856181146 | −0.025215686 |
| ENSG00000011376 | LARS2 | 6.02E−04 | 0.269847002 | −0.142543886 |
| ENSG00000104695 | PPP2CB | 7.00E−04 | 0.032333256 | −0.321922476 |
| ENSG00000115020 | PIKFYVE | 7.00E−04 | 0.874274951 | −0.045055571 |
| ENSG00000116133 | DHCR24 | 7.00E−04 | 0.242618057 | −0.119340931 |
| ENSG00000120910 | PPP3CC | 7.00E−04 | 0.20233537 | −0.200494687 |
| ENSG00000133639 | BTG1 | 7.00E−04 | 0.111632247 | −0.307842825 |
| ENSG00000143514 | TP53BP2 | 7.00E−04 | 0.283581835 | −0.117348193 |
| ENSG00000165209 | STRBP | 7.00E−04 | 0.030348179 | −0.279083308 |
| ENSG00000169905 | TOR1AIP2 | 7.00E−04 | 0.025144824 | −0.223803399 |
| ENSG00000021762 | OSBPLS | 7.01E−04 | 0.905417327 | −0.030769757 |
| ENSG00000102125 | TAZ | 7.01E−04 | 0.35021839 | 0.152681248 |
| ENSG00000111737 | RAB35 | 7.01E−04 | 0.267070813 | −0.097986528 |
| ENSG00000123983 | ACSL3 | 7.01E−04 | 0.236015207 | 0.104976406 |
| ENSG00000123213 | NLN | 7.02E−04 | 0.499519365 | 0.090955024 |
| ENSG00000060237 | WNK1 | 8.00E−04 | 0.614924113 | −0.029766546 |
| ENSG00000082212 | ME2 | 8.00E−04 | 0.795777072 | −0.028699073 |
| ENSG00000083312 | TNPO1 | 8.00E−04 | 0.96323084 | 0.002633087 |
| ENSG00000104472 | CHRAC1 | 8.00E−04 | 0.353904047 | 0.105337178 |
| ENSG00000105676 | ARMC6 | 8.00E−04 | 0.830466125 | −0.018392253 |
| ENSG00000138231 | DBR1 | 8.00E−04 | 0.870540124 | 0.016538189 |
| ENSG00000168476 | REEP4 | 8.00E−04 | 0.228797357 | −0.127937767 |
| ENSG00000169221 | TBC1DOB | 8.00E−04 | 0.845261963 | 0.037666933 |
| ENSG00000173674 | EIF1AX | 8.00E−04 | 0.780846703 | 0.072635112 |
| ENSG00000177156 | TALDO1 | 8.00E−04 | 0.975031972 | −0.001808282 |
| ENSG00000204713 | TRIM27 | 8.00E−04 | 0.790207031 | −0.026814299 |
| ENSG00000139946 | PELI2 | 8.01E−04 | 0.84869402 | −0.030488571 |
| ENSG00000174010 | KLHL15 | 8.01E−04 | 0.770357983 | −0.039693382 |
| ENSG00000171861 | RNMTL1 | 8.04E−04 | 0.07761122 | 0.249083661 |
| ENSG00000171202 | TMEM126A | 8.07E−04 | 0.731670543 | 0.060950022 |
| ENSG00000081791 | KIAA0141 | 9.00E−04 | 0.905406058 | −0.017374448 |
| ENSG00000104852 | SNRNP70 | 9.00E−04 | 0.594216034 | −0.038334929 |
| ENSG00000105486 | LIG1 | 9.00E−04 | 0.865181674 | −0.011170383 |
| ENSG00000115761 | NOL10 | 9.00E−04 | 0.442727268 | 0.090675848 |
| ENSG00000136709 | WDR33 | 9.00E−04 | 0.304508163 | −0.081701638 |
| ENSG00000180104 | EXOC3 | 9.00E−04 | 0.538380165 | −0.070376673 |
| ENSG00000184719 | RNLS | 9.00E−04 | 0.124096231 | −0.160207128 |
| ENSG00000185236 | RAB11B | 9.00E−04 | 0.250648111 | −0.08681802 |
| ENSG00000133961 | NUMB | 9.01E−04 | 0.646567716 | 0.059486807 |

TABLE 7C
rDiff genes with G-Quadruplex structure

| Gene ID | Gene Name | rDiff (p-value) | Translational Efficiency (p-value) | log2(Translational Efficiency) |
|---|---|---|---|---|
| ENSG00000009954 | BAZ1B | 1.00E−12 | 0.813245824 | 0.011597992 |
| ENSG00000139613 | SMARCC2 | 1.00E−12 | 0.334491125 | 0.087836839 |
| ENSG00000127616 | SMARCA4 | 3.00E−12 | 0.813848874 | −.012039575 |
| ENSG00000100796 | SMEK1 | 1.00E−11 | 0.021404696 | −.176469607 |
| ENSG00000130726 | TRIM28 | 1.00E−11 | 0.034098412 | −.092503983 |
| ENSG00000130724 | CHMP2A | 2.00E−11 | 0.770037849 | 0.028723436 |
| ENSG00000152601 | MBNL1 | 2.00E−11 | 0.00777836 | −.152181062 |
| ENSG00000163655 | GMPS | 2.00E−11 | 0.775716157 | 0.020676052 |
| ENSG00000198231 | DDX42 | 6.00E−11 | 0.824728919 | −.015904689 |
| ENSG00000105063 | PPP6R1 | 1.00E−10 | 0.029786388 | −.157573098 |
| ENSG00000109111 | SUPT6H | 2.00E−10 | 0.994134615 | 0.001145357 |
| ENSG00000104517 | UBR5 | 3.00E−10 | 0.744729033 | 0.031234126 |
| ENSG00000171681 | ATF7IP | 3.00E−10 | 0.209720012 | −.150308959 |
| ENSG00000137845 | ADAM10 | 9.00E−10 | 0.012053048 | −.208903322 |
| ENSG00000058668 | ATP2B4 | 1.00E−09 | 0.000680955 | −.302809666 |
| ENSG00000082641 | NFE2L1 | 1.00E−09 | 0.267959196 | −.205302853 |
| ENSG00000125755 | SYM PK | 1.00E−09 | 0.552114085 | −.054422769 |
| ENSG00000172292 | CERS6 | 1.00E−09 | 0.029552171 | −.205124483 |
| ENSG00000013810 | TACC3 | 2.00E−09 | 0.886452126 | −.009226853 |
| ENSG00000066279 | ASPM | 2.00E−09 | 0.013635649 | 0.181712013 |
| ENSG00000164190 | NIPBL | 2.00E−09 | 0.303068767 | −0.09710725 |
| ENSG00000117713 | ARID1A | 2.10E−09 | 0.944760522 | 0.009363654 |
| ENSG00000153827 | TRIP12 | 2.80E−09 | 0.04819259 | −0.171340571 |
| ENSG00000164134 | NAA15 | 4.00E−09 | 0.476257503 | −0.043457933 |
| ENSG00000149480 | MTA2 | 6.00E−09 | 2.64E−05 | −0.22354576 |
| ENSG00000120733 | KDM3B | 8.00E−09 | 0.056284307 | −0.188410685 |
| ENSG00000184009 | ACTG1 | 9.00E−09 | 0.153012011 | 0.060571337 |
| ENSG00000068024 | HDAC4 | 1.00E−08 | 0.08742941 | −0.17058278 |
| ENSG00000106628 | POLD2 | 1.00E−08 | 0.658612976 | −0.035385479 |
| ENSG00000118482 | PHF3 | 1.00E−08 | 0.873633816 | 0.012763981 |
| ENSG00000127152 | BCL11B | 1.00E−08 | 5.40E−10 | −0.517770746 |
| ENSG00000163466 | ARPC2 | 1.00E−08 | 0.883580647 | 0.005900615 |
| ENSG00000173020 | ADRBK1 | 1.00E−08 | 0.708537254 | −0.02554542 |
| ENSG00000108021 | FAM208B | 1.70E−08 | 0.236407718 | −0.130316925 |
| ENSG00000065613 | SLK | 2.00E−08 | 0.412051605 | −0.115711529 |
| ENSG00000092094 | OSGEP | 2.00E−08 | 0.977762307 | −0.002519015 |
| ENSG00000136878 | USP20 | 2.00E−08 | 0.019165529 | −0.206602358 |
| ENSG00000140332 | TLE3 | 2.00E−08 | 0.001343794 | −0.227829431 |
| ENSG00000171608 | PIK3CD | 2.00E−08 | 0.621359547 | −0.054808961 |
| ENSG00000123066 | MED13L | 2.20E−08 | 4.79E−05 | −0.415941737 |
| ENSG00000048405 | ZNF800 | 3.00E−08 | 0.020652909 | −0.271097499 |
| ENSG00000116698 | SMG7 | 3.00E−08 | 0.886915303 | −0.013938554 |
| ENSG00000102606 | ARHGEF7 | 6.00E−08 | 0.279523802 | −0.128921833 |
| ENSG00000125885 | MCM8 | 1.20E−07 | 0.740692289 | −0.028115948 |
| ENSG00000171310 | CHST11 | 1.20E−07 | 0.004536717 | −0.14604981 |
| ENSG00000184007 | PTP4A2 | 1.50E−07 | 0.00039459 | −0.142942918 |
| ENSG00000071564 | TCF3 | 1.70E−07 | 0.851040343 | −0.020991025 |
| ENSG00000171522 | PTGER4 | 2.40E−07 | 0.002288634 | −0.274827847 |
| ENSG00000106609 | TMEM248 | 2.90E−07 | 0.005316307 | −0.23621242 |
| ENSG00000079805 | DNM2 | 1.53E−06 | 0.594275253 | −0.034637408 |
| ENSG00000051523 | CYBA | 1.00E−04 | 0.816434248 | 0.0204381 |
| ENSG00000086504 | MRPL28 | 1.00E−04 | 0.056394 | −0.151444666 |
| ENSG00000104613 | INTS10 | 1.00E−04 | 0.796914737 | 0.022823898 |
| ENSG00000122882 | ECD | 1.00E−04 | 0.79331662 | 0.025252457 |
| ENSG00000136653 | RASSF5 | 1.00E−04 | 0.545754108 | −0.05276974 |
| ENSG00000141027 | NCOR1 | 1.00E−04 | 0.129071822 | −0.136116164 |
| ENSG00000143401 | ANP32E | 1.00E−04 | 0.915890957 | 0.004516508 |
| ENSG00000143870 | PDIA6 | 1.00E−04 | 0.042049362 | 0.102641235 |
| ENSG00000157593 | SLC35B2 | 1.00E−04 | 0.149940449 | −0.2407324 |
| ENSG00000160796 | NBEAL2 | 1.00E−04 | 0.231202003 | 0.128523719 |
| ENSG00000163808 | KIF15 | 1.00E−04 | 0.593535319 | 0.070077932 |
| ENSG00000166888 | STAT6 | 1.00E−04 | 0.047894287 | −0.21884119 |
| ENSG00000167978 | SRRM2 | 1.00E−04 | 0.027585188 | −0.081656945 |
| ENSG00000198911 | SREBF2 | 1.00E−04 | 0.382129128 | −0.081310955 |
| ENSG00000204227 | RING1 | 1.00E−04 | 0.123205251 | −0.184910275 |
| ENSG00000104825 | NFKBIB | 1.01E−04 | 0.438223723 | 0.114649527 |
| ENSG00000125484 | GTF3C4 | 1.01E−04 | 0.265749952 | −0.128337377 |
| ENSG00000089234 | BRAP | 2.00E−04 | 0.93786126 | −0.010542696 |
| ENSG00000105329 | TGFB1 | 2.00E−04 | 0.015301045 | −0.221315351 |
| ENSG00000108175 | ZMIZ1 | 2.00E−04 | 0.05690215 | −0.222817452 |
| ENSG00000129317 | PUS7L | 2.00E−04 | 0.653028133 | −0.054700064 |
| ENSG00000131504 | DIAPH1 | 2.00E−04 | 0.005406879 | −0.213577391 |
| ENSG00000135090 | TAOK3 | 2.00E−04 | 0.163787333 | −0.139600964 |
| ENSG00000147650 | LRP12 | 2.00E−04 | 0.590549253 | 0.057987792 |
| ENSG00000153310 | FAM49B | 2.00E−04 | 0.007506383 | −0.139159484 |
| ENSG00000160877 | NACC1 | 2.00E−04 | 0.286689117 | −0.119046325 |
| ENSG00000163349 | HIPK1 | 2.00E−04 | 0.020020123 | −0.282659771 |
| ENSG00000163904 | SENP2 | 2.00E−04 | 0.045589218 | −0.287466993 |
| ENSG00000177731 | FLII | 2.00E−04 | 0.139734019 | 0.11682969 |
| ENSG00000257103 | LSM14A | 2.00E−04 | 0.836442459 | −0.017136198 |
| ENSG00000111726 | CMAS | 2.01E−04 | 0.649986681 | 0.073406219 |
| ENSG00000113580 | NR3C1 | 2.01E−04 | 0.497681416 | 0.120970261 |
| ENSG00000130311 | DDA1 | 2.01E−04 | 0.855575658 | −0.028673083 |
| ENSG00000005007 | UPF1 | 3.00E−04 | 0.109682217 | −0.088407059 |
| ENSG00000064490 | RFXANK | 3.00E−04 | 0.015800837 | −0.324746409 |
| ENSG00000100029 | PES1 | 3.00E−04 | 0.805834098 | −0.019606907 |
| ENSG00000107164 | FUBP3 | 3.00E−04 | 0.891753365 | −0.016568849 |
| ENSG00000112308 | C6orf62 | 3.00E−04 | 0.221892591 | −0.102283924 |
| ENSG00000134371 | CDC73 | 3.00E−04 | 0.078009542 | −0.179882162 |
| ENSG00000142453 | CARM1 | 3.00E−04 | 0.397417148 | −0.08333644 |
| ENSG00000173442 | EHBP1L1 | 3.00E−04 | 0.648913241 | 0.061932902 |
| ENSG00000176619 | LMNB2 | 3.00E−04 | 0.831805089 | 0.019798531 |
| ENSG00000198952 | SMG5 | 3.00E−04 | 0.489633279 | 0.073022366 |
| ENSG00000214078 | CPNE1 | 3.00E−04 | 0.910315945 | −0.012942408 |
| ENSG00000129355 | CDKN2D | 3.01E−04 | 0.933543847 | −0.011782808 |
| ENSG00000100697 | DICER1 | 4.00E−04 | 0.937569952 | 0.007626111 |
| ENSG00000114867 | EIFG1 | 4.00E−04 | 0.49755475 | 0.028286796 |
| ENSG00000115694 | STK25 | 4.00E−04 | 0.063817944 | −0.233874856 |
| ENSG00000198087 | CD2AP | 4.00E−04 | 0.957505459 | −0.006300497 |
| ENSG00000167323 | BCR | 4.02E−04 | 0.28449305 | −0.206113331 |
| ENSG00000058063 | ATP11B | 5.00E−04 | 0.340893448 | 0.106997948 |
| ENSG00000078369 | GNB1 | 5.00E−04 | 0.011642786 | −0.133797709 |
| ENSG00000078618 | NRD1 | 5.00E−04 | 0.441231318 | −0.057082496 |
| ENSG00000106290 | TAF6 | 5.00E−04 | 0.014175182 | −0.210235711 |
| ENSG00000115548 | KDM3A | 5.00E−04 | 0.40321263 | 0.088419558 |

What is claimed is:

1. A method for identifying an agent capable of modulating cap-dependent mRNA translation, the method comprising comparing translation efficiency in the presence and absence of the agent in an in-vitro or in-vivo translation system comprising eIF4A and an mRNA having one or more eIF4A-dependent translation-controlling motifs, wherein the modulation of translation in the presence of the agent indicates the agent as capable of modulating cap-dependent mRNA translation.

2. The method of claim 1 wherein modulating is decreasing, suppressing or inhibiting cap-dependent mRNA translation.

3. The method of claim 1 wherein the agent stabilizes the binding of eIF4A to the eIF4A-dependent translation-controlling motif of the mRNA.

4. The method of claim 1 wherein the eIF4A-mRNA complex stabilizing motif of the mRNA is located in the 5′ UTR.

5. The method of claim 1 wherein the eIF4A-dependent translation-controlling motif comprises a G-quadruplex structure.

6. The method of claim 5 wherein the G-quadruplex structure comprises a (GGC/A)₄ motif.

7. The method of claim 6 wherein the (GGC/A)₄ motif comprises GGCGGCGGCGGC (SEQ ID NO:1).

8. The method of claim 1 wherein the eIF4A-dependent translation-controlling motif comprises a sequence selected from SEQ ID NO:4, SEQ ID NO:5, SEQ ID NO:6, SEQ ID NO:7, SEQ ID NO:8, SEQ ID NO:9 or SEQ ID NO:10.

9. The method of claim 1 wherein the eIF4A-dependent translation-controlling motif comprises a sequence selected from SEQ ID NO:10 to SEQ ID NO:62.

10. The method of claim 1 wherein the eIF4A-dependent translation-controlling motif is at least one sequence selected from SEQ ID NO:1 or from SEQ ID NO:4 to SEQ ID NO:62.

15. The method of claim 1 wherein the mRNA encodes a transcription factor.

16. The method of claim 1 wherein the mRNA encodes an oncogene.

17. The method of claim 1 wherein the mRNA encodes NOTCH1, BCL11B, MYC, CDK6, RUNX1, BCL2 or MDM2.

18. The method of claim 1 wherein the agent suppresses the growth of cancer cells in vitro or in vivo.

19. The method of claim 1 wherein the agent interferes with eIF4A activity.

20. The method of claim 1 wherein the agent increases eIF4A activity.

21. The method of claim 1 wherein the agent inhibits eIF4A helicase activity.

22. The method of claim 1 wherein the agent increases eIF4A helicase activity.

23. The method of claim 1 wherein the agent promotes the stabilizing the binding of eIF4A with an eIF4A-dependent translation-controlling motif.

24. The method of claim 1 wherein the agent does not trigger feedback activation of Akt.

25. The method of any one of claims 1 to 24 wherein the modulation of translation is measured by a fluorescence reporter assay.

26. The method of claim 25 wherein the assay comprises renilla luciferase expression.

27. The method of claim 1 wherein the mRNA is from a gene selected from Table 3A.

28. The method of claim 1 wherein the mRNA is from a gene selected from Table 3B.

29. The method of claim 1 wherein the mRNA is from a gene selected from Table 3C.

30. A method for identifying an agent that modulates eIF4A activity, the method comprising comparing translation efficiency in the presence and absence of the agent in an in-vitro or in-vivo translation system comprising eIF4A and an mRNA having one or more eIF4A-dependent translation-controlling motifs, wherein the increase or decrease in translation efficiency in the presence of the agent indicates the agent as capable of increasing or decreasing eIF4A activity.

31. A method for identifying an agent that inhibits eIF4A activity, the method comprising comparing translation efficiency in the presence and absence of the agent in an in-vitro or in-vivo translation system comprising eIF4A and an mRNA having one or more eIF4A-dependent translation-controlling motifs, wherein a decrease in translation efficiency in the presence of the agent indicates the agent as capable of inhibiting eIF4A activity.

32. A method for determining whether an mRNA sequence comprises at least one eIF4A-dependent translation-controlling motif, the method comprising comparing translation efficiency in the presence and absence of an agent that inhibits eIF4A activity in an in-vivo translation system comprising eIF4A and an mRNA having one or more eIF4A-dependent translation-controlling motifs, wherein a decrease in translation efficiency in the presence of the agent indicates the mRNA sequence possesses at least one eIF4A-dependent translation-controlling motif.

33. A method for determining whether a cancer or tumor is susceptible to an agent that inhibits eIF4A activity, the method comprising identifying the presence of at least one eIF4A-dependent translation-controlling motif in mRNA from the cancer or tumor, wherein the presence of the at least one eIF4A-dependent translation-controlling motif indicates susceptibility of the cancer or tumor to the agent.

34. A method for determining whether a patient having cancer or a tumor will respond to treatment with an eIF4A inhibitor comprising the steps of 1) obtaining a sample of the cancer or tumor from the patient; and 2) identifying the presence of at least one eIF4A-dependent translation-controlling motif in mRNA from the cancer or tumor, wherein the presence of the at least one eIF4A-dependent translation-controlling motif indicates that the patient will respond to the treatment.

35. The method of claim 33 or 34 wherein identifying the presence of at least one eIF4A-dependent translation-controlling motif in mRNA from the cancer or tumor is performed by comparing translation efficiency in the presence and absence of an eIF4A inhibitor agent in an in-vitro or in-vivo translation system comprising eIF4A and mRNA from the cancer or tumor, wherein a decrease in translation efficiency in the presence of the agent indicates the presence of an eIF4A-dependent translation-controlling motif in mRNA from the cancer or tumor.

36. The method of claim 33 or 34 wherein identifying the presence of at least one eIF4A-dependent translation-controlling motif in mRNA from the cancer or tumor is performed by identifying a G-quadruplex motif in at least one oncogene in the cancer or tumor.

37. The method of claim 36 wherein the motif is selected from among SEQ ID NO:1 and SEQ ID NO:4-62.

38. A method for determining whether a patient having cancer or a tumor will respond to treatment with an eIF4A inhibitor comprising the steps of 1) obtaining a sample of the cancer or tumor from the patient; and 2) identifying the presence of at least one oncogene in the cancer or tumor described in Table 3A, 3B or 3C herein, wherein the presence of said at least one oncogene indicates that the patient will respond to the treatment.

39. The method of any one of claims 33-38 where the presence of MYC does not indicate susceptibility or response to treatment.

40. The method of any one of claims 33-39 wherein two or more methods are used to determine susceptibility or response to treatment.

41. A method for preventing, treating or intervening in the recurrence of a cancer in a subject comprising administering to the subject an agent that blocks eIF4a helicase activity, thereby preventing, treating or intervening in the recurrence of the cancer.

42. The method of claim 41 wherein the agent that blocks eIF4A helicase inhibits the translation of an oncogenic mRNA.

43. The method of claim 42 wherein the oncogenic mRNA comprises an eIF4A-dependent translation-controlling motif.

44. The method of claim 43 wherein the eIF4A-dependent translation-controlling motif is a G-quadruplex motif.

45. The method of claim 43 wherein the eIF4A-dependent translation-controlling motif is selected from among SEQ ID NOs:1-62.

46. The method of claim 42 wherein the oncogenic mRNA comprises a G-quadruplex motif.

47. The method of claim 42 wherein the oncogenic mRNA is from an oncogene.

48. The method of claim 47 wherein the oncogene is selected from among Tables 3A, 3B and 3C.

49. The method of claim 47 wherein the oncogene is NOTCH1, BCL11B, MYC, CDK6, RUNX1, BCL2 or MDM2.

50. The method of claim 41 wherein the cancer is T-cell acute lymphoblastic leukemia, small cell lung cancer, renal cell carcinoma, squamous cell carcinoma of the head and neck, neuroblastoma, pancreatic cancer, transformed follicular lymphoma, mantel cell lymphoma, breast cancer, ovarian cancer, hepatocellular carcinoma, non-small cell lung cancer, gastric cancer, Ewing sarcoma or lung adenocarcinoma.

51. A method for preventing, treating or intervening in the recurrence of a cancer in a subject having an eIF4A dependent cancer, comprising administering to the subject an agent that blocks eIF4a helicase activity, thereby preventing, treating or intervening in the recurrence of the cancer.

52. The method of claim 51 wherein the agent that blocks eIF4A helicase inhibits the translation of an oncogenic mRNA.

53. The method of claim 53 wherein the oncogenic mRNA comprises an eIF4A-dependent translation-controlling motif.

54. The method of claim 53 wherein the eIF4A-dependent translation-controlling motif is a G-quadruplex motif.

55. The method of claim 53 wherein the eIF4A-dependent translation-controlling motif is selected from among SEQ ID NOs:1-62.

56. The method of claim 52 wherein the oncogenic mRNA comprises a G-quadruplex motif.

57. The method of claim 52 wherein the oncogenic mRNA is from an oncogene.

58. The method of claim 57 wherein the oncogene is selected from among Tables 3A, 3B and 3C.

59. The method of claim 57 wherein the oncogene is NOTCH1, BCL11B, MYC, CDK6, RUNX1, BCL2 or MDM2.

60. The method of claim 51 wherein the cancer is T-cell acute lymphoblastic leukemia, small cell lung cancer, renal cell carcinoma, squamous cell carcinoma of the head and neck, neuroblastoma, pancreatic cancer, transformed follicular lymphoma, mantel cell lymphoma, breast cancer, ovarian cancer, hepatocellular carcinoma, non-small cell lung cancer, gastric cancer, Ewing sarcoma or lung adenocarcinoma.

61. A method for inhibiting in a subject the translation of an oncogene that comprises an eIF4A-dependent translation-controlling motif, the method comprising administering to the subject an agent that blocks eIF4a helicase, thereby inhibiting translation of the oncogene.

62. The method of claim 61 wherein translation of the oncogene causes cancer in the subject.

63. The method of claim 61 wherein the eIF4A-dependent translation-controlling motif is a G-quadruplex motif.

64. The method of claim 5′ wherein the eIF4A-dependent translation-controlling motif is selected from among SEQ ID NOs:1-62.

65. The method of claim 61 wherein the mRNA of the oncogene comprises a G-quadruplex motif.

66. The method of claim 61 wherein the oncogene is selected from among Tables 3A, 3B and 3C.

67. The method of claim 61 wherein the oncogene is NOTCH1, BCL11B, MYC, CDK6, RUNX1, BCL2 or MDM2.

68. The method of claim 62 wherein the cancer is T-cell acute lymphoblastic leukemia, small cell lung cancer, renal cell carcinoma, squamous cell carcinoma of the head and neck, neuroblastoma, pancreatic cancer, transformed follicular lymphoma, mantel cell lymphoma, breast cancer, ovarian cancer, hepatocellular carcinoma, non-small cell lung cancer, gastric cancer, Ewing sarcoma or lung adenocarcinoma.

69. A method for inhibiting in a subject eIF4A dependent mRNA translation, the method comprising administering to the subject an agent that blocks eIF4a helicase, thereby inhibiting mRNA translation.

70. The method of claim 69 wherein the mRNA translation causes cancer in the subject.

71. The method of claim 69 wherein the mRNA comprises an eIF4A-dependent translation-controlling motif.

72. The method of claim 71 wherein the eIF4A-dependent translation-controlling motif is selected from among SEQ ID NOs:1-62.

73. The method of claim 71 wherein the eIF4A-dependent translation-controlling motif is a G-quadruplex motif.

74. The method of claim 69 wherein the mRNA encodes an oncogenic protein.

75. The method of claim 74 wherein the oncogenic protein is encoded by an oncogene selected from among Tables 3A, 3B and 3C.

76. The method of claim 74 wherein the oncogene is NOTCH1, BCL11B, MYC, CDK6, RUNX1, BCL2 or MDM2.

77. The method of claim 70 wherein the cancer is T-cell acute lymphoblastic leukemia, small cell lung cancer, renal cell carcinoma, squamous cell carcinoma of the head and neck, neuroblastoma, pancreatic cancer, transformed follicular lymphoma, mantel cell lymphoma, breast cancer, ovarian cancer, hepatocellular carcinoma, non-small cell lung cancer, gastric cancer, Ewing sarcoma or lung adenocarcinoma.

78. A method for preventing in a subject the translation of an mRNA comprising an eIF4A-dependent translation-controlling motif, the method comprising administering to the subject an agent that blocks eIF4a helicase activity, thereby inhibiting translation of the mRNA.

79. The method of claim 78 wherein the eIF4A-dependent translation-controlling motif is a G-quadruplex motif.

80. The method of claim 78 wherein the eIF4A-dependent translation-controlling motif is selected from among SEQ ID NOs:1-62.

81. The method of claim 78 wherein the mRNA is from an oncogene selected from among Tables 3A, 3B and 3C.

82. The method of claim 81 wherein the oncogene is NOTCH1, BCL11B, MYC, CDK6, RUNX1, BCL2 or MDM2.

83. The method of claim 78 wherein the translation of the mRNA causes cancer.

84. The method of claim 83 wherein the cancer is T-cell acute lymphoblastic leukemia, small cell lung cancer, renal cell carcinoma, squamous cell carcinoma of the head and neck, neuroblastoma, pancreatic cancer, transformed follicular lymphoma, mantel cell lymphoma, breast cancer, ovarian cancer, hepatocellular carcinoma, non-small cell lung cancer, gastric cancer, Ewing sarcoma or lung adenocarcinoma.

85. The method of any one of claims 41-84 wherein the agent blocks the activity of eIF4A helicase.

86. The method of any one of claims 41-84 wherein the agent blocks the translation of an mRNA comprising an eIF4A-dependent translation-controlling motif.

87. The method of claim 86 wherein the eIF4A-dependent translation-controlling motif is a G-quadruplex motif.

88. The method of any one of claims 41-87 wherein the eIF4A-dependent translation-controlling motif is selected from among SEQ ID NOs:1-62.

89. The method of any one of claims 41-88 wherein the agent is a rocaglamide.

90. The method of claim 89 wherein the rocaglamide is silvestrol, CR-31-B, or an analogue or derivative thereof.

91. The method of any one of claims 34-88 wherein the agent is hippuristanol, pateamine A, or an analogue or derivative thereof.

92. A method for measuring eIF4A helicase activity in vitro comprising contacting eIF4A helicase and ATP with a labeled oligonucleotide, said labeled oligonucleotide comprising a G-quadruplex sequence and a fluorophore at a 5′ or 3′ end and a fluorescence quencher at an other end, wherein interaction between the eIF4A helicase and the labeled oligonucleotide results in an increase in fluorescence of the labeled oligonucleotide correlated with eIF4A helicase activity.

93. The method of claim 92 wherein the G-quadruplex sequence is selected from among SEQ ID NO:1-64.

94. The method of claim 92 wherein the labeled oligonucleotide comprises SEQ ID NO:65.

95. The method of claim 92 used for 1) measuring the effect of RNA helicases on G-quadruplex unwinding; 2) investigating the effect of cofactors/inhibitors required for eIF4A activity; 3) a screening method to identify other proteins that can unwind G-quadruplexes; and 4) identifying and establishing the effect of small molecules that stabilize the G-quadruplex structure.

96. The method of claim 95 wherein the RNA helicase is eIF4A1, eIF4A2, DHX9 or DHX36.

97. A labeled oligonucleotide comprising a G-quadruplex sequence selected from among SEQ ID NO:1-64 and a fluorophore at a 5′ or 3′ end and a fluorescence quencher at an other end.

98. The labeled oligonucleotide of claim 97 comprising the sequence SEQ ID NO:65.
